(19) United States
(12) Patent Application Publication
(10) Pub. No.: US 2012/0090052 A1
(43) Pub. Date: Apr. 12, 2012

(54) Plants Having Enhanced Yield-Related Traits And A Method For Making The Same

(76) Inventors: Ana Isabel Sanz Molinero, Gentbrugge (BE); Valerie Frankard, Waterloo (BE); Yves Hatzfeld, Lille (FR)

(21) Appl. No.: 13/266,989

(22) PCT Filed: Apr. 27, 2010
(86) PCT No.: PCT/EP2010/055579

§ 371 Date: Nov. 21, 2011

Related U.S. Application Data
(60) Provisional application No. 61/180,942, filed on May 26, 2009.
(60) Provisional application No. 61/180,946, filed on May 26, 2009.
(60) Provisional application No. 61/180,945, filed on May 26, 2009.
(60) Provisional application No. 61/181,688, filed on May 28, 2009.
(60) Provisional application No. 61/181,692, filed on May 28, 2009.

(30) Foreign Application Priority Data
Apr. 29, 2009 (EP) 09100257.6
Apr. 29, 2009 (EP) 09100259.2
Apr. 29, 2009 (EP) 09100260.0
Apr. 29, 2009 (EP) 09100262.6
Apr. 29, 2009 (EP) 09100263.4

(51) Int. Cl. C12N 15/82; A01H 5/10; C12N 15/63; C12N 9/14; C12N 15/29; C07K 14/415; C12N 15/55; A01H 5/00; C12N 5/10

(52) U.S. Cl. 800/290; 800/278; 800/298; 435/320.1; 435/419; 800/320.1; 800/320.2; 800/320.3; 800/320; 536/23.6; 530/370; 536/23.2; 435/195

(57) ABSTRACT

The present invention relates generally to the field of molecular biology and concerns a method for enhancing various yield-related traits and/or plant growth characteristics in plants by modulating expression in a plant of a nucleic acid encoding a C3H-like polypeptide, or a SPATULA-like (SPT) polypeptide, or an IDI2 (Iron Deficiency Induced 2) polypeptide, or an eIF4F-like protein complex subunit, or GR-RBP (Glycine Rich-RNA Binding Protein) polypeptide. The present invention also concerns plants having modulated expression and/or activity of a nucleic acid encoding a C3H-like polypeptide, or a SPATULA-like (SPT) polypeptide, or an IDI2 (Iron Deficiency Induced 2) polypeptide, or an eIF4F-like protein complex subunit, or GR-RBP (Glycine Rich-RNA Binding Protein) polypeptide, which plants have enhanced yield-related traits and/or plant growth characteristics relative to corresponding wild type plants or other control plants. The invention also provides constructs useful in the methods of the invention.

The present invention relates generally to the field of molecular biology and concerns a method for enhancing various yield-related traits in plants by modulating expression in a plant of a nucleic acid encoding a C3H-like polypeptide. The present invention also concerns plants having modulated expression of a nucleic acid encoding a C3H-like polypeptide, which plants have enhanced yield-related traits relative to corresponding wild type plants or other control plants. The invention also provides constructs useful in the methods of the invention.

The present invention relates generally to the field of molecular biology and concerns a method for enhancing various yield-related traits by modulating expression in a plant of a nucleic acid encoding a SPATULA-like (SPT) polypeptide. The present invention also concerns plants having modulated expression of a nucleic acid encoding an SPT-like polypeptide, which plants have enhanced yield-related traits relative to corresponding wild type plants or other control plants. The invention also provides constructs useful in the methods of the invention.

The present invention relates generally to the field of molecular biology and concerns a method for improving various plant growth characteristics by modulating expression in a plant of a nucleic acid encoding an IDI2 (Iron Deficiency Induced 2) polypeptide. The present invention also concerns plants having modulated expression of a nucleic acid encoding an IDI2 polypeptide, which plants have improved growth characteristics relative to corresponding wild type plants or other control plants. The invention also provides constructs useful in the methods of the invention.

The present invention relates generally to the field of molecular biology and concerns a method for improving various plant growth characteristics by modulating the activity in a plant of an eIF4F-like protein complex. The present invention also concerns plants having modulated activity of eIF4F-like protein complex, which plants have enhanced growth characteristics relative to corresponding wild type plants or other control plants. The invention also provides constructs useful in the methods of the invention.

The present invention relates generally to the field of molecular biology and concerns a method for improving various plant growth characteristics by modulating expression in a plant of a nucleic acid encoding a GR-RBP (Glycine Rich-RNA Binding Protein) polypeptide. The present invention also concerns plants having modulated expression of a nucleic acid encoding a GR-RBP polypeptide, which plants have improved growth characteristics relative to corresponding wild type plants or other control plants. The invention also provides constructs useful in the methods of the invention.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the above-mentioned factors may therefore contribute to increasing crop yield.

Seed yield is a particularly important trait, since the seeds of many plants are important for human and animal nutrition. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. For example, poor early vigour has been a limitation to the introduction of maize (Zea mays L.) hybrids based on Corn Belt germplasm in the European Atlantic.

A further important trait is that of improved abiotic stress tolerance. Abiotic stress is a primary cause of crop loss worldwide, reducing average yields for most major crop plants by more than 50% (Wang et al., Planta (2003) 218: 1-14). Abiotic stresses may be caused by drought, salinity, extremes of temperature, chemical toxicity, excess or deficiency of nutrients (macroelements and/or microelements), radiation and oxidative stress. The ability to improve plant tolerance to abiotic stress would be of great economic advantage to farmers worldwide and would allow for the cultivation of crops during adverse conditions and in territories where cultivation of crops may not otherwise be possible.

Crop yield may therefore be increased by optimising one of the above-mentioned factors.

Depending on the end use, the modification of certain yield traits may be favoured over others. For example for applications such as forage or wood production, or bio-fuel resource, an increase in the vegetative parts of a plant may be desirable, and for applications such as flour, starch or oil production, an increase in seed parameters may be particularly desirable. Even amongst the seed parameters, some may be favoured over others, depending on the application. Various mechanisms may contribute to increasing seed yield, whether that is in the form of increased seed size or increased seed number.

One approach to increasing yield (seed yield and/or biomass) in plants may be through modification of the inherent growth mechanisms of a plant, such as the cell cycle or various signalling pathways involved in plant growth or in defense mechanisms.

It has been now found that various yield-related traits may be enhanced in plants by modulating expression in a plant of a nucleic acid encoding a C3H-like polypeptide in a plant.

It has also now been found that various yield-related traits may be enhanced in plants by modulating expression in a plant of a nucleic acid encoding an SPT-like polypeptide.

It has also now been found that various growth characteristics may be improved in plants by modulating expression in a plant of a nucleic acid encoding an IDI2 (Iron Deficiency Induced 2) in a plant.

It has also now been found that various growth characteristics may be improved in plants by modulating the activity in a plant of at least a nucleic acid encoding an eIF4F-like protein complex subunit polypeptide and/or the level of the said protein complex.

It has also now been found that various growth characteristics may be improved in plants by modulating expression in a plant of a nucleic acid encoding a GR-RBP (Glycine Rich-RNA Binding Protein) in a plant.

### BACKGROUND

### 1. C3H-Like Polypeptides

One of the most abundant domains detected in the Arabidopsis proteome is the RING-finger domain. The RING domain was originally named after the acronym for the protein in which it was first found, encoded by the Really Interesting New Gene. The RING-finger domain is related to the zinc-finger domain; however zinc fingers consist of two pairs of zinc ligands co-ordinately binding one zinc ion, whereas RING fingers consist of four pairs of ligands binding two ions. The RING domain can basically be considered a protein-interaction domain.

The RING finger domain comprises different types of subdomains, namely the C3HC4-type and C3H2C3-type, also referred to as RING-HC and RING H2, respectively.

### 2. SPATULA-Like (SPT) Polypeptides

The basic/helix-loop-helix (bHLH) transcription factors and their homologues form a large family in plant and animal genomes. Li et al., 2006 (Plant Physiol August; 141(4): 1167-84) identified 167 bHLH genes in the rice genome and reported that their phylogenetic analysis indicates that they form well-supported clades. The phylogeny of bHLH proteins from Arabidopsis thaliana have also been studied, see Toledo-Ortiz et al., 2003 (Plant Cell, August; 15(8): 1749-70); Buck and Atchley, 2003 (J Mol. Evol. June; 56(6):742-50).

SPATULA is a bHLH transcription factor. Groszmann et al., 2008 (Plant Journal, July 55(1):40-52) described the SPATULA (SPT) gene as being involved in generating the septum, style and stigma. They also identified twelve orthologues of AtSPT in eudicots, rice and a gymnosperm. They identified two conserved structural domains in addition to the BHLH domain: an amphipathic helix and an acidic domain. SPATULA has also been reported to be a light-stable repressor of seed germination, see Penfield et al. 2005 (Curr Biol. November 22; 15(22): 1988-2006).

### 3. IDI2 (Iron Deficiency Induced 2) Polypeptides

The Fe-deficiency inducible cDNA IDI2 was for the first time isolated from iron-deficient barley roots. The encoded protein had a low similarity with alpha subunits of eukaryotic initiation factor 2B (Yamaguchi et al., J. Exp. Bot. 51, 2001-2007, 2000), which is a guanine nucleotide exchange factor (GEF) that plays a key role in the regulation of protein synthesis. Translation of mRNA begins with the binding of initiator Met-tRNAᵢ to the 40 S ribosomal subunit and is mediated by eIF-2 as part of the eIF-2•GTP•Met-tRNAᵢ ternary complex. During the initiation process, the GTP bound to eIF-2 is hydrolyzed, and a binary complex consisting of eIF-2 and GDP is released from the 80 S initiation complex. Since eIF-2 has a 100-400-fold higher affinity for GDP than for GTP, the guanine nucleotide exchange factor (GEF) known as eIF-2B is required to regenerate the GTP-bound form of eIF-2, which can then participate in another cycle of translation initiation.

The eukaryotic translation initiation factor eIF-2B is a complex made up of five different subunits, alpha, beta, gamma, delta and epsilon, and catalyzes the exchange of eIF-2-bound GDP for GTP. This family includes initiation factor 2B alpha, beta and delta subunits from eukaryotes, related proteins from archaebacteria and IF-2 from prokaryotes, and also contains a subfamily of proteins in eukaryotes, archaeae, or eubacteria. The IDI2 protein is part of a family of eIF2Balpha-like proteins, which family differs from the eIF2Balpha/beta/delta family. Members of this family have also been characterised as 5-methylthioribose-1-phosphate isomerases, an enzyme of the methionine salvage pathway.

Transcription of IDI2 is induced upon iron or zinc deficiency, but not by copper or manganese deficiency (Yamaguchi et al., 2000). Expression of IDI2 did not differ significantly between boron-tolerant and boron-intolerant plants (Patterson et al., Plant Physiol. 144, 1612-1631, 2007). It was postulated that IDI2 functions in regulating the synthesis rate of proteins required for adaptation to Fe-deficiency (Yamaguchi et al., 2000), in particular in initiation of translation (Negishi et al., Plant J., 30, 83-94, 2002).

4. eIF4F-Like Protein Complex Subunits

Protein synthesis is controlled by different mechanisms in prokaryotes and eukaryotes. In eukaryotes, such mechanisms involve several multisubunit complexes including eukaryotic translation initiation factor (eIFs). Usually initiator tRNA, 40S and 60S ribosomal subunits are assembled by eIFs into an 80S ribosome at the initiation codon of mRNA. Therefore, the initiation translation mechanism is considered as to be rate-limiting for protein translation.

The two major complexes involved in translation initiation are the eIF4F, which binds to the 7mGppp cap of the mRNA and recruits the 43S complex, and the 43S complex, which bring the 40 ribosome subunit to the 5′UTR and allow 5′scanning to the correct initiation AUG codon. Both the eIF4F (complex of eIF4E+eIF4G+eIF4A) and eIF(iso)4F (complex of eIF(iso)4E+eIF(iso)4G+eIF4A) have similar activities in supporting the initiation of translation in vitro (Lax et al., Mechanisms of Development, Volume 122, Issues 7-8, July 2005, Pp. 865-876; Browning et al., J. Biol. Chem. 267 (1992), pp. 10096-10100).

The eIF4E polypeptide binds with eIF4G and eIF4A to form the eIF4F protein complex, which serves as a scaffold for the assembly of other initiation factors such as eIF4B, eIF3, and poly(A)-binding proteins.

Other factors involved in translation are eIF5, which allows the dissociation of all the 43S complex when the initiation AUG is met. Then eIF5B promotes association of the 60S and 40S subunits of the ribosome and translation actually starts. PolyA binding proteins bind to eIF4F, bringing the START and END of the CDS close to each other, for efficient recycling of the ribosome 40S subunit.

In plants, eIF4 isoF is composed of eIF4 isoE, isoG and eIF4A subunits. The “iso” subunits are functional equivalents of the “normal” subunits, usually much shorter and with little sequence homology with their normal counterpart.

In eukaryotes, eIF4F seems to play different roles; in animals, eIF4E is an oncongene which mechanism acts by suppression of apoptose. Overexpression of rice eIF4 isoG could increase susceptibility to yellow mottle virus if the allele is a sensititive allele. eIF5A is commonly associated with programmed cell death and its overexpression in plants leads to conflicting results: severe growth defects (Hopkins et al., Plant Physiology, September 2008, Vol. 148, pp. 479-489) or increased rosette size (Liu et al., Journal of Experimental Botany, Vol. 59, No. 4, pp. 939-950, 2008).

Daniel R. Gallie (Plant Molecular Biology 50: 949-970, 2002.) discloses protein-protein interactions required during translation but only focusing on those involved in the translation of nuclear genes as the translation machinery of the chloroplast and mitochondrion is prokaryotic in origin. Therefore, the role of several eIFs in the translation mechanisms is presented. Both plant eIF4G, the larger subunit of eIF4F, and eIF4A are mentioned in this document and their role during initiation in plants. However, it is also evident that little is known regarding their role on the initiation process and no relation can be established between their contributions to this process and enhanced yield-related traits.

The document Albar et al. (Mutations in the eIF(iso)4G translation initiation factor confer high resistance of rice to Rice yellow mottle virus—The Plant Journal (2006) 47, 417-426) discloses the role of isoform of IF4G in the occurring interactions in rice and virus resistance, namely regarding Rice yellow mottle virus (RYMV). Again, no relation was established between the subject matter of the referred document and enhanced yield-related traits, unless plants are severely infected by virus.

Other documents refer to plant eIF4F, such as Laura K. Mayberry et al. (Methods in Enzymology, Volume 430, Chapter 15—pp. 397-408—Elsevier 2007) referring to the expression and purification of recombinant wheat eIFs but again nothing is disclosed on the effects or application to methodologies for enhancing yield-related traits.

The method of the present invention refers to a method for obtaining plants having enhanced yield-related traits and plants thereof.

### 5. GR-RBP (Glycine Rich-RNA Binding Protein) Polypeptide

The genome of Arabidopsis thaliana encodes over 200 different RNA Binding Proteins (RBPs). These RBPs play a role in post-transcriptional gene regulation in developmental processes (reviewed by Lorković, Trends in Plant Science, 2009), as they bind to splice sites and to binding sites for splicing factors on nascent pre-mRNAs, thus competing with splicing factors to negatively control splicing. Most of the RBPs are plant specific and may be involved in plant specific functions. The group of RBPs comprises a superfamily of glycine-rich RNA-binding proteins (GR-RBPs; Wang & Brendel, Genome Biol. 5, R102, 2004). GR-RBPs typically comprise RNA recognition motifs (RRMs) at the N-terminus and a C-terminal glycine-rich domain (GD).

Although GR-RBPs are reportedly involved in diverse developmental processes, including in adaptation of plants to various environmental conditions, overexpression of GR-RBPs also resulted in adverse effects on plant growth: GR-RBP4 expression in Arabidopsis for example caused retarded germination and did not increase cold- or freezing tolerance (Kwak et al. J. Exp. Bot. 56, 3007-3016, 2005). For other RBPs, an effect on cold stress or high temperature stress was only shown in microorganisms (Kwak et al. Nucl. Ac. Res. 35, 506-516, 2007; Sahi et al., Plant Science 173, 144-155, 2007).

### SUMMARY

### 1. C3H-Like Polypeptides

Surprisingly, it has now been found that modulating expression of a nucleic acid encoding a polypeptide comprising a RING domain of the C3H2C3-type gives plants having enhanced yield-related traits relative to control plants.

According to one embodiment, there is provided a method for enhancing various yield-related traits relative to control plants, comprising modulating expression of a nucleic acid encoding a C3H-like polypeptide in a plant.

### 2. SPATULA-Like (SPT) Polypeptides

Surprisingly, it has now been found that modulating expression of a nucleic acid encoding an SPT-like polypeptide gives plants having enhanced yield-related traits relative to control plants.

According to one embodiment, there is provided a method for enhancing yield-related traits relative to control plants, comprising modulating expression in a plant of a nucleic acid encoding an SPT-like polypeptide in a plant.

### 3. IDI2 (Iron Deficiency Induced 2) Polypeptides

Surprisingly, it has now been found that modulating expression of a nucleic acid encoding an IDI2 polypeptide gives plants having enhanced yield-related traits, in particular increased yield and/or early vigour relative to control plants.

According to one embodiment, there is provided a method for improving yield-related traits of a plant relative to control plants, comprising modulating expression of a nucleic acid encoding an IDI2 polypeptide in a plant.

4. eIF4F-Like Protein Complex Subunits

The present invention relates generally to the field of molecular biology and concerns a method for improving various plant growth characteristics by modulating the activity of an eIF4F-like protein complex. The present invention also concerns plants having modulated activity of an eIF4F-like protein complex, which plants have enhanced growth characteristics relative to corresponding wild type plants or other control plants. The invention also provides constructs useful in the methods of the invention.

Surprisingly, it has now been found that modulating the activity of an eIF4F-like protein complex gives plants having enhanced yield-related traits, in particular increased yield relative to control plants.

According to one embodiment, there is provided a method for improving yield related traits of a plant relative to control plants, comprising modulating the activity of an eIF4F-like protein complex in a plant.

### 5. GR-RBP (Glycine Rich-RNA Binding Protein) Polypeptides

Surprisingly, it has now been found that modulating expression of a nucleic acid encoding a GR-RBP polypeptide gives plants having enhanced yield-related traits, in particular increased yield and/or early vigour, relative to control plants.

According to one embodiment, there is provided a method for improving yield-related traits of a plant relative to control plants, comprising modulating expression of a nucleic acid encoding a GR-RBP polypeptide in a plant.

### DEFINITIONS

### Polypeptide(s)/Protein(s)

The terms “polypeptide” and “protein” are used interchangeably herein and refer to amino acids in a polymeric form of any length, linked together by peptide bonds.

### Polynucleotide(s)/Nucleic Acid(s)/Nucleic Acid Sequence(s)/Nucleotide Sequence(s)

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

### Homologue(s)

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide and may range from 1 to 10 amino acids; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds) and Table 1 below).

TABLE 1
Examples of conserved amino acid substitutions

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

### Derivatives

“Derivatives” include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

### Orthologue(s)/Paralogue(s)

Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

### Domain, Motif/Consensus Sequence/Signature

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol. 147(1); 195-7).

### Reciprocal BLAST

Typically, this involves a first BLAST involving BLASTing a query sequence (for example using any of the sequences listed in Table A of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived. The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

### Hybridisation

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tm is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

1) DNA-DNA hybrids (Meinkoth and Wahl, Anal. Biochem., 138: 267-284, 1984):

Tₘ=81.5° C.+16.6×log₁₀[Na⁺]^{a}+0.41×%[G/C^{b}]−500×[L^{c}]⁻¹−0.61×% formamide

2) DNA-RNA or RNA-RNA hybrids:

Tₘ=79.8+18.5 (log₁₀[Na⁺]^{a})+0.58 (% G/C^{b})+11.8 (% G/C^{b})²−820/L^{c}

3) oligo-DNA or oligo-RNA^{d} hybrids:

For <20 nucleotides: Tₘ=2 (Iₙ)

For 20-35 nucleotides: Tₘ=22+1.46 (Iₙ)

^{a} or for other monovalent cation, but only accurate in the 0.01-0.4 M range.
^{b} only accurate for % GC in the 30% to 75% range.
^{c}L=length of duplex in base pairs.
^{d} oligo, oligonucleotide; Iₙ=effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3^{rd} Edition, Cold Spring Harbor Laboratory Press, CSH, New York or to Current Protocols in Molecular Biology, John Wiley & Sons, N.Y. (1989 and yearly updates).

### Splice Variant

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for predicting and isolating such splice variants are well known in the art (see for example Foissac and Schiex (2005) BMC Bioinformatics 6: 25).

### Allelic Variant

Alleles or allelic variants are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

### Endogenous Gene

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

### Gene Shuffling/Directed Evolution

Gene shuffling or directed evolution consists of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547).

### Construct

Additional regulatory elements may include transcriptional as well as translational enhancers. Those skilled in the art will be aware of terminator and enhancer sequences that may be suitable for use in performing the invention. An intron sequence may also be added to the 5′untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

### Regulatory Element/Control Sequence/Promoter

The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter, in particular at a level that is in all instances below that obtained when under the control of a 35S CaMV promoter.

### Operably Linked

The term “operably linked” as used herein refers to a functional linkage between the promoter sequence and the gene of interest, such that the promoter sequence is able to initiate transcription of the gene of interest.

### Constitutive Promoter

A “constitutive promoter” refers to a promoter that is transcriptionally active during most, but not necessarily all, phases of growth and development and under most environmental conditions, in at least one cell, tissue or organ. Table 2a below gives examples of constitutive promoters.

TABLE 2a
Examples of constitutive promoters

| Gene Source | Reference |
|---|---|
| Actin | McElroy et al, Plant Cell, 2: 163-171, 1990 |
| HMGP | WO 2004/070039 |
| CAMV 35S | Odell et al, Nature, 313: 810-812, 1985 |
| CaMV 19S | Nilsson et al., Physiol. Plant. 100: 456-462, 1997 |
| GOS2 | de Pater et al, Plant J Nov; 2(6): 837-44, 1992, |
| | WO 2004/065596 |
| Ubiquitin | Christensen et al, Plant Mol. |
| | Biol. 18: 675-689, 1992 |
| Rice cyclophilin | Buchholz et al, Plant Mol Biol. 25(5): 837-43, 1994 |
| Maize H3 histone | Lepetit et al, Mol. Gen. Genet. 231: 276-285, 1992 |
| Alfalfa H3 histone | Wu et al. Plant Mol. Biol. 11: 641-649, 1988 |
| Actin 2 | An et al, Plant J. 10(1); 107-121, 1996 |
| 34S FMV | Sanger et al., Plant. Mol. Biol., 14, 1990: 433-443 |
| Rubisco small | U.S. Pat. No. 4,962,028 |
| subunit | |
| OCS | Leisner (1988) Proc Natl Acad Sci USA 85(5): 2553 |
| SAD1 | Jain et al., Crop Science, 39 (6), 1999: 1696 |
| SAD2 | Jain et al., Crop Science, 39 (6), 1999: 1696 |
| nos | Shaw et al. (1984) Nucleic Acids |
| | Res. 12(20): 7831-7846 |
| V-ATPase | WO 01/14572 |
| Super promoter | WO 95/14098 |
| G-box proteins | WO 94/12015 |

### Ubiquitous Promoter

A ubiquitous promoter is active in substantially all tissues or cells of an organism.

### Developmentally-Regulated Promoter

A developmentally-regulated promoter is active during certain developmental stages or in parts of the plant that undergo developmental changes.

### Inducible Promoter

An inducible promoter has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens.

### Organ-Specific/Tissue-Specific Promoter

An organ-specific or tissue-specific promoter is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

Examples of root-specific promoters are listed in Table 2b below:

TABLE 2b
Examples of root-specific promoters

| Gene Source | Reference |
|---|---|
| RCc3 | Plant Mol Biol. 1995 Jan; 27(2): 237-48 |
| Arabidopsis PHT1 | Kovama et al., 2005; Mudge et al. |
| | (2002, Plant J. 31: 341) |
| Medicago phosphate | Xiao et al., 2006 |
| transporter | |
| Arabidopsis Pyk10 | Nitz et al. (2001) Plant |
| | Sci 161(2): 337-346 |
| root-expressible genes | Tingey et al., EMBO J. 6: 1, 1987. |
| tobacco auxin-inducible gene | Van der Zaal et al., Plant Mol. |
| | Biol. 16, 983, 1991. |
| β-tubulin | Oppenheimer, et al., Gene 63: 87, 1988. |
| tobacco root-specific genes | Conkling, et al., Plant |
| | Physiol. 93: 1203, 1990. |
| B. napus G1-3b gene | U.S. Pat. No. 5,401,836 |
| SbPRP1 | Suzuki et al., Plant Mol. |
| | Biol. 21: 109-119, 1993. |
| LRX1 | Baumberger et al. 2001, Genes & |
| | Dev. 15: 1128 |
| BTG-26 Brassica napus | US 20050044585 |
| LeAMT1 (tomato) | Lauter et al. (1996, PNAS 3: 8139) |
| The LeNRT1-1 (tomato) | Lauter et al. (1996, PNAS 3: 8139) |
| class I patatin gene (potato) | Liu et al., Plant Mol. |
| | Biol. 153: 386-395, 1991. |
| KDC1 (Daucus carota) | Downey et al. (2000, J. Biol. |
| | Chem. 275: 39420) |
| TobRB7 gene | W Song (1997) PhD Thesis, North |
| | Carolina State University, |
| | Raleigh, NC USA |
| OsRAB5a (rice) | Wang et al. 2002, Plant Sci. 163: 273 |
| ALF5 (Arabidopsis) | Diener et al. (2001, Plant Cell 13: 1625) |
| NRT2; 1Np (N. plumbaginifolia) | Quesada et al. (1997, Plant Mol. |
| | Biol. 34: 265) |

A seed-specific promoter is transcriptionally active predominantly in seed tissue, but not necessarily exclusively in seed tissue (in cases of leaky expression). The seed-specific promoter may be active during seed development and/or during germination. The seed specific promoter may be endosperm/aleurone/embryo specific. Examples of seed-specific promoters (endosperm/aleurone/embryo specific) are shown in Table 2c to Table 2f below. Further examples of seed-specific promoters are given in Qing Qu and Takaiwa (Plant Biotechnol. J. 2, 113-125, 2004), which disclosure is incorporated by reference herein as if fully set forth.

TABLE 2c
Examples of seed-specific promoters

| Gene source | Reference |
|---|---|
| seed-specific genes | Simon et al., Plant Mol. Biol. 5: 191, 1985; |
| | Scofield et al., J. Biol. Chem. 262: 12202, 1987.; |
| | Baszczynski et al., Plant Mol. Biol. 14: 633, 1990. |
| Brazil Nut albumin | Pearson et al., Plant Mol. Biol. 18: 235-245, 1992. |
| legumin | Ellis et al., Plant Mol. Biol. 10: 203-214, 1988. |
| glutelin (rice) | Takaiwa et al., Mol. Gen. Genet. 208: 15-22, 1986; |
| | Takaiwa et al., FEBS Letts. 221: 43-47, 1987. |
| zein | Matzke et al Plant Mol Biol, 14(3): 323-32 1990 |
| napA | Stalberg et al, Planta 199: 515-519, 1996. |
| wheat LMW and HMW glutenin-1 | Mol Gen Genet 216: 81-90, 1989; NAR 17: 461-2, 1989 |
| wheat SPA | Albani et al, Plant Cell, 9: 171-184, 1997 |
| wheat α, β, γ-gliadins | EMBO J. 3: 1409-15, 1984 |
| barley ltr1 promoter | Diaz et al. (1995) Mol Gen Genet 248(5): 592-8 |
| barley B1, C, D, hordein | Theor Appl Gen 98: 1253-62, 1999; Plant J 4: 343-55, |
| | 1993; Mol Gen Genet 250: 750-60, 1996 |
| barley DOF | Mena et al, The Plant Journal, 116(1): 53-62, 1998 |
| blz2 | EP99106056.7 |
| synthetic promoter | Vicente-Carbajosa et al., Plant J. 13: 629-640, 1998. |
| rice prolamin NRP33 | Wu et al, Plant Cell Physiology 39(8) 885-889, 1998 |
| rice a-globulin Glb-1 | Wu et al, Plant Cell Physiology 39(8) 885-889, 1998 |
| rice OSH1 | Sato et al, Proc. Natl. Acad. Sci. USA, 93: 8117-8122, |
| | 1996 |
| rice α-globulin REB/OHP-1 | Nakase et al. Plant Mol. Biol. 33: 513-522, 1997 |
| rice ADP-glucose pyrophosphorylase | Trans Res 6: 157-68, 1997 |
| maize ESR gene family | Plant J 12: 235-46, 1997 |
| sorghum α-kafirin | DeRose et al., Plant Mol. Biol 32: 1029-35, 1996 |
| KNOX | Postma-Haarsma et al, Plant Mol. Biol. 39: 257-71, 1999 |
| rice oleosin | Wu et al, J. Biochem. 123: 386, 1998 |
| sunflower oleosin | Cummins et al., Plant Mol. Biol. 19: 873-876, 1992 |
| PRO0117, putative rice 40S | WO 2004/070039 |
| ribosomal protein | |
| PRO0136, rice alanine | unpublished |
| aminotransferase | |
| PRO0147, trypsin inhibitor ITR1 | unpublished |
| (barley) | |
| PRO0151, rice WSI18 | WO 2004/070039 |
| PRO0175, rice RAB21 | WO 2004/070039 |
| PRO005 | WO 2004/070039 |
| PRO0095 | WO 2004/070039 |
| α-amylase (Amy32b) | Lanahan et al, Plant Cell 4: 203-211, 1992; Skriver et al, |
| | Proc Natl Acad Sci USA 88: 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

TABLE 2d
examples of endosperm-specific promoters

| Gene source | Reference |
|---|---|
| glutelin (rice) | Takaiwa et al. (1986) Mol Gen Genet 208: 15-22; |
| | Takaiwa et al. (1987) FEBS Letts. 221: 43-47 |
| zein | Matzke et al., (1990) Plant Mol Biol 14(3): 323-32 |
| wheat LMW and | Colot et al. (1989) Mol Gen Genet 216: 81-90, |
| HMW glutenin-1 | Anderson et al. (1989) NAR 17: 461-2 |
| wheat SPA | Albani et al. (1997) Plant Cell 9: 171-184 |
| wheat gliadins | Rafalski et al. (1984) EMBO 3: 1409-15 |
| barley Itr1 promoter | Diaz et al. (1995) Mol Gen Genet 248(5): 592-8 |
| barley B1, C, | Cho et al. (1999) Theor Appl Genet 98: 1253-62; |
| D, hordein | Muller et al. (1993) Plant J 4: 343-55; |
| | Sorenson et al. (1996) Mol Gen Genet 250: 750-60 |
| barley DOF | Mena et al, (1998) Plant J 116(1): 53-62 |
| blz2 | Onate et al. (1999) J Biol Chem 274(14): 9175-82 |
| synthetic | Vicente-Carbajosa et al. (1998) Plant J 13: 629-640 |
| promoter | |
| rice prolamin | Wu et al, (1998) Plant Cell Physiol 39(8) 885-889 |
| NRP33 | |
| rice globulin Glb-1 | Wu et al. (1998) Plant Cell Physiol 39(8) 885-889 |
| rice globulin | Nakase et al. (1997) Plant Molec Biol 33: 513-522 |
| REB/OHP-1 | |
| rice ADP-glucose | Russell et al. (1997) Trans Res 6: 157-68 |
| pyrophosphorylase | |
| maize ESR | Opsahl-Ferstad et al. (1997) Plant J 12: 235-46 |
| gene family | |
| sorghum kafirin | DeRose et al. (1996) Plant Mol Biol 32: 1029-35 |

TABLE 2e
Examples of embryo specific promoters:

| Gene source | Reference |
|---|---|
| rice OSH1 | Sato et al, Proc. Natl. Acad. Sci. USA, 93: 8117-8122, |
| | 1996 |
| KNOX | Postma-Haarsma et al, Plant Mol. Biol. 39: 257-71, 1999 |
| PRO0151 | WO 2004/070039 |
| PRO0175 | WO 2004/070039 |
| PRO005 | WO 2004/070039 |
| PRO0095 | WO 2004/070039 |

TABLE 2f
Examples of aleurone-specific promoters:

| Gene source | Reference |
|---|---|
| α-amylase (Amy32b) | Lanahan et al, Plant Cell 4: 203-211, 1992; |
| | Skriver et al, Proc Natl Acad Sci |
| | USA 88: 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

A green tissue-specific promoter as defined herein is a promoter that is transcriptionally active predominantly in green tissue, substantially to the exclusion of any other parts of a plant, whilst still allowing for any leaky expression in these other plant parts.

Examples of green tissue-specific promoters which may be used to perform the methods of the invention are shown in Table 2g below.

TABLE 2g
Examples of green tissue-specific promoters

| Gene | Expression | Reference |
|---|---|---|
| Maize Orthophosphate dikinase | Leaf specific | Fukavama et al., 2001 |
| Maize Phosphoenolpyruvate | Leaf specific | Kausch et al., 2001 |
| carboxylase | | |
| Rice Phosphoenolpyruvate | Leaf specific | Liu et al., 2003 |
| carboxylase | | |
| Rice small subunit Rubisco | Leaf specific | Nomura et al., 2000 |
| rice beta expansin EXBP9 | Shoot specific | WO 2004/070039 |
| Pigeonpea small subunit Rubisco | Leaf specific | Panguluri et al., 2005 |
| Pea RBCS3A | Leaf specific | |

Another example of a tissue-specific promoter is a meristem-specific promoter, which is transcriptionally active predominantly in meristematic tissue, substantially to the exclusion of any other parts of a plant, whilst still allowing for any leaky expression in these other plant parts. Examples of green meristem-specific promoters which may be used to perform the methods of the invention are shown in Table 2h below.

TABLE 2h
Examples of meristem-specific promoters

| Gene source | Expression pattern | Reference |
|---|---|---|
| rice OSH1 | Shoot apical meristem, | Sato et al. (1996) |
| | from embryo globular stage | Proc. Natl. Acad. |
| | to seedling stage | Sci. USA, |
| | | 93: 8117-8122 |
| Rice metallothionein | Meristem specific | BAD87835.1 |
| WAK1 & WAK 2 | Shoot and root apical | Wagner & Kohorn |
| | meristems, and in | (2001) Plant Cell |
| | expanding leaves and | 13(2): 303-318 |
| | sepals | |

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Selectable Marker (Gene)/Reporter Gene

“Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

### Transgenic/Transgene/Recombinant

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

### Modulation

The term “modulation” means in relation to expression or gene expression, a process in which the expression level is changed by said gene expression in comparison to the control plant, the expression level may be increased or decreased. The original, unmodulated expression may be of any kind of expression of a structural RNA (rRNA, tRNA) or mRNA with subsequent translation. The term “modulating the activity” shall mean any change of the expression of the inventive nucleic acid sequences or encoded proteins, which leads to increased yield and/or increased growth of the plants.

### Expression

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

### Increased Expression/Overexpression

The term “increased expression” or “overexpression” as used herein means any form of expression that is additional to the original wild-type expression level.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell. biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

### Decreased Expression

Reference herein to “decreased expression” or “reduction or substantial elimination” of expression is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants.

For the reduction or substantial elimination of expression an endogenous gene in a plant, a sufficient length of substantially contiguous nucleotides of a nucleic acid sequence is required. In order to perform gene silencing, this may be as little as 20, 19, 18, 17, 16, 15, 14, 13, 12, 11, 10 or fewer nucleotides, alternatively this may be as much as the entire gene (including the 5′ and/or 3′ UTR, either in part or in whole). The stretch of substantially contiguous nucleotides may be derived from the nucleic acid encoding the protein of interest (target gene), or from any nucleic acid capable of encoding an orthologue, paralogue or homologue of the protein of interest. Preferably, the stretch of substantially contiguous nucleotides is capable of forming hydrogen bonds with the target gene (either sense or antisense strand), more preferably, the stretch of substantially contiguous nucleotides has, in increasing order of preference, 50%, 60%, 70%, 80%, 85%, 90%, 95%, 96%, 97%, 98%, 99%, 100% sequence identity to the target gene (either sense or antisense strand). A nucleic acid sequence encoding a (functional) polypeptide is not a requirement for the various methods discussed herein for the reduction or substantial elimination of expression of an endogenous gene.

This reduction or substantial elimination of expression may be achieved using routine tools and techniques. A preferred method for the reduction or substantial elimination of endogenous gene expression is by introducing and expressing in a plant a genetic construct into which the nucleic acid (in this case a stretch of substantially contiguous nucleotides derived from the gene of interest, or from any nucleic acid capable of encoding an orthologue, paralogue or homologue of any one of the protein of interest) is cloned as an inverted repeat (in part or completely), separated by a spacer (non-coding DNA).

In such a preferred method, expression of the endogenous gene is reduced or substantially eliminated through RNA-mediated silencing using an inverted repeat of a nucleic acid or a part thereof (in this case a stretch of substantially contiguous nucleotides derived from the gene of interest, or from any nucleic acid capable of encoding an orthologue, paralogue or homologue of the protein of interest), preferably capable of forming a hairpin structure. The inverted repeat is cloned in an expression vector comprising control sequences. A non-coding DNA nucleic acid sequence (a spacer, for example a matrix attachment region fragment (MAR), an intron, a polylinker, etc.) is located between the two inverted nucleic acids forming the inverted repeat. After transcription of the inverted repeat, a chimeric RNA with a self-complementary structure is formed (partial or complete). This double-stranded RNA structure is referred to as the hairpin RNA (hpRNA). The hpRNA is processed by the plant into siRNAs that are incorporated into an RNA-induced silencing complex (RISC). The RISC further cleaves the mRNA transcripts, thereby substantially reducing the number of mRNA transcripts to be translated into polypeptides. For further general details see for example, Grierson et al, (1998) WO 98/53083; Waterhouse et al. (1999) WO 99/53050).

Performance of the methods of the invention does not rely on introducing and expressing in a plant a genetic construct into which the nucleic acid is cloned as an inverted repeat, but any one or more of several well-known “gene silencing” methods may be used to achieve the same effects.

One such method for the reduction of endogenous gene expression is RNA-mediated silencing of gene expression (downregulation). Silencing in this case is triggered in a plant by a double stranded RNA sequence (dsRNA) that is substantially similar to the target endogenous gene. This dsRNA is further processed by the plant into about 20 to about 26 nucleotides called short interfering RNAs (siRNAs). The siRNAs are incorporated into an RNA-induced silencing complex (RISC) that cleaves the mRNA transcript of the endogenous target gene, thereby substantially reducing the number of mRNA transcripts to be translated into a polypeptide. Preferably, the double stranded RNA sequence corresponds to a target gene.

Another example of an RNA silencing method involves the introduction of nucleic acid sequences or parts thereof (in this case a stretch of substantially contiguous nucleotides derived from the gene of interest, or from any nucleic acid capable of encoding an orthologue, paralogue or homologue of the protein of interest) in a sense orientation into a plant. “Sense orientation” refers to a DNA sequence that is homologous to an mRNA transcript thereof. Introduced into a plant would therefore be at least one copy of the nucleic acid sequence. The additional nucleic acid sequence will reduce expression of the endogenous gene, giving rise to a phenomenon known as co-suppression. The reduction of gene expression will be more pronounced if several additional copies of a nucleic acid sequence are introduced into the plant, as there is a positive correlation between high transcript levels and the triggering of co-suppression.

Another example of an RNA silencing method involves the use of antisense nucleic acid sequences. An “antisense” nucleic acid sequence comprises a nucleotide sequence that is complementary to a “sense” nucleic acid sequence encoding a protein, i.e. complementary to the coding strand of a double-stranded cDNA molecule or complementary to an mRNA transcript sequence. The antisense nucleic acid sequence is preferably complementary to the endogenous gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire nucleic acid sequence (in this case a stretch of substantially contiguous nucleotides derived from the gene of interest, or from any nucleic acid capable of encoding an orthologue, paralogue or homologue of the protein of interest), but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine substituted nucleotides may be used. Examples of modified nucleotides that may be used to generate the antisense nucleic acid sequences are well known in the art. Known nucleotide modifications include methylation, cyclization and ‘caps’ and substitution of one or more of the naturally occurring nucleotides with an analogue such as inosine. Other modifications of nucleotides are well known in the art.

The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention (whether introduced into a plant or generated in situ) hybridize with or bind to mRNA transcripts and/or genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using the vectors described herein.

According to a further aspect, the antisense nucleic acid sequence is an a-anomeric nucleic acid sequence. An a-anomeric nucleic acid sequence forms specific double-stranded hybrids with complementary RNA in which, contrary to the usual b-units, the strands run parallel to each other (Gaultier et al. (1987) Nucl Ac Res 15: 6625-6641). The antisense nucleic acid sequence may also comprise a 2′-o-methylribonucleotide (Inoue et al. (1987) Nucl Ac Res 15, 6131-6148) or a chimeric RNA-DNA analogue (Inoue et al. (1987) FEBS Lett. 215, 327-330).

The reduction or substantial elimination of endogenous gene expression may also be performed using ribozymes. Ribozymes are catalytic RNA molecules with ribonuclease activity that are capable of cleaving a single-stranded nucleic acid sequence, such as an mRNA, to which they have a complementary region. Thus, ribozymes (e.g., hammerhead ribozymes (described in Haselhoff and Gerlach (1988) Nature 334, 585-591) can be used to catalytically cleave mRNA transcripts encoding a polypeptide, thereby substantially reducing the number of mRNA transcripts to be translated into a polypeptide. A ribozyme having specificity for a nucleic acid sequence can be designed (see for example: Cech et al. U.S. Pat. No. 4,987,071; and Cech et al. U.S. Pat. No. 5,116,742). Alternatively, mRNA transcripts corresponding to a nucleic acid sequence can be used to select a catalytic RNA having a specific ribonuclease activity from a pool of RNA molecules (Bartel and Szostak (1993) Science 261, 1411-1418). The use of ribozymes for gene silencing in plants is known in the art (e.g., Atkins et al. (1994) WO 94/00012; Lenne et al. (1995) WO 95/03404; Lutziger et al. (2000) WO 00/00619; Prinsen et al. (1997) WO 97/13865 and Scott et al. (1997) WO 97/38116).

Gene silencing may also be achieved by insertion mutagenesis (for example, T-DNA insertion or transposon insertion) or by strategies as described by, among others, Angell and Baulcombe ((1999) Plant J 20(3): 357-62), (Amplicon VIGS WO 98/36083), or Baulcombe (WO 99/15682).

Gene silencing may also occur if there is a mutation on an endogenous gene and/or a mutation on an isolated gene/nucleic acid subsequently introduced into a plant. The reduction or substantial elimination may be caused by a non-functional polypeptide. For example, the polypeptide may bind to various interacting proteins; one or more mutation(s) and/or truncation(s) may therefore provide for a polypeptide that is still able to bind interacting proteins (such as receptor proteins) but that cannot exhibit its normal function (such as signalling ligand).

A further approach to gene silencing is by targeting nucleic acid sequences complementary to the regulatory region of the gene (e.g., the promoter and/or enhancers) to form triple helical structures that prevent transcription of the gene in target cells. See Helene, C., Anticancer Drug Res. 6, 569-84, 1991; Helene et al., Ann. N.Y. Acad. Sci. 660, 27-36 1992; and Maher, L. J. Bioassays 14, 807-15, 1992.

Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved.

Alternatively, a screening program may be set up to identify in a plant population natural variants of a gene, which variants encode polypeptides with reduced activity. Such natural variants may also be used for example, to perform homologous recombination.

Artificial and/or natural microRNAs (miRNAs) may be used to knock out gene expression and/or mRNA translation. Endogenous miRNAs are single stranded small RNAs of typically 19-24 nucleotides long. They function primarily to regulate gene expression and/or mRNA translation. Most plant microRNAs (miRNAs) have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. mRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes.

Artificial microRNAs (amiRNAs), which are typically 21 nucleotides in length, can be genetically engineered specifically to negatively regulate gene expression of single or multiple genes of interest. Determinants of plant microRNA target selection are well known in the art. Empirical parameters for target recognition have been defined and can be used to aid in the design of specific amiRNAs, (Schwab et al., Dev. Cell 8, 517-527, 2005). Convenient tools for design and generation of amiRNAs and their precursors are also available to the public (Schwab et al., Plant Cell 18, 1121-1133, 2006).

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid to be introduced.

Described above are examples of various methods for the reduction or substantial elimination of expression in a plant of an endogenous gene. A person skilled in the art would readily be able to adapt the aforementioned methods for silencing so as to achieve reduction of expression of an endogenous gene in a whole plant or in parts thereof through the use of an appropriate promoter, for example.

### Transformation

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen. Genet. 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet. 208:274-289; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

### T-DNA Activation Tagging

T-DNA activation tagging (Hayashi et al. Science (1992) 1350-1353), involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

### TILLING

The term “TILLING” is an abbreviation of “Targeted Induced Local Lesions In Genomes” and refers to a mutagenesis technology useful to generate and/or identify nucleic acids encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet. 5(2): 145-50).

### Homologous Recombination

Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offring a et al. (1990) EMBO J. 9(10): 3077-84) but also for crop plants, for example rice (Terada et al. (2002) Nat Biotech 20(10): 1030-4; lida and Terada (2004) Curr Opin Biotech 15(2): 132-8), and approaches exist that are generally applicable regardless of the target organism (Miller et al, Nature Biotechnol. 25, 778-785, 2007).

### Yield Related Traits

Yield related traits comprise one or more of yield, biomass, seed yield, early vigour, greenness index, increased growth rate, improved agronomic traits (such as improved Water Use Efficiency (WUE), Nitrogen Use Efficiency (NUE), etc.).

### Yield

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant.

Taking corn as an example, a yield increase may be manifested as one or more of the following: increase in the number of plants established per square meter, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per square meter, number of panicles per plant, panicle length, number of spikelets per panicle, number of flowers (florets) per panicle, increase in the seed filling rate (which is the number of filled seeds divided by the total number of seeds and multiplied by 100), increase in thousand kernel weight, among others. In rice, submergence tolerance may also result in increased yield.

### Early Vigour

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

### Increased Growth Rate

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as speed of germination, early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per square meter (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

### Stress Resistance

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35%, 30% or 25%, more preferably less than 20% or 15% in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi, nematodes and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 97%, 95%, 92%, 90%, 87%, 85%, 83%, 80%, 77% or 75% of the average production of such plant in a given environment. Average production may be calculated on harvest and/or season basis. Persons skilled in the art are aware of average yield productions of a crop.

Nutrient deficiency may result from a lack of nutrients such as nitrogen, phosphates and other phosphorous-containing compounds, potassium, calcium, magnesium, manganese, iron and boron, amongst others.

The term salt stress is not restricted to common salt (NaCl), but may be any one or more of: NaCl, KCl, LiCl, MgCl₂, CaCl₂, amongst others.

### Increase/Improve/Enhance

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 25%, 30%, 35% or 40% more yield and/or growth in comparison to control plants as defined herein.

### Seed Yield

Increased seed yield may manifest itself as one or more of the following: a) an increase in seed biomass (total seed weight) which may be on an individual seed basis and/or per plant and/or per square meter; b) increased number of flowers per plant; c) increased number of (filled) seeds; d) increased seed filling rate (which is expressed as the ratio between the number of filled seeds divided by the total number of seeds); e) increased harvest index, which is expressed as a ratio of the yield of harvestable parts, such as seeds, divided by the total biomass; and f) increased thousand kernel weight (TKW), which is extrapolated from the number of filled seeds counted and their total weight. An increased TKW may result from an increased seed size and/or seed weight, and may also result from an increase in embryo and/or endosperm size.

An increase in seed yield may also be manifested as an increase in seed size and/or seed volume. Furthermore, an increase in seed yield may also manifest itself as an increase in seed area and/or seed length and/or seed width and/or seed perimeter. Increased yield may also result in modified architecture, or may occur because of modified architecture.

### Greenness Index

The “greenness index” as used herein is calculated from digital images of plants. For each pixel belonging to the plant object on the image, the ratio of the green value versus the red value (in the RGB model for encoding color) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought.

### Marker Assisted Breeding

Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

### Use as Probes in (Gene Mapping)

Use of nucleic acids encoding the protein of interest for genetically and physically mapping the genes requires only a nucleic acid sequence of at least 15 nucleotides in length. These nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the nucleic acids encoding the protein of interest. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the nucleic acid encoding the protein of interest in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

### Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocaffis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

### Control Plant(s)

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation. A “control plant” as used herein refers not only to whole plants, but also to plant parts, including seeds and seed parts.

### DETAILED DESCRIPTION OF THE INVENTION

Surprisingly, it has now been found that modulating expression in a plant of a nucleic acid encoding a C3H-like polypeptide gives plants having enhanced yield-related traits relative to control plants. According to a first embodiment, the present invention provides a method for enhancing yield-related traits in plants relative to control plants, comprising modulating expression in a plant of a nucleic acid encoding a C3H-like polypeptide and optionally selecting for plants having enhanced yield-related traits.

Furthermore, it has now surprisingly been found that modulating expression in a plant of a nucleic acid encoding an SPT-like polypeptide gives plants having enhanced yield-related traits relative to control plants. According to a first embodiment, the present invention provides a method for enhancing yield-related traits in plants relative to control plants, comprising modulating expression in a plant of a nucleic acid encoding an SPT-like polypeptide and optionally selecting for plants having enhanced yield-related traits.

Furthermore, it has now surprisingly been found that modulating expression in a plant of a nucleic acid encoding an IDI2 polypeptide gives plants having enhanced yield-related traits relative to control plants. According to a first embodiment, the present invention provides a method for enhancing yield-related traits in plants relative to control plants, comprising modulating expression in a plant of a nucleic acid encoding an IDI2 polypeptide and optionally selecting for plants having enhanced yield-related traits.

The invention also provides hitherto unknown IDI2-encoding nucleic acids and IDI2 polypeptides.

According to a further embodiment of the present invention, there is therefore provided an isolated nucleic acid molecule selected from:
(i) a nucleic acid represented by any of SEQ ID NO: 139, 157, 164, 169, 171, 186;
(ii) the complement of a nucleic acid represented by any of SEQ ID NO: 139, 157, 164, 169, 171, 186;
(iii) a nucleic acid encoding a GR-RBP polypeptide having, in increasing order of preference, at least 50%, 55%, 60%, 65%, 70%, 75%, 80%, 85%, 90%, 95%, 96%, 97%, 98%, 99% or more sequence identity to the amino acid sequences represented by any of SEQ ID NO: 140, 202, 209, 214, 216, 231, and comprising one or more of the motifs 1 to 6.

According to a further embodiment of the present invention, there is also provided an isolated polypeptide selected from:
(i) an amino acid sequence represented by any of SEQ ID NO: 140, 202, 209, 214, 216, 231;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequences represented by any one of SEQ ID NO: 140, 202, 209, 214, 216, 231, and comprising one or more of the motifs 1 to 6;
(iii) derivatives of any of the amino acid sequences given in (i) or (ii) above.

Furthermore, it has now surprisingly been found that modulating the activity in a plant of an eIF4F-like protein complex gives plants having enhanced yield-related traits relative to control plants. According to a first embodiment, the present invention provides a method for enhancing yield-related traits in plants relative to control plants, comprising modulating the activity in a plant of an eIF4F-like protein complex and optionally selecting for plants having enhanced yield-related traits. An eIF4F-like protein complex is composed of eIF4E, 4A, 4G polypeptide or protein subunits.

The invention also provides hitherto unknown eIF4F protein complex subunits-encoding nucleic acids and said subunits polypeptides.

According to a further embodiment of the present invention, there is therefore provided an isolated nucleic acid molecule selected from:
(i) a nucleic acid represented by SEQ ID NO: 306;
(ii) the complement of a nucleic acid represented by SEQ ID NO: 306;
(iii) a nucleic acid encoding the polypeptide as represented by any one of SEQ ID NO: 307, preferably as a result of the degeneracy of the genetic code, said isolated nucleic acid can be derived from a polypeptide sequence as represented by SEQ ID NO: 307 and further preferably confers enhanced yield-related traits relative to control plants;
(iv) a nucleic acid having, in increasing order of preference at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with any of the nucleic acid sequences of Tables A4 and further preferably conferring enhanced yield-related traits relative to control plants;
(v) a nucleic acid molecule which hybridizes with a nucleic acid molecule of (i) to (iv) under stringent hybridization conditions and preferably confers enhanced yield-related traits relative to control plants;
(vi) a nucleic acid encoding at least an eIF4F subunit polypeptide having, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence represented by SEQ ID NO: 68 and any of the other amino acid sequences in Tables A4 and preferably conferring enhanced yield-related traits relative to control plants.

According to a further embodiment of the present invention, there is also provided an isolated polypeptide selected from:
(i) an amino acid sequence represented by SEQ ID NO: 307;
(ii) an amino acid sequence having, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence represented by SEQ ID NO: 307 and any of the other amino acid sequences in Tables A4 and preferably conferring enhanced yield-related traits relative to control plants.
(iii) derivatives of any of the amino acid sequences given in (i) or (ii) above.

Furthermore, it has now surprisingly been found that modulating expression in a plant of a nucleic acid encoding a GR-RBP polypeptide gives plants having enhanced yield-related traits relative to control plants. According to a first embodiment, the present invention provides a method for enhancing yield-related traits in plants relative to control plants, comprising modulating expression in a plant of a nucleic acid encoding a GR-RBP polypeptide and optionally selecting for plants having enhanced yield-related traits.

The invention also provides hitherto unknown GR-RBP-encoding nucleic acids and GR-RBP polypeptides.

According to a further embodiment of the present invention, there is therefore provided an isolated nucleic acid molecule selected from:
(i) a nucleic acid represented by any of SEQ ID NO: 848, 849, 851, 852, 853, 854, 857, 862, 873, 874, 875, 876, 878, 879, 893, 897, 898, 900, 901, 905, 928, 931, 932, 933, 934, 937;
(ii) the complement of a nucleic acid represented by any of SEQ ID NO: 848, 849, 851, 852, 853, 854, 857, 862, 873, 874, 875, 876, 878, 879, 893, 897, 898, 900, 901, 905, 928, 931, 932, 933, 934, 937;
(iii) a nucleic acid encoding a GR-RBP polypeptide having, in increasing order of preference, at least 50%, 55%, 60%, 65%, 70%, 75%, 80%, 85%, 90%, 95%, 96%, 97%, 98%, 99% or more sequence identity to the amino acid sequences represented by any of SEQ ID NO: 945, 946, 948, 949, 950, 951, 954, 959, 970, 971, 972, 973, 975, 976, 990, 994, 995, 997, 998, 1002, 1025, 1028, 1029, 1030, 1031, 1034, and comprising signature sequence 3 (SEQ ID NO: 830) and signature sequence 4 (SEQ ID NO: 831).

According to a further embodiment of the present invention, there is also provided an isolated polypeptide selected from:
(i) an amino acid sequence represented by any of SEQ ID NO: 945, 946, 948, 949, 950, 951, 954, 959, 970, 971, 972, 973, 975, 976, 990, 994, 995, 997, 998, 1002, 1025, 1028, 1029, 1030, 1031, 1034;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequences represented by any one of SEQ ID NO: 945, 946, 948, 949, 950, 951, 954, 959, 970, 971, 972, 973, 975, 976, 990, 994, 995, 997, 998, 1002, 1025, 1028, 1029, 1030, 1031, 1034, and comprising signature sequence 3 (SEQ ID NO: 830) and signature sequence 4 (SEQ ID NO: 831);
(iii) derivatives of any of the amino acid sequences given in (i) or (ii) above.

A preferred method for modulating (preferably, increasing) expression of a nucleic acid encoding a C3H-like polypeptide, or an SPT polypeptide, or an IDI2 polypeptide, or a GR-RBP polypeptide, is by introducing and expressing in a plant a nucleic acid encoding a C3H-like polypeptide, or an SPT polypeptide, or an IDI2 polypeptide, or a GR-RBP polypeptide.

Concerning C3H-like polypeptides, any reference herein to a “protein useful in the methods of the invention” is taken to mean a C3H-like polypeptide as defined herein. Any reference herein to a “nucleic acid useful in the methods of the invention” is taken to mean a nucleic acid capable of encoding such a C3H-like polypeptide. The nucleic acid to be introduced into a plant (and therefore useful in performing the methods of the invention) is any nucleic acid encoding the type of protein which will now be described, hereafter also named “C3H-like nucleic acid” or “C3H-like gene”.

A “C3H-like polypeptide” as defined herein refers to any polypeptide comprising Domain 4 and any one or more of Domains 1, 2, 3 and 5:

| | |
|---|---|
| Domain 1: | C-X₂-C-X₁₂₋₂₃-C-X₂-C-X₂-G-F |

wherein X is any amino acid and the underlined residues are conserved

| | |
|---|---|
| Domain 2: | Y-X₇₋₁₂-L-X₃-P-X₁₀-G |

wherein X is any amino acid and the underlined residues are conserved

| | |
|---|---|
| Domain 3: | S-K-X₆-P |

wherein X is any amino acid and the underlined residues are conserved

| | |
|---|---|
| Domain 4: | RING-C3H2C3 type |
| Domain 5: | DUF1117 |

Preferably, Domain 1 is: CYSCTRFINLSDHTL----------IVCPHCDNGF, or a domain comprising the underlined conserved residues and having, in increasing order of preference, at least 60%, 65%, 70%, 75%, 80%, 85%, 90%, 95% or more sequence identity to the non-underlined residues in Domain 1, where “-” is a gap or any residue.

Preferably, Domain 2 is: YDDGDG-----SGLRPLPPTVSEFLLGSG, or a domain comprising the underlined conserved residues and having, in increasing order of preference, at least 60%, 65%, 70%, 75%, 80%, 85%, 90%, 95% or more sequence identity to the non-underlined residues in Domain 2, where “-” is a gap or any residue.

Preferably, Domain 3 is: SKAAIESMP, or a domain comprising the underlined conserved residues and having, in increasing order of preference, at least 60%, 65%, 70%, 75%, 80%, 85%, 90%, 95% or more sequence identity to the non-underlined residues in Domain 3.

Preferably, Domain 4 is: CAVCKEEFELHAEARELPCKHLYHSDCILPWLTVRNSCPVCR, or a domain comprising the underlined conserved residues and having, in increasing order of preference, at least 60%, 65%, 70%, 75%, 80%, 85%, 90%, 95% or more sequence identity to the non-underlined residues in Domain 4.

Preferably, Domain 5 is: GLTIWRLPGGGFAVGRFSGGRSA-GESHFPVVYTEMDGGLN, or a domain having, in increasing order of preference, at least 60%, 65%, 70%, 75%, 80%, 85%, 90%, 95% or more sequence identity to Domain 5, where “-” is a gap or any residue.

Typically, the homologue of a C3H-like polypeptide has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 2, and comprises DOMAIN4 and any one or more of DOMAIN 1, 2, 3 and 5. The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered.

Preferably, the polypeptide sequence which when used in the construction of a phylogenetic tree, such as the one depicted in FIG. 2, clusters with the group of C3H-like polypeptides comprising the amino acid sequence represented by SEQ ID NO: 2 rather than with any other group.

Concerning SPT-like polypeptides, any reference hereinafter to a “protein useful in the methods of the invention” is taken to mean an SPT-like polypeptide as defined herein. Any reference hereinafter to a “nucleic acid useful in the methods of the invention” is taken to mean a nucleic acid capable of encoding such an SPT-like polypeptide. The nucleic acid to be introduced into a plant (and therefore useful in performing the methods of the invention) is any nucleic acid encoding the type of protein which will now be described, hereinafter also referred to as an “SPT-like nucleic acid” or an “SPT-like gene”.

An “SPT-like polypeptide” as defined herein refers to any polypeptide comprising each of the following, preferably from N-terminus to C-terminus:

Motif I: an amphipathic helix comprising EEISTFLHQLLH, or a motif having in increasing order of preference at least 60%, 65%, 70%, 75%, 80%, 85%, 90%, 95% or more sequence identity to motif I.

Motif II: an acidic domain comprising DLGDFSCDSEK, or a motif having in increasing order of preference at least 60%, 65%, 70%, 75%, 80%, 85%, 90%, 95% or more sequence identity to Motif II.

Motif III: a bHLH domain comprising: AAEVHNLSEKRRRSRINEKMKALQNLIPNSNKTD KASMLDEAIEYLKQL, or a motif having in increasing order of preference at least 60%, 65%, 70%, 75%, 80%, 85%, 90%, 95% or more sequence identity to Motif III.

The SPT-like polypeptide preferably further comprises one or more serine-rich regions. A serine-rich region is taken to mean, in increasing order of preference, at least 30%, 40%, 50%, 60%, 70%, 80%, 90% or more serine residues in any given stretch of contiguous amino acids. Preferably, the one or more serine-rich regions are located as shown in the alignment of FIG. 4.

Preferably, the bHLH domain further comprises one or more nuclear localisation signals (NLS), preferably in the locations indicated in the alignment of FIG. 4.

The SPT-like polypeptide preferably further comprises a beta strand adjacent the bHLH domain nearest the C-terminal region, which beta strand preferably comprises QLQVQMLTM.

Additionally or alternatively, the SPT-like polypeptide has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 97 and comprises each of motifs I to III as defined above. The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered.

Preferably, the polypeptide sequence, which when used in the construction of a phylogenetic tree, such as the one depicted in FIG. 5, clusters with the group of SPT-like polypeptides comprising the amino acid sequence represented by SEQ ID NO: 97 (indicated by an arrow) rather than with any other group.

Concerning IDI2 polypeptides, any reference hereinafter to a “protein useful in the methods of the invention” is taken to mean an IDI2 polypeptide as defined herein. Any reference hereinafter to a “nucleic acid useful in the methods of the invention” is taken to mean a nucleic acid capable of encoding such an IDI2 polypeptide. The nucleic acid to be introduced into a plant (and therefore useful in performing the methods of the invention) is any nucleic acid encoding the type of protein which will now be described, hereafter also named “IDI2 nucleic acid” or “IDI2 gene”.

A “IDI2 polypeptide” as defined herein refers to any alpha subunit of the eukaryotic translation initiation factor EIF-2B, which alpha subunit comprises an IF-2B domain (Pfam accession PF01008). Preferably, the IDI2 polypeptide also comprises one or more of the following motifs:

Motif 1 (SEQ ID NO: 141):
SL[QR]LLDQRKLPLET[IV]Y[LI][DE][IV][KR]D[SA]ADGWNAI
R[DE]MVVRGAPAIAI
Motif 2 (SEQ ID NO: 142):
HCNTGSLATAGYGTALGVIR[AS]LHS[EG]GVL[EL][RKS]A[YF]
[CA]TETRPFNQ
Motif 3 (SEQ ID NO: 143):
EAAE[TI]ML[VE]DDVA[DS]NKAIGS[HY]G
Motif 4 (SEQ ID NO: 144):
[SA]LRLLDQRKLPLE[MT][DV]YIDVK[DS]SADGWNAIRDMVVRGA
PAIAI
Motif 5 (SEQ ID NO: 145):
CNTGSLATAG[YV]GTALGV[IL]RAL[HR][SE][GT]GVLE[KS]A
[FA][CA]TETRP[FYL]NQG
Motif 6 (SEQ ID NO: 146):
M[KA][SQ]GQV[QD]AV[IV]VGADR[IV]AANGDTANKIGTY

More preferably, the IDI2 polypeptide comprises at least 2, most preferably 3 of the above motifs.

Alternatively, the homologue of an IDI2 protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 140, provided that the homologous protein comprises the conserved motifs as outlined above. The overall sequence identity is determined using a global alignment algorithm, such as the Needleman Wunsch algorithm in the program GAP (GCG Wisconsin Package, Accelrys), preferably with default parameters. Compared to overall sequence identity, the sequence identity will generally be higher when only conserved domains or motifs are considered. Preferably the motifs in an IDI2 polypeptide have, in increasing order of preference, at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the motifs represented by SEQ ID NO: 141 to SEQ ID NO: 146 (Motifs 1 to 6).

Preferably, the polypeptide sequence which when used in the construction of a phylogenetic tree, such as the one depicted in FIG. 9, clusters with the A or B group rather than with any other group, more preferably the polypeptide sequence clusters with the A group of IDI2 polypeptides, which comprises the amino acid sequence represented by SEQ ID NO: 140.

Concerning an eIF4F-like protein complex subunits, the activity of an eIF4F-like protein complex may preferably be modulated by modulating the expression of one or more of the subunits of the eIF4F-like protein complex, namely the eIF4G and/or eIF4A and/or eIF4E and/or by modulating the levels of the eIF4F-like protein complex. One preferred method for modulating activity of an eIF4F-like protein complex is by introducing and expressing in a plant a nucleic acid encoding an eIF4F-like protein complex subunit, such as one or more of eIF4E, eIF4G, and/or eIF4A and/or isoforms thereof.

An “eIF4F-like protein complex” as defined herein refers to any protein complex comprising an eIF4E, eIF4G, and/or eIF4A subunits and/or isoforms thereof. In plants, eIF4F occurrence is mainly composed of eIFiso4G, eIFiso4E and eIF4A subunits.

Functions of the constituent subunits of eIF4F-like protein complex include recognition of the mRNA 5′ cap structure (eIF4E), delivery of an RNA helicase to the 5′ region (eIF4A), bridging of the mRNA and the ribosome (eIF4G), and circularization of the mRNA via interaction with poly(A)-binding protein (eIF4G).

1. Definition of IF4 isoG:

eIF4 isoG belongs to the eIF4F-like protein complex and is a docking element for eIF4E and eIF4A, eIF4B, polyA binding protein. It is an isoform of eIF4G and its sequence has about 750-800 amino acids. “eIF4 isoG polypeptide” as defined herein refers to any polypeptide comprising the following 3 motifs:

Motif 7: KAV[LF]EPTFCPMYA[QL]LCSDLNEKLP[PS]FPS[ED]EPGGKEITFKRVLLN[NI]CQE AF or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%; 90%, 91%, 92%, 93%, 94%, 95%, 96%, 97%, 98%, or 99% or more sequence identity to Motif 7.
Motif 8: CP[AE]EENVEAIC[QH]FFNTIGKQLDE[SN]PKSRRIND[MVT]YF[SIN][RQ] LKEL[TS][TS]NPQLAPR or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 8.

Motif 9: T[AG]P[DE]QE[ML]ERRDKERLVKLRTLGNIRLIGELLKQKMVPEKIVHHIVQELLG or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 9.

Preferably, eIF4 isoG polypeptide of the invention comprises the following conserved domains: MA3 (PFam accession number: PF02847) and MIF4G (PFam accession number: PF02854).

### 2. Definition of IF4G:

eIF4G belongs to the eIF4F-like protein complex and is also a docking element for eIF4E and eIF4A, eIF4B, polyA binding protein, thus having an equivalent binding functionally as eIF4 isoG in what regards to its role in translation. Its sequence has about 1570-1900 amino acids. “eIF4G polypeptide” as defined herein refers to any polypeptide comprising the following 3 motifs:

Motif 10: TPQNF[ED][KR]LFEQVKAVNIDN[AV]VTL[TN]GVISQIF[DE]KALMEPTFCEMYANFC FH or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 10.

Motif 11: IGELYKK[RK]MLTERIMHECIKKLLGQYQ[DN]PDEE[DN][IV]E[AS]LCKLMSTIGEMI DH or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%; 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 11.

Motif 12: LSNN[MQ][KN]LSSRVRFMLKD[ASV]IDLRKNKWQQRRKVEGPKKIEEVHRDAAQE RQ or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 12.

Preferably, eIF4G polypeptide of the invention comprises the following conserved domains: MA3 (PFam accession number: PF02847) and MIF4G (PFam accession number: PF02854).

3. Definition of eIF4A polypeptide:

eIF4A polypeptide also a subunit of eIF4F-like protein complex and is the polypeptide that binds to eIF4G/isoG and recruits eIF4B at the m7 Gppp cap of the mRNA. Its sequence has about 369-414 amino acids long. “eIF4A polypeptide” as defined herein refers to any polypeptide comprising the following 3 motifs:

Motif 13: RDELTLEGIKQF[YF]V[NA]V[ED][KR]EEWK[LF][DE]TLCDLY[ED]TL[AT] ITQ[SA]VIF or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 13.

Motif 14: SLVINYDLP[TN][QN][PR]E[NL]Y[LI]HRIGRSGRFGRKGVAINF or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 14.

Motif 15: MG[LI][QK]E[ND]LLRGIYAYGFEKPSAIQQR[GA][IV]VP[FI][CI]KG[LR]DVI[QA]QAQ SGTGKT[AS][TM][FI] or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 15.

Preferably, eIF4A polypeptide of the invention comprises the following conserved domains: DEAD (PFam accession number: PF00270) and Helicase_C(PFam accession number: PF00271).

4. Definition of eIF4E polypeptide:

eIF4E polypeptide is also a subunit of eIF4F-like protein complex and is the polypeptide that binds to eIF4G/isoG and to the m7 Gppp cap of the mRNA in the translation initiation process. It has about 195-286 amino acids long. “eIF4E polypeptide” as defined herein refers to any polypeptide comprising the following 3 motifs:

Motif 16: YTFSTVE[ED]FW[SG]LYNNIH[HR]PSKLAVGADF[HY]CFK[NH]KIEPKWEDP[VI]CA NGGKW or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 16.

Motif 17: T[SC]WLYTLLA[ML]IGEQFD[HY]GD[ED]ICGAVV[NS]VR or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 17.

Motif 18: E[KR]I[AS][LI]WTKNA[AS]NE[AST]AQ[VL]SIGKQWKEFLDYN[DE][TS]IGFIFH[ED]DA or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 18.

Preferably, eIF4E polypeptide of the invention comprises the following conserved domain: IF4E (PFam accession number: PF01652).

5. Definition of eIF4 isoE Polypeptide:

eIF4 isoE polypeptide is a isoform of eIF4E and a subunit of eIF4F-like protein complex. It has the same binding activities than eIF4E and has about 189-217 amino acid long. “eIF4 isoE polypeptide” as defined herein refers to any polypeptide comprising the following 3 motifs:

Motif 19: WCLYDQ[IV]F[KR]PSKLP[GA]NADFHLFKAG[VI]EPKWEDPECANGGKW or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 19.

Motif 20: L[ED]TMWLETLMALIGEQFD[ED][AS][DE][ED]ICGWASVR or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 20.

Motif 21: QDKL[SA]LWT[KR][TN]A[AS]NEA[AV]QM[SG]IG[RK]KWKE[IV]ID or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 21.

Preferably, eIF4 isoE polypeptide of the invention comprises the following conserved domain: IF4E (PFam accession number: PF01652).

In a preferred embodiment of the present invention the expression of eIF4G and its isoform is increased, most preferably eIF4 isoG is overexpressed.

In other preferred embodiment of the present invention the expression of eIF4A is increased.

In a most preferred embodiment of the present invention eIF4 isoG and/or eIF4A are overexpressed and the expression of eIF4 isoE is decreased, being preferably eIF4 isoG and eIF4A overexpressed.

Alternatively, the homologue of the eIF4F-like protein complex subunits polypeptides has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 241, by SEQ ID NO: 301 and/or SEQ ID NO. 561 provided that the homologous protein comprises the conserved motifs as outlined above. The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered. For local alignments, the Smith-Waterman algorithm is particularly useful (Smith T F, Waterman M S (1981) J. Mol. Biol. 147(1); 195-7).

Preferably, the polypeptides sequences of the eIF4F subunits, which when used in the construction of a phylogenetic tree, such as the one depicted in FIGS. 12, 13 and 14, clusters with the group of eIF4F-like protein complex subunits, such as eIF4 isoG, eIF4A and eIF4 isoE comprising the amino acid sequences represented respectively by SEQ ID NO: 241, SEQ ID NO: 301, and/or SEQ ID NO: 561.

Most preferably the polypeptides sequences of the present invention clusters with the group of eIF4F-like protein complex subunit eIF4 isoG are codified by SEQ ID NO: 241, rather than with any other group.

Concerning GR-RBP polypeptides, any reference hereinafter to a “protein useful in the methods of the invention” is taken to mean a GR-RBP polypeptide as defined herein. Any reference hereinafter to a “nucleic acid useful in the methods of the invention” is taken to mean a nucleic acid capable of encoding such a GR-RBP polypeptide. The nucleic acid to be introduced into a plant (and therefore useful in performing the methods of the invention) is any nucleic acid encoding the type of protein which will now be described, hereafter also named “GR-RBP nucleic acid” or “GR-RBP gene”.

A “GR-RBP polypeptide” as defined herein refers to any RNA binding polypeptide comprising an RNA Recognition Motif 1 (PFam accession PF00076, RRM_{—}1). Preferably the GR-RBP polypeptide further comprises one or more of the following signature sequences:

Signature sequence 1 (SEQ ID NO: 828):
GGYGG
Signature sequence 2 (SEQ ID NO: 829):
GGYG
Signature sequence 3 (SEQ ID NO: 830):
[CLIV][FY][IV]GG[LIMV]
Signature sequence 4 (SEQ ID NO: 831):
RGF[GA]F[IV][SDHTN][FY]

Preferably the GR-RBP polypeptide comprises a HMMPanther PTHR10432:SF31 RRM_Gly_rich domain. Optionally, the GR-RBP polypeptide also comprises a glycine rich domain in the C-terminal half of the protein. The term “glycine rich domain” as used in the present invention refers to a stretch of at least 10, preferably at least 11, preferably at least 12, more preferably at least 13, most preferably at least 15 amino acids in the sequence of the GR-RBP polypeptide that comprises at least 30% glycine residues.

Further preferably, the GR-RBP polypeptide comprises one or more of the following motifs:

Motif 22 (SEQ ID NO: 832):
S[ST]KLF[VI]GGL[SA][WY]GTDD[QH]SL[RK][ED]AF[SA]S
[FY]G[ED]V[VT][ED]A[RK][VI]I[TV]DR[ED][TS]GRSRGFGF
V[TNS][FY]
Motif 23 (SEQ ID NO: 833):
S[ST]KLF[VI]GGL[SA][WY]GTDD[QH]SL[RK][ED]AF[AS]
[SK][FY]G[ED]V[VTI][ED]A[RK][VI]I[TV]DR[ED]TGRSRGF
GFV[TNS][FY]
Motif 24 (SEQ ID NO: 834):
[ML]DG[KQ][ED]L[DN]GRN[IV]RV[NS]YAN[ED]RP[SR]
Motif 25 (SEQ ID NO: 835):
[SE]E[EDA]A[KS][AS]AISAMDG[KQ][ED]LNGRN[IV]RV[NS]
YA[NT][ED]R
Motif 26 (SEQ ID NO: 836):
MA[FA]LNKLG[SG][LA]LRQSA
Motif 27 (SEQ ID NO: 837):
MA[FA][LCF]NKLG[SGN]LLRQSASS[SN]SAS

More preferably, the GR-RBP polypeptide comprises in increasing order of preference, at least 2, or at least 3 of the above motifs.

Alternatively, the homologue of a GR-RBP protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 827, provided that the homologous protein comprises one, two or three of the conserved motifs as outlined above. The overall sequence identity is determined using a global alignment algorithm, such as the Needleman Wunsch algorithm in the program GAP (GCG Wisconsin Package, Accelrys), preferably with default parameters. Compared to overall sequence identity, the sequence identity will generally be higher when only conserved domains or motifs are considered. Preferably the motifs in a GR-RBP polypeptide have, in increasing order of preference, at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the motifs represented by SEQ ID NO: 832 to SEQ ID NO: 837 (Motifs 22 to 27).

Preferably, the polypeptide sequence which when used in the construction of a phylogenetic tree, such as the one depicted in FIG. 18 clusters with the A or B group rather than with any other group, more preferably with the A group of GR-RBP polypeptides, which comprises the amino acid sequence represented by SEQ ID NO: 827.

The terms “domain”, “signature” and “motif” are defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol. 147(1); 195-7).

In addition, C3H-like polypeptides, when expressed in rice according to the methods of the present invention as outlined in the Examples section herein, give plants having increased yield-related traits, in particular increased aboveground area, and increased seed yield relative to control plants.

Additionally, C3H-like polypeptides may display a preferred subcellular localization, typically one or more of nuclear, cytoplasmic, chloroplastic, or mitochondrial. The task of protein subcellular localisation prediction is well studied. Experimental methods for protein localization range from immunolocalization to tagging of proteins using green fluorescent protein (GFP) or beta-glucuronidase (GUS). Such methods are accurate although labour-intensive compared with computational methods. Recently much progress has been made in computational prediction of protein localisation from sequence data. Among algorithms well known to a person skilled in the art are available at the ExPASy Proteomics tools hosted by the Swiss Institute for Bioinformatics, for example, PSort, TargetP, ChloroP, LocTree, Predotar, LipoP, MITOPROT, PATS, PTS1, SignalP, TMHMM, and others.

Furthermore, SPT-like polypeptides (at least in their native form) typically have DNA-binding activity. Tools and techniques for measuring DNA-binding activity are well known in the art.

In addition, SPT-like polypeptides, when expressed in rice according to the methods of the present invention as outlined in the Examples Section hereinafter, give plants having enhanced yield-related related traits, in particular increased Thousand Kernel Weight (TKW) relative to control plants.

SPT-like polypeptides are typically localised in the nucleus due to the presence of the nuclear localisation signals (see the alignment of FIG. 4) in the SPT-like polypeptides. Experimental methods for determining protein localization range from immunolocalization to tagging of proteins using green fluorescent protein (GFP) or beta-glucuronidase (GUS). Such methods are accurate although labour-intensive compared with computational methods. Recently much progress has been made in computational prediction of protein localisation from sequence data. Among algorithms well known to a person skilled in the art are available at the ExPASy Proteomics tools hosted by the Swiss Institute for Bioinformatics, for example, PSort, TargetP, ChloroP, LocTree, Predotar, LipoP, MITOPROT, PATS, PTS1, SignalP, TMHMM, and others.

Furthermore, IDI2 polypeptides, as alpha subunits of eIF2B (at least in their native form) may mediate phosphorylation of eIF2. Tools and techniques for measuring eIF2Balpha subunit activity are well known in the art, see for example Fabian et al (J. Biol. Chem. 272, 12359-12369, 1997 and Prot. Expr. Purif. 13, 16-22, 1998). Further details are provided in Example 6.

In addition, IDI2 polypeptides, when expressed in rice according to the methods of the present invention as outlined in Examples 7 and 8, give plants, when grown under nutrient limitation, having increased yield-related traits, in particular increased total weight of seeds, increased number of filled seeds and/or increased harvest index.

Furthermore, eIF4F-like protein complex subunits (at least in their native form) typically have translational activity. Tools and techniques for measuring this activity are well known in the art.

In addition, eIF4F-like protein complex subunits, when expressed in rice according to the methods of the present invention as outlined in Examples 8 and 9, give plants having increased yield related traits, in particular maximum height per plant, number of flowers (florets) per panicle and number of plants per square meter (harvested index).

Additionally, eIF4F-like protein complex subunits may display a preferred subcellular localization, typically one or more of nuclear, cytoplasmic, chloroplastic, or mitochondrial. The task of protein subcellular localisation prediction is important and well studied. Knowing a protein's localisation helps elucidate its function. Experimental methods for protein localization range from immunolocalization to tagging of proteins using green fluorescent protein (GFP) or beta-glucuronidase (GUS). Such methods are accurate although labor-intensive compared with computational methods. Recently much progress has been made in computational prediction of protein localisation from sequence data. Among algorithms well known to a person skilled in the art are available at the ExPASy Proteomics tools hosted by the Swiss Institute for Bioinformatics, for example, PSort, TargetP, ChloroP, LocTree, Predotar, LipoP, MITOPROT, PATS, PTS1, SignalP, TMHMM, and others.

Furthermore, GR-RBP polypeptides (at least in their native form) typically have RNA-binding activity. Tools and techniques for measuring RNA-binding activity are well known in the art, see for example Kwak et al. (2005) or Hirose et al. (Nucl. Ac. Res. 21, 3981-3987, 1993). Further details are provided in Example 6.

In addition, GR-RBP polypeptides, when expressed in rice according to the methods of the present invention as outlined in Examples 7 and 8, give plants having increased yield related traits, in particular increased fill rate, when the plants are grown under drought stress conditions.

Concerning C3H-like polypeptides, the present invention is illustrated by transforming plants with the nucleic acid sequence represented by SEQ ID NO: 1, encoding the polypeptide sequence of SEQ ID NO: 2. However, performance of the invention is not restricted to these sequences; the methods of the invention may advantageously be performed using any C3H-like-encoding nucleic acid or C3H-like polypeptide as defined herein.

Examples of nucleic acids encoding C3H-like polypeptides are given in Table A1 of the Examples section herein. Such nucleic acids are useful in performing the methods of the invention. The amino acid sequences given in Table A1 of the Examples section are example sequences of orthologues and paralogues of the C3H-like polypeptide represented by SEQ ID NO: 2, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A1 of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 1 or SEQ ID NO: 2, the second BLAST would therefore be against Medicago sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning SPT-like polypeptides, the present invention is illustrated by transforming plants with the nucleic acid sequence represented by SEQ ID NO: 96, encoding the polypeptide sequence of SEQ ID NO: 97. However, performance of the invention is not restricted to these sequences; the methods of the invention may advantageously be performed using any SPT-like-encoding nucleic acid or SPT-like polypeptide as defined herein.

Examples of nucleic acids encoding SPT-like polypeptides are given in Table A2 of the Examples section herein. Such nucleic acids are useful in performing the methods of the invention. The amino acid sequences given in Table A2 of the Examples section are example sequences of orthologues and paralogues of the SPT-like polypeptide represented by SEQ ID NO: 97, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A2 of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 96 or SEQ ID NO: 97, the second BLAST would therefore be against poplar sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning IDI2 polypeptides, the present invention is illustrated by transforming plants with the nucleic acid sequence represented by SEQ ID NO: 139, encoding the polypeptide sequence of SEQ ID NO: 140. However, performance of the invention is not restricted to these sequences; the methods of the invention may advantageously be performed using any IDI2-encoding nucleic acid or IDI2 polypeptide as defined herein.

Examples of nucleic acids encoding IDI2 polypeptides are given in Table A3 of the Examples section herein. Such nucleic acids are useful in performing the methods of the invention. The amino acid sequences given in Table A3 of the Examples section are example sequences of orthologues and paralogues of the IDI2 polypeptide represented by SEQ ID NO: 2, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A3 of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 139 or SEQ ID NO: 140, the second BLAST would therefore be against Saccharum officinarum sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning eIF4F-like protein complex subunits, the present invention is illustrated by transforming plants with at least a nucleic acid with the following sequences represented by: SEQ ID NO: 240, encoding the polypeptide sequence of SEQ ID NO: 241, SEQ ID NO 300, encoding the polypeptide sequence of SEQ ID NO: 301 and SEQ ID NO 560, encoding the polypeptide sequence of SEQ ID NO: 561. However, performance of the invention is not restricted to these sequences; the methods of the invention may advantageously be performed using at least one eIF4F-like protein complex subunit-encoding nucleic acid or at least one eIF4F-like protein complex subunit as defined herein.

Examples of nucleic acids encoding eIF4F-like protein complex subunits are given in Tables A4 of the Examples section herein. In the scope of the present invention, “Tables A4” comprise Table A4a, A4b and A4c. Such nucleic acids are useful in performing the methods of the invention. The amino acid sequences given in Tables A4 of the Examples section are example sequences of orthologues and paralogues of the eIF4F-like protein complex subunits represented by SEQ ID NO: 241, SEQ ID NO 301 and SEQ ID NO: 561 and by, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Tables A4 of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 240 or SEQ ID NO: 241, the second BLAST would therefore be against rice sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning GR-RBP polypeptides, the present invention is illustrated by transforming plants with the nucleic acid sequence represented by SEQ ID NO: 826, encoding the polypeptide sequence of SEQ ID NO: 827. However, performance of the invention is not restricted to these sequences; the methods of the invention may advantageously be performed using any GR-RBP-encoding nucleic acid or GR-RBP polypeptide as defined herein.

Examples of nucleic acids encoding GR-RBP polypeptides are given in Table A5 of the Examples section herein. Such nucleic acids are useful in performing the methods of the invention. The amino acid sequences given in Table A5 of the Examples section are example sequences of orthologues and paralogues of the GR-RBP polypeptide represented by SEQ ID NO: 827, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A5 of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 826 or SEQ ID NO: 827, the second BLAST would therefore be against rice sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

The task of protein subcellular localisation prediction is important and well studied. Knowing a protein's localisation helps elucidate its function. Experimental methods for protein localization range from immunolocalization to tagging of proteins using green fluorescent protein (GFP) or beta-glucuronidase (GUS). Such methods are accurate although labor-intensive compared with computational methods. Recently much progress has been made in computational prediction of protein localisation from sequence data. Among algorithms well known to a person skilled in the art are available at the ExPASy Proteomics tools hosted by the Swiss Institute for Bioinformatics, for example, PSort, TargetP, ChloroP, LocTree, Predotar, LipoP, MITOPROT, PATS, PTS1, SignalP, TMHMM, and others.

Nucleic acid variants may also be useful in practising the methods of the invention. Examples of such variants include nucleic acids encoding homologues and derivatives of any one of the amino acid sequences given in Table A1 to A5 of the Examples section, the terms “homologue” and “derivative” being as defined herein. Also useful in the methods of the invention are nucleic acids encoding homologues and derivatives of orthologues or paralogues of any one of the amino acid sequences given in Table A1 to A5 of the Examples section. Homologues and derivatives useful in the methods of the present invention have substantially the same biological and functional activity as the unmodified protein from which they are derived. Further variants useful in practising the methods of the invention are variants in which codon usage is optimised or in which miRNA target sites are removed.

Further nucleic acid variants useful in practising the methods of the invention include portions of nucleic acids encoding C3H-like polypeptides, or SPT polypeptides, or IDI2 polypeptides, or eIF4F-like protein complex subunits, or GR-RBP polypeptides, nucleic acids hybridising to nucleic acids encoding C3H-like polypeptides, or SPT polypeptides, or IDI2 polypeptides, or eIF4F-like protein complex subunits, or GR-RBP polypeptides, splice variants of nucleic acids encoding C3H-like polypeptides, or SPT polypeptides, or IDI2 polypeptides, or eIF4F-like protein complex subunits, or GR-RBP polypeptides, allelic variants of nucleic acids encoding C3H-like polypeptides, or SPT polypeptides, or IDI2 polypeptides, or eIF4F-like protein complex subunits, or GR-RBP polypeptides, and variants of nucleic acids encoding C3H-like polypeptides, or SPT polypeptides, or IDI2 polypeptides, or eIF4F-like protein complex subunits, or GR-RBP polypeptides, obtained by gene shuffling. The terms hybridising sequence, splice variant, allelic variant and gene shuffling are as described herein.

Nucleic acids encoding C3H-like polypeptides, or SPT polypeptides, or IDI2 polypeptides, or eIF4F-like protein complex subunits, or GR-RBP polypeptides, need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table A1 to A5 of the Examples section, or a portion of a nucleic acid encoding an orthologue, paralogue or homologue of any of the amino acid sequences given in Table A1 to A5 of the Examples section.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Concerning C3H-like polypeptides, portions useful in the methods of the invention, encode a C3H-like polypeptide as defined herein, and have substantially the same biological activity as the amino acid sequences given in Table A1 of the Examples section. Preferably, the portion is a portion of any one of the nucleic acids given in Table A1 of the Examples section, or is a portion of a nucleic acid encoding an orthologue or paralogue of any one of the amino acid sequences given in Table A1 of the Examples section. Preferably the portion is at least 500, 550, 600, 650, 700, 750, 800, 850, 900, 950, 1000, 1050, 1100, 1150, 1200, 1250, 1300, 1350, 1400, 1450, 1500 or more consecutive nucleotides in length, the consecutive nucleotides being of any one of the nucleic acid sequences given in Table A1 of the Examples section, or of a nucleic acid encoding an orthologue or paralogue of any one of the amino acid sequences given in Table A1 of the Examples section. Most preferably the portion is a portion of the nucleic acid of SEQ ID NO: 1. Preferably, the portion encodes a fragment of an amino acid sequence which, when used in the construction of a phylogenetic tree, such as the one depicted in FIG. 2, clusters with the group of C3H-like polypeptides comprising the amino acid sequence represented by SEQ ID NO: 2 rather than with any other group.

Concerning SPT-like polypeptides, portions useful in the methods of the invention, encode an SPT-like polypeptide as defined herein, and have substantially the same biological activity as the amino acid sequences given in Table A2 of the Examples section. Preferably, the portion is a portion of any one of the nucleic acids given in Table A2 of the Examples section, or is a portion of a nucleic acid encoding an orthologue or paralogue of any one of the amino acid sequences given in Table A2 of the Examples section. Preferably the portion is at least 500, 550, 600, 650, 700, 750, 800, 850, 900, 950, 1000, 1050, 1100, 1150, 1200, 1250, 1300, 1350, 1400, 1450, 1500 or more consecutive nucleotides in length, the consecutive nucleotides being of any one of the nucleic acid sequences given in Table A2 of the Examples section, or of a nucleic acid encoding an orthologue or paralogue of any one of the amino acid sequences given in Table A2 of the Examples section. Most preferably the portion is a portion of the nucleic acid of SEQ ID NO: 96. Preferably, the portion encodes a fragment of an amino acid sequence which, when used in the construction of a phylogenetic tree, such as the one depicted in FIG. 5, clusters with the group of SPT-like polypeptides comprising the amino acid sequence represented by SEQ ID NO: 97 rather than with any other group.

Concerning IDI2 polypeptides, portions useful in the methods of the invention, encode an IDI2 polypeptide as defined herein, and have substantially the same biological activity as the amino acid sequences given in Table A3 of the Examples section. Preferably, the portion is a portion of any one of the nucleic acids given in Table A3 of the Examples section, or is a portion of a nucleic acid encoding an orthologue or paralogue of any one of the amino acid sequences given in Table A3 of the Examples section. Preferably the portion is at least 500, 550, 600, 650, 700, 750, 800, 850, 900, 950, 1000, 1050, 1100, 1150, 1200, 1250, 1300, 1350, 1400, 1500, 1600, 1700, 1800, 1900, 2000, 2100, 2200, 2300, 2400, 2500, 2600 consecutive nucleotides in length, the consecutive nucleotides being of any one of the nucleic acid sequences given in Table A3 of the Examples section, or of a nucleic acid encoding an orthologue or paralogue of any one of the amino acid sequences given in Table A3 of the Examples section. Most preferably the portion is a portion of the nucleic acid of SEQ ID NO: 139. Preferably, the portion encodes a fragment of an amino acid sequence which, when used in the construction of a phylogenetic tree, such as the one depicted in FIG. 9, clusters with the A or B group rather than with any other group, more preferably the polypeptide sequence clusters with the A group of IDI2 polypeptides, which comprises the amino acid sequence represented by SEQ ID NO: 140.

Concerning eIF4F-like protein complex subunits, portions useful in the methods of the invention, encode an eIF4F-like protein complex subunits as defined herein, and have substantially the same biological activity as the amino acid sequences given in Tables A4 of the Examples section. Preferably, the portion is a portion of any one of the nucleic acids given in Tables A4 of the Examples section, or is a portion of a nucleic acid encoding an orthologue or paralogue of any one of the amino acid sequences given in Tables A4 of the Examples section. Preferably the portion is at least 500, 550, 600, 650, 700, 750, 800, 850, 900, 950, 1000 consecutive nucleotides in length, the consecutive nucleotides being of any one of the nucleic acid sequences given in Tables A4 of the Examples section, or of a nucleic acid encoding an orthologue or paralogue of any one of the amino acid sequences given in Tables A4 of the Examples section. Most preferably the portion is a portion of the nucleic acid of SEQ ID NO: 240, 300 or 560. Preferably, the portion encodes a fragment of an amino acid sequence which, when used in the construction of a phylogenetic tree, such as the one depicted in FIGS. 12, 13 and 14, clusters with the group of eIF4F-like subunit polypeptides comprising the amino acid sequence represented by SEQ ID NO: 241 rather than with any other group.

Concerning GR-RBP polypeptides, portions useful in the methods of the invention, encode a GR-RBP polypeptide as defined herein, and have substantially the same biological activity as the amino acid sequences given in Table A5 of the Examples section. Preferably, the portion is a portion of any one of the nucleic acids given in Table A5 of the Examples section, or is a portion of a nucleic acid encoding an orthologue or paralogue of any one of the amino acid sequences given in Table A5 of the Examples section. Preferably the portion is at least 300, 350, 400, 450, 500, 550, 600, 650, 700, 750, 800, 850, 900, 950, 1000, 1050, 1100, 1150, 1200, 1250, 1300, 1350, 1400, 1450, 1500 consecutive nucleotides in length, the consecutive nucleotides being of any one of the nucleic acid sequences given in Table A5 of the Examples section, or of a nucleic acid encoding an orthologue or paralogue of any one of the amino acid sequences given in Table A5 of the Examples section. Most preferably the portion is a portion of the nucleic acid of SEQ ID NO: 826. Preferably, the portion encodes a fragment of an amino acid sequence which, when used in the construction of a phylogenetic tree, such as the one depicted in FIG. 18 clusters with the A or B group rather than with any other group, more preferably with the A group of GR-RBP polypeptides, which comprises the amino acid sequence represented by SEQ ID NO: 827.

Another nucleic acid variant useful in the methods of the invention is a nucleic acid capable of hybridising, under reduced stringency conditions, preferably under stringent conditions, with a nucleic acid encoding a C3H-like polypeptide, or an SPT polypeptide, or an IDI2 polypeptide, or an eIF4F-like protein complex subunit, or a GR-RBP polypeptide, as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table A1 to A5 of the Examples section, or comprising introducing and expressing in a plant a nucleic acid capable of hybridising to a nucleic acid encoding an orthologue, paralogue or homologue of any of the nucleic acid sequences given in Table A1 to AS of the Examples section.

Concerning C3H-like polypeptides, hybridising sequences useful in the methods of the invention encode a C3H-like polypeptide as defined herein, having substantially the same biological activity as the amino acid sequences given in Table A1 of the Examples section. Preferably, the hybridising sequence is capable of hybridising to the complement of any one of the nucleic acids given in Table A1 of the Examples section, or to a portion of any of these sequences, a portion being as defined above, or the hybridising sequence is capable of hybridising to the complement of a nucleic acid encoding an orthologue or paralogue of any one of the amino acid sequences given in Table A1 of the Examples section. Most preferably, the hybridising sequence is capable of hybridising to the complement of a nucleic acid as represented by SEQ ID NO: 1 or to a portion thereof.

Preferably, the hybridising sequence encodes a polypeptide with an amino acid sequence which, when full-length and used in the construction of a phylogenetic tree, such as the one depicted in FIG. 2, clusters with the group of C3H-like polypeptides comprising the amino acid sequence represented by SEQ ID NO: 2 rather than with any other group.

Concerning SPT-like polypeptides, hybridising sequences useful in the methods of the invention encode an SPT-like polypeptide as defined herein, having substantially the same biological activity as the amino acid sequences given in Table A2 of the Examples section. Preferably, the hybridising sequence is capable of hybridising to the complement of any one of the nucleic acids given in Table A2 of the Examples section, or to a portion of any of these sequences, a portion being as defined above, or the hybridising sequence is capable of hybridising to the complement of a nucleic acid encoding an orthologue or paralogue of any one of the amino acid sequences given in Table A2 of the Examples section. Most preferably, the hybridising sequence is capable of hybridising to the complement of a nucleic acid as represented by SEQ ID NO:96 or to a portion thereof.

Preferably, the hybridising sequence encodes a polypeptide with an amino acid sequence which, when full-length and used in the construction of a phylogenetic tree, such as the one depicted in FIG. 5, clusters with the group of SPT-like polypeptides comprising the amino acid sequence represented by SEQ ID NO: 97 rather than with any other group.

Concerning ID2 polypeptides, hybridising sequences useful in the methods of the invention encode an IDI2 polypeptide as defined herein, having substantially the same biological activity as the amino acid sequences given in Table A3 of the Examples section. Preferably, the hybridising sequence is capable of hybridising to the complement of any one of the nucleic acids given in Table A3 of the Examples section, or to a portion of any of these sequences, a portion being as defined above, or the hybridising sequence is capable of hybridising to the complement of a nucleic acid encoding an orthologue or paralogue of any one of the amino acid sequences given in Table A3 of the Examples section. Most preferably, the hybridising sequence is capable of hybridising to the complement of a nucleic acid as represented by SEQ ID NO: 139 or to a portion thereof.

Preferably, the hybridising sequence encodes a polypeptide with an amino acid sequence which, when full-length and used in the construction of a phylogenetic tree, such as the one depicted in FIG. 9, clusters with the A or B group rather than with any other group, more preferably the polypeptide sequence clusters with the A group of IDI2 polypeptides, which comprises the amino acid sequence represented by SEQ ID NO: 140.

Concerning eIF4F-like protein complex subunits, hybridising sequences useful in the methods of the invention encode at least an eIF4F-like protein complex subunit as defined herein, having substantially the same biological activity as the amino acid sequences given in Tables A4 of the Examples section. Preferably, the hybridising sequence is capable of hybridising to the complement of any one of the nucleic acids given in Tables A4 of the Examples section, or to a portion of any of these sequences, a portion being as defined above, or the hybridising sequence is capable of hybridising to the complement of a nucleic acid encoding an orthologue or paralogue of any one of the amino acid sequences given in Tables A4 of the Examples section. Most preferably, the hybridising sequence is capable of hybridising to the complement of a nucleic acid as represented by SEQ ID NO: 240, SEQ ID NO 300 or SEQ ID NO: 560 and, in a further preferable embodiment of the present invention, the hybridising sequence is capable of hybridising to the complement of a nucleic acid as represented by SEQ ID NO: 240 or to any portion thereof.

Preferably, the hybridising sequence encodes at least a polypeptide with an amino acid sequence which, when full-length and used in the construction of a phylogenetic tree, such as the one depicted in FIGS. 12, 13 and 14, clusters with the group of eIF4F-like protein complex subunits comprising the amino acid sequence represented by SEQ ID NO: 241, SEQ ID NO: 301 or SEQ ID NO: 561, and most preferably the amino acid sequence represented by SEQ ID NO: 241, rather than with any other group.

Concerning GR-RBP polypeptides, hybridising sequences useful in the methods of the invention encode a GR-RBP polypeptide as defined herein, having substantially the same biological activity as the amino acid sequences given in Table A5 of the Examples section. Preferably, the hybridising sequence is capable of hybridising to the complement of any one of the nucleic acids given in Table A5 of the Examples section, or to a portion of any of these sequences, a portion being as defined above, or the hybridising sequence is capable of hybridising to the complement of a nucleic acid encoding an orthologue or paralogue of any one of the amino acid sequences given in Table A5 of the Examples section. Most preferably, the hybridising sequence is capable of hybridising to the complement of a nucleic acid as represented by SEQ ID NO: 826 or to a portion thereof.

Preferably, the hybridising sequence encodes a polypeptide with an amino acid sequence which, when full-length and when used in the construction of a phylogenetic tree, such as the one depicted in FIG. 18 clusters with the A or B group rather than with any other group, more preferably with the A group of GR-RBP polypeptides, which comprises the amino acid sequence represented by SEQ ID NO: 827.

Concerning C3H-like polypeptides, or SPT polypeptides, or IDI2 polypeptides, or GR-RBP polypeptides, another nucleic acid variant useful in the methods of the invention is a splice variant encoding a C3H-like polypeptide, or an SPT polypeptide, or an IDI2 polypeptide, or a GR-RBP polypeptide, as defined hereinabove, a splice variant being as defined herein.

Concerning eIF4F-like protein complex subunits, another nucleic acid variant useful in the methods of the invention is a splice variant at least encoding an eIF4F-like protein complex subunit as defined hereinabove, a splice variant being as defined herein.

Concerning C3H-like polypeptides, or SPT polypeptides, or IDI2 polypeptides, or GR-RBP polypeptides, according to the present invention, there is provided a method for enhancing yield-related traits in plants, comprising introducing and expressing in a plant a splice variant of any one of the nucleic acid sequences given in Table A1, or Table A2, or Table A3 or Table A5, of the Examples section, or a splice variant of a nucleic acid encoding an orthologue, paralogue or homologue of any of the amino acid sequences given in Table A1, or Table A2, or Table A3 or Table A5, of the Examples section.

Concerning eIF4F-like protein complex subunits, according to the present invention, there is provided a method for enhancing yield-related traits in plants, comprising introducing and expressing in a plant a splice variant of at least one of the nucleic acid sequences given in Tables A4 of the Examples section, or at least one a splice variant of a nucleic acid encoding an orthologue, paralogue or homologue of at least one of the amino acid sequences given in Tables A4 of the Examples section.

Concerning C3H-like polypeptides, preferred splice variants are splice variants of a nucleic acid represented by SEQ ID NO: 1, or a splice variant of a nucleic acid encoding an orthologue or paralogue of SEQ ID NO: 2. Preferably, the amino acid sequence encoded by the splice variant, when used in the construction of a phylogenetic tree, such as the one depicted in FIG. 2, clusters with the group of C3H-like polypeptides comprising the amino acid sequence represented by SEQ ID NO: 2 rather than with any other group.

Concerning SPT polypeptides, preferred splice variants are splice variants of a nucleic acid represented by SEQ ID NO: 96, or a splice variant of a nucleic acid encoding an orthologue or paralogue of SEQ ID NO: 97. Preferably, the amino acid sequence encoded by the splice variant, when used in the construction of a phylogenetic tree, such as the one depicted in FIG. 5, clusters with the group of SPT-like polypeptides comprising the amino acid sequence represented by SEQ ID NO: 97 rather than with any other group.

Concerning IDI2 polypeptides, preferred splice variants are splice variants of a nucleic acid represented by SEQ ID NO: 139, or a splice variant of a nucleic acid encoding an orthologue or paralogue of SEQ ID NO: 140. Preferably, the amino acid sequence encoded by the splice variant, when used in the construction of a phylogenetic tree, such as the one depicted in FIG. 9, clusters with the A or B group rather than with any other group, more preferably the polypeptide sequence clusters with the A group of IDI2 polypeptides, which comprises the amino acid sequence represented by SEQ ID NO: 140.

Concerning eIF4F-like protein complex subunits, preferred splice variants are splice variants of a nucleic acid represented by SEQ ID NO: 240, SEQ ID NO: 300 and/or SEQ ID NO: 560, or a splice variant of a nucleic acid encoding an orthologue or paralogue of S SEQ ID NO: 241, SEQ ID NO: 301 or SEQ ID NO: 561. Preferably, the amino acid sequence encoded by the splice variant, when used in the construction of a phylogenetic tree, such as the one depicted in FIGS. 12, 13 and/or 14, clusters with at least one of the group of eIF4F-like protein complex subunit, such as eIF4 isoG/G, eIF4A or eIF4E/isoE comprising at least one amino acid sequence represented by SEQ ID NO: 241, SEQ ID NO: 301 or SEQ ID NO: 561, most preferably the amino acid sequence represented by SEQ ID NO: 241, rather than with any other group.

Concerning GR-RBP polypeptides, preferred splice variants are splice variants of a nucleic acid represented by SEQ ID NO: 826, or a splice variant of a nucleic acid encoding an orthologue or paralogue of SEQ ID NO: 827. Preferably, the amino acid sequence encoded by the splice variant, when used in the construction of a phylogenetic tree, such as the one depicted in FIG. 18 clusters with the A or B group rather than with any other group, more preferably with the A group of GR-RBP polypeptides, which comprises the amino acid sequence represented by SEQ ID NO: 826.

Another nucleic acid variant useful in performing the methods of the invention is an allelic variant of a nucleic acid encoding a C3H-like polypeptide, or an SPT polypeptide, or an IDI2 polypeptide, or an eIF4F-like protein complex subunit, or a GR-RBP polypeptide, as defined hereinabove, an allelic variant being as defined herein.

According to the present invention, there is provided a method for enhancing yield-related traits in plants, comprising introducing and expressing in a plant an allelic variant of any one of the nucleic acids given in Table A1 to A5 of the Examples section, or comprising introducing and expressing in a plant an allelic variant of a nucleic acid encoding an orthologue, paralogue or homologue of any of the amino acid sequences given in Table A1 to A5 of the Examples section.

Concerning C3H-like polypeptides, the polypeptides encoded by allelic variants useful in the methods of the present invention have substantially the same biological activity as the C3H-like polypeptide of SEQ ID NO: 2 and any of the amino acids depicted in Table A1 of the Examples section. Allelic variants exist in nature, and encompassed within the methods of the present invention is the use of these natural alleles. Preferably, the allelic variant is an allelic variant of SEQ ID NO: 1 or an allelic variant of a nucleic acid encoding an orthologue or paralogue of SEQ ID NO: 2. Preferably, the amino acid sequence encoded by the allelic variant, when used in the construction of a phylogenetic tree, such as the one depicted in FIG. 2, clusters with the C3H-like polypeptides comprising the amino acid sequence represented by SEQ ID NO: 2 rather than with any other group.

Concerning SPT polypeptides, the polypeptides encoded by allelic variants useful in the methods of the present invention have substantially the same biological activity as the SPT-like polypeptide of SEQ ID NO: 97 and any of the amino acids depicted in Table A2 of the Examples section. Allelic variants exist in nature, and encompassed within the methods of the present invention is the use of these natural alleles. Preferably, the allelic variant is an allelic variant of SEQ ID NO: 96 or an allelic variant of a nucleic acid encoding an orthologue or paralogue of SEQ ID NO: 97. Preferably, the amino acid sequence encoded by the allelic variant, when used in the construction of a phylogenetic tree, such as the one depicted in FIG. 5, clusters with the SPT-like polypeptides comprising the amino acid sequence represented by SEQ ID NO: 97 rather than with any other group.

Concerning IDI2 polypeptides, the polypeptides encoded by allelic variants useful in the methods of the present invention have substantially the same biological activity as the IDI2 polypeptide of SEQ ID NO: 140 and any of the amino acids depicted in Table A3 of the Examples section. Allelic variants exist in nature, and encompassed within the methods of the present invention is the use of these natural alleles. Preferably, the allelic variant is an allelic variant of SEQ ID NO: 139 or an allelic variant of a nucleic acid encoding an orthologue or paralogue of SEQ ID NO: 140. Preferably, the amino acid sequence encoded by the allelic variant, when used in the construction of a phylogenetic tree, such as the one depicted in FIG. 3, clusters with the A or B group rather than with any other group, more preferably the polypeptide sequence clusters with the A group of IDI2 polypeptides, which comprises the amino acid sequence represented by SEQ ID NO: 140.

Concerning eIF4F-like protein complex subunits, the polypeptides encoded by allelic variants useful in the methods of the present invention have substantially the same biological activity as the eIF4F-like protein complex subunits of anyone of the sequences represented by SEQ ID NO: 241, SEQ ID NO: 301 or SEQ ID NO: 561 and any of the amino acids depicted in Tables A4 of the Examples section. Allelic variants exist in nature, and encompassed within the methods of the present invention is the use of these natural alleles. Preferably, the allelic variant is an allelic variant of SEQ ID NO: 240, SEQ ID NO: 300 and/or SEQ ID NO: 560 or an allelic variant of a nucleic acid encoding an orthologue or paralogue of SEQ ID NO: 241, SEQ ID NO: 301 and/or SEQ ID NO: 561. Preferably, the amino acid sequence encoded by the allelic variant, when used in the construction of a phylogenetic tree, such as the one depicted in FIGS. 12, 13 and/or 14, clusters with the eIF4F-like protein complex subunits, such as eIF4 isoG/G, eIF4A or eIF4E/isoE, comprising the amino acid sequence represented by SEQ ID NO: 241 rather than with any other group.

Concerning GR-RBP polypeptides, the polypeptides encoded by allelic variants useful in the methods of the present invention have substantially the same biological activity as the GR-RBP polypeptide of SEQ ID NO: 827 and any of the amino acids depicted in Table A5 of the Examples section. Allelic variants exist in nature, and encompassed within the methods of the present invention is the use of these natural alleles. Preferably, the allelic variant is an allelic variant of SEQ ID NO: 826 or an allelic variant of a nucleic acid encoding an orthologue or paralogue of SEQ ID NO: 827. Preferably, the amino acid sequence encoded by the allelic variant, when used in the construction of a phylogenetic tree, such as the one depicted in FIG. 18 clusters with the A or B group rather than with any other group, more preferably with the A group of GR-RBP polypeptides, which comprises the amino acid sequence represented by SEQ ID NO: 827.

Gene shuffling or directed evolution may also be used to generate variants of nucleic acids encoding C3H-like polypeptides, or SPT polypeptides, or IDI2 polypeptides, or eIF4F-like protein complex subunits, or GR-RBP polypeptides, as defined above; the term “gene shuffling” being as defined herein.

According to the present invention, there is provided a method for enhancing yield-related traits in plants, comprising introducing and expressing in a plant a variant of any one of the nucleic acid sequences given in Table A1 to A5 of the Examples section, or comprising introducing and expressing in a plant a variant of a nucleic acid encoding an orthologue, paralogue or homologue of any of the amino acid sequences given in Table A1 to A5 of the Examples section, which variant nucleic acid is obtained by gene shuffling.

Concerning C3H-like polypeptides, preferably, the amino acid sequence encoded by the variant nucleic acid obtained by gene shuffling, when used in the construction of a phylogenetic tree such as the one depicted in FIG. 2, clusters with the group of C3H-like polypeptides comprising the amino acid sequence represented by SEQ ID NO: 2 rather than with any other group.

Concerning SPT polypeptides, preferably, the amino acid sequence encoded by the variant nucleic acid obtained by gene shuffling, when used in the construction of a phylogenetic tree such as the one depicted in FIG. 5, clusters with the group of SPT-like polypeptides comprising the amino acid sequence represented by SEQ ID NO: 97 rather than with any other group.

Concerning IDI2 polypeptides, preferably, the amino acid sequence encoded by the variant nucleic acid obtained by gene shuffling, when used in the construction of a phylogenetic tree such as the one depicted in FIG. 9, clusters with the A or B group rather than with any other group, more preferably the polypeptide sequence clusters with the A group of IDI2 polypeptides, which comprises the amino acid sequence represented by SEQ ID NO: 140.

Concerning eIF4F-like protein complex subunits, preferably, the amino acid sequence encoded by the variant nucleic acid obtained by gene shuffling, when used in the construction of a phylogenetic tree such as the one depicted in FIGS. 12, 13 and/or 14, clusters with the group of eIF4F-like protein complex subunit comprising the amino acid sequence represented by SEQ ID NO: 241, SEQ ID NO: 301 and/or SEQ ID NO: 561, most preferably clusters with the group of eIF4F-like protein complex subunit comprising the amino acid sequence represented by SEQ ID NO: 241, rather than with any other group.

Concerning GR-RBP polypeptides, preferably, the amino acid sequence encoded by the variant nucleic acid obtained by gene shuffling, when used in the construction of a phylogenetic tree, such as the one depicted in FIG. 18 clusters with the A or B group rather than with any other group, more preferably with the A group of GR-RBP polypeptides, which comprises the amino acid sequence represented by SEQ ID NO: 827.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding C3H-like polypeptides may be derived from any natural or artificial source. The nucleic acid may be modified from its native form in composition and/or genomic environment through deliberate human manipulation. Preferably the C3H-like polypeptide-encoding nucleic acid is from a plant, preferably from the family Medicago, most preferably the nucleic acid is from Medicago truncatula.

Nucleic acids encoding SPT-like polypeptides may be derived from any natural or artificial source. The nucleic acid may be modified from its native form in composition and/or genomic environment through deliberate human manipulation. Preferably the SPT-like polypeptide-encoding nucleic acid is from a plant, further preferably from the family Salicaceae, preferably from the genus Populus, most preferably the nucleic acid is from Populus trichocarpa.

Nucleic acids encoding IDI2 polypeptides may be derived from any natural or artificial source. The nucleic acid may be modified from its native form in composition and/or genomic environment through deliberate human manipulation. Preferably the IDI2 polypeptide-encoding nucleic acid is from a plant, further preferably from a monocotyledonous plant, more preferably from the family Poaceae, most preferably the nucleic acid is from Saccharum officinarum.

Nucleic acids encoding eIF4F-like protein complex subunit may be derived from any natural or artificial source. The nucleic acids may be modified from its native form in composition and/or genomic environment through deliberate human manipulation. Preferably the eIF4F-like protein complex subunits encoding nucleic acids are from a plant, further preferably from a monocotyledonous plant, more preferably from the family Poaceae, most preferably the nucleic acid is from Oryza sativa.

Nucleic acids encoding GR-RBP polypeptides may be derived from any natural or artificial source. The nucleic acid may be modified from its native form in composition and/or genomic environment through deliberate human manipulation. Preferably the GR-RBP polypeptide-encoding nucleic acid is from a plant, further preferably from a monocotyledonous plant, more preferably from the family Poaceae, most preferably the nucleic acid is from Oryza sativa.

Concerning C3H-like polypeptides, or SPT polypeptides, or eIF4F-like protein complex subunits, performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Concerning IDI2 polypeptides, or GR-RBP polypeptides, performance of the methods of the invention gives plants having enhanced yield-related traits. In particular performance of the methods of the invention gives plants having increased yield, especially increased seed yield, increased biomass and/or increased early vigour, relative to control plants. The terms “yield” and “seed yield” and “early vigour” are described in more detail in the “definitions” section herein.

Concerning C3H-like polypeptides, or SPT polypeptides, or eIF4F-like protein complex subunits, reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of control plants.

Concerning IDI2 polypeptides, reference herein to enhanced yield-related traits is taken to mean an increase in biomass (weight) of one or more parts of a plant, which may include aboveground (harvestable) parts and/or (harvestable) parts below ground. In particular, such harvestable parts are seeds, above-ground biomass and/or roots, and performance of the methods of the invention results in plants having increased early vigour, increased seed yield, and/or increased biomass relative to control plants.

Concerning GR-RBP polypeptides, Reference herein to enhanced yield-related traits is taken to mean an increase in biomass (weight) of one or more parts of a plant, which may include aboveground (harvestable) parts and/or (harvestable) parts below ground. In particular, such harvestable parts are seeds and/or roots, and performance of the methods of the invention results in plants having increased seed yield relative to the seed yield of control plants and/or enhanced root growth, compared to control plants.

Taking corn as an example, a yield increase may be manifested as one or more of the following: increase in the number of plants established per square meter, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others.

Taking rice as an example, a yield increase may manifest itself as an increase in one or more of the following: number of plants per square meter, number of panicles per plant, panicle length, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others. In rice, submergence tolerance may also result in increased yield.

The present invention provides a method for increasing yield, especially seed yield of plants, relative to control plants, which method comprises modulating expression in a plant of a nucleic acid encoding a C3H-like polypeptide, or an SPT-like polypeptide, or an IDI2 polypeptide, as defined herein.

The present invention also provides a method for increasing yield, especially seed yield of plants, relative to control plants, which method comprises modulating the activity in a plant of an eIF4F-like protein complex by modulating the expression of at least one of its subunits nucleic acid encoding polypeptides as defined herein.

The present invention also provides a method for increasing yield, especially seed yield and/or root yield of plants, relative to control plants, which method comprises modulating expression in a plant of a nucleic acid encoding a GR-RBP polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as speed of germination, early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per square meter (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

Concerning C3H-like polypeptides, or SPT polypeptides, or IDI2 polypeptides, or GR-RBP polypeptides, according to a preferred feature of the present invention, performance of the methods of the invention gives plants having an increased growth rate relative to control plants. Therefore, according to the present invention, there is provided a method for increasing the growth rate of plants, which method comprises modulating expression in a plant of a nucleic acid encoding a C3H-like polypeptide, or an SPT polypeptide, or an IDI2 polypeptide, or an eIF4F-like protein complex, or a GR-RBP polypeptide, as defined herein.

Concerning eIF4F-like protein complex subunits, according to a preferred feature of the present invention, performance of the methods of the invention gives plants having an increased growth rate relative to control plants. Therefore, according to the present invention, there is provided a method for increasing the growth rate of plants, which method comprises modulating the activity of an eIF4F-like protein complex, in a plant, by modulating and expressing at least a nucleic acid encoding for its subunits polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35%, 30% or 25%, more preferably less than 20% or 15% in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi, nematodes and insects. Biotic stresses are typically those stresses caused by pathogens, such as bacteria, viruses, fungi, nematodes, and insects. The term “non-stress” conditions as used herein are those environmental conditions that allow optimal growth of plants. Persons skilled in the art are aware of normal soil conditions and climatic conditions for a given location. The term non-stress conditions as used herein, encompasses the occasional or everyday mild stresses to which a plant is exposed, as defined herein, but does not encompass severe stresses.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 97%, 95%, 92%, 90%, 87%, 85%, 83%, 80%, 77% or 75% of the average production of such plant in a given environment. Average production may be calculated on harvest and/or season basis. Persons skilled in the art are aware of average yield productions of a crop.

Concerning C3H-like polypeptides, or SPT polypeptides, or IDI2 polypeptides, or GR-RBP polypeptides, performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises modulating expression in a plant of a nucleic acid encoding a C3H-like polypeptide, or an SPT polypeptide, or an IDI2 polypeptide, or a GR-RBP polypeptide.

Concerning eIF4F-like protein complex subunits, performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises modulating the activity in a plant of an eIF4F-like protein complex by modulating and expressing at least one of its subunits nucleic acid encoding polypeptide.

Concerning C3H-like polypeptides, or SPT polypeptides, or IDI2 polypeptides, or GR-RBP polypeptides, performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises modulating expression in a plant of a nucleic acid encoding a C3H-like polypeptide, or an SPT polypeptide, or an IDI2 polypeptide, or an eIF4F-like protein complex, or a GR-RBP polypeptide. Nutrient deficiency may result from a lack of nutrients such as nitrogen, phosphates and other phosphorous-containing compounds, potassium, calcium, magnesium, manganese, iron and boron, amongst others. Concerning IDI2 polypeptides, the nutrient deficiency is preferably a deficiency in nitrogen.

Concerning eIF4F-like protein complex subunits, performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises modulating activity of an eIF4F-like protein complex by modulating and expressing at least one of its subunits nucleic acid encoding polypeptide. Nutrient deficiency may result from a lack of nutrients such as nitrogen, phosphates and other phosphorous-containing compounds, potassium, calcium, magnesium, manganese, iron and boron, amongst others.

Performance of the methods of the invention gives plants grown under conditions of salt stress, increased yield relative to control plants grown under comparable conditions. Therefore, according to the present invention, there is provided a method for increasing yield in plants grown under conditions of salt stress, which method comprises modulating expression in a plant of a nucleic acid encoding a C3H-like polypeptide. The term salt stress is not restricted to common salt (NaCl), but may be any one or more of: NaCl, KCl, LiCl, MgCl₂, CaCl₂, amongst others.

The present invention encompasses plants or parts thereof (including seeds) obtainable by the methods according to the present invention. The plants or parts thereof comprise a nucleic acid transgene encoding a C3H-like polypeptide, or an SPT polypeptide, or an IDI2 polypeptide, or an eIF4F-like protein complex subunit, or a GR-RBP polypeptide, as defined above.

Concerning C3H-like polypeptides, or SPT polypeptides, or IDI2 polypeptides, or eIF4F-like protein complex subunits, or GR-RBP polypeptides, the invention also provides genetic constructs and vectors to facilitate introduction and/or expression in plants of nucleic acids encoding C3H-like polypeptides, or SPT polypeptides, or IDI2 polypeptides, or eIF4F-like protein complex subunits, or GR-RBP polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

Concerning eIF4F-like protein complex subunits, the invention also provides genetic constructs and vectors to facilitate introduction and/or expression in plants of at least one nucleic acid encoding eIF4F-like protein complex subunit polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding a C3H-like polypeptide, or an SPT polypeptide, or an IDI2 polypeptide, or a GR-RBP polypeptide, or at least a nucleic acid encoding an eIF4F-like protein complex subunit polypeptide as defined above;
(b) one or more control sequences capable of driving expression of the nucleic acid sequence of (a); and optionally
(c) a transcription termination sequence.

Concerning C3H-like polypeptides, or SPT polypeptides, or IDI2 polypeptides, or GR-RBP polypeptides, the nucleic acid encoding a C3H-like polypeptide, or an SPT polypeptide, or an IDI2 polypeptide, or a GR-RBP polypeptide, is preferably as defined above. Concerning eIF4F-like protein complex subunits, the nucleic acid encoding an eIF4F-like protein complex subunit is preferably at least of the subunit polypeptide as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence, but preferably the promoter is of plant origin. A constitutive promoter is particularly useful in the methods. Preferably the constitutive promoter is also a ubiquitous promoter, or a ubiquitous promoter of medium strength. See the “Definitions” section herein for definitions of the various promoter types.

Concerning C3H-like polypeptides, it should be clear that the applicability of the present invention is not restricted to the C3H-like polypeptide-encoding nucleic acid represented by SEQ ID NO: 1, nor is the applicability of the invention restricted to expression of a C3H-like polypeptide-encoding nucleic acid when driven by a constitutive promoter.

The constitutive promoter is preferably a medium strength promoter, more preferably selected from a plant derived promoter, such as a GOS2 promoter, more preferably is the promoter GOS2 promoter from rice. Further preferably the constitutive promoter is represented by a nucleic acid sequence substantially similar to SEQ ID NO: 95, most preferably the constitutive promoter is as represented by SEQ ID NO: 95. See the “Definitions” section herein for further examples of constitutive promoters.

Optionally, one or more terminator sequences may be used in the construct introduced into a plant. Preferably, the construct comprises an expression cassette comprising a GOS2 promoter, substantially similar to SEQ ID NO: 95, and the nucleic acid encoding the C3H-like polypeptide.

Concerning SPT polypeptides, it should be clear that the applicability of the present invention is not restricted to the S PT-like polypeptide-encoding nucleic acid represented by SEQ ID NO: 96, nor is the applicability of the invention restricted to expression of an SPT-like polypeptide-encoding nucleic acid when driven by a constitutive promoter.

The constitutive promoter is preferably a medium strength promoter, more preferably selected from a plant derived promoter, such as a GOS2 promoter, more preferably is the promoter GOS2 promoter from rice. Further preferably the constitutive promoter is represented by a nucleic acid sequence substantially similar to SEQ ID NO: 135, most preferably the constitutive promoter is as represented by SEQ ID NO: 135. See the “Definitions” section herein for further examples of constitutive promoters.

Optionally, one or more terminator sequences may be used in the construct introduced into a plant. Preferably, the construct comprises an expression cassette comprising a GOS2 promoter, substantially similar to SEQ ID NO: 135, and the nucleic acid encoding the SPT-like polypeptide.

Concerning IDI2 polypeptides, it should be clear that the applicability of the present invention is not restricted to the IDI2 polypeptide-encoding nucleic acid represented by SEQ ID NO: 139, nor is the applicability of the invention restricted to expression of an IDI2 polypeptide-encoding nucleic acid when driven by a constitutive promoter.

The constitutive promoter is preferably a medium strength promoter, more preferably selected from a plant, such as a GOS2 promoter, more preferably is the promoter GOS2 promoter from rice. Further preferably the constitutive promoter is represented by a nucleic acid sequence substantially similar to SEQ ID NO: 149, most preferably the constitutive promoter is as represented by SEQ ID NO: 149. See the “Definitions” section herein for further examples of constitutive promoters.

Optionally, one or more terminator sequences may be used in the construct introduced into a plant. Preferably, the construct comprises an expression cassette comprising a rice GOS2 promoter, substantially similar to SEQ ID NO: 149, and the nucleic acid encoding the IDI2 polypeptide.

Concerning eIF4F-like protein complex subunits, it should be clear that the applicability of the present invention is not restricted to the eIF4F-like protein complex subunit polypeptide-encoding nucleic acids represented by SEQ ID NO: 240, SEQ ID NO: 300 and/or SEQ ID NO: 560, nor is the applicability of the invention restricted to expression of an eIF4F-like protein complex subunit polypeptide-encoding nucleic acids when driven by a constitutive promoter.

The constitutive promoter is preferably a medium strength promoter, more preferably selected from a plant derived promoter, such as a GOS2 promoter, more preferably is the promoter GOS2 promoter from rice. Further preferably the constitutive promoter is represented by a nucleic acid sequence substantially similar to SEQ ID NO: 818 and/or SEQ ID NO: 819, most preferably the constitutive promoter is as represented by SEQ ID NO: 818. See the “Definitions” section herein for further examples of constitutive promoters.

Optionally, one or more terminator sequences may be used in the construct introduced into a plant. Preferably, the construct comprises an expression cassette comprising a GOS2 promoter, substantially similar to SEQ ID NO: 818, and at least a nucleic acid encoding an eIF4F-like protein complex subunit polypeptide.

Concerning GR-RBP polypeptides, it should be clear that the applicability of the present invention is not restricted to the GR-RBP polypeptide-encoding nucleic acid represented by SEQ ID NO: 826, nor is the applicability of the invention restricted to expression of a GR-RBP polypeptide-encoding nucleic acid when driven by a constitutive promoter.

The constitutive promoter is preferably a medium strength promoter, more preferably selected from a plant derived promoter, such as a GOS2 promoter; more preferably the promoter is the GOS2 promoter from rice. Further preferably the constitutive promoter is represented by a nucleic acid sequence substantially similar to SEQ ID NO: 840, most preferably the constitutive promoter is as represented by SEQ ID NO: 840. See the “Definitions” section herein for further examples of constitutive promoters.

Optionally, one or more terminator sequences may be used in the construct introduced into a plant. Preferably, the construct comprises an expression cassette comprising a rice GOS2 promoter, substantially similar to SEQ ID NO: 840, and the nucleic acid encoding the GR-RBP polypeptide.

Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits relative to control plants, comprising introduction and expression in a plant of any nucleic acid encoding a C3H-like polypeptide, or a SPT polypeptide, or an IDI2 polypeptide, or an eIF4F-like protein complex subunit, or a GR-RBP polypeptide, as defined hereinabove.

More specifically, the present invention provides a method for the production of transgenic plants having enhanced yield-related traits, particularly increased (seed) yield, which method comprises:
(i) introducing and expressing in a plant or plant cell nucleic acid encoding a C3H-like polypeptide, or a SPT polypeptide, or an IDI2 polypeptide, or an eIF4F-like protein complex subunit, or a GR-RBP polypeptide; and
(ii) cultivating the plant cell under conditions promoting plant growth and development.

The nucleic acid of (i) may be any of the nucleic acids capable of encoding a C3H-like polypeptide, or a SPT polypeptide, or an IDI2 polypeptide, or an eIF4F-like protein complex subunit, or a GR-RBP polypeptide, as defined herein.

The invention also provides a method for the production of transgenic plants having enhanced yield-related traits relative to control plants, comprising introduction and expression in a plant of any nucleic acid encoding a C3H-like polypeptide, or a SPT polypeptide, or an IDI2 polypeptide, or an eIF4F-like protein complex subunit, or a GR-RBP polypeptide, as defined hereinabove.

More specifically, the present invention provides a method for the production of transgenic plants having enhanced yield-related traits, particularly increased yield and/or increased early vigour, which method comprises:
(i) introducing and expressing in a plant or plant cell nucleic acid encoding a C3H-like polypeptide, or a SPT polypeptide, or an IDI2 polypeptide, or an eIF4F-like protein complex subunit, or a GR-RBP polypeptide; and
(ii) cultivating the plant cell under conditions promoting plant growth and development.

The nucleic acid of (i) may be any of the nucleic acids capable of encoding a C3H-like polypeptide, or a SPT polypeptide, or an IDI2 polypeptide, or an eIF4F-like protein complex subunit, or a GR-RBP polypeptide, as defined herein.

The invention also provides a method for the production of transgenic plants having enhanced yield-related traits relative to control plants, comprising introduction and expression in a plant of at least a nucleic acid encoding an eIF4F-like protein complex subunit polypeptide, as defined hereinabove.

More specifically, the present invention provides a method for the production of transgenic plants having enhanced yield-related traits, particularly increased (seed) yield, which method comprises:
(i) introducing and expressing in a plant or plant cell at least an eIF4F-like protein complex subunit polypeptide-encoding nucleic acid; and
(ii) cultivating the plant cell under conditions promoting plant growth and development.

The nucleic acid of (i) may be any of the nucleic acids capable of encoding an eIF4F-like protein complex subunit polypeptides as defined herein.

The nucleic acid may be introduced directly into a plant cell or into the plant itself (including introduction into a tissue, organ or any other part of a plant). According to a preferred feature of the present invention, the nucleic acid is preferably introduced into a plant, by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a C3H-like polypeptide, or an SPT polypeptide, or an IDI2 polypeptide, or an el F4F-like protein complex subunit polypeptide, or a GR-RBP polypeptide, as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, linseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum, emmer, spelt, secale, einkorn, teff, milo and oats.

The invention also extends to harvestable parts of a plant such as, but not limited to seeds, leaves, fruits, flowers, stems, roots, rhizomes, tubers and bulbs, which harvestable parts comprise a recombinant nucleic acid encoding a C3H-like polypeptide, or an SPT polypeptide, or an IDI2 polypeptide, or an eIF4F-like protein complex subunit polypeptide, or a GR-RBP polypeptide. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating expression of a nucleic acid encoding a C3H-like polypeptide, or an SPT polypeptide, or an IDI2 polypeptide, or an eIF4F-like protein complex subunit polypeptide, or a GR-RBP polypeptide, is by introducing and expressing in a plant a nucleic acid encoding a C3H-like polypeptide, or an SPT polypeptide, or an IDI2 polypeptide, or an eIF4F-like protein complex subunit polypeptide, or a GR-RBP polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The present invention also encompasses use of nucleic acids encoding C3H-like polypeptides as described herein and use of these C3H-like polypeptides, or SPT polypeptides, or IDI2 polypeptides, or eIF4F-like protein complex subunit polypeptides, or GR-RBP polypeptides, in enhancing any of the aforementioned yield-related traits in plants.

Nucleic acids encoding a C3H-like polypeptide, or an SPT polypeptide, or an IDI2 polypeptide, or an eIF4F-like protein complex subunit polypeptide, or a GR-RBP polypeptide, described herein, or the C3H-like polypeptides, or SPT polypeptides, or IDI2 polypeptides, or eIF4F-like protein complex subunit polypeptides, or GR-RBP polypeptides, themselves, may find use in breeding programmes in which a DNA marker is identified which may be genetically linked to gene encoding a C3H-like polypeptide, or an SPT polypeptide, or an IDI2 polypeptide, or an eIF4F-like protein complex subunit polypeptide, or a GR-RBP polypeptide. The nucleic acids/genes, or the C3H-like polypeptides, or SPT polypeptides, or IDI2 polypeptides, or eIF4F-like protein complex subunit polypeptides, or GR-RBP polypeptides, themselves may be used to define a molecular marker. This DNA or protein marker may then be used in breeding programmes to select plants having enhanced yield-related traits as defined hereinabove in the methods of the invention.

Allelic variants of a C3H-like polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding C3H-like polypeptides, or SPT polypeptides, or IDI2 polypeptides, or eIF4F-like protein complex subunit polypeptides, or GR-RBP polypeptides, may also be used as probes for genetically and physically mapping the genes that they are a part of, and as markers for traits linked to those genes. Such information may be useful in plant breeding in order to develop lines with desired phenotypes. Such use of nucleic acids encoding C3H-like polypeptide, or SPT polypeptide, or IDI2 polypeptide, or eIF4F-like protein complex subunit polypeptide, or GR-RBP polypeptide, requires only a nucleic acid sequence of at least 15 nucleotides in length. The nucleic acids encoding C3H-like polypeptide, or SPT polypeptide, or IDI2 polypeptide, or eIF4F-like protein complex subunit polypeptide, or GR-RBP polypeptide, may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the nucleic acids encoding C3H-like polypeptide, or SPT polypeptide, or IDI2 polypeptide, or eIF4F-like protein complex subunit polypeptide, or GR-RBP polypeptide. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the nucleic acid encoding C3H-like polypeptide, or SPT polypeptide, or IDI2 polypeptide, or eIF4F-like protein complex subunit polypeptide, or GR-RBP polypeptide, in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### Items

### 1. C3H-like Polypeptides

1. A method for enhancing yield-related traits in plants relative to control plants, comprising modulating expression in a plant of a nucleic acid encoding a C3H-like polypeptide,
   wherein said C3H-like polypeptide comprises Domain 4 and any one or more of Domains 1, 2, 3 and 5:

| | |
|---|---|
| Domain 1: | C-X₂-C-X₁₂₋₂₃-C-X₂-C-X₂-G-F |

wherein X is any amino acid and the underlined residues are conserved

| | |
|---|---|
| Domain 2: | Y-X₇₋₁₂-L-X₃-P-X₁₀-G |

wherein X is any amino acid and the underlined residues are conserved

| | |
|---|---|
| Domain 3: | S-K-X₆-P |

wherein X is any amino acid and the underlined residues are conserved

| | |
|---|---|
| Domain 4: | RING-C3H2C3 type |

Domain 5: DUF1117
2. Method according to item 1, wherein Domain1 is: CYSCTRFINLSDHTL----------IVCPHCDNGF, or a domain comprising the underlined conserved residues and having, in increasing order of preference, at least 60%, 65%, 70%, 75%, 80%, 85%, 90%, 95% or more sequence identity to the non-underlined residues in Domain 1, where “-” is a gap or any residue.
3. Method according to item 1 or 2, wherein, Domain 2 is: YDDGDG-----SGLRPLPPTVSEFLLGSG, or a domain comprising the underlined conserved residues and having, in increasing order of preference, at least 60%, 65%, 70%, 75%, 80%, 85%, 90%, 95% or more sequence identity to the non-underlined residues in Domain2, where “-” is a gap or any residue.
4. Method according to any one of items 1 to 3, wherein Domain 3 is: SKAAIESMP, or a domain comprising the underlined conserved residues and having, in increasing order of preference, at least 60%, 65%, 70%, 75%, 80%, 85%, 90%, 95% or more sequence identity to the non-underlined residues in Domain3.
5. Method according to any one of items 1 to 4, wherein Domain 4 is: CAVCKEEFELHAEARELPCKHLYHSDCILPWLTVRNSCPVCR, or a domain comprising the underlined conserved residues and having, in increasing order of preference, at least 60%, 65%, 70%, 75%, 80%, 85%, 90%, 95% or more sequence identity to the non-underlined residues in Domain4.
6. Method according to any one of items 1 to 5, wherein Domain 5 is: GLTIWRLPGGGFAVGRFSGGRSA-GESHFPVVYTEMDGGLN, or a domain having, in increasing order of preference, at least 60%, 65%, 70%, 75%, 80%, 85%, 90%, 95% or more sequence identity to Domain 5, where “-” is a gap or any residue.
7. Method according to any one of items 1 to 6, wherein said modulated expression is effected by introducing and expressing in a plant a nucleic acid encoding a C3H-like polypeptide.
8. Method according to any one of items 1 to 7, wherein said nucleic acid encoding a C3H-like polypeptide encodes any one of the proteins listed in Table A1 or is a portion of such a nucleic acid, or a nucleic acid capable of hybridising with such a nucleic acid.
9. Method according to any one of items 1 to 8, wherein said nucleic acid sequence encodes an orthologue or paralogue of any of the proteins given in Table A1.
10. Method according to any preceding item, wherein said enhanced yield-related traits comprise increased yield, preferably increased biomass and/or increased seed yield relative to control plants.
11. Method according to any one of items 1 to 10, wherein said enhanced yield-related traits are obtained under conditions of drought stress.
12. Method according to any one of items 7 to 11, wherein said nucleic acid is operably linked to a constitutive promoter, preferably to a GOS2 promoter, most preferably to a GOS2 promoter from rice.
13. Method according to any one of items 1 to 12, wherein said nucleic acid encoding a C3H-like polypeptide is of plant origin, preferably the family Medicago, more preferably from Medicago truncatula.
14. Plant or part thereof, including seeds, obtainable by a method according to any one of items 1 to 13, wherein said plant or part thereof comprises a recombinant nucleic acid encoding a C3H-like polypeptide.
15. Construct comprising:
   (i) nucleic acid encoding a C3H-like polypeptide as defined in any one of items 1 to 6;
   (ii) one or more control sequences capable of driving expression of the nucleic acid sequence of (a); and optionally
   (iii) a transcription termination sequence.
16. Construct according to item 15, wherein one of said control sequences is a constitutive promoter, preferably a GOS2 promoter, most preferably a GOS2 promoter from rice.
17. Use of a construct according to item 15 or 16 in a method for making plants having increased yield, particularly increased biomass and/or increased seed yield relative to control plants.
18. Plant, plant part or plant cell transformed with a construct according to item 15 or 16.
19. Method for the production of a transgenic plant having increased yield, particularly increased biomass and/or increased seed yield relative to control plants, comprising:
   (i) introducing and expressing in a plant a nucleic acid encoding a C3H-like polypeptide as defined in any one or more of items 1 to 6; and
   (ii) cultivating the plant cell under conditions promoting plant growth and development.
20. Transgenic plant having increased yield, particularly increased biomass and/or increased seed yield, relative to control plants, resulting from modulated expression of a nucleic acid encoding a C3H-like polypeptide as defined in any one or more of items 1 to 6, or a transgenic plant cell derived from said transgenic plant.
21. Transgenic plant according to item 14, 18 or 20, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
22. Harvestable parts of a plant according to item 21, wherein said harvestable parts are preferably shoot biomass and/or seeds.
23. Products derived from a plant according to item 21 and/or from harvestable parts of a plant according to item 22.
24. Use of a nucleic acid encoding a C3H-like polypeptide in increasing yield, particularly in increasing seed yield and/or shoot biomass in plants, relative to control plants.

### 2. SPATULA-Like (SPT) Polypeptides

1. A method for enhancing yield-related traits in plants relative to control plants, comprising modulating expression in a plant of a nucleic acid encoding an SPT-like polypeptide comprising: each of the following, preferably from N-terminus to C-terminus:
   Motif I: an amphipathic helix comprising EEISTFLHQLLH, or a motif having in increasing order of preference at least 60%, 65%, 70%, 75%, 80%, 85%, 90%, 95% or more sequence identity to Motif I; and
   Motif II: an acidic domain comprising DLGDFSCDSEK or a motif having in increasing order of preference at least 60%, 65%, 70%, 75%, 80%, 85%, 90%, 95% or more sequence identity to Motif II; and
   Motif III: a bHLH domain comprising: AAEVHNLSEKRRRSRINEKMKALQNLIPNSNKT DKASMLDEAIEYLKQL or a motif having in increasing order of preference at least 60%, 65%, 70%, 75%, 80%, 85%, 90%, 95% or more sequence identity to Motif III.
2. Method according to item 1, wherein the SPT-like polypeptide further comprises one or more serine-rich regions.
3. Method according to item 1 or 2, wherein the bHLH domain further comprises one or more nuclear localisation signals (NLS).
4. Method according to any one of items 1 to 3, wherein the SPT-like polypeptide comprises a beta strand adjacent the bHLH domain nearest the C-terminal region, which beta strand preferably comprises QLQVQMLTM.
5. Method according to any one of items 1 to 4, wherein said modulated expression is effected by introducing and expressing in a plant a nucleic acid encoding an SPT-like polypeptide.
6. Method according to any one of items 1 to 5, wherein said nucleic acid encoding an SPT-like polypeptide encodes any one of the proteins listed in Table A2 or is a portion of such a nucleic acid, or a nucleic acid capable of hybridising with such a nucleic acid.
7. Method according to any one of items 1 to 6, wherein said nucleic acid sequence encodes an orthologue or paralogue of any of the proteins given in Table A2.
8. Method according to any preceding item, wherein said enhanced yield-related traits comprise increased yield, preferably increased biomass and/or increased seed yield relative to control plants.
9. Method according to any one of items 1 to 8, wherein said enhanced yield-related traits are obtained under non-stress conditions.
10. Method according to any one of items 1 to 9, wherein said enhanced yield-related traits are obtained under conditions of drought stress, salt stress or nitrogen deficiency.
11. Method according to any one of items 3 to 8, wherein said nucleic acid is operably linked to a constitutive promoter, preferably to a GOS2 promoter, most preferably to a GOS2 promoter from rice.
12. Method according to any one of items 1 to 11, wherein said nucleic acid encoding an SPT-like polypeptide is of plant origin, preferably from the family Salicaceae, more preferably from the genus Populus, most preferably from Populus trichocarpa.
13. Plant or part thereof, including seeds, obtainable by a method according to any one of items 1 to 12, wherein said plant or part thereof comprises a recombinant nucleic acid encoding an SPT-like polypeptide as defined in any one of items 1 to 4.
14. Construct comprising:
   (i) nucleic acid encoding an SPT-like polypeptide as defined in any one of items 1 to 4;
   (ii) one or more control sequences capable of driving expression of the nucleic acid sequence of (a); and optionally
   (iii) a transcription termination sequence.
15. Construct according to item 14, wherein one of said control sequences is a constitutive promoter, preferably a GOS2 promoter, most preferably a GOS2 promoter from rice.
16. Use of a construct according to item 14 or 15 in a method for making plants having increased yield, particularly increased biomass and/or increased seed yield relative to control plants.
17. Plant, plant part or plant cell transformed with a construct according to item 14 or 15.
18. Method for the production of a transgenic plant having increased yield, particularly increased biomass and/or increased seed yield relative to control plants, comprising:
   (i) introducing and expressing in a plant a nucleic acid encoding an SPT-like polypeptide as defined in any one of items 1 to 4; and
   (ii) cultivating the plant cell under conditions promoting plant growth and development.
19. Transgenic plant having increased yield, particularly increased biomass and/or increased seed yield, relative to control plants, resulting from modulated expression of a nucleic acid encoding an SPT-like polypeptide as defined in any one of items 1 to 4, or a transgenic plant cell derived from said transgenic plant.
20. Transgenic plant according to item 13, 17 or 19, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
21. Harvestable parts of a plant according to item 20, wherein said harvestable parts are preferably shoot biomass and/or seeds.
22. Products derived from a plant according to item 20 and/or from harvestable parts of a plant according to item 21.
23. Use of a nucleic acid encoding an SPT-like polypeptide as defined in any one of items 1 to 4 in increasing yield, particularly in increasing seed yield and/or shoot biomass in plants, relative to control plants.

### 3. IDI2 (Iron Deficiency Induced 2) Polypeptides

1. A method for enhancing yield-related traits in plants relative to control plants, comprising modulating expression in a plant of a nucleic acid encoding an IDI2 polypeptide, wherein said IDI2 polypeptide comprises an IF-2B domain.
2. Method according to item 1, wherein said IDI2 polypeptide comprises one or more of the motifs represented by any of SEQ ID NO: 141 to SEQ ID NO: 146.
3. Method according to item 1 or 2, wherein said modulated expression is effected by introducing and expressing in a plant a nucleic acid encoding an IDI2 polypeptide.
4. Method according to any one of items 1 to 3, wherein said nucleic acid encoding an IDI2 polypeptide encodes any one of the proteins listed in Table A3 or is a portion of such a nucleic acid, or a nucleic acid capable of hybridising with such a nucleic acid.
5. Method according to any one of items 1 to 4, wherein said nucleic acid sequence encodes an orthologue or paralogue of any of the proteins given in Table A3.
6. Method according to any preceding item, wherein said enhanced yield-related traits comprise increased yield, preferably increased seed yield relative to control plants.
7. Method according to any one of items 1 to 6, wherein said enhanced yield-related traits are obtained under conditions of nitrogen deficiency.
8. Method according to any one of items 3 to 7, wherein said nucleic acid is operably linked to a constitutive promoter, preferably to a GOS2 promoter, most preferably to a GOS2 promoter from rice.
9. Method according to any one of items 1 to 8, wherein said nucleic acid encoding an IDI2 polypeptide is of plant origin, preferably from a monocotyledonous plant, further preferably from the family Poaceae, more preferably from the genus Saccharum, most preferably from Saccharum officinarum.
10. Plant or part thereof, including seeds, obtainable by a method according to any one of items 1 to 9, wherein said plant or part thereof comprises a recombinant nucleic acid encoding an IDI2 polypeptide.
11. Construct comprising:
   (i) nucleic acid encoding an IDI2 polypeptide as defined in items 1 or 2;
   (ii) one or more control sequences capable of driving expression of the nucleic acid sequence of (a); and optionally
   (iii) a transcription termination sequence,
12. Construct according to item 11, wherein one of said control sequences is a constitutive promoter, preferably a GOS2 promoter, most preferably a GOS2 promoter from rice.
13. Use of a construct according to item 11 or 12 in a method for making plants having increased yield, particularly increased seed yield relative to control plants.
14. Plant, plant part or plant cell transformed with a construct according to item 11 or 12.
15. Method for the production of a transgenic plant having increased yield, particularly increased seed yield relative to control plants, comprising:
   (i) introducing and expressing in a plant a nucleic acid encoding an IDI2 polypeptide as defined in item 1 or 2; and
   (ii) cultivating the plant cell under conditions promoting plant growth and development.
16. Transgenic plant having increased yield, particularly increased seed yield, relative to control plants, resulting from modulated expression of a nucleic acid encoding an IDI2 polypeptide as defined in item 1 or 2, or a transgenic plant cell derived from said transgenic plant.
17. Transgenic plant according to item 10, 14 or 16, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
18. Harvestable parts of a plant according to item 17, wherein said harvestable parts preferably are seeds.
19. Products derived from a plant according to item 17 and/or from harvestable parts of a plant according to item 18.
20. Use of a nucleic acid encoding an IDI2 polypeptide in increasing yield, particularly in increasing seed yield in plants, relative to control plants.
21. An isolated nucleic acid molecule selected from:
   (i) a nucleic acid represented by any of SEQ ID NO: 139, 157, 164, 169, 171, 186;
   (ii) the complement of a nucleic acid represented by any of SEQ ID NO: 139, 157, 164, 169, 171, 186;
   (iii) a nucleic acid encoding an IDI2 polypeptide having, in increasing order of preference, at least 50%, 55%, 60%, 65%, 70%, 75%, 80%, 85%, 90%, 95%, 96%, 97%, 98%, 99% or more sequence identity to the amino acid sequences represented by any of SEQ ID NO: 140, 202, 209, 214, 216, 231, and comprising one or more of the motifs 1 to 6.
22. An isolated polypeptide selected from:
   (i) an amino acid sequence represented by any of SEQ ID NO: 140, 202, 209, 214, 216, 231;
   (ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequences represented by any one of SEQ ID NO: 140, 202, 209, 214, 216, 231, and comprising one or more of the motifs 1 to 6;
   derivatives of any of the amino acid sequences given in (i) or (ii) above.
      4. eIF4F-Like Protein Complex
1. A method for enhancing yield-related traits in plants relative to control plants, comprising modulating the activity of eIF4F-like protein complex by modulation and expression of its subunit polypeptides and/or isoforms thereof and/or by modulating the level of the eIF4F-like protein complex, wherein said eIF4F-like protein complex comprises the subunits eIF4G, eIF4A and eIF4E or isoforms thereof, comprising respectively the following CC domains with the PFam accession numbers:
   (i) for eIF4G polypeptides: MA3 (PFam accession number: PF02847) and MIF4G (PFam accession number: PF02854);
   (ii) for eIF4A polypeptides: DEAD (PFam accession number: PF00270) and Helicase_C(PFam accession number: PF00271);
   (iii) for eIF4E polypeptydes: IF4E (PFam accession number: PF01652).
2. A method, according to item 1, wherein said eIF4G subunit polypeptide comprises a CC domain
   (i) as represented by SEQ ID NO: 240, and/or
   (ii) preferably having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to eIF4G polypeptides represented by SEQ ID NO: 241.
3. A method, according to item 1, wherein said eIF4A subunit polypeptide comprises a CC domain
   (i) as represented by SEQ ID NO: 300, and/or
   (ii) preferably having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to eIF4G polypeptides represented by SEQ ID NO: 301.
4. A method, according to item 1, wherein said eIF4E subunit polypeptide comprises a CC domain
   (i) as represented by SEQ ID NO: 560, and/or
   (ii) preferably having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to eIF4G polypeptides represented by SEQ ID NO: 561.
5. Method according to items 1 or 2, wherein said eIF4G subunit polypeptides comprise the following motifs:
   Motif 7: KAV[LF]EPTFCPMYA[QL]LCSDLNEKLP[PS]FPS[ED]EPGGKEITFKRVLLN[NI]C QEAF or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 7;
   Motif 8: CP[AE]EENVEAIC[QH]FFNTIGKQLDE[SN]PKSRRIND[MVT]YF[SIN][RQ]LKEL [TS][TS]NPQLAPR or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 8.
   Motif 9: T[AG]P[DE]QE[ML]ERRDKERLVKLRTLGNIRLIGELLKQKMVPEKIVHHIVQEL LG or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 9;
   or
   Motif 10: TPQNF[ED][KR]LFEQVKAVNIDN[AV]VTL[TN]GVISQIF[DE]KALMEPTFCEMY ANFCFH or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 10;
   Motif 11: IGELYKK[RK]MLTERIMHECIKKLLGQYQ[DN]PDEE[DN][IV]E[AS]LCKLMSTIG EMIDH or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 11;
   Motif 12: LSNN[MQ][KN]LSSRVRFMLKD[ASV]IDLRKNKWQQRRKVEGPKKIEEVHRDA AQERQ or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 12.
6. Method according to item 5, wherein said eIF4G subunit polypeptides is preferably a eIF4 isoG polypeptide and comprise the following motifs:
   Motif 7: KAV[LF]EPTFCPMYA[QL]LCSDLNEKLP[PS]FPS[ED]EPGGKEITFKRVLLN[NI]CQEAF or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 7;
   Motif 8: CP[AE]EENVEAIC[QH]FFNTIGKQLDE[SN]PKSRRIND[MVT]YF[SIN][RQ]LKEL [TS][TS]NPQLAPR or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 8.
   Motif 9: T[AG]P[DE]QE[ML]ERRDKERLVKLRTLGNIRLIGELLKQKMVPEKIVHHIVQEL LG or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 9.
7. Method according to item 1 or 3, wherein said eIF4A subunit polypeptides comprise the following motifs:
   Motif 13: RDELTLEGIKQF[YF]V[NA]V[ED][KR]EEWK[LF][DE]TLCDLY[ED]TL [AT]ITQ [SA]VIF or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 13.
   Motif 14: SLVINYDLP[TN][QN][PR]E[NL]Y[LI]HRIGRSGRFGRKGVAINF or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 14.
   Motif 15: MG[LI][QK]E[ND]LLRGIYAYGFEKPSAIQQR[GA][IV]VP[FI][CI]KG[LR]DVI[QA]QAQSGTGKT[AS][TM][FI] or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 15.
8. Method according to item 1 or 4, wherein said eIF4E subunit polypeptides comprise the following motifs:
   Motif 16: YTFSTVE[ED]FW[SG]LYNNIH[HR]PSKLAVGADF[HY]CFK[NH]KIEPKWEDP [VI]CANGGKW or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 16;
   Motif 17: T[SC]WLYTLLA[ML]IGEQFD[HY]GD[ED]ICGAVV[NS]VR or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 17;
   Motif 18: E[KR]I[AS][LI]WTKNA[AS]NE[AST]AQ[VL]SIGKQWKEFLDYN[DE][TS]IGFIFH [ED]DA or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 18;
   or
   Motif 19: WCLYDQ[IV]F[KR]PSKLP[GA]NADFHLFKAG[VI]EPKWEDPECANGGKW or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 19;
   Motif 20: L[ED]TMWLETLMALIGEQFD[ED][AS][DE][ED]ICGWASVR or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 20;
   Motif 21: QDKL[SA]LWT[KR][TN]A[AS]NEA[AV]QM[SG]IG[RK]KWKE[IV]ID or a motif having in an increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to Motif 21.
9. Method, according to any of items 1 to 8, wherein said modulating expression of at least one of the said subunits eIF4E, eIF4G and eIF4A is effected by introducing and expressing of at least a nucleic acid encoding one of the eIF4F subunits polypeptides or a portion of at least such nucleic acids, or a nucleic acid capable of hybridising with such a nucleic acid.
10. Method, according to items 1, 2, 5 or 6, wherein said nucleic acid encodes the eIF4G subunit polypeptide and/or its isoforms or a portion of such a nucleic acid, or a nucleic acid capable of hybridising with such a nucleic acid, being the eIF4F subunit polypeptide preferably the eIF4 isoG subunit.
11. Method, according to item 1, 3 or 7, wherein said nucleic acid encodes the eIF4A subunit polypeptide and/or its isoforms or a portion of such a nucleic acid, or a nucleic acid capable of hybridising with such a nucleic acid, being the eIF4F subunit preferably the eIF4A subunit.
12. Method, according to item 1, 4 or 8, wherein said nucleic acid encodes the eIF4E subunit polypeptide and/or its isoforms, subunit or a portion of such a nucleic acid, or a nucleic acid capable of hybridising with such a nucleic acid, being the eIF4F subunit preferably the eIF4 isoE subunit.
13. Method, according to any of the items 1 to 12, wherein said nucleic acids, or a portion of such a nucleic acid, or a nucleic acid capable of hybridising with such a nucleic acid encoding for eIF4F subunits polypeptides are overexpressed, preferably those encoding for eIF4G and/or eIF4A and/or their isoforms, particularly those encoding for eIF4 isoG and/or eIF4A.
14. Method according to any one of items 1 to 13, wherein said nucleic acids sequences encodes an orthologue or paralogue of any of the polypeptides given in Tables A4.
15. Method according to any of items 1 to 14, wherein said enhanced yield-related traits comprise increased yield, preferably increased biomass and/or increased seed yield relative to control plants.
16. Method according to any one of items 1 to 15, wherein said enhanced yield-related traits are obtained under non-stress conditions.
17. Method according to any one of items 1 to 16, wherein said enhanced yield-related traits are obtained under conditions of drought stress, salt stress or nitrogen deficiency.
18. Method according to any one of items 3 to 17, wherein said nucleic acid is operably linked to a constitutive promoter, preferably to a GOS2 promoter, most preferably to a GOS2 promoter from rice.
19. Method according to any one of items 1 to 18, wherein said nucleic acid encoding at least an eIF4F polypeptide subunit is of plant origin, preferably from a dicotyledonous plant, further preferably from the family Brassicaceae, more preferably from the genus Arabidopsis, most preferably from Arabidopsis thaliana.
20. Plant or part thereof, including seeds, obtainable by a method according to any one of items 1 to 19, wherein said plant or part thereof comprises at least a recombinant nucleic acid encoding an eIF4F polypeptide subunit.
21. Construct comprising:
   (i) nucleic acid encoding at least an eIF4F polypeptide subunit as defined in items 1 or 2;
   (ii) one or more control sequences capable of driving expression of the nucleic acid sequence of (a); and optionally
   (iii) a transcription termination sequence.
22. Construct according to item 21, wherein one of said control sequences is a constitutive promoter, preferably a GOS2 promoter, most preferably a GOS2 promoter from rice.
23. Use of a construct according to item 21 or 22 in a method for making plants having increased yield, particularly increased biomass and/or increased seed yield relative to control plants.
24. Plant, plant part or plant cell transformed with a construct according to item 21 or 22.
25. Method for the production of a transgenic plant having increased yield, particularly increased biomass and/or increased seed yield relative to control plants, comprising:
   (i) introducing and expressing in a plant a nucleic acid encoding at least an eIF4F polypeptide subunit as defined in item 1 or 2; and
   (ii) cultivating the plant cell under conditions promoting plant growth and development.
26. Transgenic plant having increased yield, particularly increased biomass and/or increased seed yield, relative to control plants, resulting from modulated expression of at least a nucleic acid encoding a at least an eIF4F polypeptide subunit as defined in item 1 or 2, or a transgenic plant cell derived from said transgenic plant.
27. Transgenic plant according to item 20, 24 or 26, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
28. Harvestable parts of a plant according to item 27, wherein said harvestable parts are preferably shoot biomass and/or seeds.
29. Products derived from a plant according to item 27 and/or from harvestable parts of a plant according to item 28.
30. Use of a nucleic acid encoding at least an eIF4F polypeptide subunit in increasing yield, particularly in increasing seed yield and/or shoot biomass in plants, relative to control plants.
31. An isolated nucleic acid molecule selected from:
   (i) a nucleic acid represented by SEQ ID NO: 306;
   (ii) the complement of a nucleic acid represented by SEQ ID NO: 306;
   (iii) a nucleic acid encoding the polypeptide as represented by any one of SEQ ID NO: 307, preferably as a result of the degeneracy of the genetic code, said isolated nucleic acid can be derived from a polypeptide sequence as represented by SEQ ID NO: 307 and further preferably confers enhanced yield-related traits relative to control plants;
   (iv) a nucleic acid having, in increasing order of preference at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with any of the nucleic acid sequences of Tables A4 and further preferably conferring enhanced yield-related traits relative to control plants;
   (v) a nucleic acid molecule which hybridizes with a nucleic acid molecule of (i) to (iv) under stringent hybridization conditions and preferably confers enhanced yield-related traits relative to control plants;
   (vi) a nucleic acid encoding at least an eIF4F subunit polypeptide having, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence represented by SEQ ID NO: 307 and any of the other amino acid sequences in Tables A4 and preferably conferring enhanced yield-related traits relative to control plants.
32. An isolated polypeptide selected from:
   (i) an amino acid sequence represented by SEQ ID NO: 307;
   (ii) an amino acid sequence having, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence represented by SEQ ID NO: 307 and any of the other amino acid sequences in Tables A4 and preferably conferring enhanced yield-related traits relative to control plants.
   (iii) derivatives of any of the amino acid sequences given in (i) or (ii) above.

### 5. GR-RBP (Glycine Rich-RNA Binding Protein) Polypeptides

1. A method for enhancing yield-related traits in plants relative to control plants, comprising modulating expression in a plant of a nucleic acid encoding a Glycine-Rich RNA Binding Protein (GR-RBP polypeptide), wherein said GR-RBP polypeptide comprises a RNA Recognition Motif 1 (PFam accession PF00076, RRM_{—}1).
2. Method according to item 1, wherein said GR-RBP polypeptide comprises one or more of the signature sequences or motifs given in SEQ ID NO: 828 to SEQ ID NO: 837.
3. Method according to item 1 or 2, wherein said modulated expression is effected by introducing and expressing in a plant a nucleic acid encoding a GR-RBP polypeptide.
4. Method according to any one of items 1 to 3, wherein said nucleic acid encoding a GR-RBP polypeptide encodes any one of the proteins listed in Table A5 or is a portion of such a nucleic acid, or a nucleic acid capable of hybridising with such a nucleic acid.
5. Method according to any one of items 1 to 4, wherein said nucleic acid sequence encodes an orthologue or paralogue of any of the proteins given in Table A5.
6. Method according to any preceding item, wherein said enhanced yield-related traits comprise increased early vigour and/or increased yield, preferably increased biomass and/or increased seed yield relative to control plants.
7. Method according to any one of items 1 to 6, wherein said enhanced yield-related traits are obtained under conditions of drought stress.
8. Method according to any one of items 1 to 6, wherein said enhanced yield-related traits are obtained under non-stress conditions.
9. Method according to any one of items 3 to 8, wherein said nucleic acid is operably linked to a constitutive promoter, preferably to a GOS2 promoter, most preferably to a GOS2 promoter from rice.
10. Method according to any one of items 1 to 9, wherein said nucleic acid encoding a GR-RBP polypeptide is of plant origin, preferably from a monocotyledonous plant, further preferably from the family Poaceae, more preferably from the genus Oryza, most preferably from Oryza sativa.
11. Plant or part thereof, including seeds, obtainable by a method according to any one of items 1 to 10, wherein said plant or part thereof comprises a recombinant nucleic acid encoding a GR-RBP polypeptide.
12. Construct comprising:
   (i) nucleic acid encoding a GR-RBP polypeptide as defined in items 1 or 2;
   (ii) one or more control sequences capable of driving expression of the nucleic acid sequence of (a); and optionally
   (iii) a transcription termination sequence.
13. Construct according to item 12, wherein one of said control sequences is a constitutive promoter, preferably a GOS2 promoter, most preferably a GOS2 promoter from rice.
14. Use of a construct according to item 12 or 13 in a method for making plants having increased yield, particularly increased biomass and/or increased seed yield relative to control plants.
15. Plant, plant part or plant cell transformed with a construct according to item 12 or 13.
16. Method for the production of a transgenic plant having increased yield, particularly increased biomass and/or increased seed yield relative to control plants, comprising:
   (i) introducing and expressing in a plant a nucleic acid encoding a GR-RBP polypeptide as defined in item 1 or 2; and
   (ii) cultivating the plant cell under conditions promoting plant growth and development.
17. Transgenic plant having increased yield, particularly increased early vigour, increased biomass and/or increased seed yield, relative to control plants, resulting from modulated expression of a nucleic acid encoding a GR-RBP polypeptide as defined in item 1 or 2, or a transgenic plant cell derived from said transgenic plant.
18. Transgenic plant according to item 11, 15 or 17, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
19. Harvestable parts of a plant according to item 18, wherein said harvestable parts are preferably shoot biomass and/or seeds.
20. Products derived from a plant according to item 18 and/or from harvestable parts of a plant according to item 19.
21. Use of a nucleic acid encoding a GR-RBP polypeptide in increasing yield, particularly in increasing early vigour, seed yield and/or shoot biomass in plants, relative to control plants.
22. An isolated nucleic acid molecule selected from:
   (i) a nucleic acid represented by any of SEQ ID NO: 848, 849, 851, 852, 853, 854, 857, 862, 873, 874, 875, 876, 878, 879, 893, 897, 898, 900, 901, 905, 928, 931, 932, 933, 934, 937;
   (ii) the complement of a nucleic acid represented by any of SEQ ID NO: 848, 849, 851, 852, 853, 854, 857, 862, 873, 874, 875, 876, 878, 879, 893, 897, 898, 900, 901, 905, 928, 931, 932, 933, 934, 937;
   (iii) a nucleic acid encoding a GR-RBP polypeptide having, in increasing order of preference, at least 50%, 55%, 60%, 65%, 70%, 75%, 80%, 85%, 90%, 95%, 96%, 97%, 98%, 99% or more sequence identity to the amino acid sequences represented by any of SEQ ID NO: 945, 946, 948, 949, 950, 951, 854, 959, 970, 971, 972, 973, 975, 976, 990, 994, 995, 997, 998, 1002, 1025, 1028, 1029, 1030, 1031, 1034, and comprising signature sequence 3 (SEQ ID NO: 830) and signature sequence 4 (SEQ ID NO: 831).
23. An isolated polypeptide selected from:
   (i) an amino acid sequence represented by any of SEQ ID NO: 945, 946, 948, 949, 950, 951, 854, 959, 970, 971, 972, 973, 975, 976, 990, 994, 995, 997, 998, 1002, 1025, 1028, 1029, 1030, 1031, 1034;
   (ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequences represented by any one of SEQ ID NO: 945, 946, 948, 949, 950, 951, 854, 959, 970, 971, 972, 973, 975, 976, 990, 994, 995, 997, 998, 1002, 1025, 1028, 1029, 1030, 1031, 1034, and comprising signature sequence 3 (SEQ ID NO: 830) and signature sequence 4 (SEQ ID NO: 831);
   (iii) derivatives of any of the amino acid sequences given in (i) or (ii) above.

### DESCRIPTION OF FIGURES

The present invention will now be described with reference to the following figures in which:

FIG. 1 is a multiple alignment of C3H-like polypeptide squences. Alignment of polypeptide sequences was performed using the ClustalW 2.0 algorithm of progressive alignment (Thompson et al. (1997) Nucleic Acids Res 25:4876-4882; Chema et al. (2003). Nucleic Acids Res 31:3497-3500) with standard setting (slow alignment, similarity matrix: Gonnet, gap opening penalty 10, gap extension penalty: 0.2). Minor manual editing was done to further optimise the alignment.

FIG. 2 shows a phylogenetic tree. The phylogenetic tree was constructed using a neighbour-joining clustering algorithm as provided in the AlignX programme from the Vector NTI (Invitrogen).

FIG. 3 represents the binary vector used for increased expression in Oryza sativa of a C3H-like-encoding nucleic acid under the control of a rice GOS2 promoter (pGOS2).

FIG. 4 shows a multiple alignment. Alignment of polypeptide sequences was performed using the ClustalW 2.0 algorithm of progressive alignment (Thompson et al. (1997) Nucleic Acids Res 25:4876-4882; Chema et al. (2003). Nucleic Acids Res 31:3497-3500) with standard setting (slow alignment, similarity matrix: Gonnet, gap opening penalty 10, gap extension penalty: 0.2). Minor manual editing was done to further optimise the alignment.

FIG. 5 shows a phylogenetic tree of SPT-like polypeptides. The tree was constructed using a neighbour-joining clustering algorithm as provided in the AlignX programme from the Vector NTI (Invitrogen).

FIG. 6 represents the binary vector used for increased expression in Oryza sativa of an SPT-like-encoding nucleic acid under the control of a rice GOS2 promoter (pGOS2).

FIG. 7 represents the domain structure of SEQ ID NO: 140 with the IF-2B (PF01008) domain indicated in italics and the conserved motifs 4 to 6 underlined.

FIG. 8 represents a multiple alignment of IDI2 polypeptides from the A and B group.

FIG. 9 shows phylogenetic tree of IDI2 polypeptides, SEQ ID NO: 140 corresponds to Saccof_IDI2 in the A group. The sequences were aligned using MAFFT and were visualised with Dendroscope (Huson et al. (2007), BMC Bioinformatics 8(1):460). The corresponding SEQ ID Nos can be found in Table A3.

FIG. 10 represents the binary vector used for increased expression in Oryza sativa of an IDI2-encoding nucleic acid under the control of a rice GOS2 promoter (pGOS2)

FIG. 11 represents the composition eIF4F polypeptide with its main subunits eIF4G, eIF4E and eIF4A.

FIG. 12 represents the circular phylogram of selected eIF4G and isoG proteins. The proteins were aligned using MUSCLE 3.7 (Edgar (2004), Nucleic Acids Research 32(5): 1792-97). A neighbor-joining tree was calculated using QuickTree 1.1 (Howe et al. (2002), Bioinformatics 18(11): 1546-7). Support of the major branching after 100 bootstrap repetitions is indicated. A circular phylogram was drawn using Dendroscope 2.0.1 (Huson et al. (2007), BMC Bioinformatics 8(1):460). O. sativa eIF4 isoG, indicated in bold black.

FIG. 13 shows the phylogenetic tree of selected eIF4E and isoE proteins. The alignment was generated using MAFFT (Katoh and Toh (2008) Briefings in Bioinformatics 9:286-298). A neighbour-joining tree was calculated using QuickTree (Howe et al. (2002), Bioinformatics 18(11): 1546-7), 100 bootstrap repetitions. The circular phylogram was drawn using Dendroscope (Huson et al. (2007), BMC Bioinformatics 8(1):460). Confidence for 100 bootstrap repetitions is indicated for major branching. See the sequence listing for species abbreviations.

FIG. 14 represents the phylogenetic tree of selected eIF4A polypeptides. The alignment was generated using MAFFT (Katoh and Toh (2008) Briefings in Bioinformatics 9:286-298). A neighbour-joining tree was calculated using QuickTree (Howe et al. (2002), Bioinformatics 18(11): 1546-7), 100 bootstrap repetitions. The circular phylogram was drawn using Dendroscope (Huson et al. (2007), BMC Bioinformatics 8(1):460). Confidence for 100 bootstrap repetitions is indicated for major branching. See the sequence listing for species abbreviations.

FIG. 15 represents the binary vector used for increased expression in Oryza sativa of an eIF4 isoG or eIF4A encoding nucleic acid under the control of a rice GOS2 promoter (pGOS2).

FIG. 16 represents the domain structure of SEQ ID NO: 827 with the conserved RRM_{—}1 domain (PF00076, in bold italics) and the gly-rich region in bold. The GGYGG and GGYG signature sequences are underlined.

FIG. 17 represents a multiple alignment of various GR-RBP polypeptides constructed using VNTI. Conserved amino acids are shaded and a consensus sequences is reproduced below the alignment.

FIG. 18 shows phylogenetic tree of GR-RBP polypeptides, SEQ ID NO: 827 (boxed) is part of Glade A. The sequences were aligned using MAFFT and were visualised with Dendroscope (Huson et al. (2007), BMC Bioinformatics 8(1):460).

FIG. 19 represents the binary vector used for increased expression in Oryza sativa of a GR-RBP-encoding nucleic acid under the control of a rice GOS2 promoter (pGOS2).

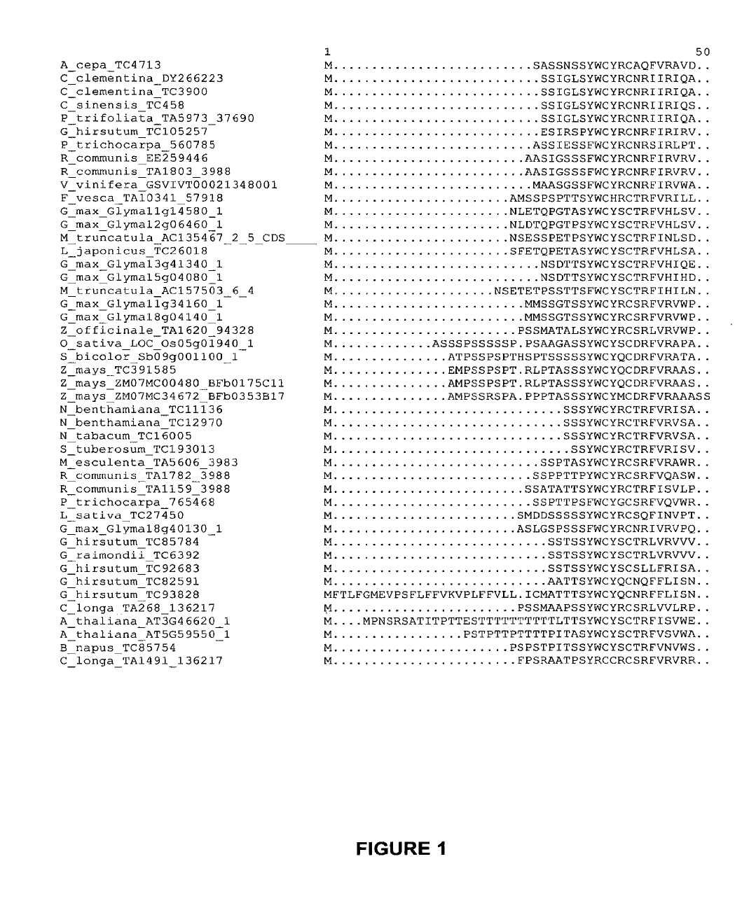

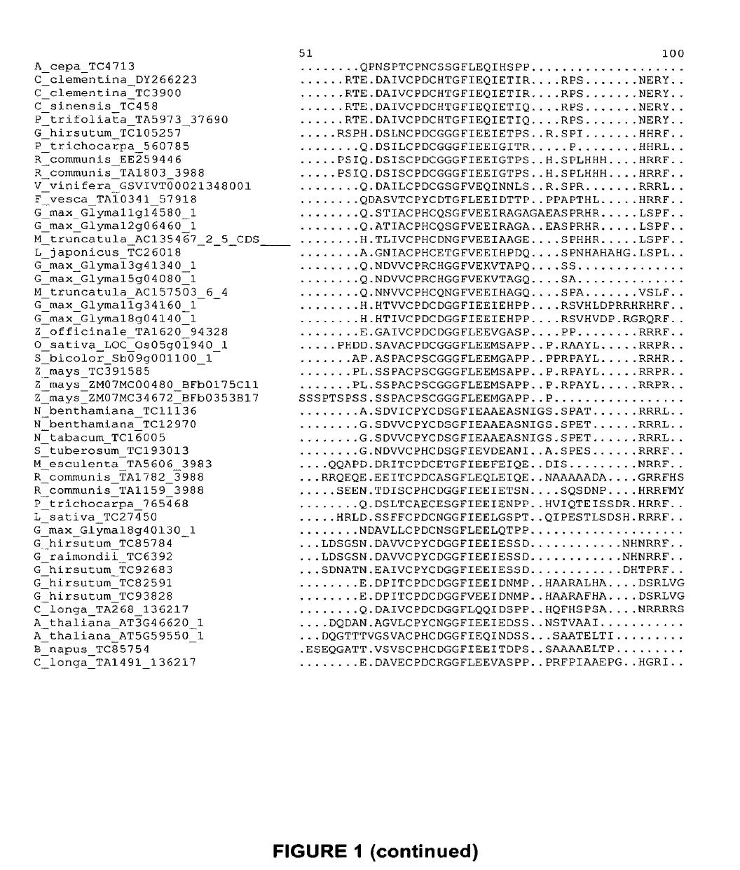

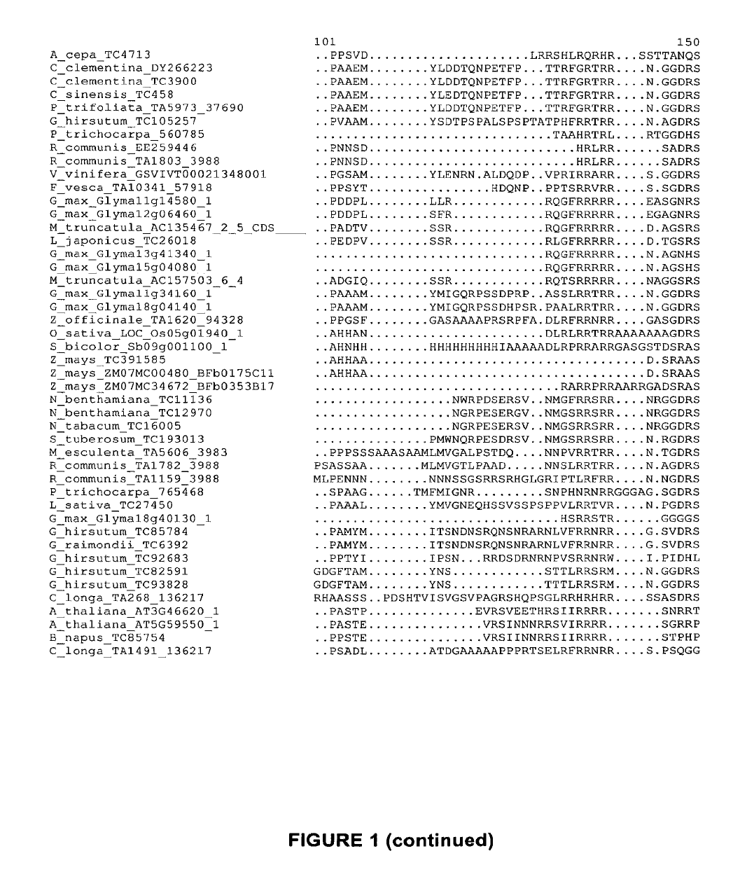

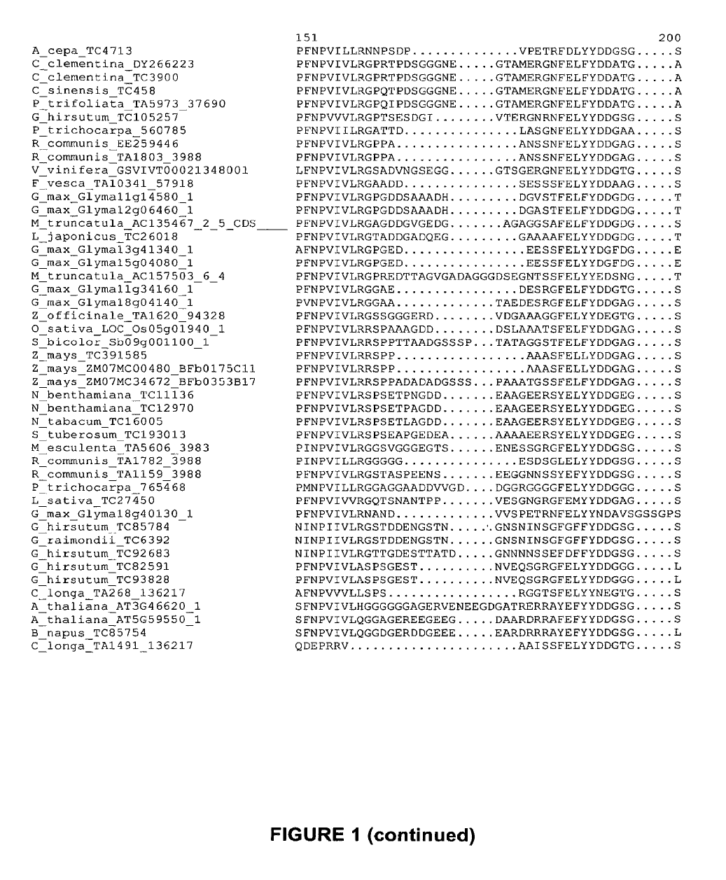

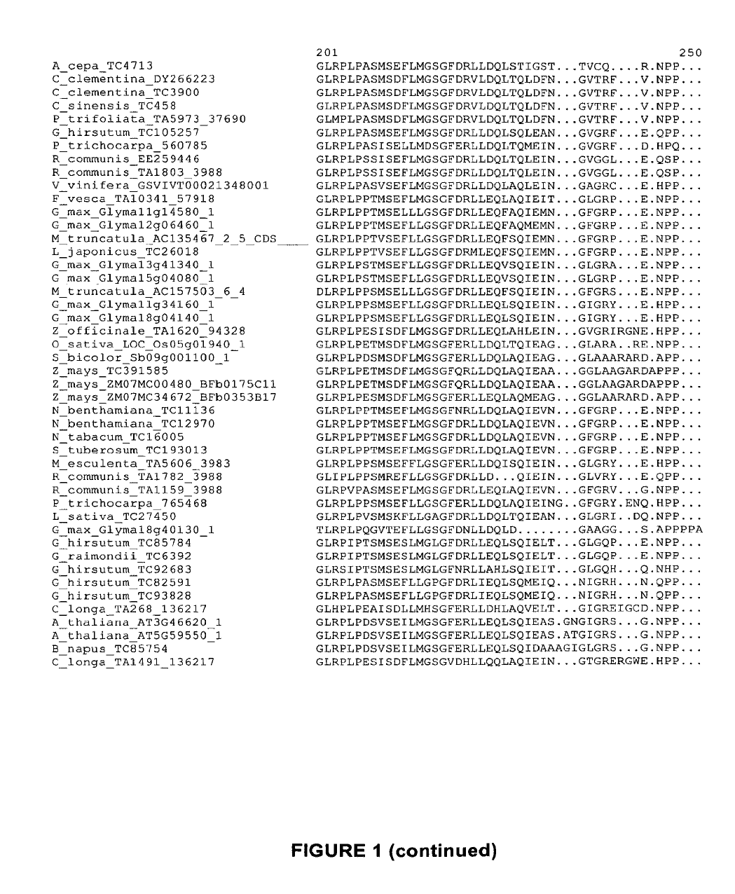

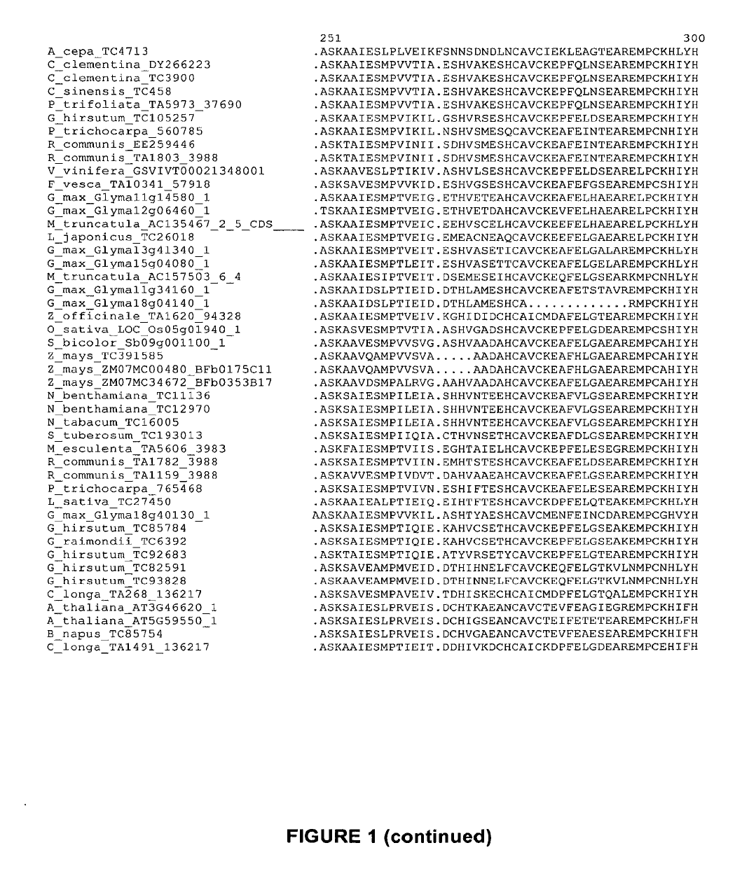

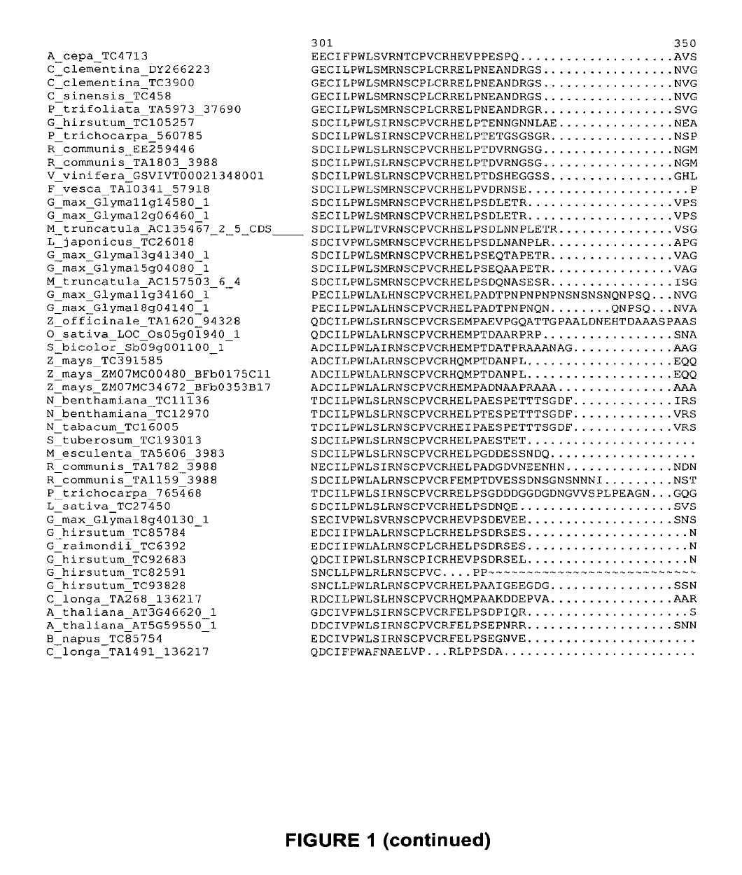

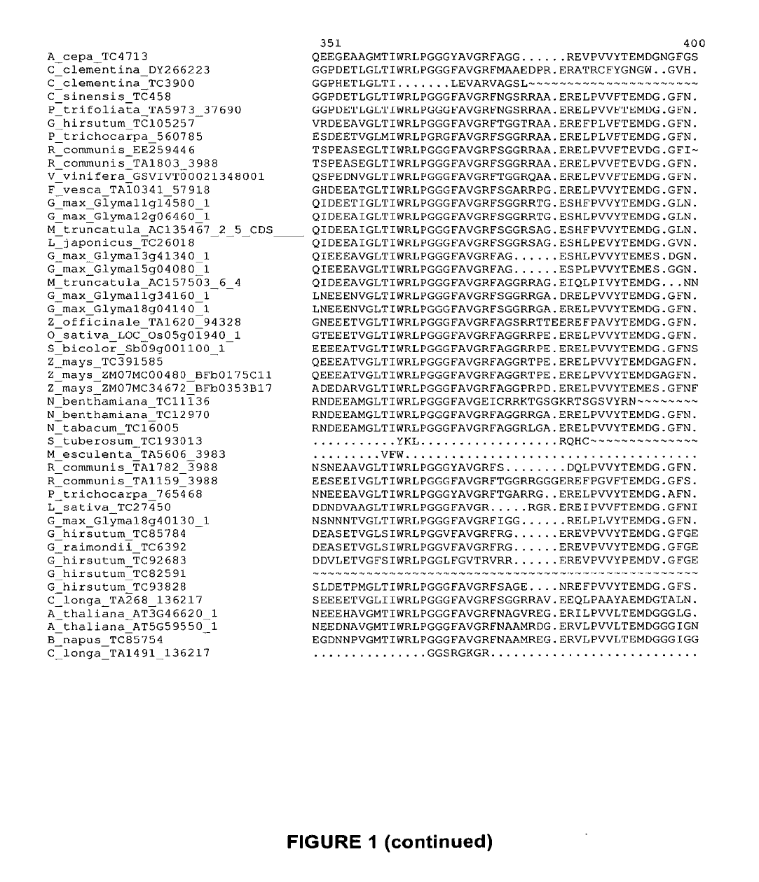

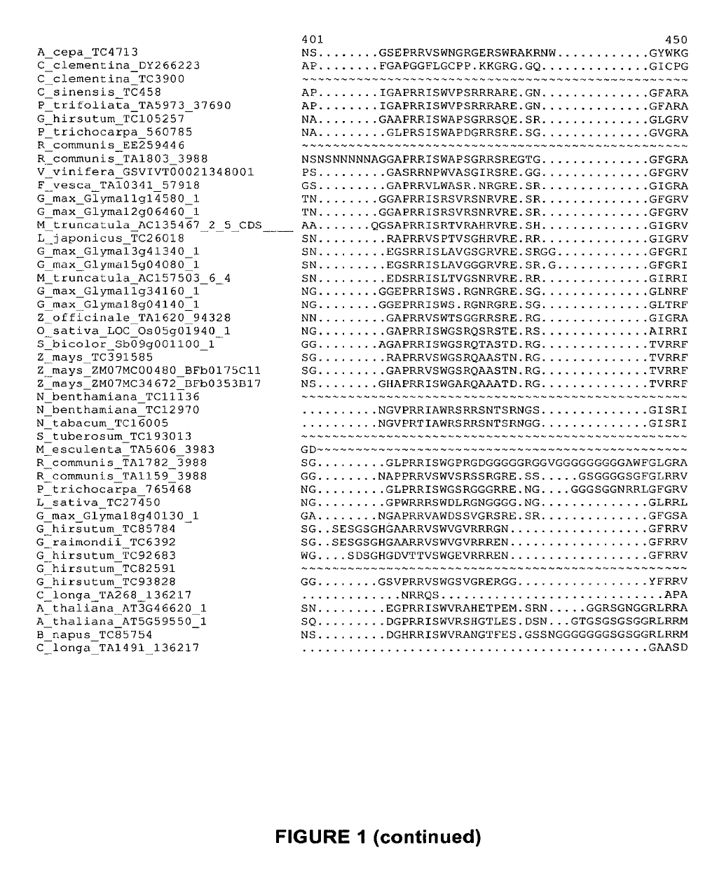

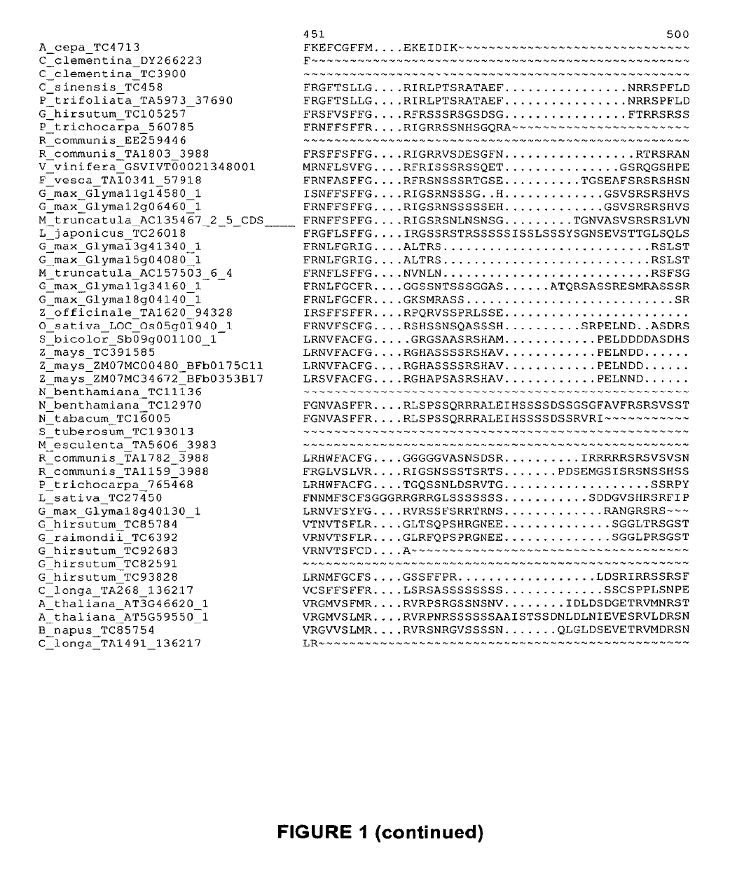

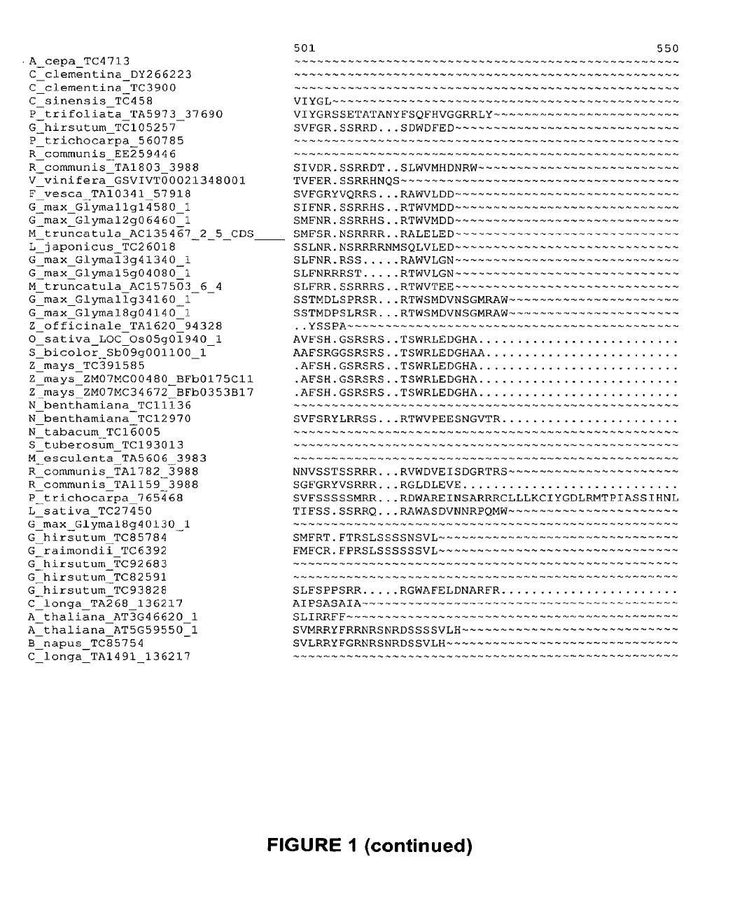

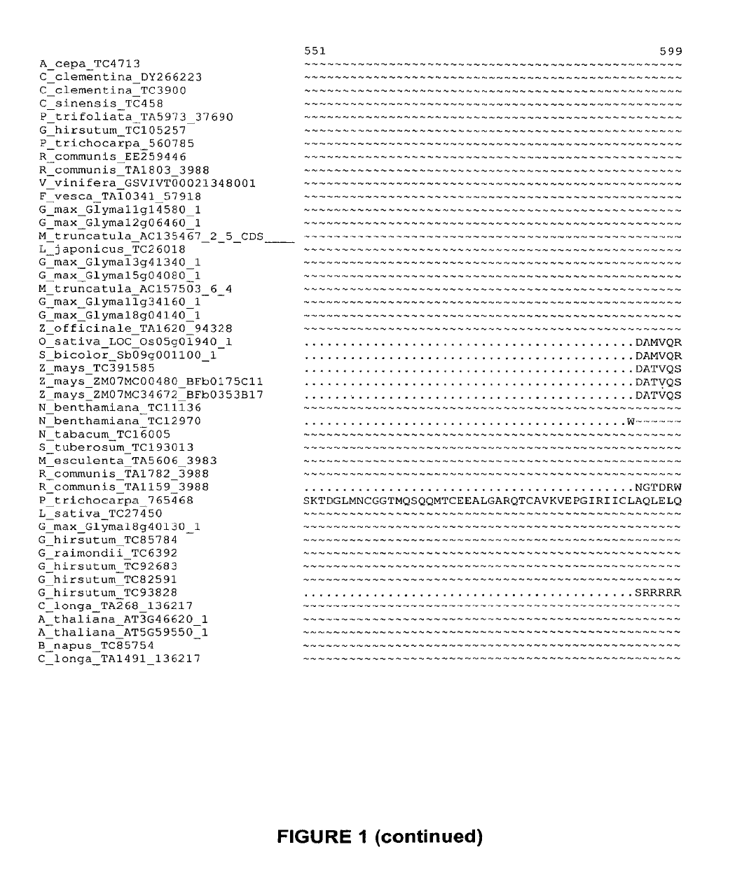

### EXAMPLES

The present invention will now be described with reference to the following examples, which are by way of illustration alone. The following examples are not intended to completely define or otherwise limit the scope of the invention.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

### Identification of Sequences Related to the Nucleic Acid Sequence Used in the Methods of the Invention

Sequences (full length cDNA, ESTs or genomic) related to SEQ ID NO: 1 and SEQ ID NO: 2 were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program finds regions of local similarity between sequences by comparing nucleic acid or polypeptide sequences to sequence databases and by calculating the statistical significance of matches. For example, the polypeptide encoded by SEQ ID NO: 1 was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

### 1.1. C3H-Like Polypeptides

Table A1 provides a list of nucleic acid sequences related to SEQ ID NO: 1 and SEQ ID NO: 2.

TABLE A1
Examples of C3H-like sequences:

| | | Nucleic | |
|---|---|---|---|
| | | acid | Polypeptide |
| Name | Organism | SEQ ID NO: | SEQ ID NO: |
| M_truncatula_AC135467_2_5_CDS | Medicago truncatula | 1 | 2 |
| A_cepa_TC4713 | Allium cepa | 3 | 4 |
| A_thaliana_AT3G46620_1 | Arabidopsis thaliana | 5 | 6 |
| A_thaliana_AT5G59550_1 | Arabidopsis thaliana | 7 | 8 |
| B_napus_TC85754 | Brassica napus | 9 | 10 |
| C_clementina_DY266223 | Citrus clementina | 11 | 12 |
| C_clementina_TC3900 | Citrus clementina | 13 | 14 |
| C_longa_TA1491_136217 | Curcuma longa | 15 | 16 |
| C_longa_TA268_136217 | Curcuma longa | 17 | 18 |
| C_sinensis_TC458 | Citrus sinensis | 19 | 20 |
| F_vesca_TA10341_57918 | Fragaria vesca | 21 | 22 |
| G_hirsutum_TC105257 | Gossypium hirsutum | 23 | 24 |
| G_hirsutum_TC82591 | Gossypium hirsutum | 25 | 26 |
| G_hirsutum_TC85784 | Gossypium hirsutum | 27 | 28 |
| G_hirsutum_TC92683 | Gossypium hirsutum | 29 | 30 |
| G_hirsutum_TC93828 | Gossypium hirsutum | 31 | 32 |
| G_max_Glyma11g14580_1 | Glycine max | 33 | 34 |
| G_max_Glyma11g34160_1 | Glycine max | 35 | 36 |
| G_max_Glyma12g06460_1 | Glycine max | 37 | 38 |
| G_max_Glyma13g41340_1 | Glycine max | 39 | 40 |
| G_max_Glyma15g04080_1 | Glycine max | 41 | 42 |
| G_max_Glyma18g04140_1 | Glycine max | 43 | 44 |
| G_max_Glyma18g40130_1 | Glycine max | 45 | 46 |
| G_raimondii_TC6392 | Gossypium raimondii | 47 | 48 |
| L_japonicus_TC26018 | Lotus japonica | 49 | 50 |
| L_sativa_TC27450 | Lactuca sativa | 51 | 52 |
| M_esculenta_TA5606_3983 | Manihot esculenta | 53 | 54 |
| M_truncatula_AC157503_6_4 | Medicago truncatula | 55 | 56 |
| N_benthamiana_TC11136 | Nicotiana benthamiana | 57 | 58 |
| N_benthamiana_TC12970 | Nicotiana benthamiana | 59 | 60 |
| N_tabacum_TC16005 | Nicotiana tabacum | 61 | 62 |
| O_sativa_LOC_Os05g01940_1 | Oryza sativa | 63 | 64 |
| P_trichocarpa_560785 | Populus trichocarpa | 65 | 66 |
| P_trichocarpa_765468 | Populus trichocarpa | 67 | 68 |
| P_trifoliata_TA5973_37690 | Poncirus trifoliata | 69 | 70 |
| R_communis_EE259446 | Ricinus communis | 71 | 72 |
| R_communis_TA1159_3988 | Ricinus communis | 73 | 74 |
| R_communis_TA1782_3988 | Ricinus communis | 75 | 76 |
| R_communis_TA1803_3988 | Ricinus communis | 77 | 78 |
| S_bicolor_Sb09g001100_1 | Sorghum bicolor | 79 | 80 |
| S_tuberosum_TC193013 | Solanum tuberosum | 81 | 82 |
| V_vinifera_GSVIVT00021348001 | Vitis vinifera | 83 | 84 |
| Z_mays_TC391585 | Zea mays | 85 | 86 |
| Z_mays_ZM07MC00480_BFb0175C11 | Zea mays | 87 | 88 |
| Z_mays_ZM07MC34672_BFb0353B17 | Zea mays | 89 | 90 |
| Z_officinale_TA1620_94328 | Zingiber officinale | 91 | 92 |

In some instances, related sequences have tentatively been assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. In other instances, special nucleic acid sequence databases have been created for particular organisms, such as by the Joint Genome Institute. Further, access to proprietary databases, has allowed the identification of novel nucleic acid and polypeptide sequences.

### 1.2. SPATULA-Like (SPT) Polypeptides

Table A2 provides a list of sequences related to SEQ ID NO: 96 and SEQ ID NO: 97

TABLE A2
Examples of SPT-like sequences:

| | | Nucleic | |
|---|---|---|---|
| | | acid | |
| | | SEQ ID | Polypeptide |
| Name | Source organism | NO: | SEQ ID NO: |
| Poptr_SPT | Populus trichocarpa | 96 | 97 |
| Arath_SPT | Arabidopsis thaliana | 98 | 99 |
| Brana_SPT | Brassica napus | 100 | 101 |
| Carpa_SPT | Carica papaya | 102 | 103 |
| Cicle_SPT | Citrus clementina | 104 | 105 |
| Escca_SPT | Eschscholzia californica | | 106 |
| Fra x ana_SPT_{—} | Fragaria x ananassa | 107 | 108 |
| Glyma_SPT_{—} | Glycine max | 109 | 110 |
| Goshi_SPT | Gossypium hirsutum | 111 | 112 |
| Medtr_SPT | Medicago truncatula | 113 | 114 |
| Nicta_SPT | Nicotiana tabacum | 115 | 116 |
| Solly_SPT | Solanium lycopersicum | 117 | 118 |
| Vinvi_SPT | Vitis vinifera | 119 | 120 |
| Glyma_SPT like 1 | Glycine max | 121 | 122 |
| Glyma_SPT like 2 | Glycine max | 123 | 124 |
| Solly_SPT like 1 | Solanium lycopersicum | 125 | 126 |
| Solly_SPT like 2 | Solanium lycopersicum | 127 | 128 |
| Orysa_SPT like | Oryza sativa | 129 | 130 |
| Arath_ALC | Arabidopsis thaliana | 131 | 132 |

In some instances, related sequences have tentatively been assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. On other instances, special nucleic acid sequence databases have been created for particular organisms, such as by the Joint Genome Institute.

### 1.3. IDI2 (Iron Deficiency Induced 2) Polypeptides

Table A3 provides a list of nucleic acid sequences related to the nucleic acid sequence used in the methods of the present invention.

TABLE A3
Examples of IDI2 polypeptides:

| | Nucleic | |
|---|---|---|
| | acid | |
| | SEQ | Polypeptide |
| Plant Source | ID NO: | SEQ ID NO: |
| Saccharum officinarum IDI2 | 139 | 140 |
| Chlorella 140697 | 150 | 195 |
| Hordeum vulgare TA31842_4513 | 151 | 196 |
| Ostreococcus RCC809 | 152 | 197 |
| Oryza sativa BGIOSIBCE033922 | 153 | 198 |
| Oryza sativa BGIOSIBSE037940 | 154 | 199 |
| Oyrza sativa CR292756 | 155 | 200 |
| Oyryza sativa CX116019 | 156 | 201 |
| Oryza sativa Os11g0216900 | 157 | 202 |
| Ostreococcus taurii 8569 | 158 | 203 |
| Pinus pinaster TA4183_71647 | 159 | 204 |
| Phaeodactylum tricornutum 23811 | 160 | 205 |
| Sorghum bicolour Sb05g008680.1 | 161 | 206 |
| Sorghum bicolour VTA25485_4558 | 162 | 207 |
| Saccharum officinarum TA35690_4547 | 163 | 208 |
| Triticum aestivum c54899571@13348 | 164 | 209 |
| Triticum aestivum CV772651 | 165 | 210 |
| Triticum aestivum TA67133_4565 | 166 | 211 |
| Thalassiosira pseudonana 35896 | 167 | 212 |
| Volvox carteri 59470 | 168 | 213 |
| Zea mays c57808725gm030403@2572 | 169 | 214 |
| Zea mays DQ244248 | 170 | 215 |
| Zea mays ZM07MC01636_57808725@1630 | 171 | 216 |
| Arabidopsis thaliana AT2G05830.1 | 172 | 217 |
| Brassica napus DY025654 | 173 | 218 |
| Citrus clementina DY262513 | 174 | 219 |
| Citrus clementina DY262587 | 175 | 220 |
| Citrus clementina DY263526 | 176 | 221 |
| Citrus clementina DY268933 | 177 | 222 |
| Citrus clementina TA2451_85681 | 178 | 223 |
| Carthamus tinctorius TA325_4222 | 179 | 224 |
| Glycine max Glyma09g08190.1 | 180 | 225 |
| Helianthus tuberosus TA3030_4233 | 181 | 226 |
| Ipomoea nil CJ747673 | 182 | 227 |
| Lactuca virosa TA2328_75947 | 183 | 228 |
| Phyllostachys nigra TA2942_3691 | 184 | 229 |
| Populus trichocarpa 832064 | 185 | 230 |
| Populus trichocarpa scaff_VI.1535 | 186 | 231 |
| Vitis shuttleworthii CN604099 | 187 | 232 |
| Vitis vinifera GSVIVT00016416001 | 188 | 233 |
| Vitis vinifera TA40906_29760 | 189 | 234 |
| Aquilegia formosa x pubescens TA9033_338618 | 190 | 235 |
| Gossypium hirsutum TA24273_3635 | 191 | 236 |
| Gossypium raimondii TA11759_29730 | 192 | 237 |
| Nicotiana tabacum TA17086_4097 | 193 | 238 |
| Solanum tuberosum TA31637_4113 | 194 | 239 |

In some instances, related sequences have tentatively been assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. On other instances, special nucleic acid sequence databases have been created for particular organisms, such as by the Joint Genome Institute. Further, access to proprietary databases, has allowed the identification of novel nucleic acid and polypeptide sequences.

1.4. eIF4F-Like Protein Complex

Tables A4a, A4b and A4c provide a list of nucleic acid sequences related to the nucleic acid sequence used in the methods of the present invention. Table(s) A4, as referred herein, means anyone or more of Tables A4a, A4b and A4c.

TABLE A4a
Examples of eIF4isoG-like polypeptides:

| | Nucleic acid | Polypeptide |
|---|---|---|
| Name | SEQ ID NO: | SEQ ID NO: |
| O. sativa_eIF4isoG | 240 | 241 |
| A. thaliana_AT5G57870.1 | 242 | 243 |
| G. max_Gm0025x00623 | 244 | 245 |
| G. max_Gm0071x00063 | 246 | 247 |
| M. truncatula_AC140546_1.5 | 248 | 249 |
| O. sativa_Os02g0611500 | 250 | 251 |
| P. trichocarpa_835841 | 252 | 253 |
| P. trichocarpa_835945 | 254 | 255 |
| R. communis_TA1365_3988 | 256 | 257 |
| S. bicolor_5286647 | 258 | 259 |
| S. tuberosum_TA26995_4113 | 260 | 261 |
| T. aestivum_TA60686_4565 | 262 | 263 |
| V. vinifera_GSVIVT00017187001 | 264 | 265 |
| V. vinifera_GSVIVT00032790001 | 266 | 267 |
| A. thaliana_AT3G60240.2 | 268 | 269 |
| G. max_Gm0072x00069 | 270 | 271 |
| G. max_Gm0119x00255 | 272 | 273 |
| M. truncatula_AC153354_6.5 | 274 | 275 |
| O. sativa_LOC_Os07g36940.1 | 276 | 277 |
| P. trichocarpa_scaff_40.82 | 278 | 279 |
| P. trichocarpa_scaff_II.1294 | 280 | 281 |
| V. vinifera_GSVIVT00019025001 | 282 | 283 |
| C. reinhardtii_147254 | 284 | 285 |
| Chlorella_142387 | 286 | 287 |
| P. patens_183347 | 288 | 289 |
| S. moellendorffii_437322 | 290 | 291 |
| V. carteri_103732 | 292 | 293 |
| O. RCC809_60557 | 294 | 295 |
| O. taurii_23433 | 296 | 297 |
| A. thaliana_AT2G24050.1 | 298 | 299 |

TABLE A4b
Examples of eIF4A-like polypeptides:

| | Nucleic acid | Polypeptide |
|---|---|---|
| Name | SEQ ID NO: | SEQ ID NO: |
| O. sativa_LOC_Os06g48750.1-mutant | 300 | 301 |
| O. sativa_LOC_Os06g48750.1 | 302 | 303 |
| O. sativa_LOC_Os02g05330.1 | 304 | 305 |
| A. aestivalis_676 | 306 | 307 |
| A. cepa_TA4214_4679 | 308 | 309 |
| A. formosa_x_pubescens_TA10474_338618 | 310 | 311 |
| A. formosa_x_pubescens_TA9867_338618 | 312 | 313 |
| A. officinalis_TA1117_4686 | 314 | 315 |
| A. thaliana_AT1G54270.1 | 316 | 317 |
| A. thaliana_AT1G72730.1 | 318 | 319 |
| A. thaliana_AT3G13920.1 | 320 | 321 |
| B. napus_TA25077_3708 | 322 | 323 |
| B. oleracea_TA5257_3712 | 324 | 325 |
| B. oleracea_TA5508_3712 | 326 | 327 |
| C. canephora_TA6150_49390 | 328 | 329 |
| C. reinhardtii_188942 | 330 | 331 |
| C. richardii_TA518_49495 | 332 | 333 |
| C. rumphii_TA1001_58031 | 334 | 335 |
| C. sinensis_TA11500_2711 | 336 | 337 |
| C. sinensis_TA12133_2711 | 338 | 339 |
| Citrus_x_paradisi_x_Poncirus_trifoliata_TA2371_309804 | 340 | 341 |
| E. huxleyi_413872 | 342 | 343 |
| F. vesca_TA10930_57918 | 344 | 345 |
| F. vesca_TA9647_57918 | 346 | 347 |
| G. hirsutum_TA20166_3635 | 348 | 349 |
| G. max_Gm0010x00368.1 | 350 | 351 |
| G. max_Gm0025x00441 | 352 | 353 |
| G. max_Gm0026x00612 | 354 | 355 |
| G. raimondii_TA10187_29730 | 356 | 357 |
| H. brasiliensis_TA107_3981 | 358 | 359 |
| H. exilis_TA612_400408 | 360 | 361 |
| H. vulgare_TA29331_4513 | 362 | 363 |
| L. japonicus_TA1252_34305 | 364 | 365 |
| L. japonicus_TA494_34305 | 366 | 367 |
| L. perennis_TA1391_43195 | 368 | 369 |
| L. sativa_TA1046_4236 | 370 | 371 |
| M. crystallinum_TA3938_3544 | 372 | 373 |
| M. domestica_TA24974_3750 | 374 | 375 |
| M. esculenta_TA5134_3983 | 376 | 377 |
| M. polymorpha_TA364_3197 | 378 | 379 |
| M. truncatula_AC136955_3.5 | 380 | 381 |
| M. truncatula_TA20612_3880 | 382 | 383 |
| Micromonas_TA67_392814 | 384 | 385 |
| N. benthamiana_TA9701_4100 | 386 | 387 |
| N. tabacum_TA13194_4097 | 388 | 389 |
| N. tabacum_TA14720_4097 | 390 | 391 |
| N. tabacum_X79004 | 392 | 393 |
| O. lucimarinus_32748 | 394 | 395 |
| O. taurii_28625 | 396 | 397 |
| P. abies_TA1392_3329 | 398 | 399 |
| P. deltoides_TA2215_3696 | 400 | 401 |
| P. engelmannii_x_glauca_TA4735_373101 | 402 | 403 |
| P. glauca_TA14843_3330 | 404 | 405 |
| P. glauca_TA15121_3330 | 406 | 407 |
| P. glaucum_TA261_4543 | 408 | 409 |
| P. patens_233331 | 410 | 411 |
| P. persica_TA3219_3760 | 412 | 413 |
| P. persica_TA4099_3760 | 414 | 415 |
| P. sativum_AY167671 | 416 | 417 |
| P. sitchensis_TA10673_3332 | 418 | 419 |
| P. taeda_TA1570_3352 | 420 | 421 |
| P. taeda_TA1624_3352 | 422 | 423 |
| P. taeda_TA6267_3352 | 424 | 425 |
| P. tremuloides_TA2301_3693 | 426 | 427 |
| P. trichocarpa_645764 | 428 | 429 |
| P. tricornutum_25743 | 430 | 431 |
| P. vulgaris_TA3080_3885 | 432 | 433 |
| S. bicolor_5283853 | 434 | 435 |
| S. bicolor_5291391 | 436 | 437 |
| S. henryi_TA238_13258 | 438 | 439 |
| S. lycopersicum_TA36357_4081 | 440 | 441 |
| S. moellendorffii_116103 | 442 | 443 |
| S. moellendorffii_143895 | 444 | 445 |
| S. propinquum_TA3625_132711 | 446 | 447 |
| S. tuberosum_TA24247_4113 | 448 | 449 |
| T. aestivum_TA61187_4565 | 450 | 451 |
| T. pratense_TA857_57577 | 452 | 453 |
| T. pseudonana_9716 | 454 | 455 |
| V. carteri_120953 | 456 | 457 |
| V. riparia_TA568_96939 | 458 | 459 |
| V. shuttleworthii_TA1952_246827 | 460 | 461 |
| V. vinifera_GSVIVT00032180001 | 462 | 463 |
| V. vinifera_TA37483_29760 | 464 | 465 |
| W. mirabilis_TA508_3377 | 466 | 467 |
| Z. mays_TA10949_4577999 | 468 | 469 |
| Z. mays_TA169666_4577 | 470 | 471 |
| Z. officinale_TA1475_94328 | 472 | 473 |
| A. anophagefferens_58937 | 474 | 475 |
| A. anophagefferens_70371 | 476 | 477 |
| A. formosa_x_pubescens_TA10839_338618 | 478 | 479 |
| A. formosa_x_pubescens_TA12575_338618 | 480 | 481 |
| A. formosa_x_pubescens_TA18893_338618 | 482 | 483 |
| A. thaliana_AT1G51380.1 | 484 | 485 |
| A. thaliana_AT3G19760.1 | 486 | 487 |
| C. reinhardtii_608 | 488 | 489 |
| C. sinensis_TA12419_2711 | 490 | 491 |
| Chlorella_29172 | 492 | 493 |
| Citrus_x_paradisi_x_Poncirus_trifoliata_TA3394_309804 | 494 | 495 |
| E. huxleyi_451926 | 496 | 497 |
| G. hirsutum_TA21351_3635 | 498 | 499 |
| G. max_Gm0120x00197 | 500 | 501 |
| G. raimondii_TA11359_29730 | 502 | 503 |
| H. vulgare_TA35263_4513 | 504 | 505 |
| L. serriola_TA2428_75943 | 506 | 507 |
| M. truncatula_TA22494_3880 | 508 | 509 |
| N. tabacum_TA14127_4097 | 510 | 511 |
| O. basilicum_TA2248_39350 | 512 | 513 |
| O. lucimarinus_26958 | 514 | 515 |
| O. RCC809_27976 | 516 | 517 |
| O. sativa_LOC_Os01g45190.1 | 518 | 519 |
| O. sativa_LOC_Os03g36930.1 | 520 | 521 |
| O. taurii_20289 | 522 | 523 |
| P. patens_109347 | 524 | 525 |
| P. patens_60709 | 526 | 527 |
| P. sativum_Y17186 | 528 | 529 |
| P. taeda_TA11536_3352 | 530 | 531 |
| P. taeda_TA13727_3352 | 532 | 533 |
| P. trichocarpa_832316 | 534 | 535 |
| P. trichocarpa_TA24057_3694 | 536 | 537 |
| P. tricornutum_41785 | 538 | 539 |
| R. communis_TA1264_3988 | 540 | 541 |
| S. bicolor_5285388 | 542 | 543 |
| S. lycopersicum_TA39656_4081 | 544 | 545 |
| S. moellendorffii_164382 | 546 | 547 |
| S. officinarum_TA29446_4547 | 548 | 549 |
| S. tuberosum_TA27838_4113 | 550 | 551 |
| T. aestivum_TA68913_4565 | 552 | 553 |
| T. pseudonana_354 | 554 | 555 |
| V. vinifera_GSVIVT00037338001 | 556 | 557 |
| Z. mays_TA13571_4577999 | 558 | 559 |

TABLE A4C
Examples of eIF4isoE-like polypeptides:

| | Nucleic acid | Polypeptide |
|---|---|---|
| Name | SEQ ID NO: | SEQ ID NO: |
| A. thaliana_AT1G29590.1#1 | 560 | 561 |
| A. thaliana_AT4G18040.1#1 | 562 | 563 |
| O. sativa_LOC_Os10g32970.1#1 | 564 | 565 |
| O. sativa_LOC_Os01g73880.1#1 | 566 | 567 |
| A. thaliana_AT1G29550.1#1 | 568 | 569 |
| B. napus_TA35293_3708#1 | 570 | 571 |
| B. rapa_DY010188#1 | 572 | 573 |
| B. rapa_TA7617_3711#1 | 574 | 575 |
| C. annuum_TA4459_4072#1 | 576 | 577 |
| C. clementina_TA6340_85681#1 | 578 | 579 |
| C. endivia_TA2025_114280#1 | 580 | 581 |
| C. intybus_TA694_13427#1 | 582 | 583 |
| C. maculosa_TA364_215693#1 | 584 | 585 |
| C. maculosa_TA5711_215693#1 | 586 | 587 |
| C. sinensis_TA13750_2711#1 | 588 | 589 |
| C. solstitialis_EH773887#1 | 590 | 591 |
| C. tinctorius_TA5460_4222#1 | 592 | 593 |
| E. esula_TA10897_3993#1 | 594 | 595 |
| F. vesca_TA13426_57918#1 | 596 | 597 |
| G. hirsutum_TA27730_3635#1 | 598 | 599 |
| G. hybrid_TA3072_18101#1 | 600 | 601 |
| G. max_Gm0030x00263#1 | 602 | 603 |
| G. raimondii_CO095282#1 | 604 | 605 |
| H. annuus_DY913126#1 | 606 | 607 |
| H. annuus_TA12162_4232#1 | 608 | 609 |
| H. annuus_TA15745_4232#1 | 610 | 611 |
| H. paradoxus_EL473808#1 | 612 | 613 |
| H. paradoxus_EL478502#1 | 614 | 615 |
| H. petiolaris_DY950915#1 | 616 | 617 |
| H. vulgare_gi_24285258#1 | 618 | 619 |
| I. nil_TA6867_35883#1 | 620 | 621 |
| L. perennis_TA3336_43195#1 | 622 | 623 |
| L. saligna_TA2850_75948#1 | 624 | 625 |
| L. sativa_TA3652_4236#1 | 626 | 627 |
| L. serriola_TA2077_75943#1 | 628 | 629 |
| L. virosa_DW170719#1 | 630 | 631 |
| M. domestica_TA27491_3750#1 | 632 | 633 |
| M. esculenta_TA8185_3983#1 | 634 | 635 |
| M. truncatula_AC183923_21.4#1 | 636 | 637 |
| N. tabacum_TA15108_4097#1 | 638 | 639 |
| N. tabacum_TA15811_4097#1 | 640 | 641 |
| N. tabacum_TA18292_4097#1 | 642 | 643 |
| P. deltoides_TA3856_3696#1 | 644 | 645 |
| P. persica_TA3622_3760#1 | 646 | 647 |
| P. sativum_TA561_3888#1 | 648 | 649 |
| P. trichocarpa_660574#1 | 650 | 651 |
| R. hybrid_TA805_128735#1 | 652 | 653 |
| S. bicolor_5283641#1 | 654 | 655 |
| S. habrochaites_TA2269_62890#1 | 656 | 657 |
| S. habrochaites_TA2286_62890#1 | 658 | 659 |
| S. lycopersicum_TA41869_4081#1 | 660 | 661 |
| S. lycopersicum_TA46570_4081#1 | 662 | 663 |
| S. officinarum_CA085501#1 | 664 | 665 |
| S. officinarum_TA33018_4547#1 | 666 | 667 |
| S. tuberosum_TA38547_4113#1 | 668 | 669 |
| S. tuberosum_TA40790_4113#1 | 670 | 671 |
| T. aestivum_TA62358_4565#1 | 672 | 673 |
| T. officinale_TA4584_50225#1 | 674 | 675 |
| V. vinifera_GSVIVT00007223001#1 | 676 | 677 |
| Z. aethiopica_TA1464_69721#1 | 678 | 679 |
| Z. mays_TA10333_4577999#1 | 680 | 681 |
| Z. mays_TA10334_4577999#1 | 682 | 683 |
| Z. officinale_TA1360_94328#1 | 684 | 685 |
| C. japonica_TA2318_3369#1 | 686 | 687 |
| M. polymorpha_TA2032_3197#1 | 688 | 689 |
| O. lucimarinus_35895#1 | 690 | 691 |
| O. taurii_27582#1 | 692 | 693 |
| P. glauca_TA18620_3330#1 | 694 | 695 |
| P. menziesii_TA2852_3357#1 | 696 | 697 |
| P. patens_162107#1 | 698 | 699 |
| P. patens_180874#1 | 700 | 701 |
| P. patens_227546#1 | 702 | 703 |
| P. patens_56790#1 | 704 | 705 |
| P. pinaster_TA3549_71647#1 | 706 | 707 |
| P. sitchensis_TA12910_3332#1 | 708 | 709 |
| P. taeda_TA117_3352#1 | 710 | 711 |
| P. taeda_TA8879_3352#1 | 712 | 713 |
| S. moellendorffii_119068#1 | 714 | 715 |
| A. majus_TA4832_4151#1 | 716 | 717 |
| A. thaliana_AT5G35620.1#1 | 718 | 719 |
| A. trichopoda_TA1884_13333#1 | 720 | 721 |
| B. napus_TA23235_3708#1 | 722 | 723 |
| B. oleracea_TA7150_3712#1 | 724 | 725 |
| B. oleracea_TA9753_3712#1 | 726 | 727 |
| C. annuum_TA4905_4072#1 | 728 | 729 |
| C. endivia_TA3234_114280#1 | 730 | 731 |
| C. intybus_EH698519#1 | 732 | 733 |
| C. sinensis_TA15477_2711#1 | 734 | 735 |
| C. tinctorius_EL398837#1 | 736 | 737 |
| C. tinctorius_TA3701_4222#1 | 738 | 739 |
| C. tinctorius_TA3908_4222#1 | 740 | 741 |
| E. esula_TA11865_3993#1 | 742 | 743 |
| F. arundinacea_TA4879_4606#1 | 744 | 745 |
| F. vesca_TA10417_57918#1 | 746 | 747 |
| G. arboreum_BF275433#1 | 748 | 749 |
| G. hirsutum_TA25457_3635#1 | 750 | 751 |
| G. max_TA47310_3847#1 | 752 | 753 |
| G. raimondii_CO091100#1 | 754 | 755 |
| H. annuus_TA11250_4232#1 | 756 | 757 |
| H. ciliaris_EL412673#1 | 758 | 759 |
| H. exilis_TA4524_400408#1 | 760 | 761 |
| H. paradoxus_TA2272_73304#1 | 762 | 763 |
| H. petiolaris_TA3720_4234#1 | 764 | 765 |
| H. tuberosus_TA3600_4233#1 | 766 | 767 |
| H. vulgare_BF265202#1 | 768 | 769 |
| I. batatas_TA3257_4120#1 | 770 | 771 |
| J. hindsii_x_regia_TA854_432290#1 | 772 | 773 |
| L. perennis_DW100049#1 | 774 | 775 |
| L. sativa_TA3660_4236#1 | 776 | 777 |
| L. serriola_DW115219#1 | 778 | 779 |
| L. serriola_TA139_75943#1 | 780 | 781 |
| L. tulipifera_TA1346_3415#1 | 782 | 783 |
| M. truncatula_AC174281_23.4#1 | 784 | 785 |
| N. tabacum_TA16190_4097#1 | 786 | 787 |
| O. basilicum_TA868_39350#1 | 788 | 789 |
| P. deltoides_CV130917#1 | 790 | 791 |
| P. trichocarpa_scaff_VIII.1581#1 | 792 | 793 |
| P. trichocarpa_scaff_X.371#1 | 794 | 795 |
| R. communis_TA2161_3988#1 | 796 | 797 |
| S. bicolor_5277963#1 | 798 | 799 |
| S. lycopersicum_TA42439_4081#1 | 800 | 801 |
| S. miltiorrhiza_TA1369_226208#1 | 802 | 803 |
| S. officinarum_TA33209_4547#1 | 804 | 805 |
| S. tuberosum_CK245580#1 | 806 | 807 |
| T. aestivum_TA69126_4565#1 | 808 | 809 |
| T. kok-saghyz_TA1330_333970#1 | 810 | 811 |
| V. vinifera_EE097579#1 | 812 | 813 |
| Z. mays_EE041506#1 | 814 | 815 |
| Z. mays_TA12850_4577999#1 | 816 | 817 |

In some instances, related sequences have tentatively been assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. On other instances, special nucleic acid sequence databases have been created for particular organisms, such as by the Joint Genome Institute. Further, access to proprietary databases, has allowed the identification of novel nucleic acid and polypeptide sequences.

### 1.5. GR-RBP (Glycine Rich-RNA Binding Protein) Polypeptides

Table A5 provides a list of nucleic acid sequences related to the nucleic acid sequence used in the methods of the present invention.

TABLE A5
Examples of GR-RBP polypeptides:

| | Nucleic | |
|---|---|---|
| | acid | |
| | SEQ | Polypeptide |
| Name | ID NO: | SEQ ID NO: |
| O. sativa_LOC_Os12g31800.1 | 826 | 827 |
| A. thaliana_AT4G13850.2 | 841 | 938 |
| A. thaliana_AT4G13850.3 | 842 | 939 |
| A. thaliana_AT4G13850.4 | 843 | 940 |
| Arabidopsis_thaliana_AJ002892 | 844 | 941 |
| Arabidopsis_thaliana_AY097374 | 845 | 942 |
| Arabidopsis_thaliana_BT002197 | 846 | 943 |
| Arabidopsis_thaliana_BT006315 | 847 | 944 |
| B. napus_BN06MC14993_44009177@14945 | 848 | 945 |
| B. napus_BN06MC33239_51481786@33086 | 849 | 946 |
| Bambusa_oldhamii_EU076902 | 850 | 947 |
| E. lagascae_s_el01277_t@402 | 851 | 948 |
| G. max_GM06MC37402_su20g11@36531 | 852 | 949 |
| H. vulgare_c63432693hv270303@6212 | 853 | 950 |
| H. vulgare_HV04MC07973_63432693@7969 | 854 | 951 |
| Hordeum_vulgare_subsp_vulgare_AK249796 | 855 | 952 |
| Hordeum_vulgare_subsp_vulgare_AK252775 | 856 | 953 |
| L. usitatissimum_c62280695@9780 | 857 | 954 |
| Nicotiana_sylvestris_D28862 | 858 | 955 |
| Nicotiana_tabacum_AY048972 | 859 | 956 |
| O. sativa_LOC_Os07g41120.1 | 860 | 957 |
| O. sativa_LOC_Os10g17454.2 | 861 | 958 |
| Oryza_sativa_Indica_Group_CT830471 | 862 | 959 |
| P. patens_167311 | 863 | 960 |
| P. patens_208328 | 864 | 961 |
| P. trichocarpa_707174 | 865 | 962 |
| Picea_sitchensis_EF083658 | 866 | 963 |
| Picea_sitchensis_EF086676 | 867 | 964 |
| Pisum_sativum_U81287 | 868 | 965 |
| Populus_trichocarpa_EF148189 | 869 | 966 |
| S. bicolor_Sb03g043760.1 | 870 | 967 |
| S. bicolor_Sb08g015580.1 | 871 | 968 |
| Solanum_lycopersicum_BT012756 | 872 | 969 |
| T. aestivum_c54626433@14323 | 873 | 970 |
| T. aestivum_c55526991@11638 | 874 | 971 |
| T. aestivum_c56257751@11019 | 875 | 972 |
| T. aestivum_TA06MC00270_56599813@270 | 876 | 973 |
| V. vinifera_GSVIVT00016201001 | 877 | 974 |
| Z. mays_ZM07MC15190_65293483@15154 | 878 | 975 |
| Z. mays_ZM07MC16747_65163049@16705 | 879 | 976 |
| Zea_mays_DQ245645 | 880 | 977 |
| Zea_mays_DQ245844 | 881 | 978 |
| Zea_mays_EU968589 | 882 | 979 |
| Zea_mays_BT033345 | 883 | 980 |
| Mesembryanthemum_crystallinum_AB294247 | 884 | 981 |
| Nicotiana_plumbaginifolia_X65117 | 885 | 982 |
| Nicotiana_sylvestris_X53942 | 886 | 983 |
| Nicotiana_sylvestris_X61113 | 887 | 984 |
| O. sativa_LOC_Os07g43810.1 | 888 | 985 |
| Persea_americana_AJ421780 | 889 | 986 |
| Picea_sitchensis_EF084744 | 890 | 987 |
| Picea_sitchensis_EF085266 | 891 | 988 |
| Spinacia_oleracea_U34742 | 892 | 989 |
| A. aestivalis_CON_13b-CS_AdonisPetal- | 893 | 990 |
| 11A15.b1@770 | | |
| Chorispora_bungeana_FJ356060 | 894 | 991 |
| Cryptomeria_japonica_AB254811 | 895 | 992 |
| Dianthus_caryophyllus_AB276043 | 896 | 993 |
| G. max_GM06MC14574_59593118@14360 | 897 | 994 |
| G. max_GM06MC35719_sq55b10@34881 | 898 | 995 |
| Glycine_max_AF169205 | 899 | 996 |
| H. vulgare_gi_13098745 | 900 | 997 |
| H. vulgare_gi_24273475 | 901 | 998 |
| Hordeum_vulgare_subsp_vulgare_U49482 | 902 | 999 |
| Nicotiana_glutinosa_AF005359 | 903 | 1000 |
| Nicotiana_tabacum_EU569289 | 904 | 1001 |
| O. sativa_LOC_Os12g43600.1 | 905 | 1002 |
| Oryza_rufipogon_CU405585 | 906 | 1003 |
| Oryza_rufipogon_CU405925 | 907 | 1004 |
| Oryza_rufipogon_CU406510 | 908 | 1005 |
| Oryza_sativa_Indica_Group_AF009411 | 909 | 1006 |
| Oryza_sativa_Indica_Group_AJ302060 | 910 | 1007 |
| Oryza_sativa_Indica_Group_CT828032 | 911 | 1008 |
| Oryza_sativa_Indica_Group_CT828687 | 912 | 1009 |
| Oryza_sativa_Japonica_Group_AF010580 | 913 | 1010 |
| Oryza_sativa_Japonica_Group_AJ002893 | 914 | 1011 |
| Oryza_sativa_Japonica_Group_AK059164 | 915 | 1012 |
| Oryza_sativa_Japonica_Group_AK111046 | 916 | 1013 |
| Oryza_sativa_Japonica_Group_AK119238 | 917 | 1014 |
| Picea_glauca_AF109917 | 918 | 1015 |
| Picea_sitchensis_EF082522 | 919 | 1016 |
| Populus_trichocarpa_EF144619 | 920 | 1017 |
| Prunus_avium_AY050483 | 921 | 1018 |
| Ricinus_communis_AJ245939 | 922 | 1019 |
| Rumex_obtusifolius_AJ441311 | 923 | 1020 |
| S. bicolor_Sb01g012300.1 | 924 | 1021 |
| Sinapis_alba_L31374 | 925 | 1022 |
| Sinapis_alba_L31377 | 926 | 1023 |
| Solanum_commersonii_Y12424 | 927 | 1024 |
| Solanum_tuberosum_Z49197 | 928 | 1025 |
| Sorghum_bicolor_AF310215 | 929 | 1026 |
| Sorghum_bicolor_X57662 | 930 | 1027 |
| T. aestivum_c50852885@10711 | 931 | 1028 |
| T. aestivum_c54623722@14648 | 932 | 1029 |
| T. aestivum_c57139332@11252 | 933 | 1030 |
| T. aestivum_c59884010@9282 | 934 | 1031 |
| Triticum_aestivum_AB272227 | 935 | 1032 |
| Triticum_aestivum_U32310 | 936 | 1033 |
| Z. mays_ZM07MC37068_60778288@36943 | 937 | 1034 |

In some instances, related sequences have tentatively been assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. On other instances, special nucleic acid sequence databases have been created for particular organisms, such as by the Joint Genome Institute. Further, access to proprietary databases, has allowed the identification of novel nucleic acid and polypeptide sequences.

### Example 2

### Alignment of Sequences Related to the Polypeptide Sequences Used in the Methods of the Invention

### 2.1. C3H-Like Polypeptides

Alignment of polypeptide sequences was performed using the ClustalW 2.0 algorithm of progressive alignment (Thompson et al. (1997) Nucleic Acids Res 25:4876-4882; Chema et al. (2003). Nucleic Acids Res 31:3497-3500) with standard setting (slow alignment, similarity matrix: Gonnet, gap opening penalty 10, gap extension penalty: 0.2). Minor manual editing was done to further optimise the alignment.

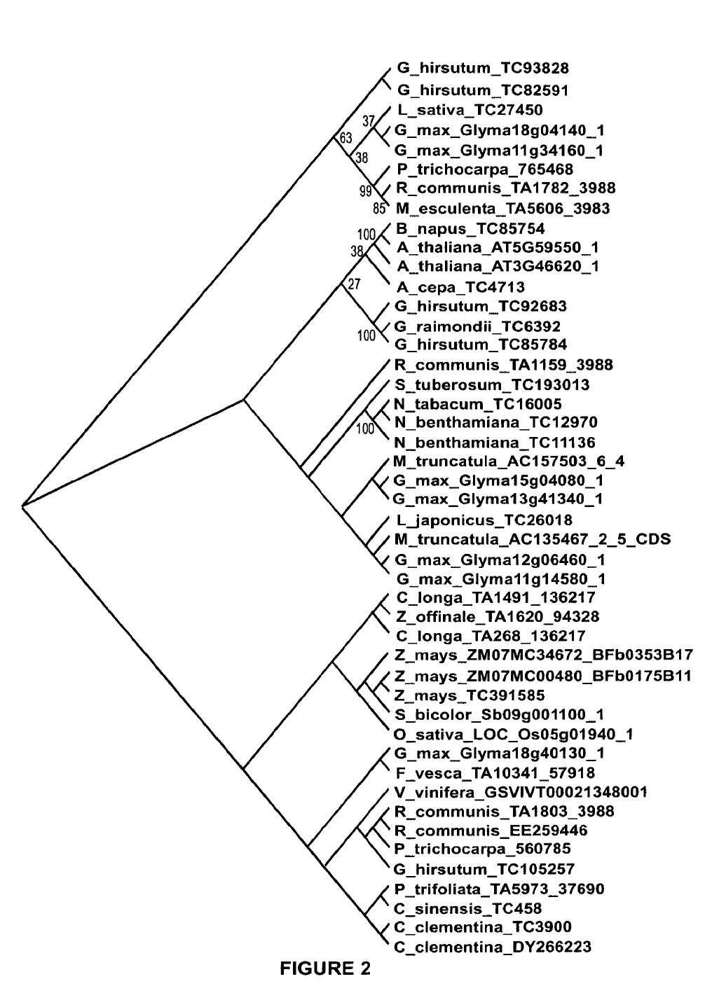

A phylogenetic tree of C3H-like polypeptides (FIG. 2) was constructed using a neighbour-joining clustering algorithm as provided in the AlignX programme from the Vector NTI (Invitrogen).

### 2.2. SPATULA-Like (SPT) Polypeptides

Alignment of polypeptide sequences was performed using the ClustalW 2.0 algorithm of progressive alignment (Thompson et al. (1997) Nucleic Acids Res 25:4876-4882; Chema et al. (2003). Nucleic Acids Res 31:3497-3500) with standard setting (slow alignment, similarity matrix: Gonnet, gap opening penalty 10, gap extension penalty: 0.2). Minor manual editing was done to further optimise the alignment.

A phylogenetic tree of SPATULA-LIKE polypeptides (FIG. 5) was constructed using a neighbour-joining clustering algorithm as provided in the AlignX programme from the Vector NTI (Invitrogen).

### 2.3. IDI2 (Iron Deficiency Induced 2) Polypeptides

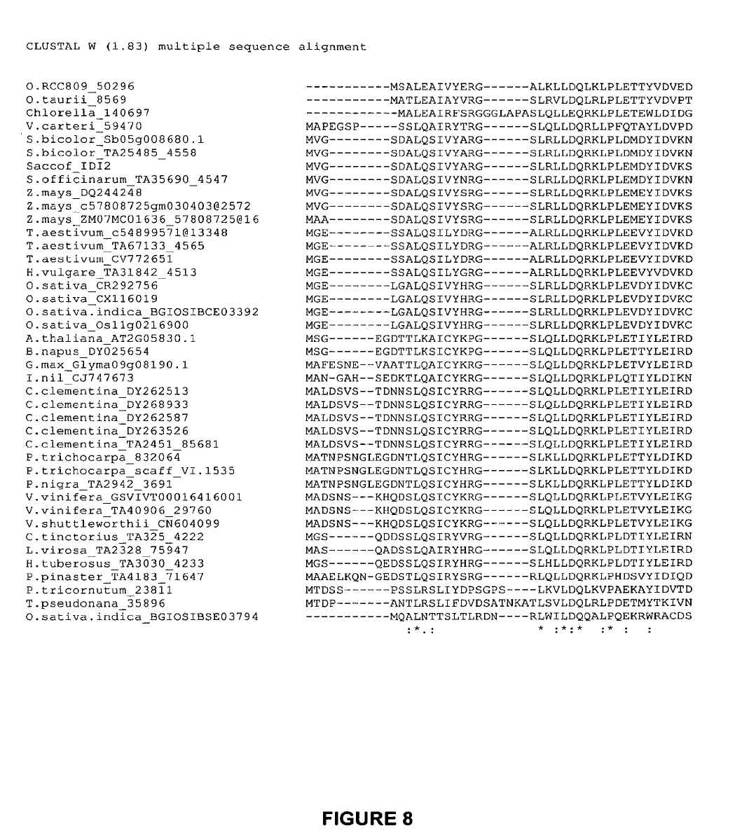

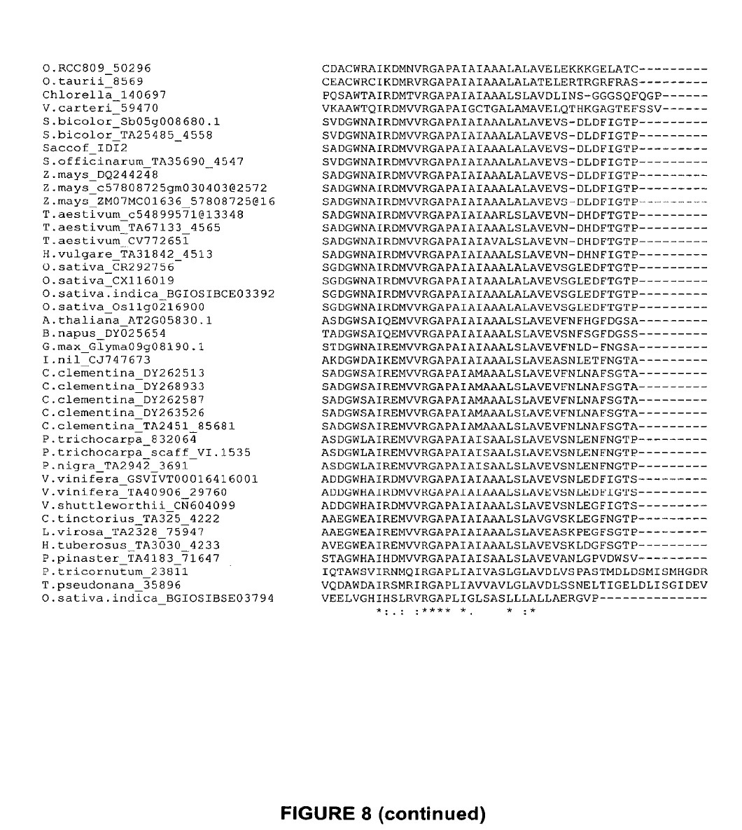

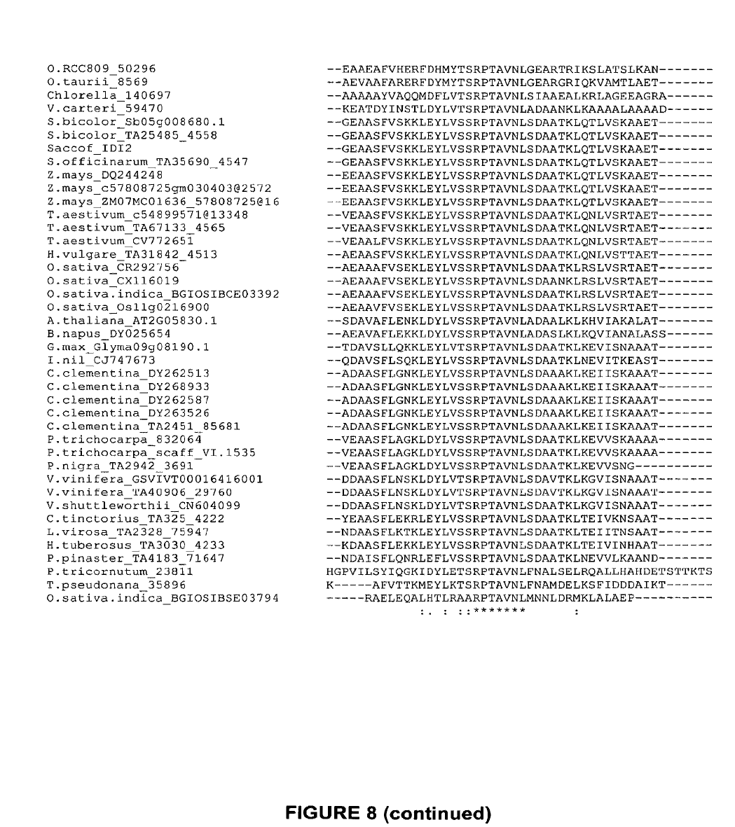

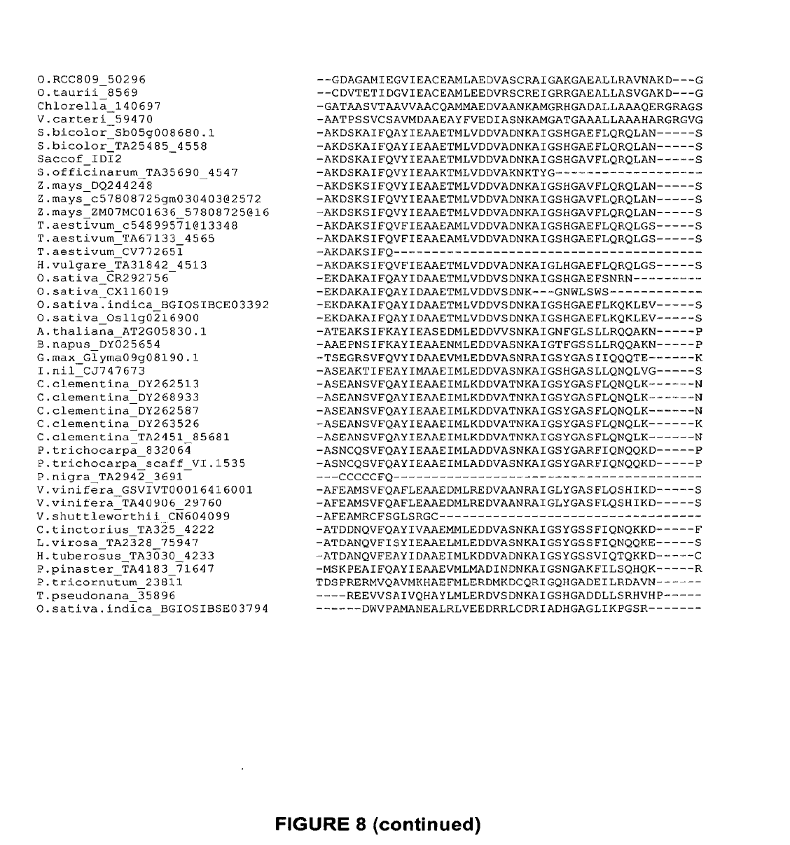

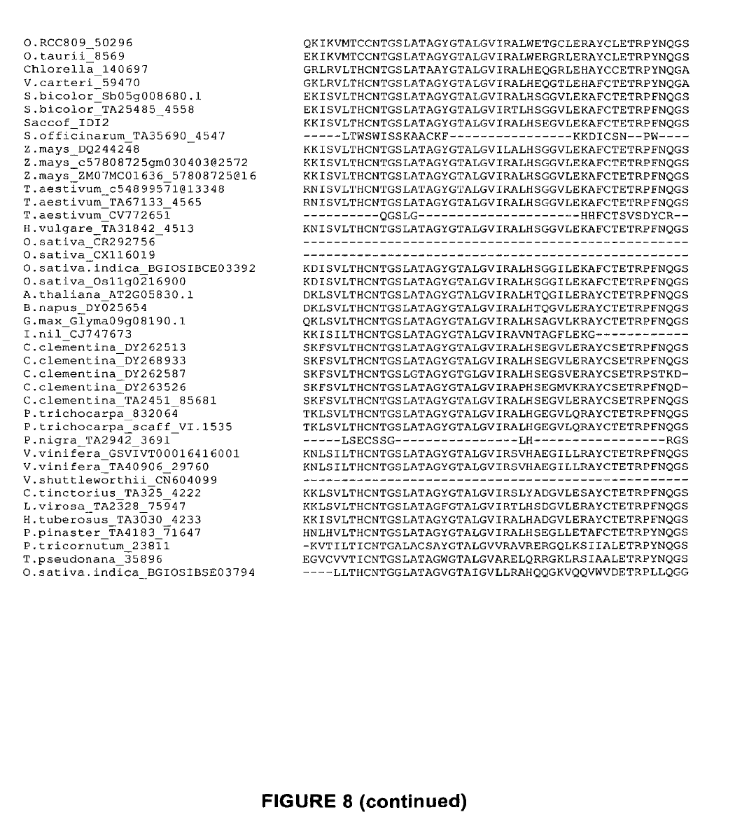

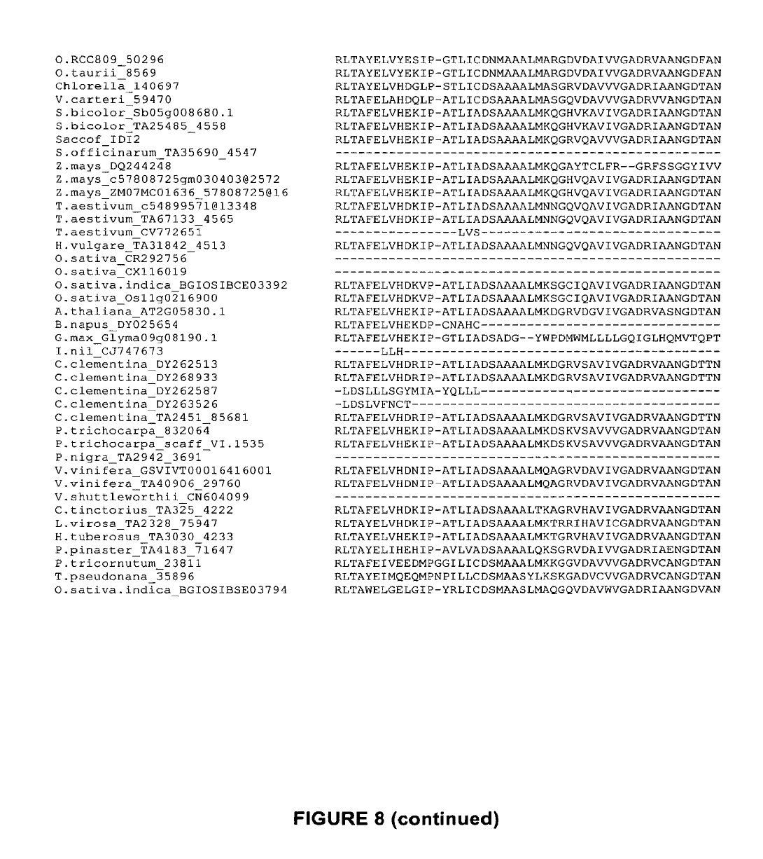

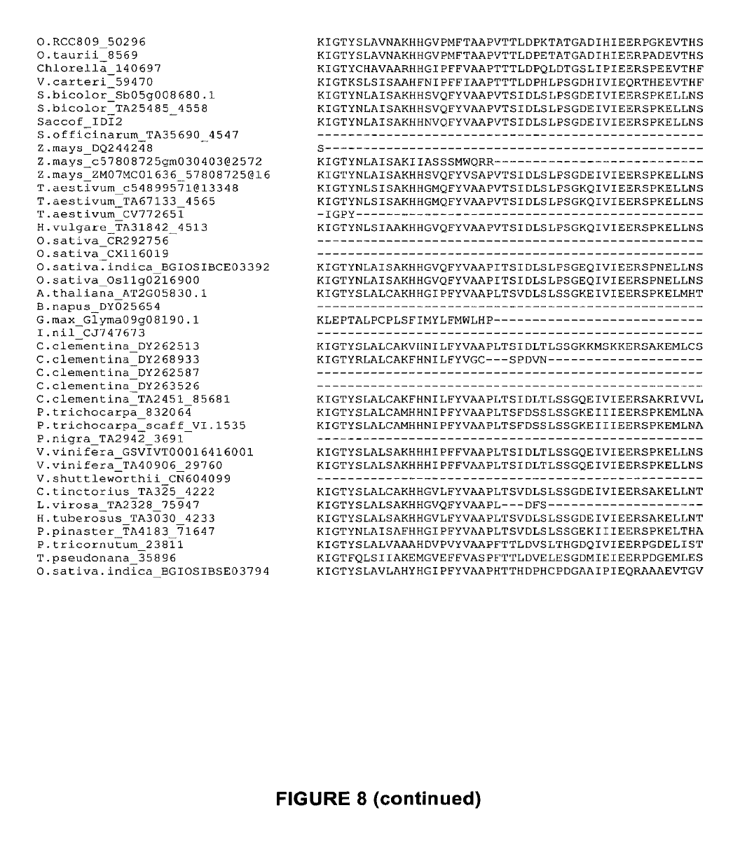

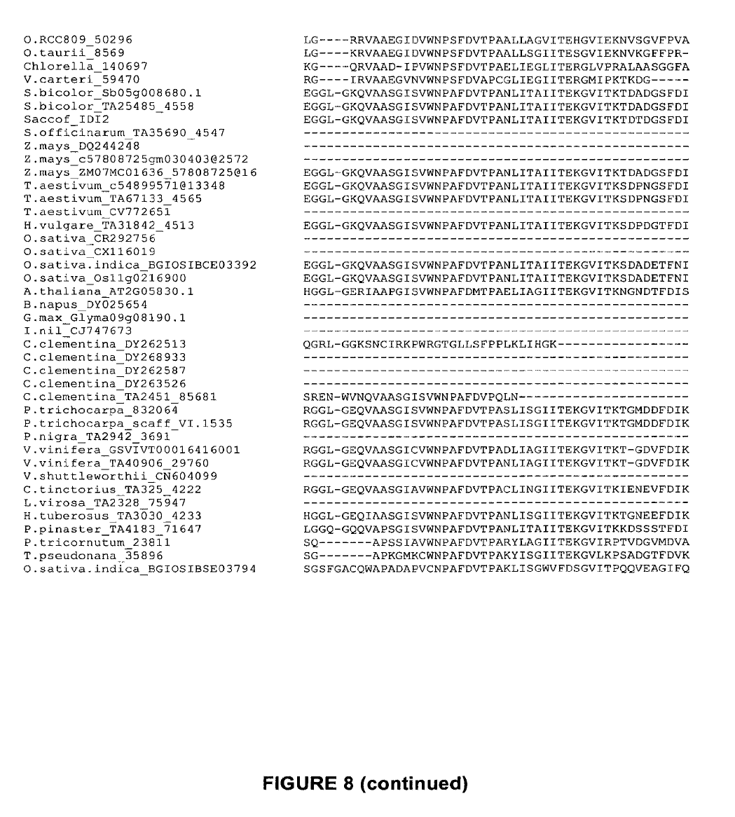

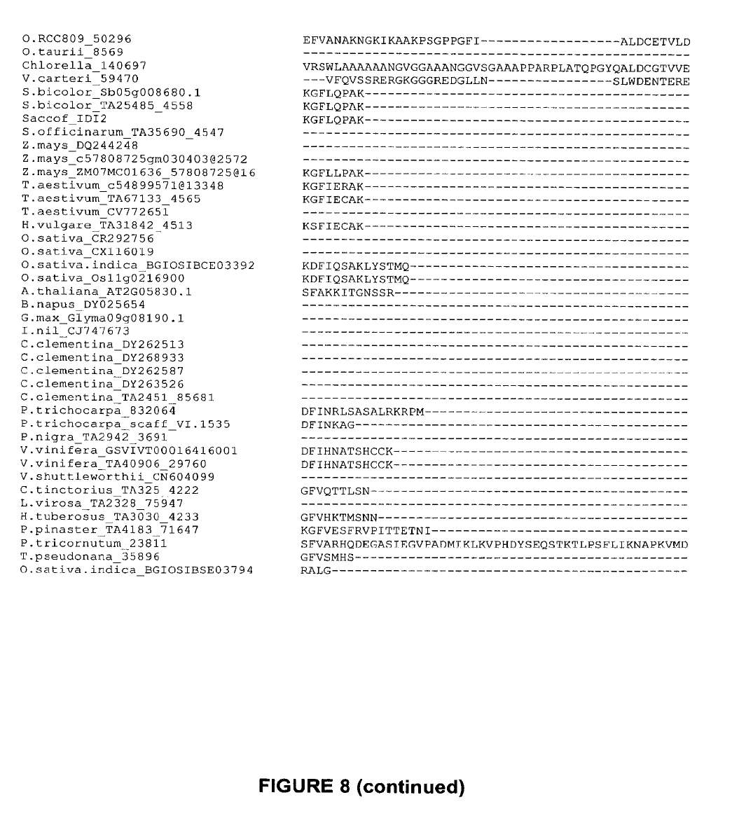

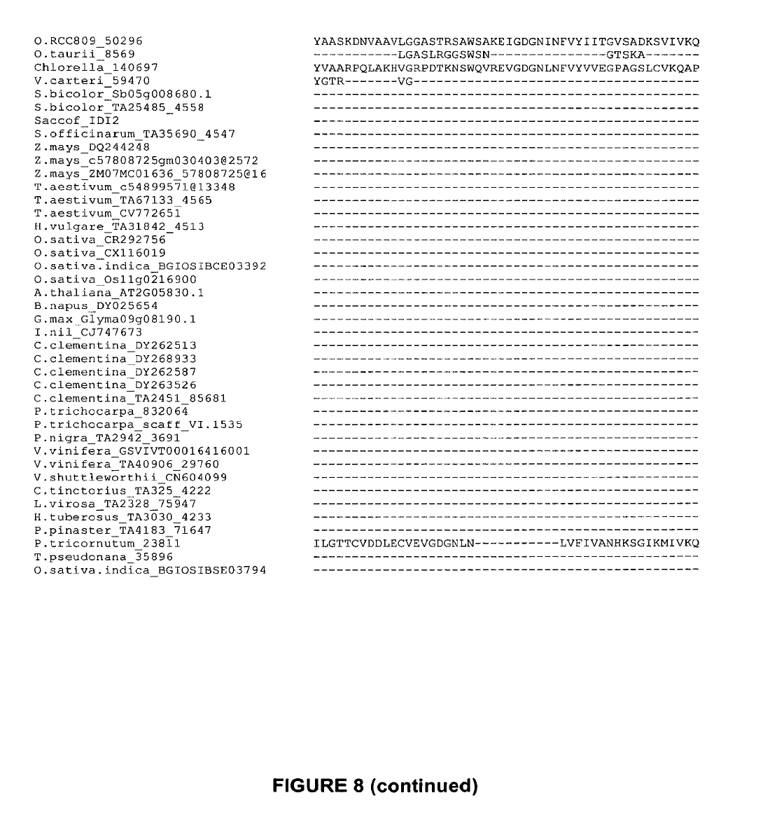

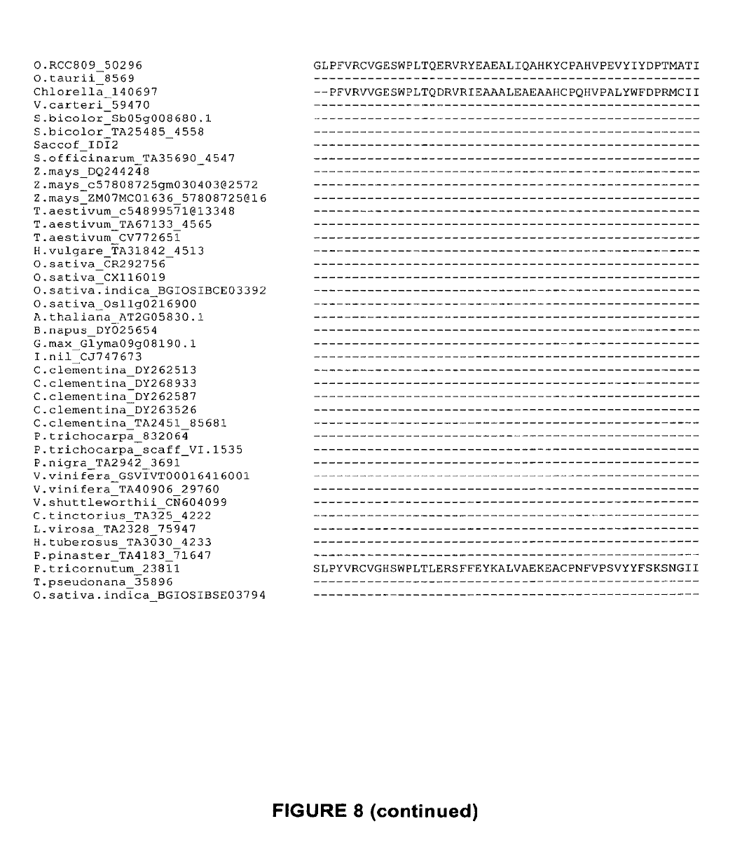

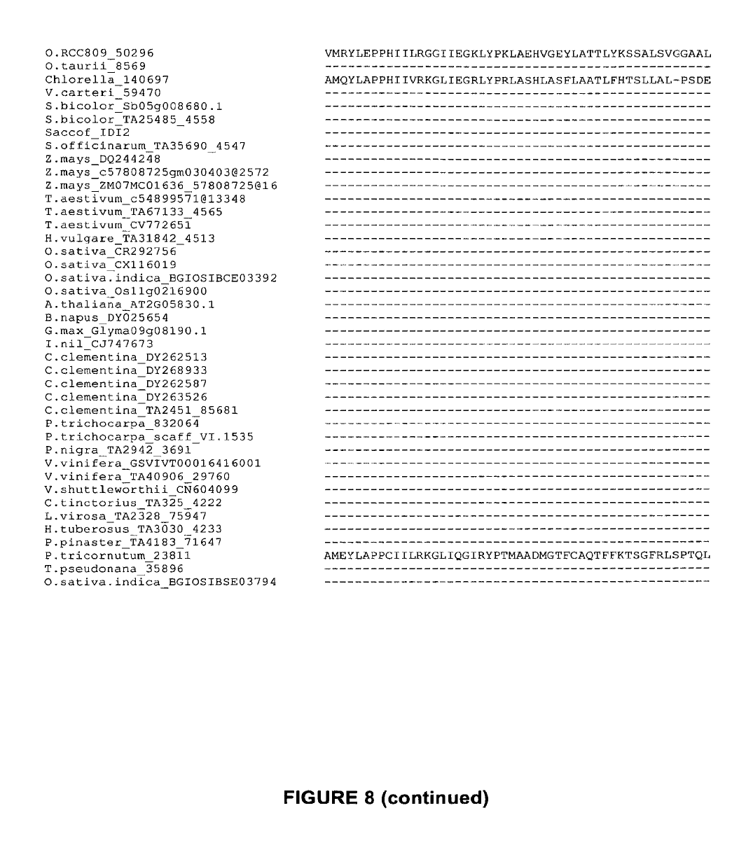

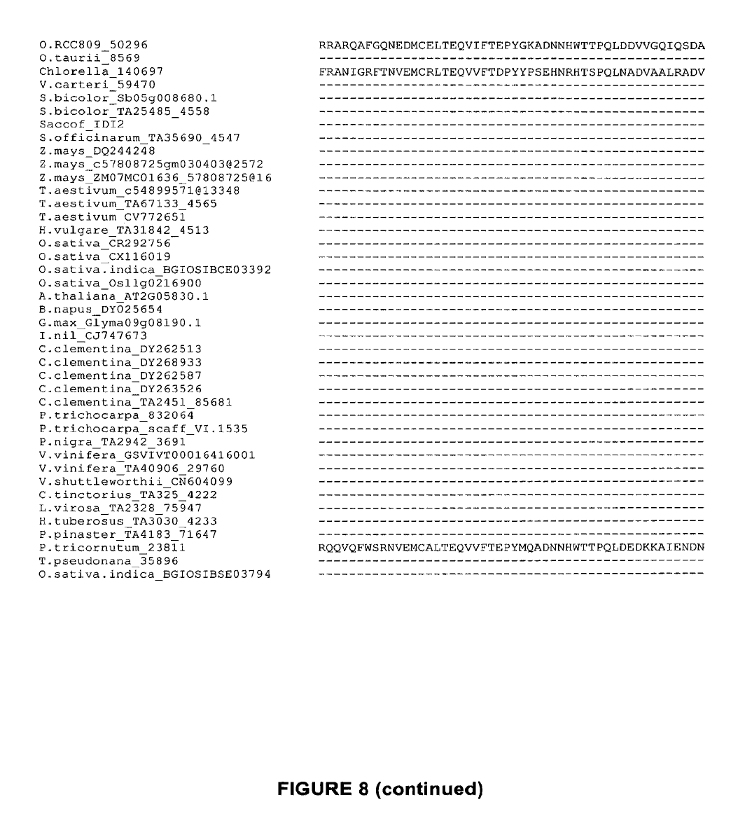

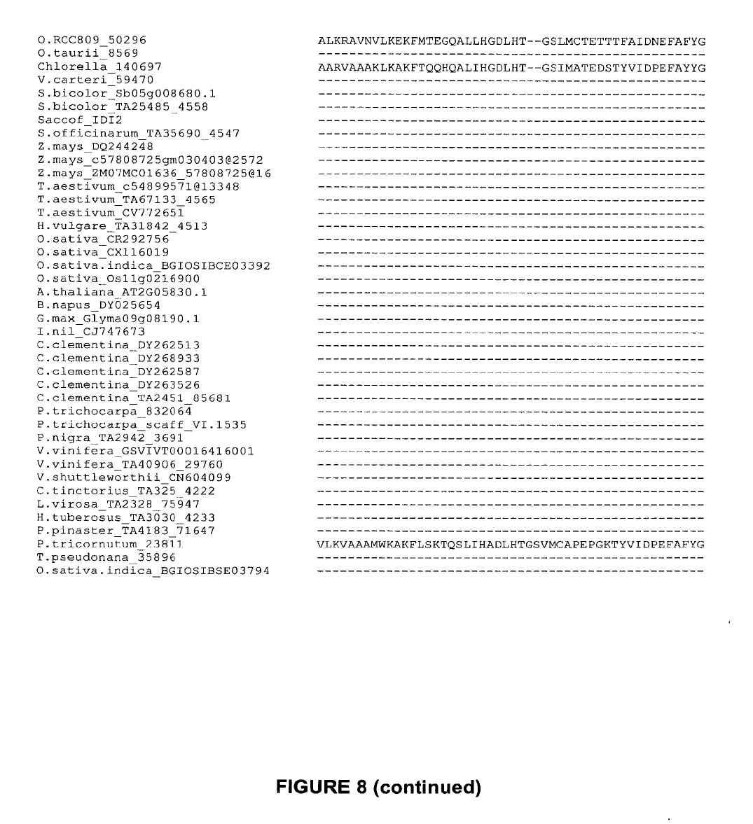

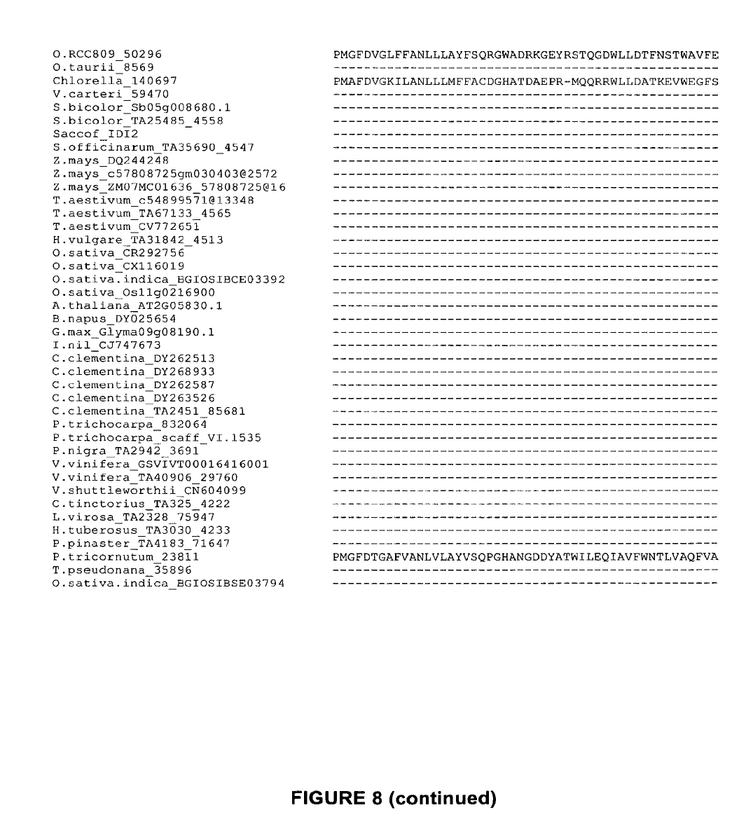

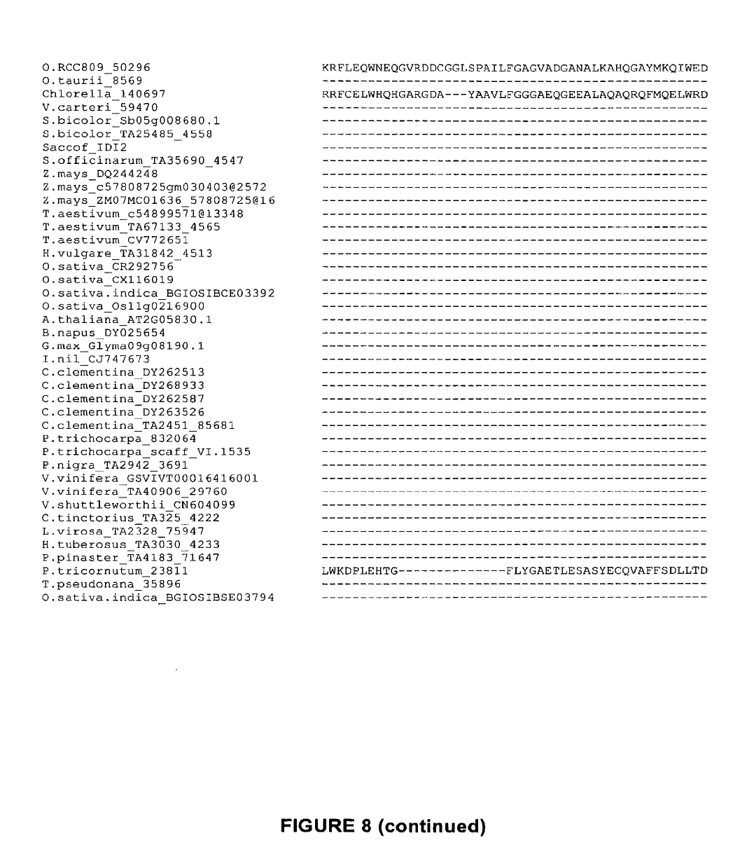

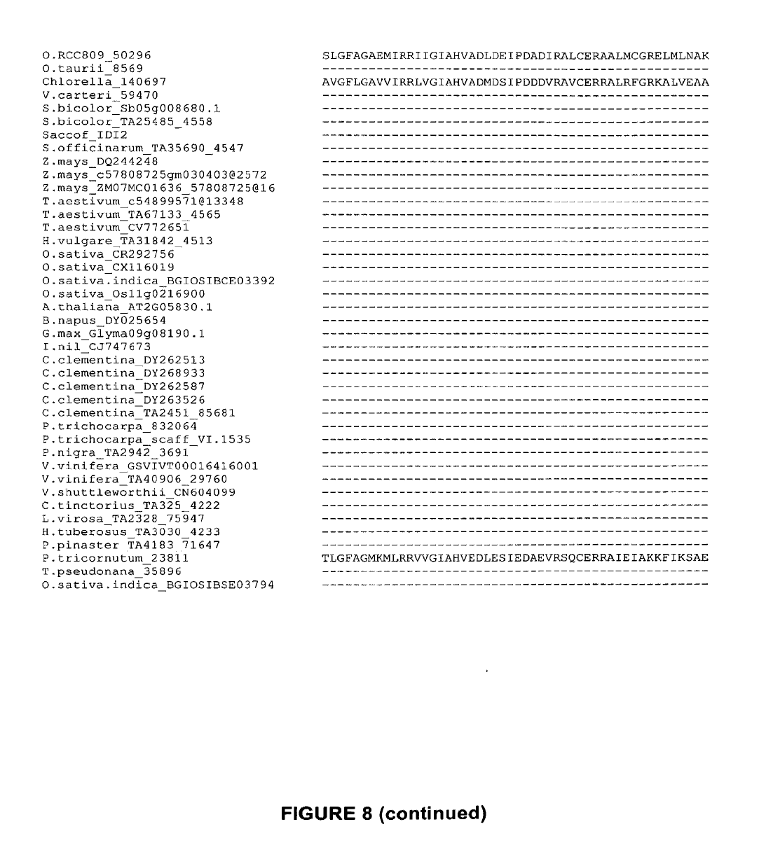

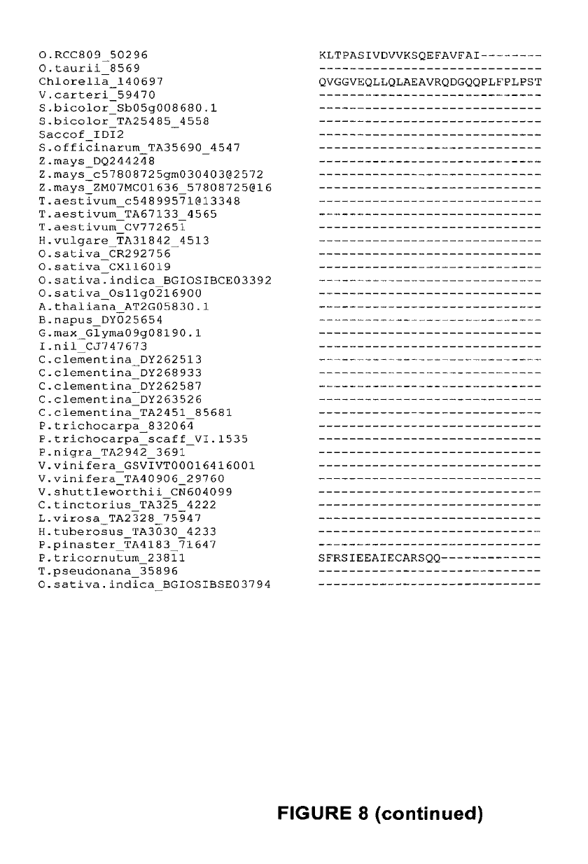

Alignment of polypeptide sequences was performed using the ClustalW 2.0 algorithm of progressive alignment (Thompson et al. (1997) Nucleic Acids Res 25:4876-4882; Chema et al. (2003). Nucleic Acids Res 31:3497-3500) with standard setting (slow alignment, similarity matrix: Gonnet, gap opening penalty 10, gap extension penalty: 0.2). Minor manual editing was done to further optimise the alignment. The IDI2 polypeptides of group A are aligned in FIG. 8. The highest degree of conservation is found in the N-terminal half of the protein sequence, the C-terminal part is variable in length. This alignment can be used for determining conserved signature sequences of about 5 to 10 amino acids in length. Preferably the conserved regions of the proteins are used, recognisable by the asterisks (identical residues), the colons (highly conserved substitutions) and the dots (conserved substitutions).

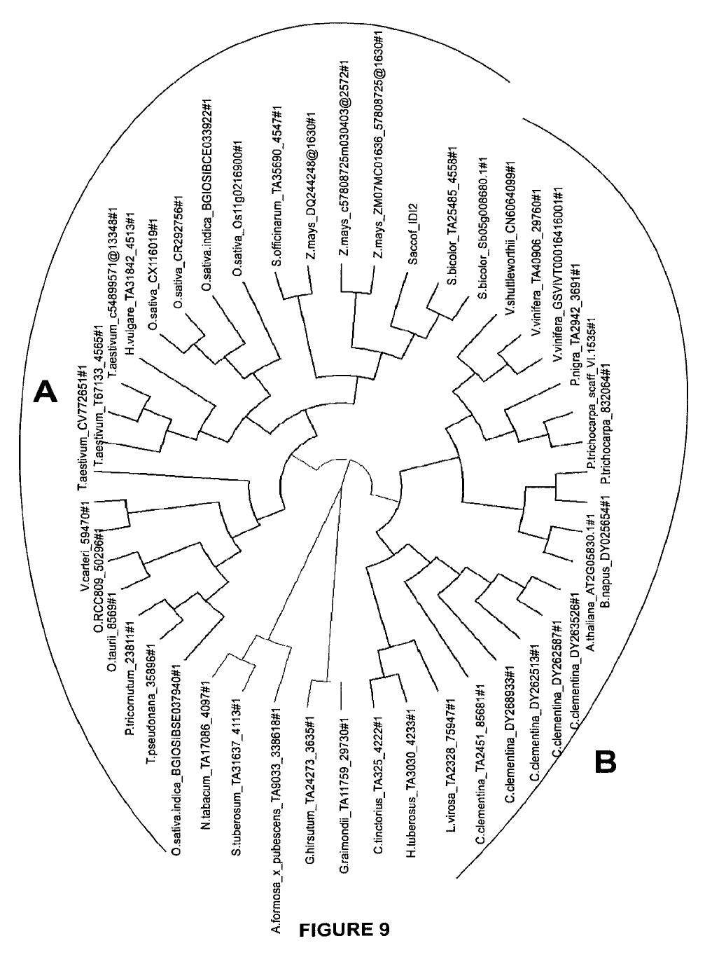

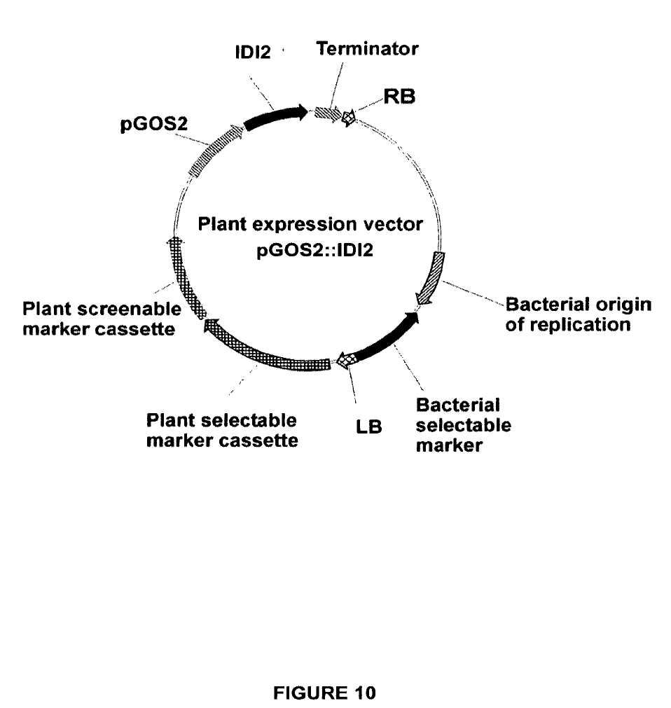

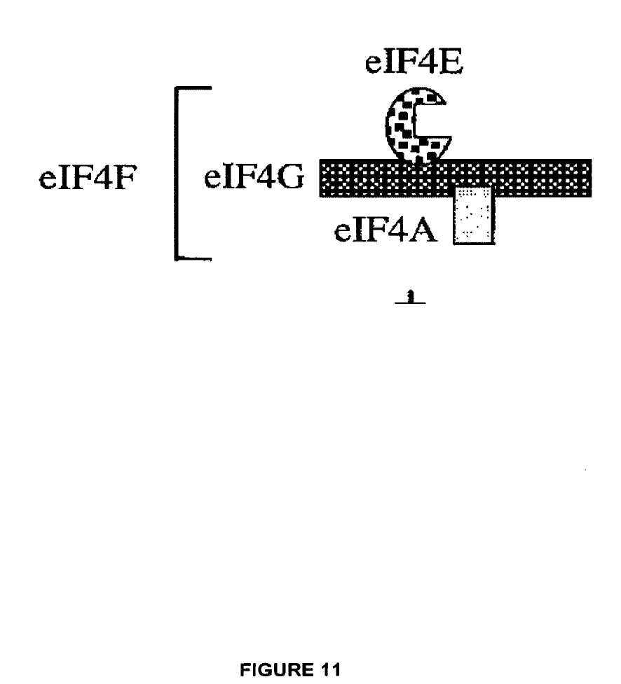

A phylogenetic tree of GR-RBP polypeptides (FIG. 9) was constructed using MAFFT (Katoh and Toh (2008) Briefings in Bioinformatics 9:286-298) for aligning the sequences. A neighbour-joining tree was calculated using QuickTree (Howe et al. (2002), Bioinformatics 18(11): 1546-7). Support of the major branching after 100 bootstrap repetitions is indicated. Visualisation of the tree was done with Dendroscope (Huson et al. (2007), BMC Bioinformatics 8(1):460). The tree shows a clear delineation of 2 subgroups (A and B) within the IDI2 polypeptides with a few outliers, SEQ ID NO: 140 clusters with the sequences within group A.

2.4. eIF4F-Like Protein Complex

Alignment of polypeptide sequences was performed using the ClustalW 2.0 algorithm of progressive alignment (Thompson et al. (1997) Nucleic Acids Res 25:4876-4882; Chema et al. (2003). Nucleic Acids Res 31:3497-3500) with standard setting (slow alignment, similarity matrix: Gonnet (or Blosum 62 (if polypeptides are aligned), gap opening penalty 10, gap extension penalty: 0.2). Minor manual editing was done to further optimise the alignment.

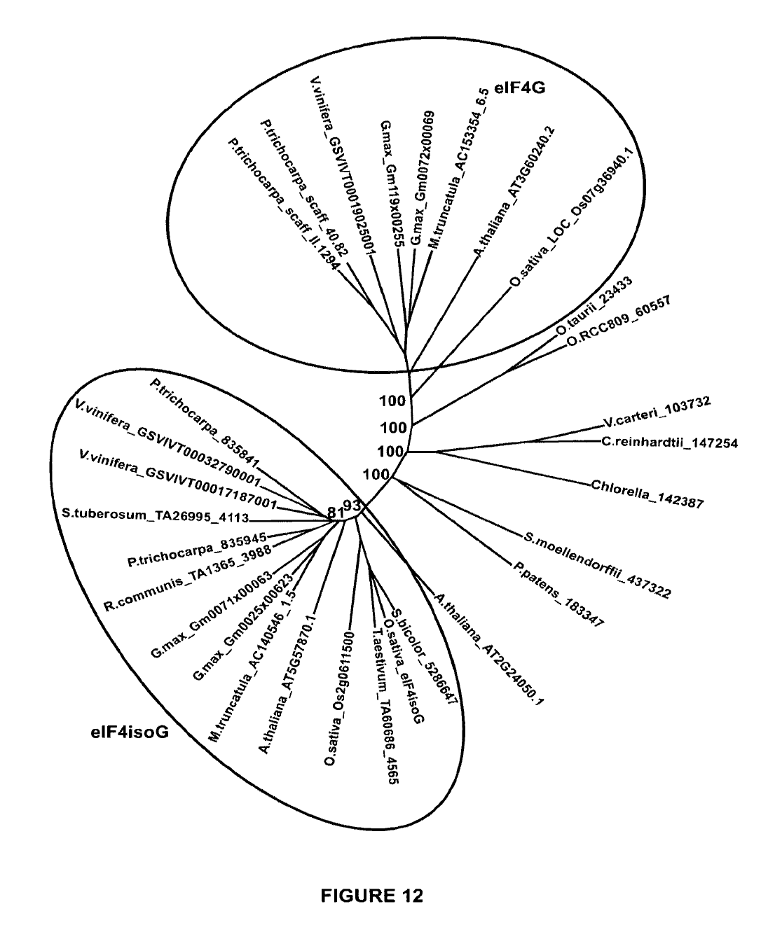

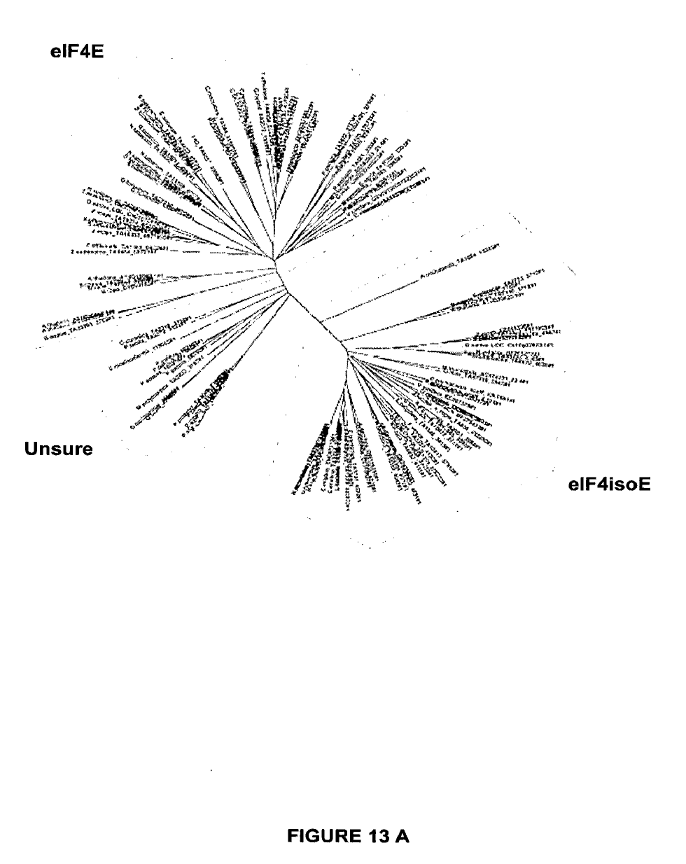

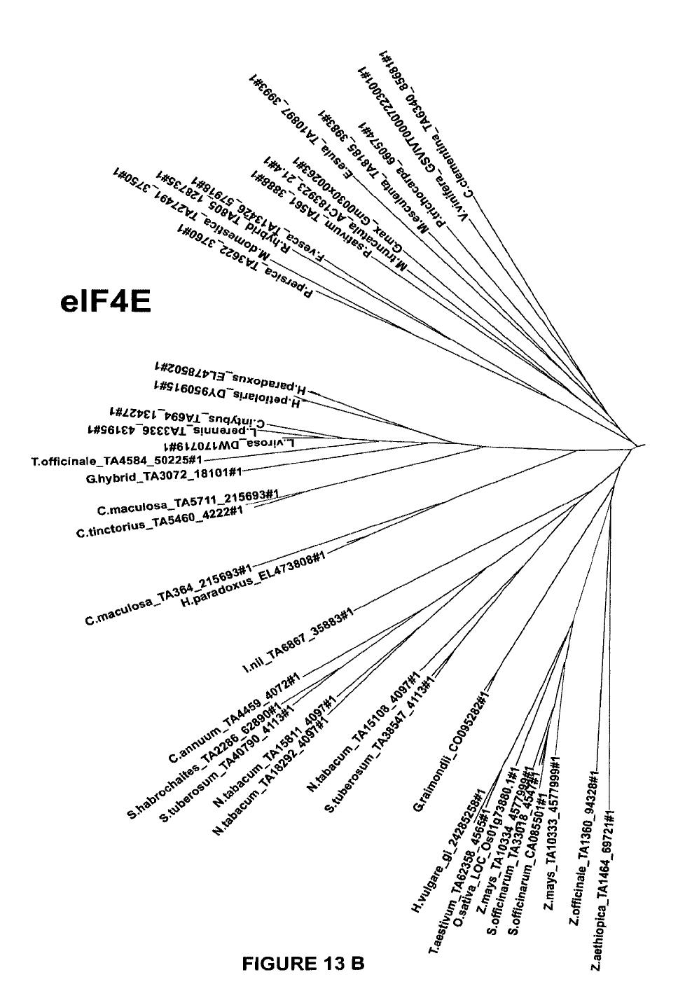

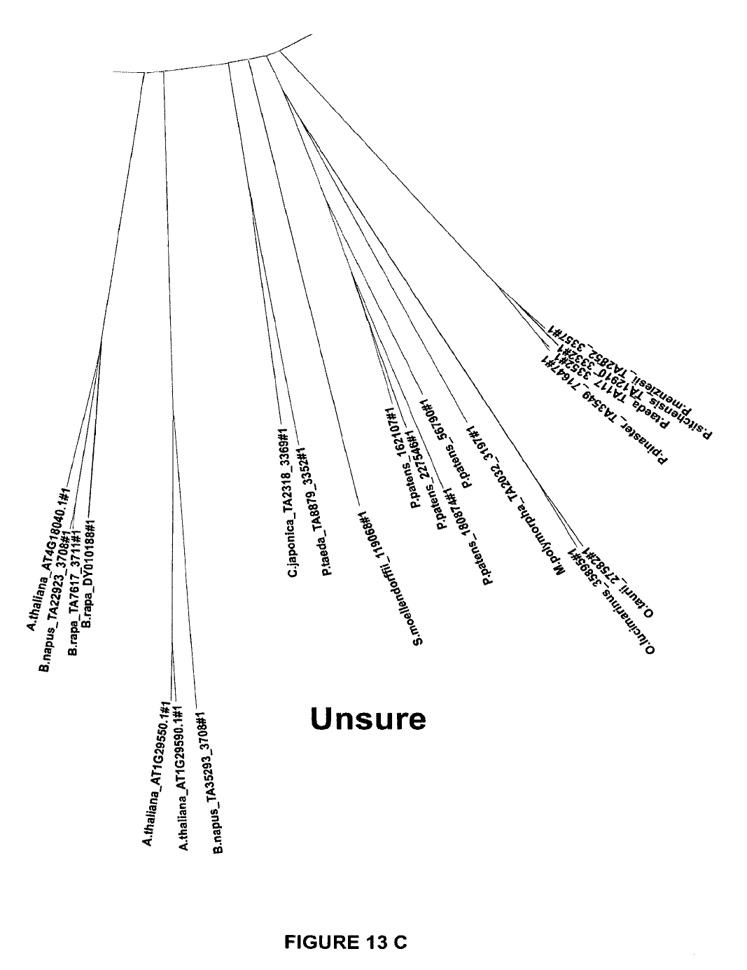

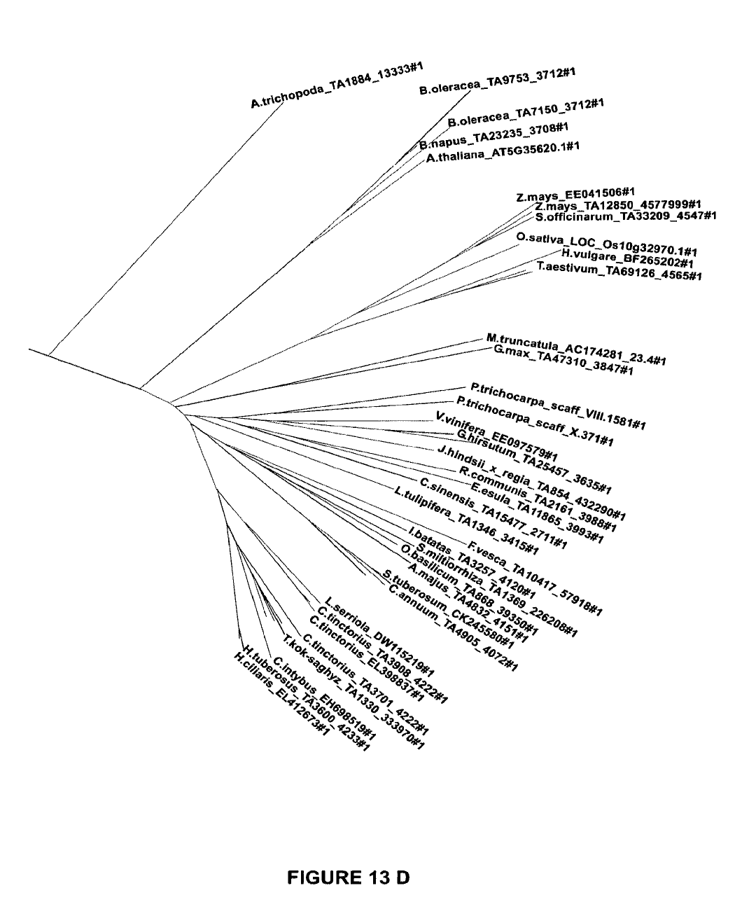

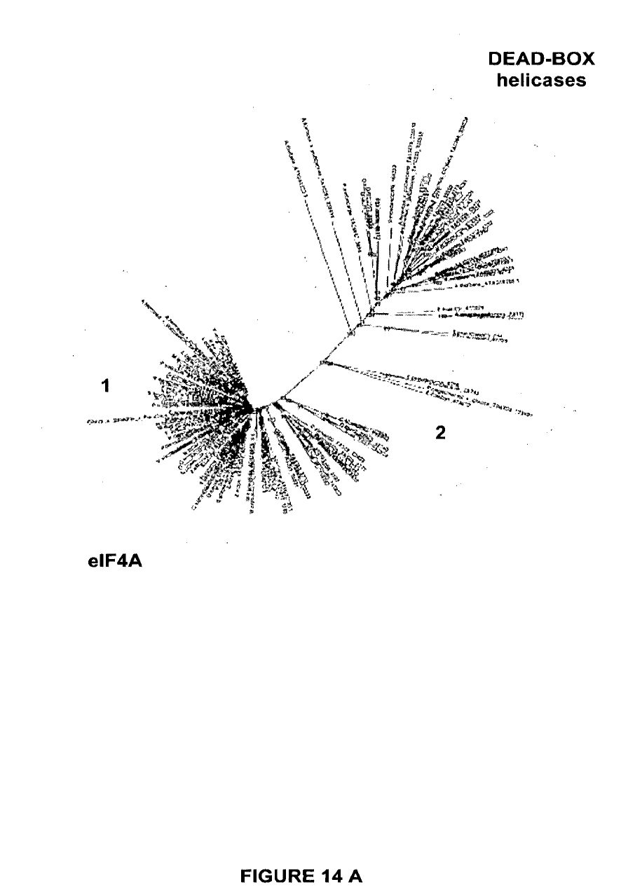

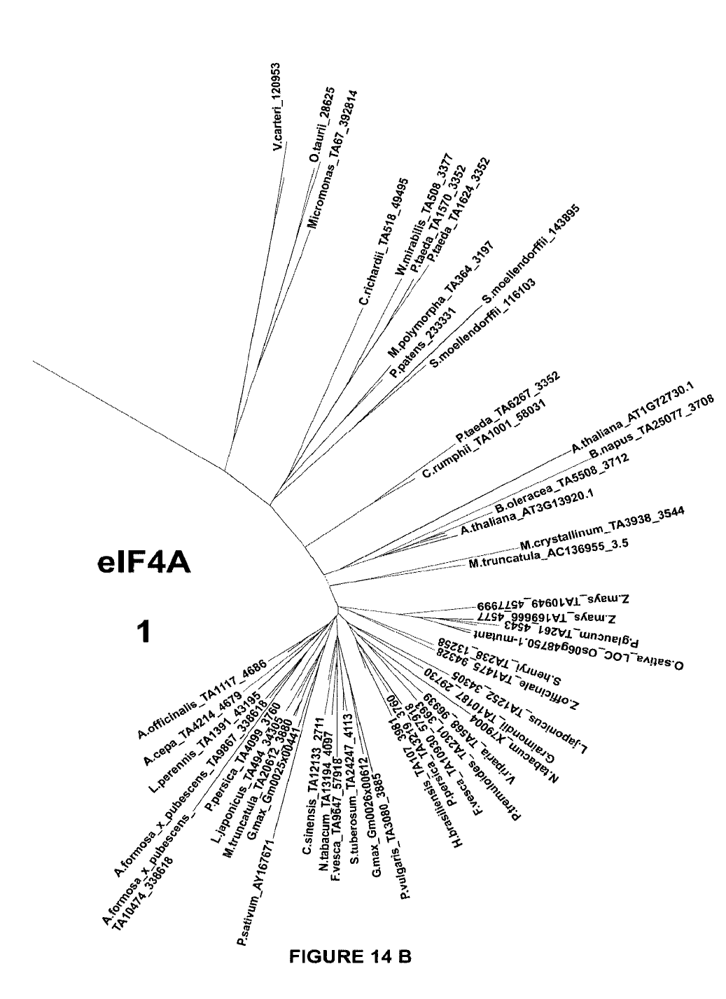

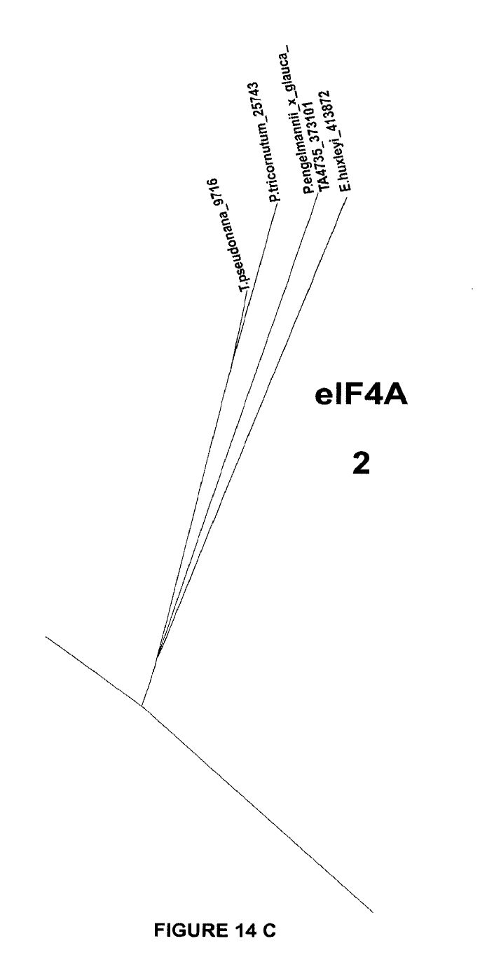

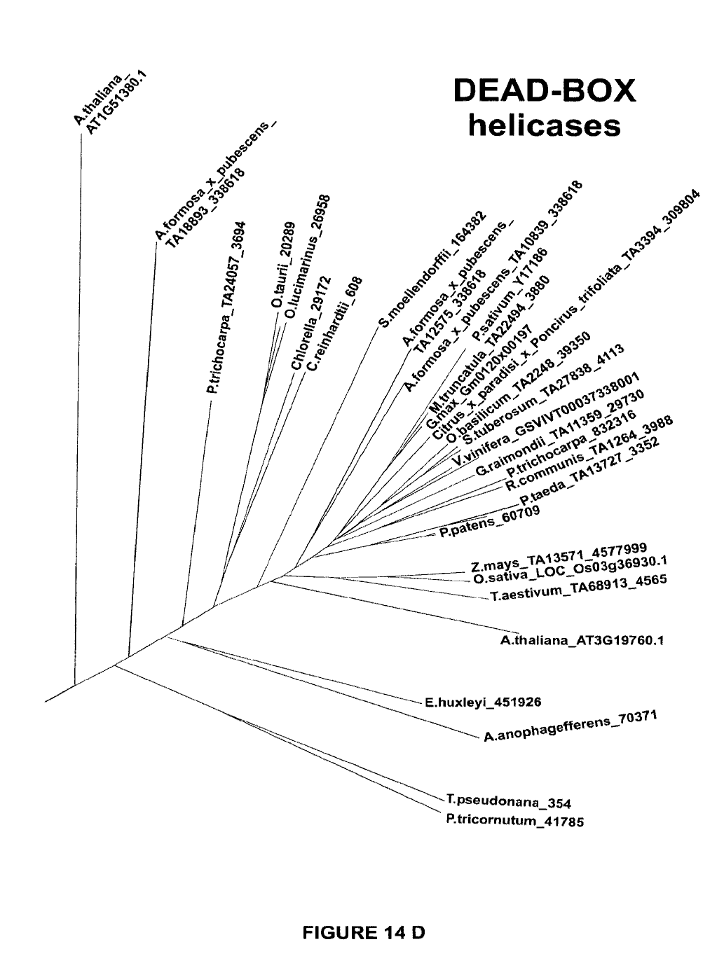

A phylogenetic tree of eIF4F-like protein complex subunits-polypeptides, eIF4G/isoG, eIG4A and eIF4E/iso (FIGS. 12, 13 and 14) were constructed using a neighbour-joining clustering algorithm as provided in the AlignX programme from the Vector NTI (Invitrogen).

Alignment of polypeptide sequences was performed using the ClustalW 2.0 algorithm of progressive alignment (Thompson et al. (1997) Nucleic Acids Res 25:4876-4882; Chema et al. (2003). Nucleic Acids Res 31:3497-3500) with standard setting (slow alignment, similarity matrix: Gonnet, gap opening penalty 10, gap extension penalty: 0.2). Minor manual editing was done to further optimise the alignment.

### 2.5. GR-RBP (Glycine Rich-RNA Binding Protein) Polypeptides

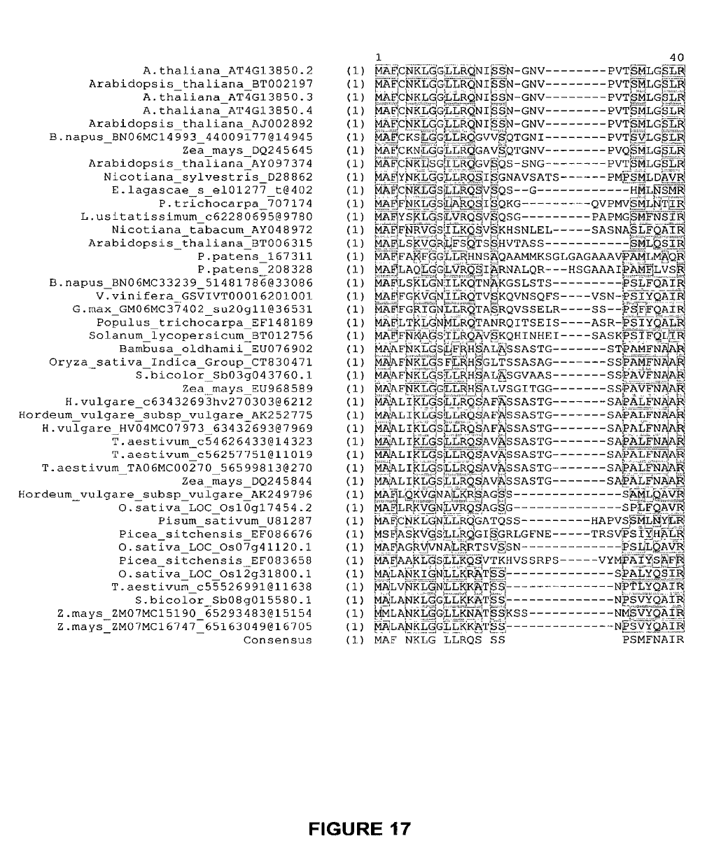

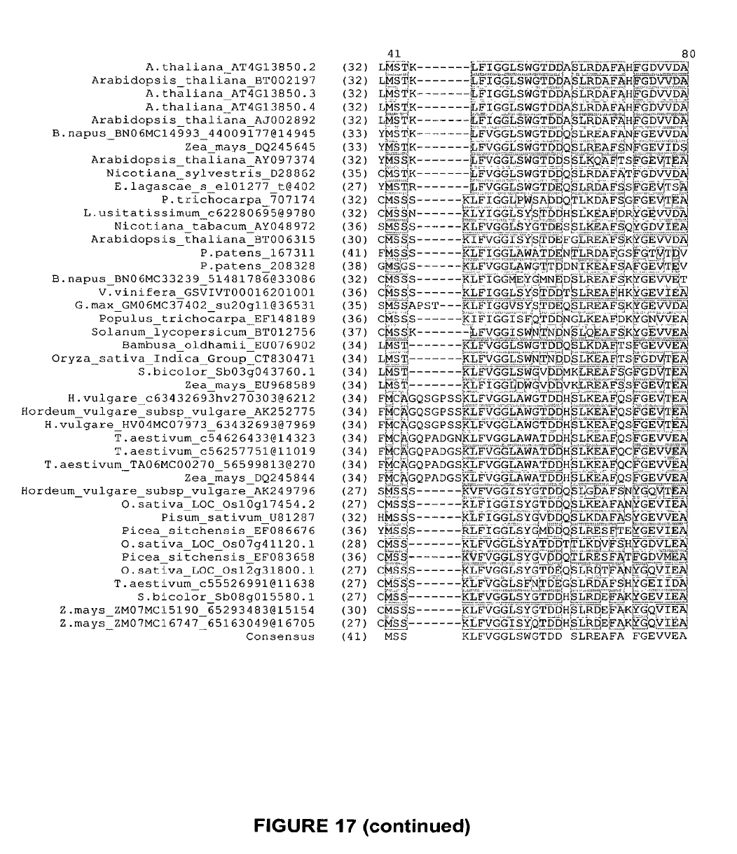

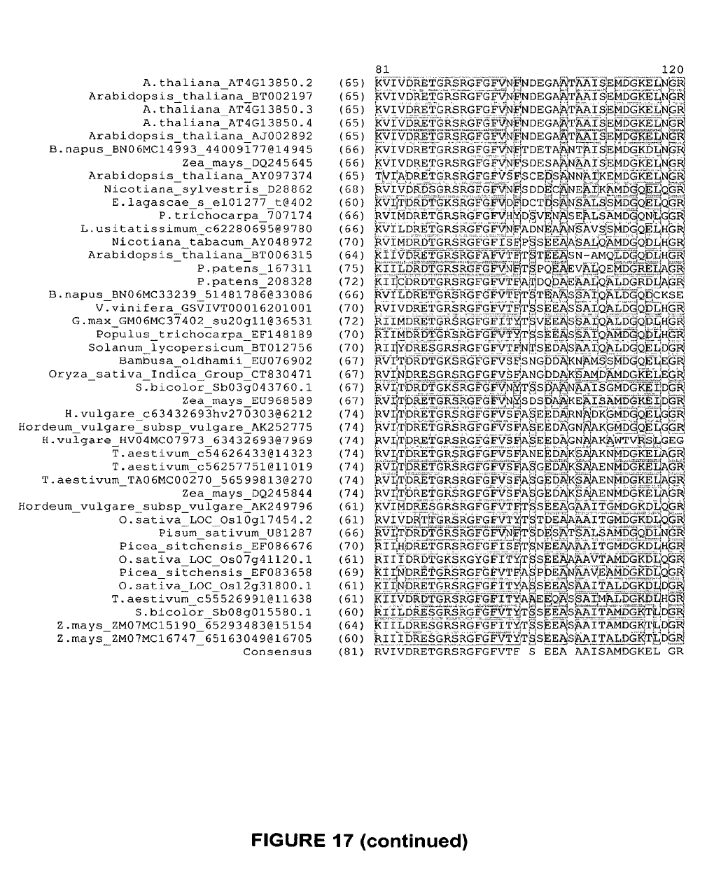

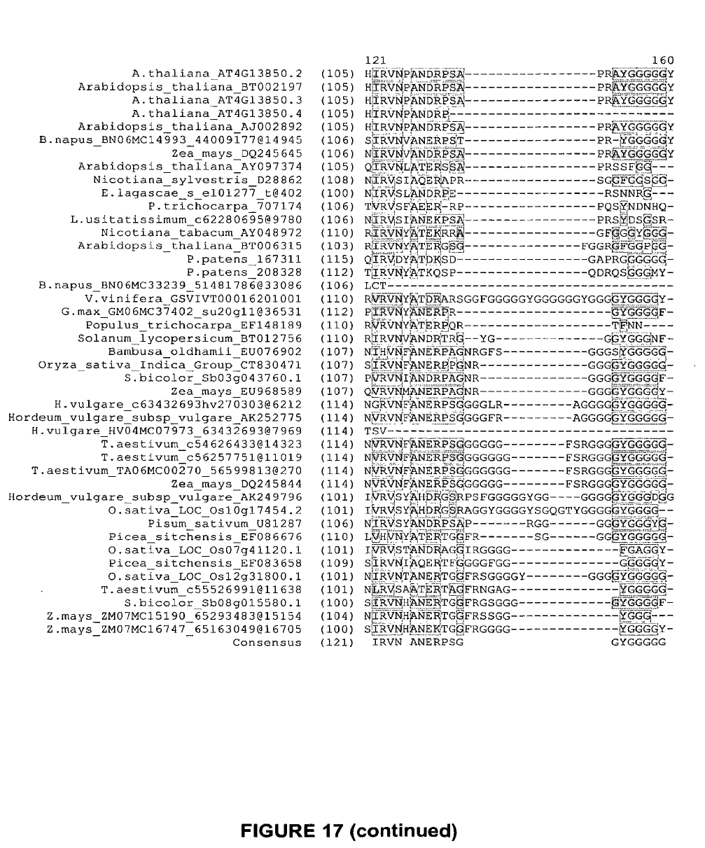

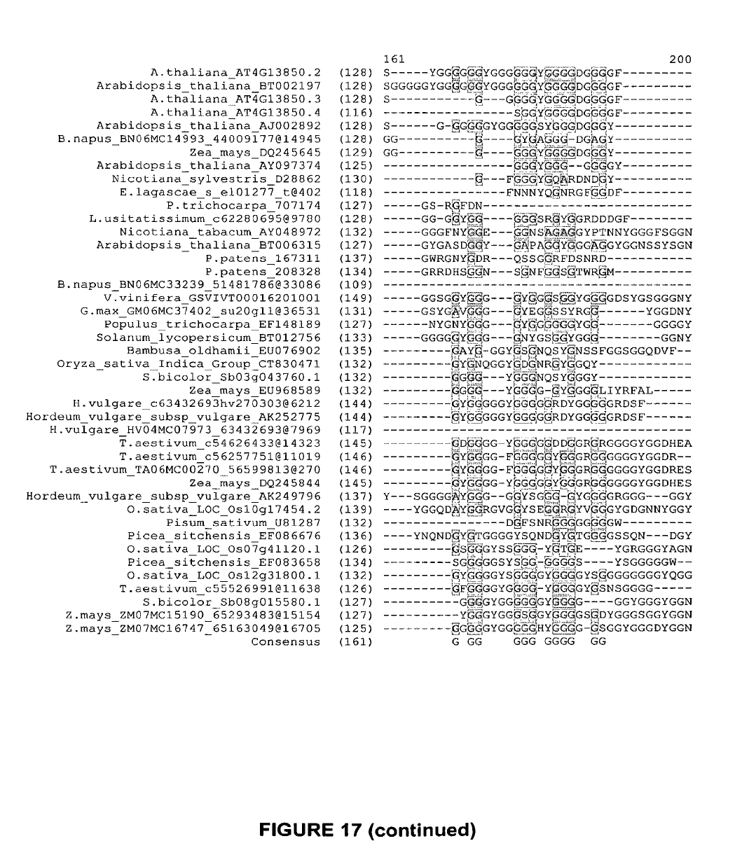

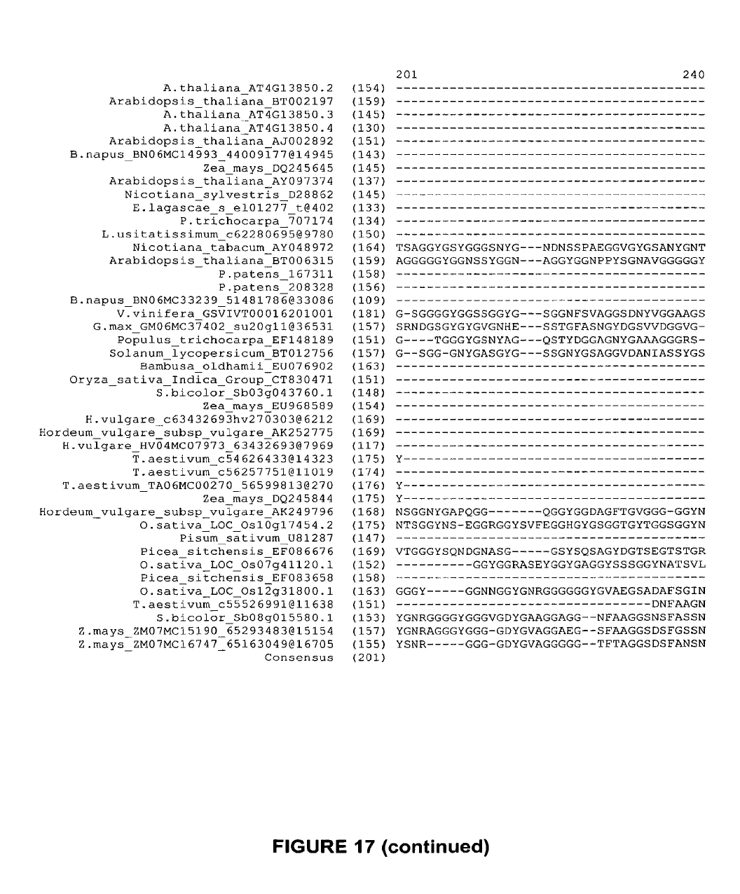

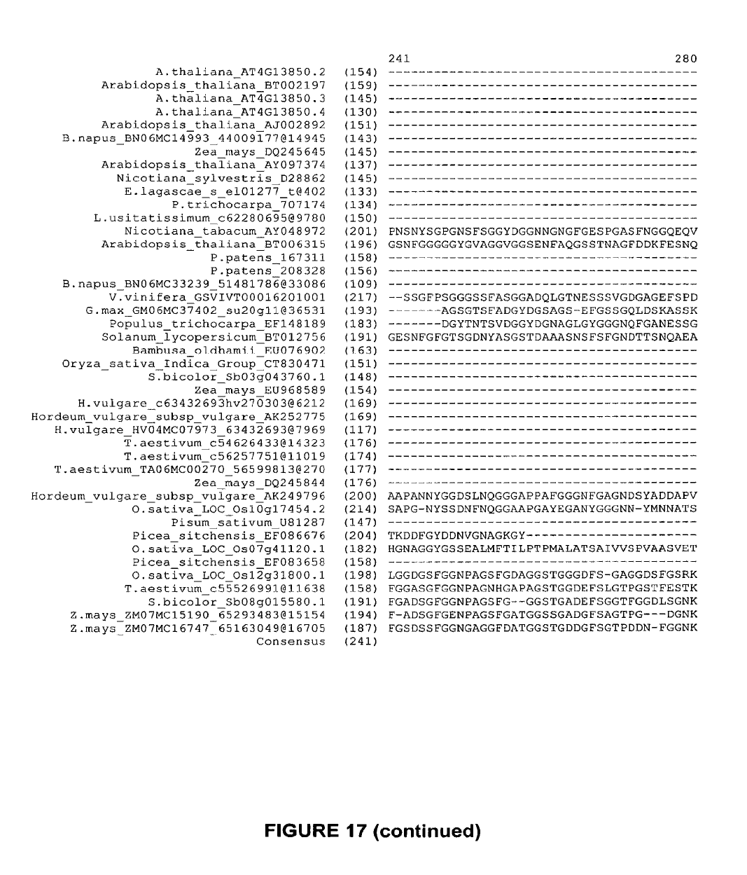

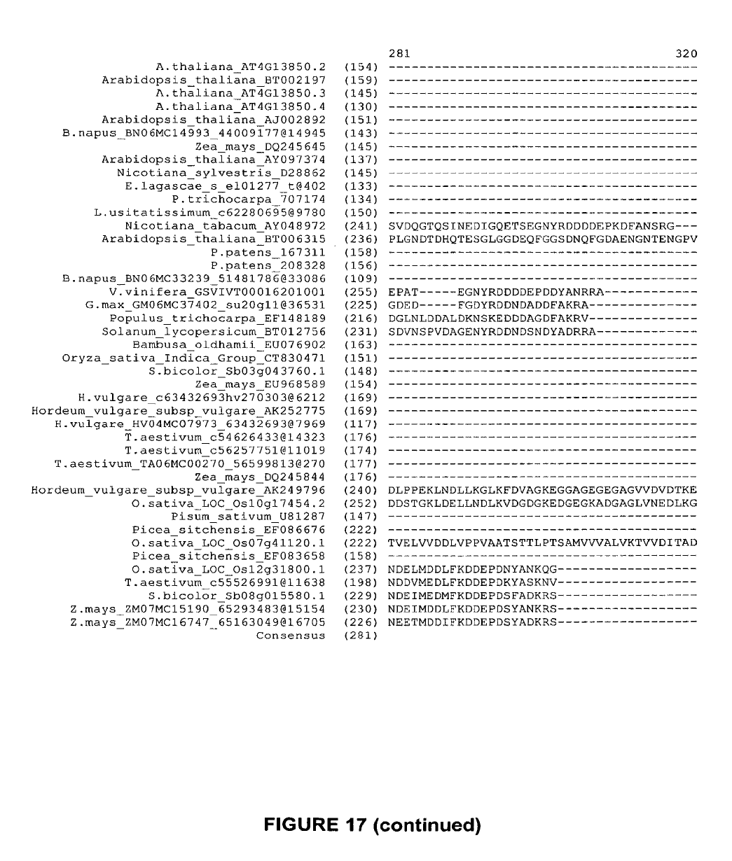

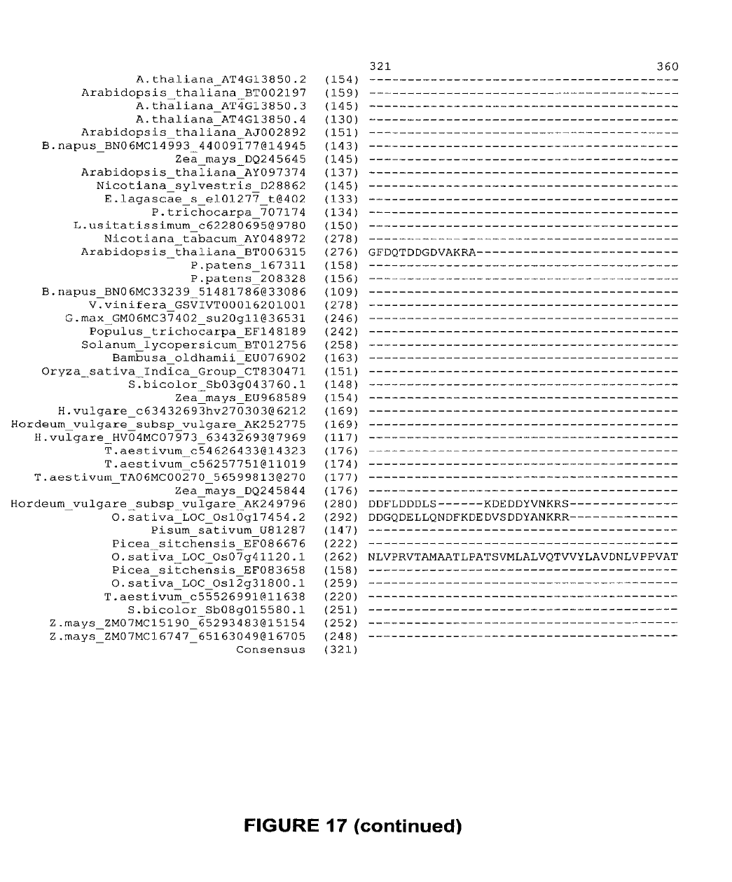

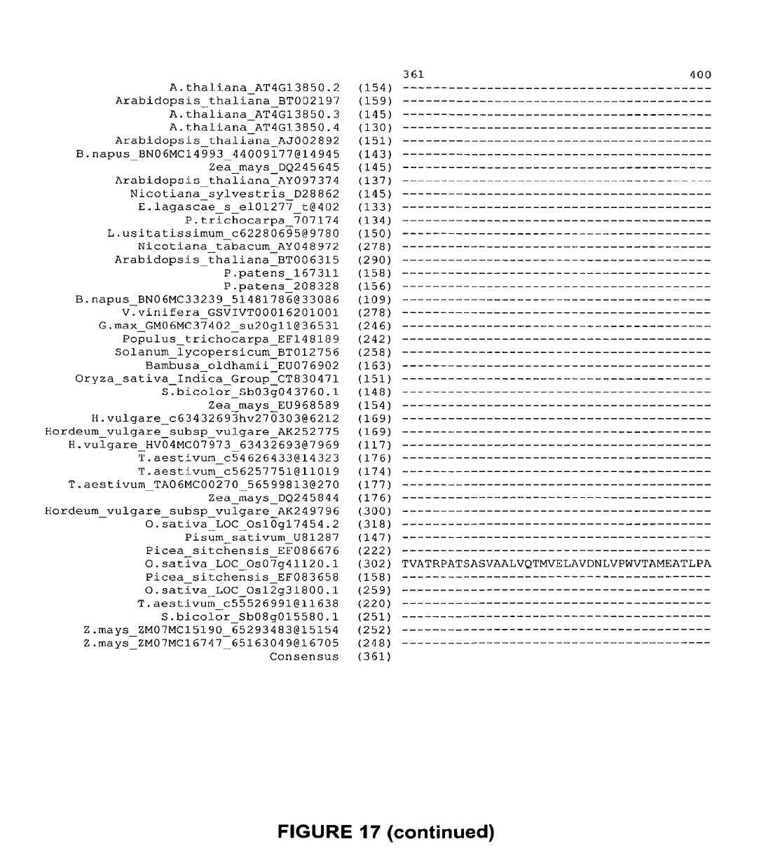

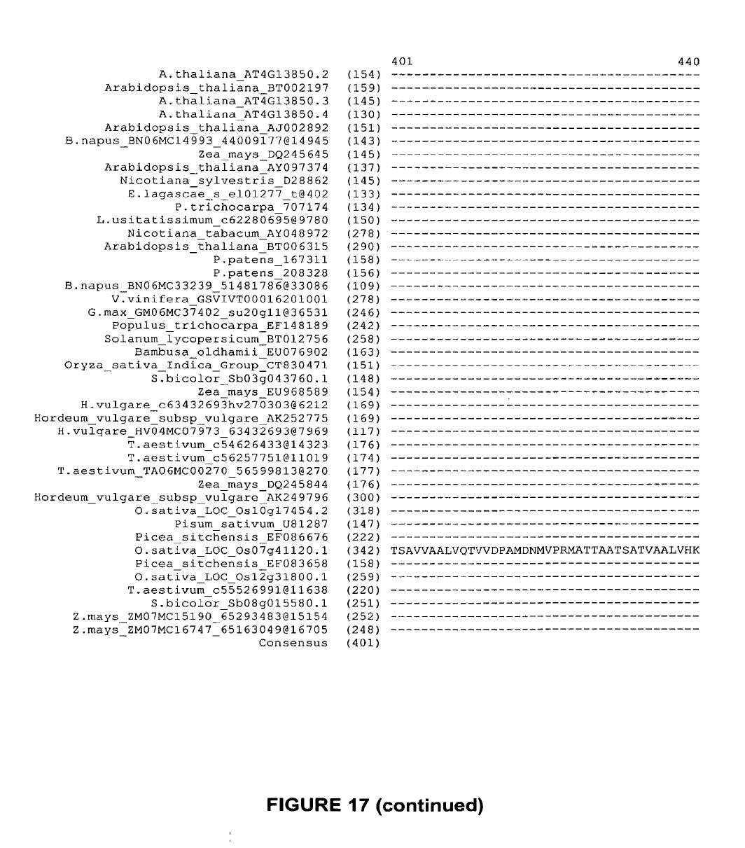

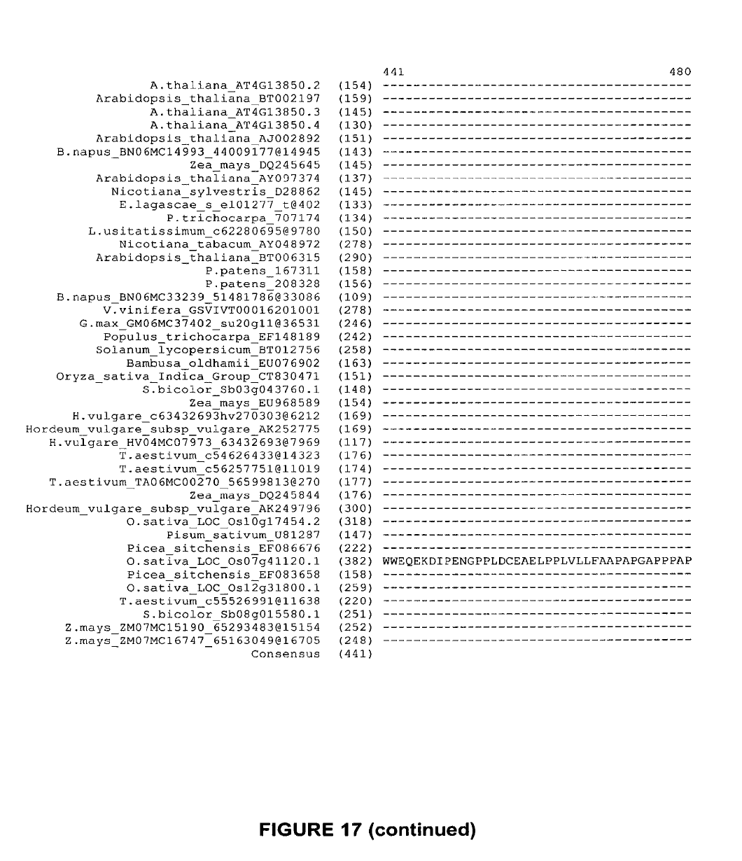

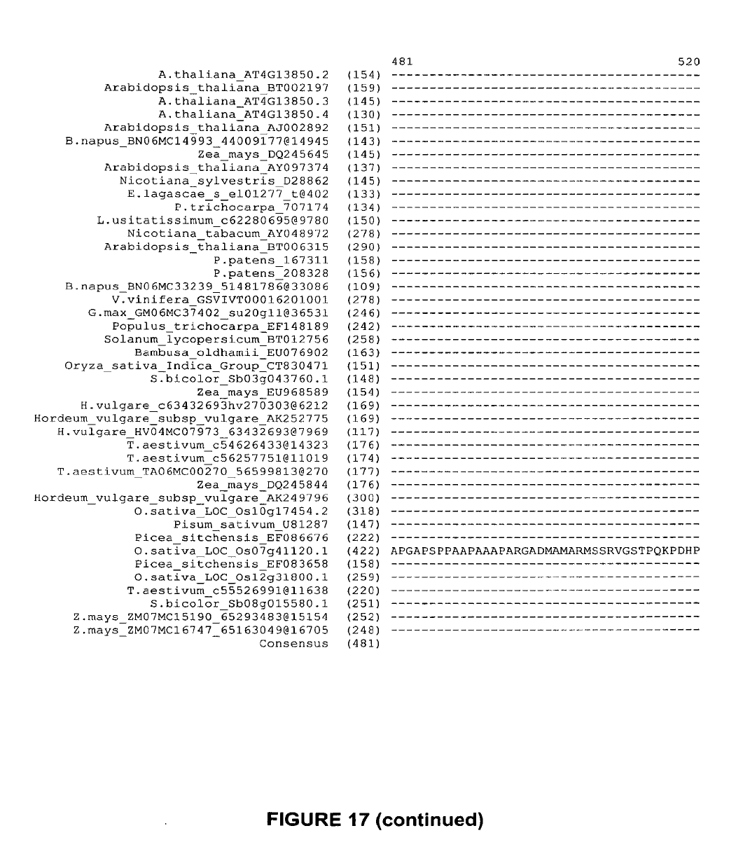

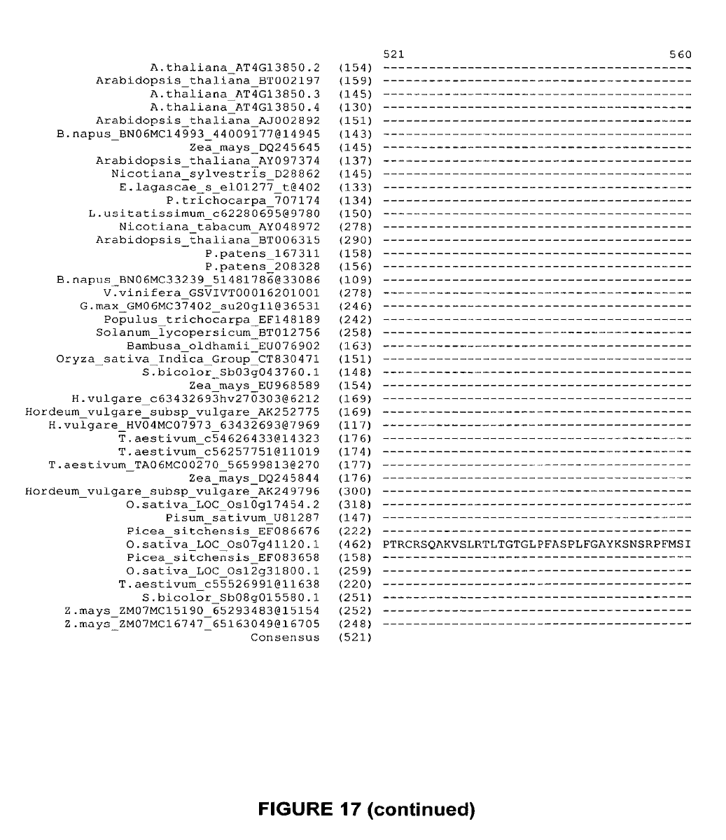

Alignment of polypeptide sequences was performed using VNTI (Advance 10, Invitrogen), with default settings. The alignment is created using the Clustal W algorithm (Nucleic Acid Research, 22 (22): 4673-4680, 1994). The GR-RBP polypeptides are aligned in FIG. 17. The highest degree of conservation is found in the N-terminal half of the protein sequence, the Glycine-rich domain, although variable in length, is readily recognisable.

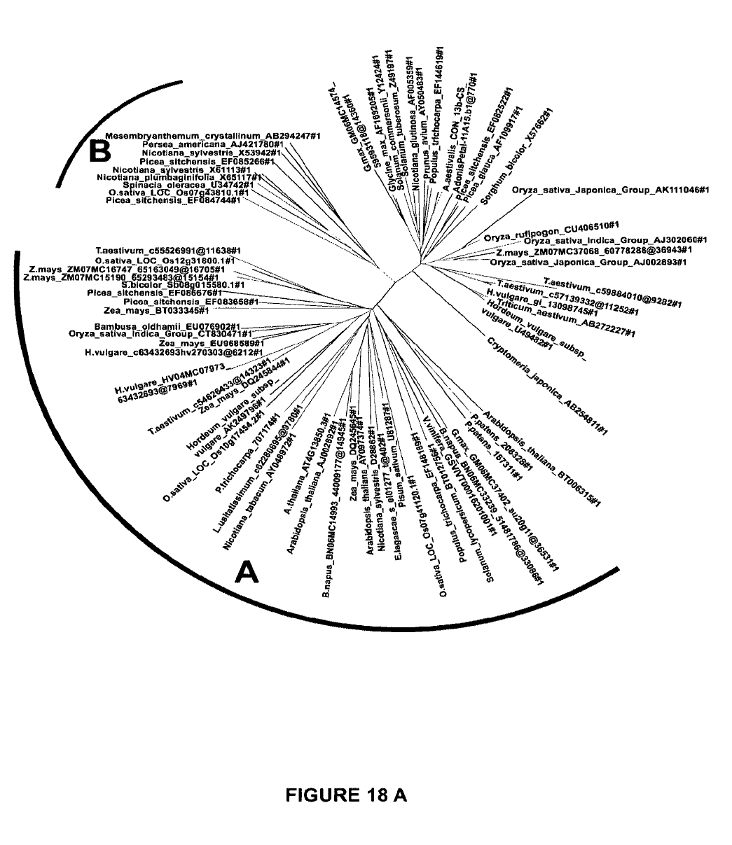

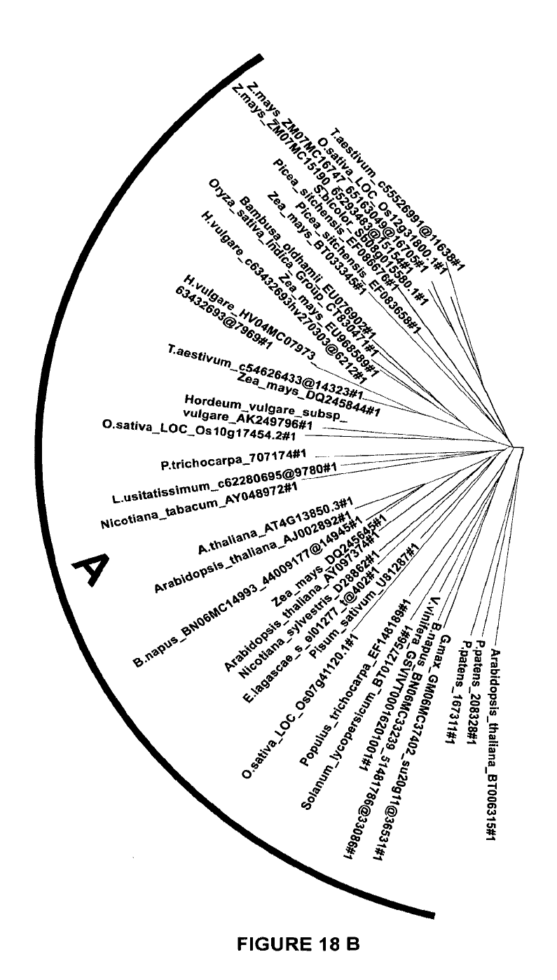

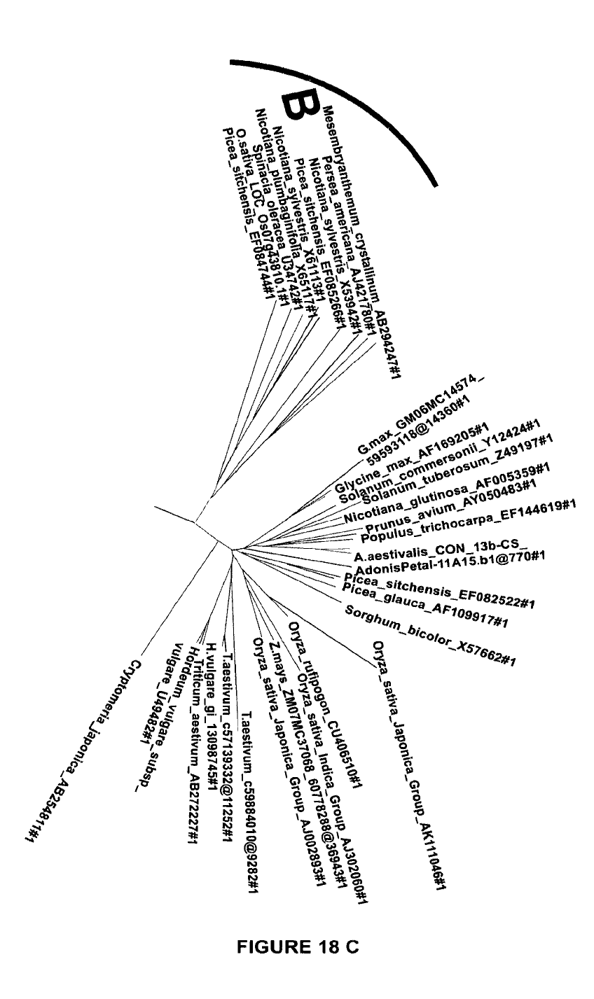

A phylogenetic tree of GR-RBP polypeptides (FIG. 18) was constructed using MAFFT (Katoh and Toh (2008) Briefings in Bioinformatics 9:286-298) for aligning the sequences. A neighbour-joining tree was calculated using QuickTree (Howe et al. (2002), Bioinformatics 18(11): 1546-7). Support of the major branching after 100 bootstrap repetitions is indicated. Visualisation of the tree was done with Dendroscope (Huson et al. (2007), BMC Bioinformatics 8(1):460). The tree shows a clear delineation of 2 subgroups within the GR-RBP polypeptides, group A and a smaller group B. SEQ ID NO: 827 clusters with the sequences within group A.

### Example 3

### Calculation of Global Percentage Identity Between Polypeptide Sequences Useful in Performing the Methods of the Invention

### 3.1. C3H-Like Polypeptides

Global percentages of similarity and identity between full length polypeptide sequences were determined using the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table B1 for the global similarity and identity over the full length of the polypeptide sequences. Percentage identity is given above the diagonal and percentage similarity is given below the diagonal.

TABLE B1
MatGAT results for global similarity and identity over the full length of the polypeptide sequences.

| | | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 |
|---|---|---|---|---|---|---|---|---|---|---|---|---|---|---|---|---|---|---|---|---|---|---|---|---|
| 1. | A_cepa_TC4713 | | 41 | 39.6 | 40.4 | 41.1 | 35.9 | 32.8 | 40.6 | 43.5 | 46.4 | 45.8 | 32 | 42.9 | 43.5 | 40.4 | 42.8 | 42.4 | 43.6 | 47.7 | 47.4 | 43.8 | 47.6 | 43.4 |
| 2. | A_thaliana_AT3G46620_1 | 52.4 | | 67.8 | 64.5 | 36.9 | 31.9 | 30.2 | 38.3 | 42.3 | 45.1 | 46.2 | 28.7 | 45.3 | 39.4 | 41.4 | 44.6 | 43.7 | 45.3 | 45.4 | 43.6 | 42.6 | 39.9 | 45 |
| 3. | A_thaliana_AT5G59550_1 | 51.1 | 80.6 | | 76.3 | 36.3 | 32.1 | 27.9 | 37.3 | 41.6 | 47.8 | 45.7 | 27.7 | 45.8 | 38.4 | 40.3 | 45.1 | 44.1 | 45.8 | 45 | 45 | 42.4 | 40 | 46.1 |
| 4. | B_napus_TC85754 | 52.9 | 79 | 86.7 | | 37.3 | 32.3 | 29.3 | 36.2 | 42.5 | 48.4 | 47.2 | 27.3 | 46.1 | 40.6 | 39.7 | 45.5 | 44.1 | 45.7 | 44.3 | 44 | 42.4 | 42.9 | 46.1 |
| 5. | C_clementina_DY266223 | 59.3 | 50.4 | 49.4 | 48.9 | | 80.8 | 35 | 39.7 | 78.8 | 47.2 | 52 | 35.3 | 43.6 | 42.2 | 39.5 | 42.7 | 42.5 | 43 | 45.2 | 45.1 | 43.7 | 41.3 | 43.3 |
| 6. | C_clementina_TC3900 | 52.4 | 44.8 | 44.5 | 43 | 81.7 | | 40.1 | 35.3 | 72.2 | 40.5 | 46.1 | 42.3 | 39.3 | 39.2 | 32.3 | 37.1 | 37.1 | 37.7 | 39.8 | 39.3 | 38.1 | 36.5 | 38.8 |
| 7. | C_longa_TA1491_136217 | 49.5 | 39.7 | 37.6 | 39.2 | 48.3 | 56.6 | | 37.3 | 31.1 | 33.7 | 34.6 | 38.8 | 31.2 | 33.2 | 28.8 | 31.6 | 33.5 | 32 | 33.8 | 33.5 | 33.5 | 30.5 | 30.7 |
| 8. | C_longa_TA268_136217 | 59.3 | 57 | 55.5 | 54.7 | 55.6 | 51.4 | 48 | | 41.2 | 44.6 | 44.8 | 32.7 | 40.5 | 36.4 | 41 | 43 | 43.1 | 42.8 | 41.5 | 41 | 43.4 | 38.1 | 40.3 |
| 9. | C_sinensis_TC458 | 59.8 | 58.2 | 55.8 | 55.9 | 81.6 | 73.7 | 43.3 | 63.8 | | 54.9 | 60.9 | 31.7 | 49.2 | 42.7 | 43.3 | 49.5 | 50.6 | 50.5 | 50.6 | 49.9 | 51.8 | 48.2 | 50 |
| 10. | F_vesca_TA10341_57918 | 62.6 | 58.7 | 60 | 59.5 | 59.6 | 51.7 | 44.4 | 64 | 70.8 | | 60.1 | 34.9 | 51.5 | 44.5 | 49.5 | 60.8 | 55.8 | 62 | 57.1 | 57 | 54.3 | 52 | 51.9 |
| 11. | G_hirsutum_TC105257 | 62.3 | 59.2 | 59.2 | 59.5 | 61 | 54.2 | 45.8 | 63.7 | 72.6 | 75.3 | | 33.6 | 51 | 43.2 | 47.2 | 56.1 | 55.2 | 55.8 | 51.1 | 51.1 | 55.6 | 50.1 | 52.1 |
| 12. | G_hirsutum_TC82591 | 46.6 | 37 | 35.6 | 36.7 | 47.3 | 56.6 | 54.7 | 44.4 | 42.8 | 44.7 | 44.4 | | 30.3 | 32.7 | 55.3 | 31.6 | 32.1 | 31.5 | 34.8 | 34.8 | 33.6 | 28.9 | 30.3 |
| 13. | G_hirsutum_TC85784 | 57.7 | 61.3 | 61.7 | 63.3 | 57.1 | 50.5 | 42.9 | 62.6 | 64.2 | 68.3 | 68 | 41.8 | | 63.8 | 42.3 | 49.4 | 48.4 | 48.6 | 47.9 | 48.4 | 47.6 | 41.2 | 97 |
| 14. | G_hirsutum_TC92683 | 59.9 | 52.9 | 49.6 | 53.4 | 59 | 52.8 | 47.8 | 55.4 | 59.5 | 60.7 | 57.7 | 46.9 | 73.5 | | 37.7 | 41.5 | 40 | 41.3 | 42.9 | 43.2 | 42.2 | 37.4 | 64.6 |
| 15. | G_hirsutum_TC93828 | 53.5 | 56.2 | 56.8 | 57.5 | 51.2 | 43.7 | 38.8 | 56.6 | 57.8 | 62.2 | 63 | 56.8 | 58.1 | 53.2 | | 45.4 | 48.1 | 45.7 | 46.4 | 46.4 | 48.2 | 42 | 43.2 |
| 16. | G_max_Glyma11g14580_1 | 59 | 58 | 60 | 61.3 | 57.6 | 49.9 | 43.5 | 62.6 | 65.1 | 74.8 | 69.6 | 40.7 | 66.9 | 57.9 | 58.9 | | 52.5 | 93.4 | 61.7 | 61.9 | 51.4 | 45.2 | 49.1 |
| 17. | G_max_Glyma11g34160_1 | 56.7 | 59 | 61.2 | 59.5 | 55.5 | 48.9 | 42.5 | 60.8 | 65.6 | 68.2 | 69.7 | 41 | 62.6 | 53.7 | 64.9 | 64.9 | | 53.8 | 49.7 | 49.9 | 83.9 | 45 | 48.2 |
| 18. | G_max_Glyma12g06460_1 | 59 | 59.5 | 60.7 | 60.5 | 56.8 | 50.1 | 43.5 | 61.2 | 65.7 | 75.3 | 70.7 | 40.2 | 66.9 | 59 | 57.1 | 96.7 | 65.1 | | 62.3 | 62.2 | 52.7 | 46.3 | 48.6 |
| 19. | G_max_Glyma13g41340_1 | 65.6 | 56.7 | 55.3 | 58.2 | 62.5 | 55.1 | 47.5 | 58.8 | 63.5 | 68 | 64.2 | 47.1 | 63.7 | 63.4 | 57.8 | 72.9 | 60.1 | 72.9 | | 94.3 | 49.7 | 48.9 | 49.3 |
| 20. | G_max_Glyma15g04080_1 | 65.6 | 56.7 | 54.8 | 56.7 | 62.1 | 54.5 | 47.1 | 57.1 | 62.6 | 68 | 63.4 | 47.1 | 63.1 | 62.7 | 57.6 | 73.4 | 59.8 | 73.4 | 96.5 | | 49.9 | 48.5 | 49.2 |
| 21. | G_max_Glyma18g04140_1 | 61.6 | 55.2 | 56.8 | 57.2 | 57.6 | 50.6 | 44.9 | 63.8 | 68.9 | 70.2 | 70.7 | 43.2 | 63.4 | 58.8 | 61.2 | 65.7 | 86.5 | 66.2 | 64.7 | 63.6 | | 45.2 | 48.1 |
| 22. | G_max_Glyma18g40130_1 | 66 | 53.4 | 51.6 | 54.7 | 61.5 | 54.8 | 44.2 | 55.4 | 64 | 63.5 | 64.5 | 42 | 57.1 | 59 | 54 | 58.2 | 57.3 | 58.4 | 64 | 63.7 | 59.3 | | 41.7 |
| 23. | G_raimondii_TC6392 | 58.7 | 61.8 | 61.4 | 62.3 | 57.9 | 50.5 | 42.6 | 62 | 65 | 68.6 | 69.1 | 41.8 | 97.3 | 74.3 | 58.9 | 65.8 | 62.3 | 66.4 | 63.4 | 63.1 | 63.9 | 57.7 | |
| 24. | L_japonicus_TC26018 | 55.9 | 59.7 | 61.9 | 60.8 | 52.2 | 45.6 | 42 | 59.6 | 60.7 | 69.4 | 67.5 | 39.6 | 64.4 | 54.4 | 59.4 | 77.3 | 65.9 | 78.4 | 64.6 | 65.2 | 63.9 | 54.9 | 63.9 |
| 25. | L_sativa_TC27450 | 57.1 | 62.5 | 63.6 | 60.8 | 55.8 | 49.4 | 41.9 | 57.6 | 62 | 65.9 | 69.5 | 42.4 | 67.2 | 57.1 | 64 | 64.6 | 69.2 | 65.4 | 59.4 | 58.9 | 68.2 | 57.6 | 64.9 |
| 26. | M_esculenta_TA5606_3983 | 53.7 | 44.6 | 40.8 | 43.5 | 55.8 | 65.7 | 56.5 | 50.3 | 50.7 | 51.7 | 49.6 | 63.5 | 49.7 | 54.3 | 45.2 | 46.8 | 49.4 | 47.9 | 53.2 | 53.2 | 53.7 | 48.4 | 49.7 |
| 27. | M_truncatula_AC135467_2_5_CDS | 59.1 | 62.5 | 62.2 | 61.3 | 55 | 48.2 | 41.7 | 62.6 | 64.8 | 71.8 | 71.3 | 40.4 | 65 | 56.9 | 60.7 | 83.2 | 64.4 | 84.3 | 72.4 | 72.4 | 64 | 58.3 | 64.8 |
| 28. | M_truncatula_AC157503_6_4 | 61.4 | 59.7 | 58 | 58.5 | 57.7 | 52.1 | 41.1 | 60.8 | 66.2 | 72.8 | 67.8 | 43.7 | 66.9 | 60 | 60.4 | 78.4 | 62.8 | 78.7 | 73.8 | 73.8 | 66.2 | 60.3 | 66.7 |
| 29. | N_benthamiana_TC11136 | 60.6 | 49.9 | 49.6 | 49.9 | 61.5 | 65.5 | 50.9 | 54.5 | 56.9 | 57.3 | 55 | 55.9 | 56.6 | 61.2 | 49.9 | 59.6 | 53.2 | 59 | 65 | 64.3 | 56.2 | 57.1 | 56.3 |
| 30. | N_benthamiana_TC12970 | 56.4 | 60.3 | 62.9 | 60.8 | 55.1 | 48 | 39.7 | 59.3 | 64.5 | 69.5 | 66.8 | 39.9 | 64.8 | 57.2 | 60.7 | 70.5 | 68.4 | 70 | 64 | 64.2 | 65.5 | 56.4 | 64.2 |
| 31. | N_tabacum_TC16005 | 62.8 | 59 | 59.2 | 59.2 | 60.8 | 53 | 43.2 | 61.9 | 66.9 | 69.9 | 65.9 | 44.1 | 64.8 | 62 | 60.2 | 69.5 | 64.4 | 70.1 | 67.7 | 65.4 | 68.9 | 61.7 | 65.8 |
| 32. | O_sativa_LOC_Os05g01940_1 | 59.3 | 61.8 | 59.5 | 59.5 | 54.9 | 47.9 | 44 | 61.9 | 62.7 | 67.9 | 66.3 | 39.1 | 63.2 | 55.4 | 63 | 65.3 | 66.2 | 65.3 | 59.8 | 59.1 | 64.5 | 60.6 | 63.2 |
| 33. | P_trichocarpa_560785 | 67.2 | 54.9 | 51.8 | 52.4 | 63.4 | 57.9 | 46.3 | 59.6 | 67.7 | 66.3 | 69.6 | 49.8 | 60.1 | 62.7 | 57.8 | 63.4 | 63.4 | 63.4 | 68.8 | 69.1 | 66.4 | 70.5 | 60.1 |
| 34. | P_trichocarpa_765468 | 46 | 51.3 | 55.3 | 53 | 46.2 | 39.7 | 34.2 | 51.1 | 53.2 | 55.5 | 55.5 | 34.6 | 52.1 | 46.6 | 55.7 | 54.9 | 60.8 | 56.3 | 49.4 | 48.5 | 56.3 | 47.7 | 52.3 |
| 35. | P_trifoliata_TA5973_37690 | 55.9 | 60 | 58 | 58 | 75.7 | 68.4 | 41.4 | 61.2 | 93.3 | 68.2 | 72.5 | 39.8 | 65 | 55.6 | 58.6 | 62.8 | 65.4 | 64.2 | 59.6 | 59.4 | 65.5 | 61.5 | 66.3 |
| 36. | R_communis_EE259446 | 63.2 | 46.8 | 46.9 | 47.3 | 61.8 | 67 | 55.9 | 57.1 | 59.8 | 59.8 | 61.8 | 56.7 | 56 | 59 | 49.6 | 56.8 | 54.5 | 56 | 59.6 | 59.9 | 57.6 | 59 | 56 |
| 37. | R_communis_TA1159_3988 | 52.6 | 64 | 66.7 | 65.5 | 51.8 | 44.3 | 40.1 | 59.9 | 60.3 | 68.1 | 64.2 | 38.9 | 64.7 | 55 | 59.9 | 64.2 | 63.3 | 64.7 | 58.2 | 58.2 | 61.6 | 54.7 | 65.2 |
| 38. | R_communis_TA1782_3988 | 54.9 | 58.7 | 59.7 | 62 | 54.1 | 46.4 | 38.9 | 58 | 61.1 | 62.9 | 64.2 | 38.7 | 58.8 | 51.8 | 62 | 63.4 | 67.9 | 63.1 | 57.7 | 57.5 | 64.7 | 56.7 | 59.5 |
| 39. | R_communis_TA1803_3988 | 61.8 | 55.2 | 57.2 | 58.5 | 58.7 | 51.1 | 44.4 | 64.6 | 71.1 | 73.6 | 76.7 | 43 | 66.4 | 58.7 | 59.4 | 70.4 | 65.6 | 70.4 | 64 | 64.6 | 69.7 | 63.2 | 66.7 |
| 40. | S_bicolor_Sb09g001100_1 | 54.7 | 57.1 | 56.9 | 55.7 | 49.9 | 43.1 | 37.5 | 55.9 | 57.9 | 62.7 | 61.5 | 35.1 | 61.3 | 52.1 | 56.4 | 59.8 | 61.3 | 59.6 | 54.2 | 54.2 | 57.4 | 54 | 60 |
| 41. | S_tuberosum_TC193013 | 52.1 | 42 | 43.5 | 42.8 | 54.9 | 64.5 | 61.6 | 48 | 49.3 | 51.7 | 48.5 | 65.4 | 49.5 | 53.4 | 42.9 | 51.2 | 45.5 | 51.2 | 55.4 | 54.5 | 48.3 | 48.7 | 49.7 |
| 42. | V_vinifera_GSVIVT00021348001 | 60.1 | 57.5 | 59.5 | 61.3 | 63.2 | 55.1 | 44.6 | 64.5 | 74 | 72.6 | 76.4 | 44 | 67.5 | 60.1 | 61.7 | 70.9 | 68.7 | 70.4 | 63.7 | 62 | 70.4 | 64.3 | 67.5 |
| 43. | Z_mays_TC391585 | 60.9 | 56.2 | 57 | 56.2 | 52.6 | 46.6 | 41.7 | 60.5 | 61.8 | 69.4 | 64.2 | 37.7 | 60.1 | 54.9 | 56.8 | 64.5 | 59 | 63.2 | 60.9 | 62.3 | 64.1 | 60.3 | 60.9 |
| 44. | Z_mays_ZM07MC00480_BFb0175C11 | 61.1 | 56.2 | 57 | 56.7 | 52.9 | 46.9 | 41.7 | 60.5 | 62.3 | 69.9 | 64.5 | 38 | 60.4 | 54.9 | 57.1 | 64.8 | 59.3 | 63.4 | 60.9 | 62.3 | 64.4 | 61.1 | 61.2 |
| 45. | Z_mays_ZM07MC34672_BFb0353B17 | 57 | 59.5 | 58.2 | 58 | 51.2 | 44.6 | 40.6 | 58 | 57.8 | 66.5 | 64.9 | 37.2 | 62.3 | 54.4 | 57.1 | 63.1 | 60.8 | 64.1 | 59.6 | 59.6 | 60.4 | 59.4 | 62.8 |
| 46. | Z_officinale_TA1620_94328 | 59.7 | 59.2 | 58 | 58 | 57.3 | 50.7 | 47.1 | 67.1 | 67.1 | 68.8 | 67.2 | 41.9 | 66.9 | 57.8 | 60.4 | 68.5 | 65.1 | 66.3 | 60.8 | 61.9 | 67.7 | 60.8 | 66.9 |
| | | 24 | 25 | 26 | 27 | 28 | 29 | 30 | 31 | 32 | 33 | 34 | 35 | 36 | 37 | 38 | 39 | 40 | 41 | 42 | 43 | 44 | 45 | 46 |
| 1. | A_cepa_TC4713 | 42.4 | 41.3 | 36 | 43 | 42.6 | 42.9 | 42.5 | 46.7 | 44.4 | 46.6 | 34.2 | 40.4 | 45.5 | 38.6 | 41.4 | 44.8 | 41.8 | 36.4 | 43.8 | 45.5 | 45.5 | 42.7 | 44.5 |
| 2. | A_thaliana_AT3G46620_1 | 45 | 43.4 | 34.9 | 47 | 45.8 | 38.1 | 44.3 | 45.6 | 42.5 | 42.6 | 38.3 | 42.6 | 38.1 | 46.5 | 42.5 | 43.6 | 41.4 | 33.3 | 44.6 | 41.7 | 41.7 | 42.1 | 43 |
| 3. | A_thaliana_AT5G59550_1 | 48.2 | 44.8 | 31.7 | 46.6 | 44.7 | 39.4 | 48 | 45.8 | 42.9 | 40.7 | 42.2 | 42.7 | 36.9 | 48.5 | 42.6 | 43.3 | 40.7 | 32.8 | 44.4 | 41.6 | 41.6 | 41.7 | 41.6 |
| 4. | B_napus_TC85754 | 47.9 | 42.7 | 33.5 | 46.9 | 44.3 | 39.1 | 46.5 | 45.6 | 43.2 | 40.9 | 41.1 | 42.4 | 38.5 | 48.7 | 45.3 | 44.8 | 40.3 | 33.9 | 44.7 | 39.4 | 40 | 41.3 | 42.4 |
| 5. | C_clementina_DY266223 | 41.2 | 41.9 | 44.8 | 43.4 | 43.6 | 50.2 | 44.9 | 48.2 | 43.5 | 48.7 | 35.9 | 73.5 | 50.6 | 40.4 | 42.1 | 45.4 | 40.1 | 45.2 | 50.7 | 37.9 | 37.9 | 38.4 | 46.3 |
| 6. | C_clementina_TC3900 | 35.6 | 36.4 | 52.6 | 37.1 | 39 | 51 | 38.1 | 41.2 | 37.8 | 43.4 | 30.4 | 67.4 | 50 | 34.9 | 36 | 39.2 | 33.8 | 52.4 | 43.6 | 32.4 | 32.4 | 32.4 | 40.4 |
| 7. | C_longa_TA1491_136217 | 30.1 | 31.7 | 43 | 30.7 | 30.4 | 38.6 | 31.1 | 33.5 | 35.4 | 31.7 | 26.5 | 29.9 | 41 | 29.8 | 31.1 | 33.3 | 30.8 | 45.1 | 33.3 | 32.5 | 32.5 | 32.4 | 41 |
| 8. | C_longa_TA268_136217 | 41.1 | 40.3 | 35.5 | 42.9 | 41.2 | 40.6 | 42.4 | 44 | 46.2 | 44.3 | 36 | 40.5 | 43 | 41.2 | 40.6 | 44.3 | 41.4 | 36.2 | 45.5 | 42.3 | 42.8 | 41.9 | 53.4 |
| 9. | C_sinensis_TC458 | 47.8 | 47 | 40.9 | 49.5 | 49.6 | 45.7 | 51.3 | 53.3 | 49.2 | 56.2 | 41.5 | 92.5 | 49.4 | 48.2 | 45.1 | 55.5 | 46.3 | 40.9 | 58.5 | 43.6 | 43.8 | 43.8 | 51.8 |
| 10. | F_vesca_TA10341_57918 | 56.1 | 50.4 | 40.4 | 60.1 | 56.6 | 46.1 | 57.1 | 56.3 | 57.1 | 55.1 | 45.7 | 53.1 | 50.3 | 55.1 | 50.4 | 58.7 | 53.8 | 41.8 | 59.3 | 55.6 | 56.1 | 52.1 | 51.6 |
| 11. | G_hirsutum_TC105257 | 53.6 | 55 | 41 | 57.1 | 51.9 | 46 | 54.8 | 54.4 | 52.3 | 60.9 | 44.6 | 59.6 | 53.3 | 54.6 | 50.4 | 63.8 | 49.5 | 41.6 | 63.7 | 47.8 | 48 | 48.4 | 52 |
| 12. | G_hirsutum_TC82591 | 30.5 | 31.5 | 50.8 | 31.1 | 33.6 | 43.2 | 30.9 | 34.1 | 28.3 | 33.9 | 26.4 | 29.7 | 42.4 | 31 | 29.8 | 32.7 | 26.2 | 49.2 | 33.1 | 27.2 | 27.5 | 27.6 | 31.9 |
| 13. | G_hirsutum_TC85784 | 46.1 | 48.9 | 38.9 | 48.2 | 47.5 | 43.5 | 49.9 | 50.3 | 45.5 | 43.2 | 40.2 | 48.6 | 42.9 | 50.5 | 42.9 | 48.5 | 44.2 | 40.4 | 51.3 | 43 | 43.2 | 44 | 46.9 |
| 14. | G_hirsutum_TC92683 | 38.7 | 41.9 | 40.9 | 41.6 | 42.1 | 44.1 | 42.2 | 44.1 | 40.2 | 43.6 | 34.8 | 40.4 | 43.1 | 41.2 | 37.3 | 41.2 | 37.3 | 39.1 | 43.4 | 37.2 | 37.2 | 37.6 | 42.9 |
| 15. | G_hirsutum_TC93828 | 45.4 | 45.4 | 35.4 | 46.8 | 46.3 | 39.1 | 44.5 | 46.4 | 45.2 | 43.7 | 40.5 | 42.5 | 39.2 | 44.9 | 45 | 44.3 | 42.1 | 32.7 | 46.2 | 42 | 42 | 41.2 | 44.7 |
| 16. | G_max_Glyma11g14580_1 | 67.7 | 48.4 | 37.1 | 75.1 | 63.5 | 48 | 57.1 | 56.1 | 53.1 | 50.8 | 43.7 | 48.4 | 45.4 | 50.8 | 47.9 | 54.5 | 48.4 | 42 | 54 | 48.6 | 48.8 | 47.9 | 51.8 |
| 17. | G_max_Glyma11g34160_1 | 50.2 | 54.8 | 39.2 | 51.2 | 48.1 | 41.7 | 52.8 | 51.6 | 51.9 | 50.8 | 49 | 49 | 46.3 | 51.2 | 52.4 | 53.9 | 47.8 | 36.7 | 54.3 | 46.6 | 46.8 | 45.4 | 51.8 |
| 18. | G_max_Glyma12g06460_1 | 68.4 | 48.6 | 37.2 | 75.8 | 62.9 | 46.9 | 56.8 | 55.9 | 52.8 | 50.8 | 44.5 | 49.5 | 44.8 | 51.1 | 48 | 54.5 | 49.4 | 40.7 | 55 | 47.9 | 48.2 | 49 | 50.3 |
| 19. | G_max_Glyma13g41340_1 | 55.4 | 44.7 | 40.1 | 60.6 | 63.7 | 49.7 | 54.1 | 55.6 | 48.3 | 52.3 | 40.5 | 47.7 | 45.5 | 47.4 | 47.6 | 50.7 | 44.1 | 42.7 | 48.8 | 47.1 | 47.1 | 47 | 48.4 |
| 20. | G_max_Glyma15g04080_1 | 55.8 | 45.1 | 40.6 | 61.4 | 64.7 | 49.7 | 54.5 | 54.4 | 48.3 | 52.1 | 39.7 | 47.6 | 46.1 | 46.7 | 47.3 | 52 | 43.8 | 42.7 | 48.3 | 47.9 | 47.9 | 48.3 | 49.2 |
| 21. | G_max_Glyma18g04140_1 | 48.4 | 54.6 | 40.9 | 50.3 | 48.6 | 42.5 | 50.9 | 52.9 | 51.6 | 51.5 | 44.9 | 50.4 | 47.4 | 49.6 | 50.2 | 53.8 | 46.2 | 38 | 54.9 | 48.8 | 49.1 | 46 | 52 |
| 22. | G_max_Glyma18g40130_1 | 43.6 | 44.6 | 34 | 46.1 | 46.9 | 40 | 43.8 | 48.2 | 47.5 | 55.8 | 37.6 | 45.6 | 46.3 | 42.3 | 43.3 | 51.1 | 43.5 | 33.8 | 50.5 | 46 | 46.6 | 43.7 | 46 |
| 23. | G_raimondii_TC6392 | 46.2 | 48.3 | 38.9 | 48.7 | 47.6 | 43.5 | 49.6 | 50.4 | 45.9 | 43.2 | 40.8 | 48 | 42.9 | 50.7 | 43.3 | 49.2 | 44.1 | 40.4 | 51 | 42.9 | 43.1 | 44.2 | 47.5 |
| 24. | L_japonicus_TC26018 | | 46.6 | 36.9 | 70.6 | 60.6 | 42.5 | 51.6 | 49.9 | 49.5 | 45.7 | 43.3 | 48.6 | 42.2 | 48.6 | 46.2 | 48.9 | 45 | 37.2 | 51.5 | 46.1 | 45.8 | 45.1 | 47.7 |
| 25. | L_sativa_TC27450 | 63.3 | | 38.8 | 49.5 | 44.9 | 40.8 | 50.7 | 49 | 47.7 | 46.2 | 43.7 | 47.9 | 43.8 | 48.2 | 49.8 | 51.2 | 43.9 | 35.8 | 50.6 | 44.4 | 44.7 | 43.3 | 45.3 |
| 26. | M_esculenta_TA5606_3983 | 47 | 50.1 | | 37.2 | 37.3 | 49.2 | 38 | 40.3 | 36.4 | 38.7 | 35.5 | 37.6 | 46.2 | 36.2 | 45.5 | 36.6 | 31.3 | 56.8 | 40.2 | 31.8 | 31.8 | 31.4 | 41.1 |
| 27. | M_truncatula_AC135467_2_5_CDS | 80.5 | 65.9 | 47.7 | | 63.3 | 45.8 | 53.9 | 53.9 | 51.3 | 51.8 | 46.1 | 49.9 | 45.1 | 51.6 | 47.3 | 53.4 | 46.3 | 38.5 | 53.8 | 47 | 47 | 46.9 | 49.9 |
| 28. | M_truncatula_AC157503_6_4 | 74.4 | 62.3 | 49.3 | 78.3 | | 45.6 | 52.4 | 53 | 48.9 | 49.7 | 42.2 | 47.4 | 44 | 46.9 | 43.9 | 49.1 | 43.9 | 39 | 51.7 | 43 | 43 | 43.1 | 47.4 |
| 29. | N_benthamiana_TC11136 | 54.9 | 53.2 | 61.9 | 58.5 | 58.9 | | 67.9 | 74.1 | 41.6 | 45.3 | 35.8 | 42.5 | 53.2 | 43.1 | 40.2 | 42.9 | 37.4 | 69.2 | 45.9 | 40.7 | 40.7 | 39.6 | 45.6 |
| 30. | N_benthamiana_TC12970 | 65.8 | 66.9 | 46.2 | 70.5 | 66.3 | 70.8 | | 88 | 52.2 | 49.4 | 46.2 | 50.5 | 45.5 | 54.3 | 48.7 | 53 | 45.4 | 52.1 | 53.3 | 48 | 48 | 47 | 50.1 |
| 31. | N_tabacum_TC16005 | 63.3 | 62.8 | 50.1 | 69.6 | 68.2 | 76.4 | 88.3 | | 52 | 53.5 | 44.7 | 52 | 49.3 | 52.1 | 47.7 | 53.5 | 46.4 | 57.4 | 55.1 | 49.2 | 49.2 | 48.1 | 53.3 |
| 32. | O_sativa_LOC_Os05g01940_1 | 64 | 62.8 | 45.3 | 64.2 | 63.5 | 51.6 | 67.1 | 63.5 | | 49.9 | 42.8 | 49 | 43.8 | 47.8 | 45.1 | 50.9 | 70.1 | 36.2 | 51 | 70.1 | 70.6 | 66.6 | 52.6 |
| 33. | P_trichocarpa_560785 | 57.5 | 59.4 | 53.4 | 63.7 | 62.5 | 60.8 | 61.4 | 67.7 | 60.4 | | 41.5 | 53.8 | 61.4 | 46.5 | 43.9 | 65.4 | 45.8 | 38.3 | 56.5 | 45.7 | 46.3 | 45.5 | 54.5 |
| 34. | P_trichocarpa_765468 | 56.1 | 57.6 | 42.6 | 58.4 | 54.6 | 43.2 | 55.3 | 53.4 | 54.6 | 50.2 | | 42.5 | 37.2 | 44 | 50 | 43.6 | 41.7 | 29.8 | 45.3 | 39.1 | 39.3 | 38.8 | 43.4 |
| 35. | P_trifoliata_TA5973_37690 | 63.1 | 64.6 | 47.9 | 64.4 | 63.4 | 53.2 | 66.1 | 66.3 | 64.2 | 63.9 | 55.5 | | 46.6 | 47.3 | 45.4 | 55.3 | 45.8 | 38 | 57.4 | 42.7 | 42.9 | 43.2 | 49.1 |
| 36. | R_communis_EE259446 | 53.6 | 53.7 | 63 | 54.2 | 57.2 | 65.8 | 53.8 | 58.5 | 53.6 | 70.7 | 44.5 | 56.1 | | 43.8 | 41.4 | 75.6 | 42.2 | 49.1 | 53 | 43.6 | 43.8 | 42.6 | 47.5 |
| 37. | R_communis_TA1159_3988 | 65.5 | 65.7 | 44.8 | 65.2 | 62.3 | 51.3 | 65.7 | 61.8 | 60.6 | 58.4 | 58.9 | 60.6 | 55.2 | | 47.8 | 51.2 | 46.3 | 38.2 | 51.7 | 45.3 | 45.3 | 45 | 48.8 |
| 38. | R_communis_TA1782_3988 | 63.7 | 68.8 | 54.1 | 62.9 | 60.6 | 50 | 63.1 | 61.6 | 61.1 | 57.7 | 60.1 | 62.6 | 51.5 | 64.2 | | 49.5 | 43.6 | 36 | 47.4 | 44 | 44.2 | 41.7 | 46.2 |
| 39. | R_communis_TA1803_3988 | 64.9 | 64.9 | 48.3 | 67.2 | 67.7 | 53.7 | 65.5 | 66.6 | 64.2 | 73.3 | 54.2 | 68.4 | 75.6 | 66.7 | 63.7 | | 47.3 | 38 | 60.2 | 48.3 | 48.8 | 48.5 | 52 |
| 40. | S_bicolor_Sb09g001100_1 | 59.6 | 58.6 | 39.5 | 58.1 | 59.1 | 46.2 | 60 | 57.4 | 78.5 | 56.2 | 53.8 | 58.1 | 51.3 | 63.2 | 56.2 | 60.5 | | 32.7 | 47.6 | 70.9 | 71.4 | 72.5 | 49.8 |
| 41. | S_tuberosum_TC193013 | 48 | 46.5 | 68.2 | 49.6 | 49.9 | 76.5 | 56.7 | 62.5 | 45.9 | 51.8 | 37.3 | 46.8 | 62.6 | 45.5 | 44.8 | 47.5 | 41.4 | | 40.3 | 34.8 | 34.8 | 33.7 | 39.2 |
| 42. | V_vinifera_GSVIVT00021348001 | 65.2 | 66.9 | 49.6 | 68.3 | 69 | 58.4 | 68.4 | 70.1 | 64.5 | 67 | 57.6 | 72.5 | 60.9 | 65.2 | 62.1 | 73.7 | 60 | 50.7 | | 46.8 | 47 | 47.1 | 53.5 |
| 43. | Z_mays_TC391585 | 62 | 59.4 | 44.6 | 62.9 | 62 | 53.7 | 63.7 | 66.6 | 77.7 | 60.3 | 48.7 | 58.8 | 54.9 | 58.4 | 58.8 | 63.8 | 76.3 | 46.6 | 62 | | 99.4 | 76 | 48.4 |
| 44. | Z_mays_ZM07MC00480_BFb0175C11 | 62 | 59.9 | 44.6 | 62.9 | 62 | 53.7 | 63.7 | 66.6 | 78.2 | 60.9 | 49.2 | 59.4 | 55.1 | 58.4 | 59 | 64.3 | 76.8 | 46.6 | 62.3 | 99.4 | | 76.3 | 48.6 |
| 45. | Z_mays_ZM07MC34672_BFb0353B17 | 63.9 | 60.7 | 42.2 | 62 | 60.9 | 51.2 | 64.8 | 63.1 | 78.5 | 59.6 | 49.4 | 58.6 | 52.2 | 60.3 | 57.7 | 61.7 | 80.1 | 44.9 | 62.5 | 82.1 | 82.3 | | 49 |
| 46. | Z_officinale_TA1620_94328 | 63.3 | 63 | 51.5 | 66.7 | 64.7 | 57 | 65 | 67.1 | 65.5 | 67.7 | 54.9 | 65 | 57.8 | 62.3 | 63.4 | 68.2 | 62.2 | 47.7 | 69.3 | 63 | 63.3 | 64.6 | |

A MATGAT table for local alignment of a specific domain, or data on % identity/similarity between specific domains may also be performed.

### 3.2. SPATULA-Like (SPT) Polypeptides

Global percentages of similarity and identity between full length polypeptide sequences useful in performing the methods of the invention were determined using MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table B2 for the global similarity and identity over the full length of the polypeptide sequences. Percentage identity is given above the diagonal and percentage similarity is given below the diagonal.

TABLE B2
MatGAT results for global similarity and identity over the full length of the polypeptide sequences.

| | | 1 | 2 | 3 | 4 | 5 | 6 | 7 | 8 | 9 | 10 |
|---|---|---|---|---|---|---|---|---|---|---|---|
| 1. | Poptr_SPT | | 34 | 35.2 | 53.4 | 54.6 | 37.4 | 38.6 | 38 | 46.8 | 38.5 |
| 2. | Arath_SPT | 49.1 | | 61.5 | 35.3 | 33.8 | 37.6 | 42.1 | 37.7 | 39.4 | 33.5 |
| 3. | Brana_SPT | 52.9 | 67.8 | | 32.7 | 30.8 | 32.5 | 43.8 | 34.9 | 38.1 | 32.1 |
| 4. | Carpa_SPT | 66.7 | 53.4 | 49.1 | | 50.6 | 39.4 | 37.2 | 36.3 | 44.3 | 34.6 |
| 5. | Cicle_SPT | 68.4 | 50.1 | 51.6 | 63.7 | | 38.6 | 37.7 | 37.9 | 48.1 | 40.3 |
| 6. | Escca_SPT | 51.5 | 51.3 | 42.7 | 53.6 | 50.8 | | 36.5 | 40.9 | 40.3 | 39 |
| 7. | Fra\x\ana_SPT | 55.8 | 52.8 | 60.4 | 51.2 | 56.3 | 47.1 | | 39 | 44.7 | 41.1 |
| 8. | Glyma_SPT | 50.7 | 55.4 | 47.5 | 51.7 | 50.7 | 52.7 | 52 | | 43.1 | 57.6 |
| 9. | Goshi_SPT | 63.5 | 53.1 | 59.3 | 57.9 | 62.8 | 48.3 | 60.7 | 51.4 | | 45.6 |
| 10. | Medtr_SPT | 53.8 | 52.8 | 48.3 | 50.6 | 56.1 | 51 | 54.9 | 69.2 | 56.4 | |
| 11. | Nicta_SPT | 50.3 | 46.9 | 57 | 48 | 51.6 | 41.1 | 58.7 | 42.8 | 50.2 | 47.7 |
| 12. | Solly_SPT | 52.1 | 55 | 47.4 | 52.6 | 51.3 | 56.1 | 48.4 | 56.9 | 51.1 | 57.1 |
| 13. | Vinvi_SPT | 61.3 | 52.8 | 60.3 | 56.1 | 63.8 | 49.9 | 64.1 | 52.7 | 70 | 57 |
| 14. | Glyma_SPT\like\1 | 50.7 | 56.7 | 48.8 | 52.2 | 50.1 | 53.4 | 51.4 | 96.6 | 50.9 | 70.1 |
| 15. | Glyma_SPT\like\2 | 53.5 | 51.5 | 61.6 | 48.5 | 53.9 | 44.5 | 60.1 | 66.3 | 54.5 | 57.3 |
| 16. | Solly_SPT\like\1 | 62.4 | 49.1 | 50 | 60.5 | 61.5 | 50.6 | 50.6 | 50.9 | 58.6 | 52.3 |
| 17. | Solly_SPT\like\2 | 43.2 | 41.8 | 53.2 | 42.1 | 43.4 | 36.7 | 49.3 | 39.9 | 45.5 | 41.9 |
| 18. | Orysa_SPT\like | 49.7 | 42.4 | 39.4 | 43.9 | 47.2 | 39.7 | 44.1 | 44.1 | 46.9 | 49.4 |
| 19. | Arath_ALC | 44.8 | 38.3 | 47.5 | 41.2 | 42.8 | 34.3 | 47.3 | 37.3 | 46.1 | 41.6 |

| | | 11 | 12 | 13 | 14 | 15 | 16 | 17 | 18 | 19 |
|---|---|---|---|---|---|---|---|---|---|---|
| 1. | Poptr_SPT | 36.3 | 38.1 | 46.2 | 39.3 | 38.1 | 43.4 | 32.5 | 30.7 | 33.2 |
| 2. | Arath_SPT | 35.7 | 35 | 41 | 37.5 | 40.8 | 32.5 | 31.1 | 27.2 | 30.6 |
| 3. | Brana_SPT | 42.5 | 31.9 | 41.9 | 35.8 | 45 | 32.5 | 40 | 23.4 | 36.6 |
| 4. | Carpa_SPT | 35.3 | 35.7 | 44.1 | 35.8 | 34.8 | 43.4 | 32.5 | 29.2 | 30.4 |
| 5. | Cicle_SPT | 36 | 36.5 | 45.7 | 36.8 | 37.2 | 43.9 | 29.8 | 29 | 33.4 |
| 6. | Escca_SPT | 31.4 | 39.2 | 40 | 41 | 34.8 | 35.1 | 29.3 | 27.5 | 26 |
| 7. | Fra\x\ana_SPT | 43.4 | 35.4 | 50.5 | 40.1 | 44.2 | 35 | 36.9 | 26.6 | 36 |
| 8. | Glyma_SPT | 31.3 | 37.9 | 42 | 95.1 | 62.1 | 36.3 | 29.3 | 31 | 30.1 |
| 9. | Goshi_SPT | 37.1 | 37.9 | 55 | 42.1 | 40 | 40.9 | 33.6 | 28.8 | 34 |
| 10. | Medtr_SPT | 33.7 | 39.2 | 44.7 | 59.2 | 43.2 | 33.9 | 30.2 | 32.9 | 30.5 |
| 11. | Nicta_SPT | | 49 | 45.3 | 31.8 | 41.3 | 34 | 65.8 | 24.9 | 38 |
| 12. | Solly_SPT | 55.3 | | 36.4 | 38.2 | 34.8 | 34.5 | 57.1 | 29.4 | 29.1 |
| 13. | Vinvi_SPT | 58 | 51.3 | | 41.3 | 43.8 | 39.2 | 38.6 | 28.6 | 39.3 |
| 14. | Glyma_SPT\like\1 | 43.8 | 55.6 | 51.4 | | 58.3 | 37.1 | 29.3 | 30.9 | 29.5 |
| 15. | Glyma_SPT\like\2 | 57 | 49.2 | 58.3 | 63.8 | | 35 | 40.6 | 26.8 | 38.7 |
| 16. | Solly_SPT\like\1 | 45.5 | 48.9 | 58.3 | 52 | 54.1 | | 30.4 | 29.8 | 30.1 |
| 17. | Solly_SPT\like\2 | 75.6 | 57.1 | 51.5 | 38.6 | 55.2 | 42.4 | | 21.2 | 35.1 |
| 18. | Orysa_SPT\like | 38.4 | 43.4 | 45.6 | 44.4 | 43.1 | 48.1 | 32.2 | | 23.7 |
| 19. | Arath_ALC | 53.7 | 36.8 | 48.1 | 36.7 | 48.4 | 39.8 | 53 | 35.9 | |

A MATGAT table for local alignment of a specific domain, or data on % identity/similarity between specific domains may also be performed.

### 3.3. IDI2 (Iron Deficiency Induced 2) Polypeptides

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table B3 for the global similarity and identity over the full length of the polypeptide sequences.

The percentage identity between the IDI2 polypeptide sequences useful in performing the methods of the invention can be as low as 24% amino acid identity compared to SEQ ID NO: 140.

TABLE B3
MatGAT results for global similarity and identity over the full length of the polypeptide sequences.

| | | 1 | 2 | 3 | 4 | 5 | 6 | 7 | 8 | 9 | 10 | 11 | 12 | 13 |
|---|---|---|---|---|---|---|---|---|---|---|---|---|---|---|
| 1. | Saccof_IDI2 | | 25.3 | 88 | 26.1 | 84.8 | 40.1 | 35.6 | 33.7 | 84.8 | 53.3 | 68 | 24.1 | 96.7 |
| 2. | Chlorella_140697 | 33.5 | | 25.3 | 50.9 | 24.9 | 20.2 | 9.6 | 9.2 | 24.7 | 26.4 | 24.7 | 41.1 | 25.3 |
| 3. | H. vulgare_TA31842_4513 | 93.5 | 33.3 | | 26.3 | 84.5 | 40.1 | 34.5 | 32.3 | 84.5 | 53.3 | 66.1 | 23 | 87.5 |
| 4. | O. RCC809_50296 | 34.1 | 68.1 | 34 | | 25.7 | 19.3 | 10 | 9.5 | 25.6 | 37.1 | 24.8 | 42.9 | 26.2 |
| 5. | O. sativa.indica_BGIOSIBCE033922 | 91.2 | 33.5 | 92 | 34 | | 39.2 | 41.4 | 39.3 | 99.7 | 52.3 | 66.1 | 23.1 | 85.3 |
| 6. | O. sativa.indica_BGIOSIBSE037940 | 56.7 | 26 | 55.6 | 26.2 | 53.7 | | 16.5 | 14.2 | 39.1 | 42.1 | 40.8 | 18.4 | 39.1 |
| 7. | O. sativa_CR292756 | 38.1 | 13.3 | 39 | 13.6 | 41.4 | 23.7 | | 92.4 | 41.2 | 19.7 | 25.3 | 8.1 | 35.9 |
| 8. | O. sativa_CX116019 | 37.1 | 12.8 | 37.1 | 13.4 | 40.1 | 21.9 | 93.7 | | 39 | 19.5 | 23.4 | 8 | 33.7 |
| 9. | O. sativa_Os11g0216900 | 90.9 | 33.4 | 91.7 | 33.9 | 99.7 | 54 | 41.2 | 39.8 | | 52.1 | 66.1 | 23.1 | 85.3 |
| 10. | O. taurii_8569 | 68.7 | 33.7 | 69 | 40.2 | 68.2 | 56.9 | 25.9 | 25.3 | 67.9 | | 47.7 | 23 | 53 |
| 11. | P. pinaster_TA4183_71647 | 79.4 | 32.9 | 78.4 | 33.1 | 79.9 | 54.9 | 31.3 | 29.7 | 79.7 | 64.8 | | 22.7 | 67.7 |
| 12. | P. tricornutum_23811 | 31.7 | 59.4 | 31.7 | 62.2 | 32.2 | 25.9 | 12.4 | 12.2 | 32.1 | 30.7 | 32.3 | | 24.1 |
| 13. | S. bicolor_Sb05g008680.1 | 98.4 | 33.6 | 93.7 | 34.5 | 92.2 | 55.3 | 38.7 | 37.3 | 92 | 68.5 | 79.7 | 32.4 | |
| 14. | S. bicolor_TA25485_4558 | 98.1 | 33.5 | 93.5 | 34.4 | 92 | 55.3 | 38.7 | 37.3 | 91.7 | 68.2 | 79.4 | 32.3 | 99.7 |
| 15. | S. officinarum_TA35690_4547 | 42.2 | 14.8 | 39.5 | 15 | 38.8 | 26.6 | 81.2 | 78.8 | 38.5 | 28 | 32.6 | 14.7 | 42.2 |
| 16. | T. aestivum_c54899571@13348 | 93.5 | 33 | 97.5 | 34.5 | 92 | 55.3 | 39 | 36.8 | 91.7 | 68.7 | 78.9 | 31.7 | 93.5 |
| 17. | T. aestivum_CV772651 | 34.3 | 12.9 | 36.8 | 14.1 | 34.2 | 23.1 | 76.6 | 78.3 | 34.5 | 25.6 | 28.1 | 13 | 34.6 |
| 18. | T. aestivum_TA67133_4565 | 93.7 | 33 | 98.1 | 34.6 | 92.2 | 54.8 | 39.2 | 37.1 | 92 | 69 | 79.2 | 31.9 | 93.7 |
| 19. | T. pseudonana_35896 | 66 | 28.7 | 65.4 | 28.9 | 65.5 | 52.8 | 27.1 | 25.7 | 65.2 | 60.9 | 64.3 | 33.9 | 66.8 |
| 20. | V. carteri_59470 | 66.3 | 37.1 | 65.8 | 35.9 | 66.6 | 50.6 | 25.9 | 25.9 | 66.6 | 65.1 | 69.1 | 31.6 | 66.3 |
| 21. | Z. mays_c57808725gm030403@2572 | 75.2 | 25.7 | 71.1 | 26.7 | 69.5 | 47.7 | 49.5 | 47.7 | 69.3 | 54.2 | 59.9 | 24.6 | 74.9 |
| 22. | Z. mays_DQ244248 | 66.8 | 23.4 | 62.4 | 23.7 | 61.5 | 40.4 | 54.4 | 51.7 | 61.2 | 46.9 | 52.6 | 21.8 | 66.2 |
| 23. | Z. mays_ZM07MC01636_57808725 | 98.1 | 33.3 | 93.5 | 34.2 | 91.4 | 55.6 | 38.1 | 37.1 | 91.2 | 69 | 78.6 | 31.6 | 98.1 |
| 24. | A. thaliana_AT2G05830.1 | 83.2 | 33.1 | 84.5 | 34.4 | 82.6 | 55.1 | 32.9 | 32.4 | 82.4 | 69.5 | 80.2 | 31.6 | 83.2 |
| 25. | B. napus_DY025654 | 52 | 19.9 | 52.3 | 21 | 50.3 | 35.7 | 53.2 | 51.1 | 50 | 44.2 | 48.4 | 19.3 | 52.3 |
| 26. | C. clementina_DY262513 | 78.7 | 30.7 | 77.9 | 29.8 | 75.4 | 53.7 | 37.4 | 35.6 | 75.1 | 61.2 | 70.8 | 28.8 | 78.5 |
| 27. | C. clementina_DY262587 | 51.5 | 19.3 | 51.5 | 19.6 | 48.7 | 35.4 | 55.8 | 52.4 | 48.4 | 39.6 | 46.4 | 18.2 | 51.8 |
| 28. | C. clementina_DY263526 | 51.5 | 18.9 | 50.1 | 18.9 | 49.5 | 33.3 | 56.9 | 54.2 | 49.2 | 39.4 | 46.9 | 17.7 | 51.5 |
| 29. | C. clementina_DY268933 | 68.4 | 26 | 67.6 | 25.7 | 65.2 | 48.5 | 44.7 | 41.6 | 65 | 53.6 | 60.9 | 24 | 68.1 |
| 30. | C. clementina_TA2451_85681 | 79.6 | 29.7 | 79 | 29.5 | 76.2 | 56 | 38.2 | 36.4 | 75.9 | 62.3 | 71.4 | 28.2 | 79.3 |
| 31. | C. tinctorius_TA325_4222 | 85.4 | 32.5 | 84.1 | 33.4 | 84.2 | 55.8 | 34.5 | 32.6 | 84 | 69.5 | 80.2 | 31.8 | 84.6 |
| 32. | G. max_Glyma09g08190.1 | 60.5 | 23.9 | 60.5 | 25.7 | 58.6 | 42.1 | 44.1 | 42.7 | 58.3 | 50.7 | 56.5 | 23.1 | 61 |
| 33. | H. tuberosus_TA3030_4233 | 86.3 | 33.3 | 84.9 | 33.9 | 86.4 | 56.7 | 35.2 | 33.6 | 86.1 | 71 | 80.2 | 31.5 | 86.3 |
| 34. | I. nil_CJ747673 | 46.9 | 17.7 | 45.5 | 17.4 | 44.9 | 30.4 | 61.5 | 57.2 | 44.7 | 36.9 | 42.7 | 16.8 | 46.9 |
| 35. | L. virosa_TA2328_75947 | 67.6 | 25.8 | 67.3 | 26.2 | 66.3 | 48.5 | 44.1 | 41.7 | 66 | 55 | 62 | 24 | 67 |
| 36. | P. nigra_TA2942_3691 | 30.5 | 11.7 | 30 | 12.3 | 29.4 | 21.9 | 67.1 | 68.4 | 29.1 | 25.1 | 28.4 | 12.4 | 30 |
| 37. | P. trichocarpa_832064 | 83.1 | 34 | 81.3 | 35.1 | 82.8 | 54.2 | 34.6 | 33.1 | 82.6 | 67.4 | 79.7 | 32.7 | 82.8 |
| 38. | P. trichocarpa_scaff_VI.1535 | 85.1 | 33 | 83.5 | 34 | 84.3 | 54.7 | 35.5 | 33.9 | 84 | 68 | 78.9 | 31.6 | 84.8 |
| 39. | V. shuttleworthii_CN604099 | 30.8 | 12.6 | 30.8 | 13.3 | 28.9 | 20.8 | 65.8 | 68.4 | 28.6 | 24.5 | 28.4 | 11.8 | 30.8 |
| 40. | V. vinifera_GSVIVT00016416001 | 85.9 | 33.4 | 85.4 | 34.7 | 85.6 | 54 | 34.6 | 33.2 | 85.4 | 68.6 | 79.7 | 31.6 | 85.4 |
| 41. | V. vinifera_TA40906_29760 | 85.9 | 33.3 | 85.4 | 34.7 | 85.6 | 54 | 34.6 | 33.2 | 85.4 | 68.6 | 79.7 | 31.6 | 85.4 |
| | | 14 | 15 | 16 | 17 | 18 | 19 | 20 | 21 | 22 | 23 | 24 | 25 | 26 |
| 1. | Saccof_IDI2 | 96.5 | 40.3 | 88 | 31.3 | 88.3 | 46.9 | 52.3 | 73.3 | 65.1 | 96.5 | 67.2 | 41.5 | 64.7 |
| 2. | Chlorella_140697 | 25.2 | 10.1 | 25.3 | 9.8 | 25.3 | 20.7 | 30.1 | 20.1 | 17.8 | 25.4 | 26 | 15.4 | 23.3 |
| 3. | H. vulgare_TA31842_4513 | 87.2 | 35.1 | 95.6 | 35.7 | 96.2 | 45.3 | 51.6 | 66.8 | 58.9 | 88.6 | 67.5 | 41.2 | 63.4 |
| 4. | O. RCC809_50296 | 26.1 | 10.9 | 26.1 | 10.3 | 26.2 | 20.4 | 27.3 | 20.2 | 17.9 | 26 | 26.6 | 16 | 22 |
| 5. | O. sativa.indica_BGIOSIBCE033922 | 85 | 35 | 84.2 | 30.7 | 84.5 | 46.6 | 50.6 | 63.9 | 56.1 | 84.5 | 65.7 | 39 | 59.7 |
| 6. | O. sativa.indica_BGIOSIBSE037940 | 39.1 | 16.2 | 40.4 | 15.7 | 39.9 | 37.1 | 37.8 | 31 | 24.7 | 39.7 | 39.2 | 22.3 | 35.8 |
| 7. | O. sativa_CR292756 | 35.9 | 73.7 | 35.3 | 69.6 | 35.6 | 19.2 | 18.7 | 45.4 | 49.6 | 35.1 | 24.9 | 39.5 | 29.6 |
| 8. | O. sativa_CX116019 | 33.7 | 71.3 | 32.6 | 69.9 | 32.9 | 17.9 | 18.6 | 43 | 46.5 | 33.2 | 24.1 | 38.2 | 28.2 |
| 9. | O. sativa_Os11g0216900 | 85 | 35 | 84.2 | 30.7 | 84.5 | 46.4 | 50.6 | 63.9 | 56.1 | 84.5 | 65.4 | 38.7 | 59.7 |
| 10. | O. taurii_8569 | 52.8 | 20.8 | 52.6 | 18.1 | 52.5 | 44.3 | 51.1 | 42.1 | 36.3 | 53.3 | 53.8 | 33.1 | 44.4 |
| 11. | P. pinaster_TA4183_71647 | 67.4 | 26 | 66.9 | 23.7 | 67.2 | 43.6 | 52.5 | 51 | 43.8 | 67.2 | 64.3 | 36.9 | 56.8 |
| 12. | P. tricornutum_23811 | 24 | 10.3 | 23.2 | 8.8 | 23.2 | 25.2 | 23.7 | 18.4 | 15.8 | 24.1 | 22.6 | 13.3 | 20.3 |
| 13. | S. bicolor_Sb05g008680.1 | 99.7 | 40.6 | 87.7 | 31.3 | 88 | 47.4 | 52.1 | 72.2 | 63.8 | 95.9 | 67.2 | 41.2 | 63.9 |
| 14. | S. bicolor_TA25485_4558 | | 40.6 | 87.5 | 31.3 | 87.7 | 47.4 | 51.9 | 71.9 | 63.5 | 95.6 | 66.9 | 41 | 63.6 |
| 15. | S. officinarum_TA35690_4547 | 42.2 | | 34.9 | 63.5 | 35.1 | 19.5 | 20.4 | 51.2 | 55.2 | 39.2 | 25.4 | 41.2 | 31.6 |
| 16. | T. aestivum_c54899571@13348 | 93.2 | 39.2 | | 36.2 | 99.5 | 45.3 | 52.1 | 67 | 58.6 | 88.6 | 66.7 | 40.4 | 63.1 |
| 17. | T. aestivum_CV772651 | 34.6 | 72.4 | 37.3 | 36.5 | 16.4 | 19.5 | 41 | 44.4 | 31.6 | 23.3 | 36.1 | 27.9 | |
| 18. | T. aestivum_TA67133_4565 | 93.5 | 39.5 | 99.5 | 37.6 | | 45.3 | 51.9 | 67 | 58.9 | 88.8 | 66.9 | 41 | 63.4 |
| 19. | T. pseudonana_35896 | 66.8 | 29.8 | 66.2 | 27.6 | 66.2 | | 41.3 | 35.5 | 31.2 | 46.6 | 44.3 | 28.1 | 39.6 |
| 20. | V. carteri_59470 | 66.1 | 27.4 | 66.6 | 26.2 | 66.6 | 56.9 | | 42.1 | 36.9 | 51.6 | 52.5 | 33.9 | 45.7 |
| 21. | Z. mays_c57808725gm030403@2572 | 74.7 | 54.4 | 71.4 | 44.5 | 71.1 | 51.5 | 52.1 | | 86.6 | 74.7 | 51.6 | 54 | 61 |
| 22. | Z. mays_DQ244248 | 65.9 | 59.1 | 62.1 | 48.3 | 62.4 | 47.2 | 45.6 | 88 | | 65.9 | 45.5 | 58.9 | 53.9 |
| 23. | Z. mays_ZM07MC01636_57808725 | 97.8 | 41.4 | 93.5 | 34.3 | 93.7 | 66.8 | 66.6 | 75.5 | 66.8 | | 66.9 | 41.5 | 64.5 |
| 24. | A. thaliana_AT2G05830.1 | 82.9 | 35.8 | 84.5 | 31.6 | 84.5 | 65.8 | 67.8 | 63.6 | 56.4 | 82.4 | | 54.7 | 64.8 |
| 25. | B. napus_DY025654 | 52 | 55.4 | 51.8 | 48.5 | 52.3 | 41 | 42.9 | 67.1 | 73.7 | 51.8 | 57.8 | | 48.6 |
| 26. | C. clementina_DY262513 | 78.2 | 40.5 | 78.2 | 35.3 | 78.5 | 57.9 | 62.1 | 71.8 | 63.2 | 78.7 | 79.9 | 60.9 | |
| 27. | C. clementina_DY262587 | 51.5 | 58.4 | 51 | 51.5 | 51.2 | 37.3 | 41.4 | 66.8 | 71.4 | 51.8 | 53.2 | 82.4 | 63.5 |
| 28. | C. clementina_DY263526 | 51.2 | 60.9 | 50.1 | 53.8 | 50.4 | 37.3 | 40.6 | 65 | 71.8 | 51.5 | 52.9 | 82.8 | 63.2 |
| 29. | C. clementina_DY268933 | 67.8 | 48.1 | 67.8 | 42 | 68.1 | 51.5 | 54.4 | 81.9 | 75.8 | 68.1 | 70.1 | 71.3 | 82.8 |
| 30. | C. clementina_TA2451_85681 | 79 | 41.1 | 79.3 | 35.9 | 79.6 | 58.2 | 63.6 | 72.3 | 65 | 79.3 | 80.2 | 60.9 | 92.8 |
| 31. | C. tinctorius_TA325_4222 | 84.6 | 35.8 | 84.6 | 32.1 | 84.9 | 66.5 | 67.3 | 64.7 | 57.4 | 84.9 | 84.2 | 53.6 | 78.4 |
| 32. | G. max_Glyma09g08190.1 | 60.8 | 47.2 | 60.5 | 41.3 | 60.8 | 48.3 | 47.9 | 73.8 | 73.8 | 61 | 62.6 | 73.1 | 68.1 |
| 33. | H. tuberosus_TA3030_4233 | 86 | 36 | 85.8 | 32 | 85.8 | 68.1 | 66.8 | 65.6 | 58.1 | 86 | 85.3 | 53 | 78.2 |
| 34. | I. nil_CJ747673 | 46.6 | 63 | 46 | 56.3 | 46.3 | 35.7 | 37.2 | 60.4 | 65.6 | 47.4 | 45.7 | 73.8 | 52.6 |
| 35. | L. virosa_TA2328_75947 | 67.3 | 45.5 | 67.8 | 41 | 68.1 | 54.2 | 53.6 | 80.7 | 72.1 | 67.6 | 66.6 | 67.9 | 73.9 |
| 36. | P. nigra_TA2942_3691 | 30 | 62.4 | 30 | 69.3 | 30.2 | 24.9 | 24.2 | 38.9 | 42.9 | 30.2 | 29.9 | 49.4 | 32.8 |
| 37. | P. trichocarpa_832064 | 82.6 | 36.5 | 82 | 31.8 | 82 | 62.5 | 68.6 | 64.3 | 57.3 | 83.1 | 84.6 | 52.9 | 77.1 |
| 38. | P. trichocarpa_scaff_VI.1535 | 84.5 | 37.9 | 84.3 | 32.5 | 84.3 | 63.7 | 68.3 | 65.3 | 58.7 | 84.8 | 85.6 | 54.1 | 78.9 |
| 39. | V. shuttleworthii_CN604099 | 30.8 | 65.3 | 30.8 | 71.3 | 31.6 | 23.1 | 23.7 | 40.3 | 44 | 31.3 | 29.9 | 47.6 | 33 |
| 40. | V. vinifera_GSVIVT00016416001 | 85.4 | 36.7 | 84.6 | 31.9 | 84.8 | 64.9 | 67.8 | 66 | 58.8 | 85.4 | 85.4 | 52.9 | 79 |
| 41. | V. vinifera_TA40906_29760 | 85.4 | 36.7 | 84.6 | 31.9 | 84.8 | 64.9 | 67.8 | 66 | 58.8 | 85.4 | 85.1 | 52.9 | 79 |

| | | 27 | 28 | 29 | 30 | 31 | 32 | 33 | 34 |
|---|---|---|---|---|---|---|---|---|---|
| 1. | Saccof_IDI2 | 42.2 | 42.2 | 58.6 | 67.6 | 73.7 | 48.8 | 75.1 | 38.1 |
| 2. | Chlorella_140697 | 14.6 | 14.3 | 20.3 | 22.9 | 24.7 | 18.3 | 24.8 | 13.4 |
| 3. | H. vulgare_TA31842_4513 | 41.7 | 40.4 | 57 | 65.7 | 71.5 | 47.2 | 72.7 | 36.7 |
| 4. | O. RCC809_50296 | 13.8 | 13.5 | 19.7 | 22.2 | 24.8 | 19 | 26 | 12.4 |
| 5. | O. sativa.indica_BGIOSIBCE033922 | 38.9 | 38.9 | 53.7 | 62.6 | 69.9 | 45 | 72.1 | 36.4 |
| 6. | O. sativa.indica_BGIOSIBSE037940 | 20.5 | 20.2 | 32.1 | 37.7 | 40.9 | 26 | 40.5 | 18.1 |
| 7. | O. sativa_CR292756 | 44.2 | 45.8 | 35.5 | 30.3 | 28 | 34.5 | 29 | 50 |
| 8. | O. sativa_CX116019 | 42.1 | 43.6 | 33.4 | 28.6 | 26.4 | 33.8 | 27.4 | 47.1 |
| 9. | O. sativa_Os11g0216900 | 38.9 | 38.9 | 53.7 | 62.6 | 69.9 | 45 | 72.1 | 36.4 |
| 10. | O. taurii_8569 | 27 | 27 | 39.4 | 44.9 | 51.3 | 36.1 | 52.1 | 27.5 |
| 11. | P. pinaster_TA4183_71647 | 36.7 | 36.7 | 50.5 | 58.5 | 65.4 | 43.8 | 66.7 | 33.6 |
| 12. | P. tricornutum_23811 | 12.3 | 11.8 | 17.8 | 20.3 | 23.4 | 16.3 | 23.6 | 11.3 |
| 13. | S. bicolor_Sb05g008680.1 | 41.7 | 41.7 | 57.8 | 66.8 | 73.9 | 47.5 | 75.7 | 38.3 |
| 14. | S. bicolor_TA25485_4558 | 41.4 | 41.4 | 57.5 | 66.5 | 73.9 | 47.2 | 75.4 | 38.1 |
| 15. | S. officinarum_TA35690_4547 | 45.9 | 48 | 36.9 | 31.8 | 28.8 | 36.7 | 29 | 49 |
| 16. | T. aestivum_c54899571@13348 | 40.9 | 39.6 | 56.4 | 65.2 | 71.5 | 47.7 | 73.2 | 36.5 |
| 17. | T. aestivum_CV772651 | 40.3 | 41.8 | 33.1 | 28.3 | 25.9 | 33.2 | 26.6 | 40.9 |
| 18. | T. aestivum_TA67133_4565 | 41.2 | 39.8 | 56.7 | 65.4 | 71.8 | 48 | 73.5 | 36.7 |
| 19. | T. pseudonana_35896 | 26 | 25.7 | 35.7 | 39.9 | 44.9 | 31.5 | 45.9 | 25.1 |
| 20. | V. carteri_59470 | 30.4 | 29.6 | 41.7 | 47.4 | 50.9 | 36.6 | 50.9 | 27.5 |
| 21. | Z. mays_c57808725gm030403@2572 | 54.5 | 53.4 | 71.4 | 62.5 | 55.3 | 60.6 | 57.1 | 48.8 |
| 22. | Z. mays_DQ244248 | 58.3 | 58.3 | 64.3 | 55.5 | 48.8 | 60.3 | 49.9 | 52.8 |
| 23. | Z. mays_ZM07MC01636_57808725 | 42.2 | 42.2 | 58.3 | 67 | 73.1 | 48.5 | 74.5 | 38.3 |
| 24. | A. thaliana_AT2G05830.1 | 42.1 | 41.5 | 58.7 | 66.8 | 72.2 | 50.5 | 74.6 | 38.5 |
| 25. | B. napus_DY025654 | 63.7 | 64.6 | 57.6 | 49.3 | 44.4 | 59.9 | 44 | 59.3 |
| 26. | C. clementina_DY262513 | 62.4 | 61.8 | 82.5 | 88 | 66.6 | 57.9 | 67.8 | 43.8 |
| 27. | C. clementina_DY262587 | | 90.6 | 74.1 | 63.3 | 43.2 | 57.8 | 44.7 | 63.2 |
| 28. | C. clementina_DY263526 | 92.7 | | 73.4 | 62.4 | 43.5 | 58.5 | 44.7 | 65 |
| 29. | C. clementina_DY268933 | 75.8 | 75.8 | | 84.3 | 60.5 | 65.5 | 61.7 | 51.4 |
| 30. | C. clementina_TA2451_85681 | 64.4 | 63.8 | 84.5 | | 70.2 | 57 | 70.3 | 43.9 |
| 31. | C. tinctorius_TA325_4222 | 52.3 | 52 | 69 | 80.3 | | 49.1 | 88.4 | 38.8 |
| 32. | G. max_Glyma09g08190.1 | 68.9 | 68.2 | 78.5 | 67.6 | 62.3 | | 49.5 | 51.7 |
| 33. | H. tuberosus_TA3030_4233 | 52.2 | 51.6 | 68.8 | 79.3 | 93.8 | 62.4 | | 38.9 |
| 34. | I. nil_CJ747673 | 76.8 | 79.1 | 62.1 | 53.1 | 47.7 | 63.3 | 47.3 | |
| 35. | L. virosa_TA2328_75947 | 66.6 | 67.2 | 85.7 | 74.6 | 72.8 | 74.8 | 73.1 | 61 |
| 36. | P. nigra_TA2942_3691 | 49.4 | 50.7 | 39.6 | 33.8 | 31 | 40.9 | 30.6 | 54.8 |
| 37. | P. trichocarpa_832064 | 51.6 | 51.3 | 67.7 | 78.6 | 84.1 | 60.4 | 84.9 | 45.8 |
| 38. | P. trichocarpa_scaff_VI.1535 | 53.1 | 51.5 | 69.3 | 80.3 | 85.9 | 61.6 | 86.4 | 46.9 |
| 39. | V. shuttleworthii_CN604099 | 50.2 | 51.6 | 39.9 | 33.8 | 30.2 | 40.9 | 29.8 | 54.3 |
| 40. | V. vinifera_GSVIVT00016416001 | 52.7 | 51.9 | 68.9 | 79.5 | 84.8 | 59.8 | 86.2 | 46.5 |
| 41. | V. vinifera_TA40906_29760 | 52.7 | 51.9 | 68.9 | 79.5 | 84.8 | 61.2 | 86.2 | 46.5 |

| | | 35 | 36 | 37 | 38 | 39 | 40 | 41 |
|---|---|---|---|---|---|---|---|---|
| 1. | Saccof_IDI2 | 56.5 | 24.5 | 72.2 | 74.2 | 25.2 | 71.7 | 72 |
| 2. | Chlorella_140697 | 20.3 | 9.1 | 25.7 | 25.2 | 10 | 25.8 | 25.8 |
| 3. | H. vulgare_TA31842_4513 | 55.9 | 23.9 | 69.4 | 71.3 | 24.1 | 71.2 | 71.4 |
| 4. | O. RCC809_50296 | 20.1 | 8.7 | 26.2 | 25.6 | 9.2 | 25.4 | 25.4 |
| 5. | O. sativa.indica_BGIOSIBCE033922 | 54.5 | 24.3 | 68.8 | 69.4 | 23.2 | 68.9 | 69.1 |
| 6. | O. sativa.indica_BGIOSIBSE037940 | 32.7 | 14.2 | 39.9 | 41.2 | 13.5 | 40.1 | 40.1 |
| 7. | O. sativa_CR292756 | 34.1 | 54.2 | 28.6 | 29.1 | 52.1 | 26.6 | 26.6 |
| 8. | O. sativa_CX116019 | 32.4 | 55 | 26.8 | 27.5 | 52.9 | 25.3 | 25.3 |
| 9. | O. sativa_Os11g0216900 | 54.5 | 24.3 | 68.8 | 69.4 | 23.2 | 68.9 | 69.1 |
| 10. | O. taurii_8569 | 41.6 | 17 | 51.9 | 50.8 | 17.4 | 51.3 | 51.3 |
| 11. | P. pinaster_TA4183_71647 | 49.7 | 23.1 | 67 | 67 | 22.7 | 63.8 | 64.1 |
| 12. | P. tricornutum_23811 | 16.7 | 8.6 | 23.9 | 23.2 | 8.5 | 23.2 | 23.2 |
| 13. | S. bicolor_Sb05g008680.1 | 56.2 | 23.9 | 71.4 | 73.4 | 24.7 | 71.2 | 71.4 |
| 14. | S. bicolor_TA25485_4558 | 56.5 | 23.9 | 71.2 | 73.1 | 24.7 | 71.2 | 71.4 |
| 15. | S. officinarum_TA35690_4547 | 34.8 | 47.5 | 28.9 | 29.9 | 51.1 | 28.7 | 28.7 |
| 16. | T. aestivum_c54899571@13348 | 55.7 | 23.7 | 69.4 | 71 | 24.4 | 70.6 | 70.9 |
| 17. | T. aestivum_CV772651 | 32.4 | 52.2 | 25.5 | 26.1 | 54.5 | 25.5 | 25.5 |
| 18. | T. aestivum_TA67133_4565 | 55.9 | 23.9 | 69.4 | 71.3 | 24.9 | 70.9 | 71.2 |
| 19. | T. pseudonana_35896 | 36.3 | 18.7 | 44.9 | 45.7 | 16.1 | 45 | 45 |
| 20. | V. carteri_59470 | 41.8 | 18.7 | 52.7 | 52.2 | 19.1 | 53.2 | 53.2 |
| 21. | Z. mays_c57808725gm030403@2572 | 67.9 | 31.2 | 55.2 | 56.3 | 32.5 | 53.7 | 53.7 |
| 22. | Z. mays_DQ244248 | 60.3 | 34.3 | 49.5 | 50.7 | 35.5 | 47.3 | 47.3 |
| 23. | Z. mays_ZM07MC01636_57808725@1630 | 56.8 | 24.2 | 71.9 | 73.4 | 25.5 | 71.7 | 72 |
| 24. | A. thaliana_AT2G05830.1 | 57.2 | 25 | 72.7 | 73.4 | 24.9 | 71.9 | 71.9 |
| 25. | B. napus_DY025654 | 56.4 | 38.9 | 44.7 | 45.7 | 39 | 43.2 | 43.2 |
| 26. | C. clementina_DY262513 | 65.9 | 27.1 | 67.2 | 68.8 | 29.6 | 67.6 | 67.6 |
| 27. | C. clementina_DY262587 | 56.1 | 40.9 | 44.3 | 45.6 | 44.6 | 44 | 44 |
| 28. | C. clementina_DY263526 | 56.5 | 41 | 44.5 | 44.3 | 45.8 | 43.8 | 43.8 |
| 29. | C. clementina_DY268933 | 76.5 | 32.5 | 60.7 | 62.1 | 35.5 | 60.2 | 60.2 |
| 30. | C. clementina_TA2451_85681 | 66.6 | 27.8 | 69.6 | 71 | 30 | 70.1 | 70.4 |
| 31. | C. tinctorius_TA325_4222 | 67.9 | 25.5 | 74.7 | 76.1 | 25.9 | 74.5 | 74.5 |
| 32. | G. max_Glyma09g08190.1 | 62.6 | 33.9 | 51.8 | 52.5 | 36.2 | 49.9 | 50.7 |
| 33. | H. tuberosus_TA3030_4233 | 68.5 | 25.7 | 75.3 | 76.7 | 26.1 | 74.3 | 74.5 |
| 34. | I. nil_CJ747673 | 50.9 | 46.7 | 39.2 | 39.9 | 46.6 | 38 | 38 |
| 35. | L. virosa_TA2328_75947 | | 32.1 | 58.1 | 59.5 | 33.1 | 57.4 | 57.4 |
| 36. | P. nigra_TA2942_3691 | 40 | | 34.1 | 34.9 | 71.1 | 26.6 | 26.6 |
| 37. | P. trichocarpa_832064 | 66.9 | 34.9 | | 96.9 | 27.1 | 75.5 | 75.5 |
| 38. | P. trichocarpa_scaff_VI.1535 | 68.5 | 35.7 | 97.1 | | 27.2 | 76.1 | 76.1 |
| 39. | V. shuttleworthii_CN604099 | 39.3 | 77.5 | 30.5 | 30.7 | | 35.4 | 35.4 |
| 40. | V. vinifera_GSVIVT00016416001 | 67.6 | 30.3 | 85.2 | 86.7 | 35.4 | | 99.7 |
| 41. | V. vinifera_TA40906_29760 | 67.6 | 30.3 | 85.4 | 87 | 35.4 | 100 | |

3.4. eIF4F-Like Protein Complex

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence sequence identity is shown in the top half of the diagonal dividing line.

Parameters used in the comparison were:
Scoring matrix: Blosum62
First Gap: 12
Extending gap: 2

Results of the software analysis are shown in Table B4 for the global identity over the full length of the polypeptide sequences. Percentage identity is given above the diagonal and percentage similarity is given below the diagonal.

The percentage identity between eIF4 isoG polypeptide sequences useful in performing the methods of the invention can be as low as 56.4% amino acid identity compared to SEQ ID NO: 241.

TABLE B4
MatGAT results for global identity over the full length of the polypeptide sequences -
Examples of identity with eIF4isoG-like polypeptides

| | | 1 | 2 | 3 | 4 | 5 | 6 | 7 | 8 | 9 | 10 | 11 | 12 | 13 | 14 |
|---|---|---|---|---|---|---|---|---|---|---|---|---|---|---|---|
| 1. | Os_eIF4isoG | | 56.4 | 61.6 | 59.7 | 61 | 67 | 60.8 | 60.3 | 61.2 | 80.2 | 58.9 | 77.2 | 62.5 | 62.1 |
| 2. | A. thaliana_AT5G57870.1 | | | 66.8 | 63.9 | 63.7 | 54.6 | 57.9 | 65.9 | 68.3 | 58.5 | 60.7 | 55.3 | 65.8 | 62.1 |
| 3. | G. max_Gm0025x00623 | | | | 76.4 | 80.8 | 58.9 | 65.8 | 75.2 | 74 | 61 | 69 | 59.1 | 75.1 | 69.5 |
| 4. | G. max_Gm0071x00063 | | | | | 73.5 | 57.3 | 65.2 | 70.4 | 70.1 | 59.8 | 65 | 56.9 | 71.3 | 68.3 |
| 5. | M. truncatula_AC140546_1.5 | | | | | | 58.6 | 64.3 | 69.1 | 69.1 | 60 | 67.5 | 58.9 | 71 | 67.4 |
| 6. | O. sativa_Os02g0611500 | | | | | | | 59.8 | 58.7 | 58.5 | 69.4 | 58 | 64.8 | 60.6 | 61.9 |
| 7. | P. trichocarpa_835841 | | | | | | | | 66.7 | 64.9 | 61.4 | 64.2 | 58.9 | 69.8 | 71.2 |
| 8. | P. trichocarpa_835945 | | | | | | | | | 82.4 | 61.2 | 69.2 | 57.9 | 75 | 70.4 |
| 9. | R. communis_TA1365_3988 | | | | | | | | | | 62.6 | 67.7 | 58.9 | 75 | 69.4 |
| 10. | S. bicolor_5286647 | | | | | | | | | | | 59.6 | 77.8 | 63.2 | 61.9 |
| 11. | S. tuberosum_TA26995_4113 | | | | | | | | | | | | 58.1 | 70.6 | 66.3 |
| 12. | T. aestivum_TA60686_4565 | | | | | | | | | | | | | 59.9 | 60.2 |
| 13. | V. vinifera_GSVIVT00017187001 | | | | | | | | | | | | | | 75.8 |
| 14. | V. vinifera_GSVIVT00032790001 | | | | | | | | | | | | | | |

### 3.5. GR-RBP (Glycine Rich-RNA Binding Protein) Polypeptides

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table B5 for the global similarity and identity over the full length of the polypeptide sequences of the group A GR-RBP proteins. The percentage identity between the GR-RBP polypeptide sequences useful in performing the methods of the invention can be as low as 10.3% amino acid identity compared to SEQ ID NO: 827. This percentage remains the same when also the sequences of group B GR-RBP proteins are included in the analysis.

TABLE B5
MatGAT results for global similarity and identity over the full length of the polypeptide sequences.

| | | 1 | 2 | 3 | 4 | 5 | 6 | 7 | 8 | 9 |
|---|---|---|---|---|---|---|---|---|---|---|
| 1. | O. sativa_LOC_Os12g31800.1 | | 35 | 32.3 | 28.9 | 35 | 28.9 | 38 | 50.3 | 31.1 |
| 2. | A. thaliana_AT4G13850.2 | 46.5 | | 94.1 | 84.3 | 96.1 | 64.7 | 96.8 | 30.8 | 73.4 |
| 3. | A. thaliana_AT4G13850.3 | 43.8 | 94.1 | | 89.6 | 94 | 68.1 | 91.1 | 28.8 | 77.9 |
| 4. | A. thaliana_AT4G13850.4 | 39.5 | 84.3 | 89.6 | | 84 | 67.6 | 81.6 | 25 | 73.2 |
| 5. | Arabidopsis_thaliana_AJ002892 | 46.1 | 96.7 | 94.7 | 84.7 | | 66.7 | 93 | 30.5 | 76.8 |
| 6. | Arabidopsis_thaliana_AY097374 | 39.1 | 75.8 | 79.2 | 78.7 | 76.7 | | 62 | 26.7 | 70.1 |
| 7. | Arabidopsis_thaliana_BT002197 | 49.2 | 96.8 | 91.1 | 81.6 | 93.7 | 72.2 | | 32.5 | 71.1 |
| 8. | Arabidopsis_thaliana_BT006315 | 62.3 | 41.2 | 39.1 | 34.6 | 39.8 | 37.4 | 42.9 | | 28.7 |
| 9. | B. napus_BN06MC14993_44009177@14945 | 40.7 | 80.4 | 85.4 | 80.3 | 83.3 | 81 | 77.8 | 36.3 | |
| 10. | B. napus_BN06MC33239_51481786@33086 | 32.9 | 51.6 | 54.9 | 61.2 | 52.7 | 58.1 | 50 | 30.4 | 54.2 |
| 11. | Bambusa_oldhamii_EU076902 | 49.6 | 70.4 | 70.4 | 64.8 | 72.2 | 61.1 | 71.6 | 42.6 | 68.5 |
| 12. | E. lagascae_s_el01277_t@402 | 41.1 | 66.7 | 70.8 | 75.8 | 67.3 | 74.3 | 64.6 | 33.6 | 71.8 |
| 13. | G. max_GM06MC37402_su20g11@36531 | 62.4 | 46.9 | 44.5 | 40.4 | 46.1 | 40.8 | 48.6 | 60.2 | 43.3 |
| 14. | H. vulgare_c63432693hv270303@6212 | 48.1 | 67.9 | 66.1 | 59.5 | 68.5 | 62.5 | 72 | 41.9 | 63.7 |
| 15. | H. vulgare_HV04MC07973_63432693@7969 | 30.6 | 50.3 | 52.1 | 58.1 | 50 | 55.9 | 49.4 | 27 | 54.2 |
| 16. | Hordeum_vulgare_subsp_vulgare_AK249796 | 64.2 | 39.8 | 37.5 | 33.4 | 39.1 | 33.8 | 41.5 | 61.2 | 35.8 |
| 17. | Hordeum_vulgare_subsp_vulgare_AK252775 | 49.2 | 69 | 66.1 | 60.1 | 68.5 | 63.1 | 72.6 | 42.6 | 64.3 |
| 18. | L. usitatissimum_c62280695@9780 | 45.7 | 77.8 | 78.5 | 73.2 | 80.7 | 73.2 | 77.2 | 39.1 | 78.5 |
| 19. | Nicotiana_sylvestris_D28862 | 43 | 75.8 | 78.5 | 75.7 | 76.7 | 75.7 | 73.4 | 38.4 | 76.4 |
| 20. | Nicotiana_tabacum_AY048972 | 61.7 | 45.8 | 43 | 39 | 44.4 | 40.4 | 46.2 | 62.6 | 40.1 |
| 21. | O. sativa_LOC_Os07g41120.1 | 35.1 | 23 | 21.6 | 19.8 | 22.4 | 19.4 | 23.6 | 34.7 | 20.8 |
| 22. | O. sativa_LOC_Os10g17454.2 | 59.6 | 36.9 | 34.4 | 31.5 | 37.2 | 31.9 | 38.5 | 57.7 | 34.4 |
| 23. | Oryza_sativa_Indica_Group_CT830471 | 46.1 | 73.2 | 73.3 | 66 | 74.7 | 67.3 | 70.9 | 39.4 | 67.3 |
| 24. | P. patens_167311 | 39.5 | 63.7 | 63.7 | 60.5 | 64.3 | 65.6 | 64.6 | 39.1 | 63.7 |
| 25. | P. patens_208328 | 41.5 | 63.2 | 62.6 | 61.9 | 63.9 | 63.9 | 62.7 | 35.3 | 61.9 |
| 26. | P. trichocarpa_707174 | 37.2 | 62.1 | 66 | 69.9 | 64 | 65.4 | 60.1 | 34.3 | 66.9 |
| 27. | Picea_sitchensis_EF083658 | 51.6 | 69.4 | 69.4 | 63.1 | 72.6 | 65 | 71.5 | 42.2 | 66.9 |
| 28. | Picea_sitchensis_EF086676 | 61.6 | 53.4 | 50.2 | 44.3 | 51.6 | 47.1 | 54.3 | 50.9 | 48.9 |
| 29. | Pisum_sativum_U81287 | 45.7 | 79.7 | 82.2 | 75.3 | 80 | 76.7 | 77.2 | 39.1 | 82.2 |
| 30. | Populus_trichocarpa_EF148189 | 64.3 | 47.3 | 44.8 | 40.7 | 46.5 | 43.6 | 49.8 | 59.5 | 43.2 |
| 31. | S. bicolor_Sb03g043760.1 | 43.8 | 74.5 | 73.5 | 70.1 | 75.3 | 72.1 | 72.8 | 38.8 | 72.8 |
| 32. | S. bicolor_Sb08g015580.1 | 82.2 | 48.4 | 44.8 | 40.8 | 46 | 40.8 | 49.6 | 62.6 | 42.4 |
| 33. | Solanum_lycopersicum_BT012756 | 67.1 | 45.5 | 43.2 | 39.7 | 44.4 | 41.6 | 48.2 | 60.9 | 42 |
| 34. | T. aestivum_c54626433@14323 | 50.8 | 65.7 | 62.3 | 58.3 | 65.1 | 57.7 | 69.1 | 43.9 | 62.3 |
| 35. | T. aestivum_c55526991@11638 | 70.5 | 54.3 | 51.6 | 47.5 | 54.3 | 48.4 | 55.7 | 58.1 | 48.9 |
| 36. | T. aestivum_c56257751@11019 | 52.3 | 68.8 | 63 | 57.8 | 64.7 | 57.8 | 71.1 | 45 | 62.4 |
| 37. | T. aestivum_TA06MC00270_56599813@270 | 53.5 | 67.6 | 61.9 | 56.8 | 64.2 | 56.8 | 69.9 | 45.7 | 61.4 |
| 38. | V. vinifera_GSVIVT00016201001 | 67.9 | 42.6 | 39.4 | 36.1 | 40.8 | 38.3 | 44.4 | 66.4 | 39 |
| 39. | Z. mays_ZM07MC15190_65293483@15154 | 76.7 | 46.6 | 43.4 | 39.8 | 45.4 | 39.8 | 48.2 | 59.5 | 44.2 |
| 40. | Z. mays_ZM07MC16747_65163049@16705 | 78.7 | 47.8 | 44.5 | 40.5 | 47 | 40.5 | 49.8 | 60.9 | 44.1 |
| 41. | Zea_mays_DQ245645 | 43.8 | 86.3 | 91 | 81.3 | 88 | 81.9 | 83.5 | 38.1 | 93.8 |
| 42. | Zea_mays_DQ245844 | 52.7 | 68 | 62.3 | 57.1 | 65.1 | 57.7 | 70.3 | 45.3 | 62.3 |
| 43. | Zea_mays_EU968589 | 45.3 | 76.5 | 72.5 | 68 | 75.8 | 68 | 75.3 | 39.8 | 69.9 |

| | | | 10 | 11 | 12 | 13 | 14 | 15 | 16 | 17 |
|---|---|---|---|---|---|---|---|---|---|---|
| 1. | O. sativa_LOC_Os12g31800.1 | | 23.6 | 34 | 28.3 | 47.8 | 36.2 | 20.3 | 51.8 | 36.5 |
| 2. | A. thaliana_AT4G13850.2 | | 32.3 | 54.9 | 53.6 | 35.5 | 55 | 34.8 | 30.1 | 55 |
| 3. | A. thaliana_AT4G13850.3 | | 34.2 | 54.3 | 56.9 | 33.1 | 53.2 | 36.8 | 28.6 | 52.4 |
| 4. | A. thaliana_AT4G13850.4 | | 38.2 | 50.6 | 57.7 | 29.3 | 47.1 | 40 | 24.7 | 47.1 |
| 5. | Arabidopsis_thaliana_AJ002892 | | 32.9 | 56.1 | 53.3 | 35.1 | 54.7 | 34.8 | 29.2 | 53.8 |
| 6. | Arabidopsis_thaliana_AY097374 | | 37.2 | 52.1 | 60.4 | 27.8 | 53 | 44.1 | 25.8 | 53 |
| 7. | Arabidopsis_thaliana_BT002197 | | 31.3 | 55.4 | 51.9 | 37.1 | 56.8 | 33.5 | 32.6 | 56.8 |
| 8. | Arabidopsis_thaliana_BT006315 | | 22.9 | 29.8 | 23.8 | 48.3 | 31 | 17.1 | 46.8 | 31 |
| 9. | B. napus_BN06MC14993_44009177@14945 | | 36.1 | 51.2 | 56.3 | 34.7 | 51.8 | 39.5 | 26.6 | 51.8 |
| 10. | B. napus_BN06MC33239_51481786@33086 | | | 30.1 | 37 | 29.8 | 34.5 | 48.3 | 19.1 | 34.5 |
| 11. | Bambusa_oldhamii_EU076902 | | 51.2 | | 53.7 | 37.1 | 67.3 | 45 | 28.2 | 67.8 |
| 12. | E. lagascae_s_el01277_t@402 | | 59.8 | 66 | | 28.6 | 45.8 | 40.4 | 25.4 | 45.9 |
| 13. | G. max_GM06MC37402_su20g11@36531 | | 37.6 | 52.7 | 42.4 | | 37.9 | 23.4 | 39.4 | 38.3 |
| 14. | H. vulgare_c63432693hv270303@6212 | | 45.2 | 80.4 | 59.5 | 51.4 | | 62.5 | 32.1 | 97.6 |
| 15. | H. vulgare_HV04MC07973_63432693@7969 | | 62.9 | 58 | 59.8 | 34.7 | 63.7 | | 19.9 | 64.9 |
| 16. | Hordeum_vulgare_subsp_vulgare_AK249796 | | 27.4 | 41.1 | 35.1 | 55.2 | 42.5 | 28.1 | | 32.7 |
| 17. | Hordeum_vulgare_subsp_vulgare_AK252775 | | 45.2 | 80.4 | 60.1 | 51.8 | 98.2 | 64.9 | 43.5 | |
| 18. | L. usitatissimum_c62280695@9780 | | 55.7 | 71 | 74.5 | 47.8 | 66.1 | 52.3 | 37.1 | 66.7 |
| 19. | Nicotiana_sylvestris_D28862 | | 57.6 | 69.8 | 70.8 | 45.7 | 63.7 | 56.3 | 34.8 | 64.3 |
| 20. | Nicotiana_tabacum_AY048972 | | 33.2 | 46.2 | 37.9 | 64.6 | 45.1 | 29.6 | 60.2 | 44.4 |
| 21. | O. sativa_LOC_Os07g41120.1 | | 17.8 | 23.6 | 19.2 | 32.9 | 23 | 15.8 | 36.9 | 23.2 |
| 22. | O. sativa_LOC_Os10g17454.2 | | 26.8 | 39.1 | 32.8 | 57.4 | 39.1 | 24.9 | 74.1 | 39.7 |
| 23. | Oryza_sativa_Indica_Group_CT830471 | | 52.7 | 83.3 | 69.3 | 48.2 | 76.2 | 59.3 | 38.1 | 76.2 |
| 24. | P. patens_167311 | | 52.2 | 65.4 | 58 | 46.5 | 62.5 | 49.7 | 36.8 | 63.1 |
| 25. | P. patens_208328 | | 51 | 66 | 60 | 46.1 | 63.1 | 53.5 | 36.1 | 63.7 |
| 26. | P. trichocarpa_707174 | | 57.9 | 59.3 | 79.7 | 42.4 | 54.2 | 55.6 | 33.1 | 54.8 |
| 27. | Picea_sitchensis_EF083658 | | 54.1 | 72.8 | 64.3 | 51.8 | 70.2 | 51 | 40.8 | 70.8 |
| 28. | Picea_sitchensis_EF086676 | | 38.5 | 56.6 | 45.7 | 64.1 | 53.4 | 37.1 | 50.2 | 54.3 |
| 29. | Pisum_sativum_U81287 | | 55.5 | 72.2 | 73.3 | 46.5 | 67.9 | 55.5 | 38.1 | 68.5 |
| 30. | Populus_trichocarpa_EF148189 | | 38.2 | 51 | 41.5 | 69 | 51.5 | 34.4 | 56.5 | 52.3 |
| 31. | S. bicolor_Sb03g043760.1 | | 56.5 | 78.4 | 70.7 | 47.3 | 72.6 | 58.5 | 37.8 | 73.2 |
| 32. | S. bicolor_Sb08g015580.1 | | 35.6 | 50.4 | 41.2 | 66.4 | 48.8 | 31.6 | 60.5 | 49.6 |
| 33. | Solanum_lycopersicum_BT012756 | | 33.9 | 47.5 | 40.1 | 71.2 | 47.1 | 31.1 | 58.9 | 47.9 |
| 34. | T. aestivum_c54626433@14323 | | 43.4 | 76.6 | 56 | 53.9 | 85.1 | 57.7 | 44.1 | 86.3 |
| 35. | T. aestivum_c55526991@11638 | | 39.3 | 56.6 | 44.3 | 62.9 | 55.3 | 36.5 | 54.5 | 56.6 |
| 36. | T. aestivum_c56257751@11019 | | 43.9 | 77.5 | 56.1 | 52.7 | 83.2 | 56.1 | 43.8 | 84.4 |
| 37. | T. aestivum_TA06MC00270_56599813@270 | | 43.2 | 77.8 | 55.7 | 53.9 | 83.5 | 55.7 | 44.8 | 84.7 |
| 38. | V. vinifera_GSVIVT00016201001 | | 33.2 | 45.1 | 35 | 67.5 | 45.5 | 30 | 65.2 | 45.8 |
| 39. | Z. mays_ZM07MC15190_65293483@15154 | | 34.7 | 50.2 | 41.4 | 65.3 | 47 | 30.7 | 59.5 | 47.8 |
| 40. | Z. mays_ZM07MC16747_65163049@16705 | | 34.8 | 50.6 | 39.3 | 66 | 47.4 | 30.4 | 60.5 | 48.2 |
| 41. | Zea_mays_DQ245645 | | 52.1 | 70.4 | 72.9 | 44.1 | 67.3 | 56.9 | 37.8 | 67.3 |
| 42. | Zea_mays_DQ245844 | | 43.4 | 78.9 | 56.6 | 53.5 | 84.6 | 56.6 | 45.5 | 85.7 |
| 43. | Zea_mays_EU968589 | | 52.3 | 77.8 | 66 | 46.9 | 75 | 55.6 | 39.1 | 75 |
| | | 18 | 19 | 20 | 21 | 22 | 23 | 24 | 25 | 26 |
| 1. | O. sativa_LOC_Os12g31800.1 | 31.6 | 31.2 | 47.2 | 26.8 | 48.1 | 33.2 | 26.5 | 26.4 | 25.5 |
| 2. | A. thaliana_AT4G13850.2 | 61 | 62.8 | 31.4 | 16.6 | 28.6 | 57.6 | 48.8 | 45 | 46.1 |
| 3. | A. thaliana_AT4G13850.3 | 61.7 | 65.5 | 28.9 | 15.2 | 25.8 | 57.4 | 50.6 | 45.8 | 49 |
| 4. | A. thaliana_AT4G13850.4 | 57 | 64.6 | 25.3 | 13.2 | 23 | 51.3 | 49 | 46.5 | 54.1 |
| 5. | Arabidopsis_thaliana_AJ002892 | 62.1 | 64.1 | 30.3 | 16 | 28.4 | 58 | 51.6 | 45.6 | 47 |
| 6. | Arabidopsis_thaliana_AY097374 | 57 | 61 | 29.6 | 13.7 | 23.9 | 57.3 | 47.8 | 46.5 | 51.1 |
| 7. | Arabidopsis_thaliana_BT002197 | 61 | 60.9 | 32.5 | 17.2 | 29.8 | 56.2 | 48.5 | 43 | 44.7 |
| 8. | Arabidopsis_thaliana_BT006315 | 30.5 | 27.5 | 44.4 | 24.6 | 42.5 | 26.5 | 27.6 | 25.8 | 24.3 |
| 9. | B. napus_BN06MC14993_44009177 | 62 | 61.9 | 28.8 | 14.4 | 26.6 | 54.7 | 47.8 | 46.5 | 48.3 |
| 10. | B. napus_BN06MC33239_51481786 | 38.3 | 37.9 | 24.9 | 10.9 | 19.9 | 34.4 | 33.1 | 34.2 | 40.6 |
| 11. | Bambusa_oldhamii_EU076902 | 52.1 | 54 | 35 | 15.6 | 28.1 | 72.6 | 44.7 | 46.7 | 46.4 |
| 12. | E. lagascae_s_el01277_t@402 | 56.4 | 55.9 | 27.1 | 12.6 | 22.7 | 51.3 | 45.2 | 43.2 | 57.6 |
| 13. | G. max_GM06MC37402_su20g11 | 35.5 | 34.1 | 49.3 | 22.9 | 41 | 34.6 | 32.3 | 30.5 | 31.2 |
| 14. | H. vulgare_c63432693hv270303 | 54.1 | 49.7 | 37 | 16.9 | 30.6 | 64.9 | 45.7 | 45.7 | 44.4 |
| 15. | H. vulgare_HV04MC07973_63432693 | 38.4 | 41.1 | 22.8 | 10.3 | 19.1 | 46.5 | 35 | 39.8 | 42.7 |
| 16. | H._vulgare_subsp_vulgare_AK249796 | 25.3 | 26.1 | 43.3 | 28.7 | 61.3 | 28.1 | 25.2 | 24.5 | 23.7 |
| 17. | H._vulgare_subsp_vulgare_AK252775 | 53.5 | 49.7 | 35.9 | 17.3 | 30.9 | 64.9 | 45.7 | 45.7 | 45 |
| 18. | L. usitatissimum_c62280695@9780 | | 59.9 | 32.5 | 14.4 | 27 | 52.8 | 46.6 | 45 | 54.4 |
| 19. | Nicotiana_sylvestris_D28862 | 81.2 | | 30 | 15.7 | 25.2 | 57 | 46.9 | 46.5 | 53.1 |
| 20. | Nicotiana_tabacum_AY048972 | 43 | 42.2 | | 25 | 43.1 | 33.2 | 28.4 | 28.7 | 26.7 |
| 21. | O. sativa_LOC_Os07g41120.1 | 22.8 | 21.4 | 34.5 | | 28 | 14.6 | 14.1 | 14.3 | 14 |
| 22. | O. sativa_LOC_Os10g17454.2 | 35.3 | 35 | 58 | 38.5 | | 26.5 | 23 | 23.5 | 23 |
| 23. | Oryza_sativa_Indica_Group_CT830471 | 75.3 | 74.7 | 41.2 | 22 | 36.3 | | 46.9 | 47.8 | 45.8 |
| 24. | P. patens_167311 | 67.5 | 66.9 | 42.6 | 20.4 | 34.4 | 70.1 | | 51.9 | 42 |
| 25. | P. patens_208328 | 67.1 | 67.7 | 42.2 | 21 | 32.5 | 69 | 72 | | 43.2 |
| 26. | P. trichocarpa_707174 | 72.5 | 66.7 | 37.2 | 19.6 | 30.9 | 64 | 58.6 | 60.6 | |
| 27. | Picea_sitchensis_EF083658 | 73.2 | 72 | 46.6 | 23.8 | 38.8 | 73.2 | 64.3 | 70.1 | 59.9 |
| 28. | Picea_sitchensis_EF086676 | 52.5 | 50.2 | 56.3 | 30.7 | 49.2 | 52 | 49.3 | 48.4 | 44.3 |
| 29. | Pisum_sativum_U81287 | 78.5 | 76.7 | 42.6 | 21.6 | 36.6 | 76 | 65 | 62.6 | 68.5 |
| 30. | Populus_trichocarpa_EF148189 | 48.5 | 46.1 | 65 | 31.9 | 53.9 | 50.2 | 47.7 | 45.6 | 40.7 |
| 31. | S. bicolor_Sb03g043760.1 | 75.8 | 73.5 | 41.2 | 21.6 | 36.3 | 82 | 66.2 | 64.5 | 60.5 |
| 32. | S. bicolor_Sb08g015580.1 | 46.4 | 44 | 63.2 | 34.1 | 59 | 45.6 | 42.4 | 44.4 | 36.8 |
| 33. | Solanum_lycopersicum_BT012756 | 45.9 | 44 | 67.5 | 33.7 | 56.8 | 44.7 | 41.6 | 43.6 | 38.1 |
| 34. | T. aestivum_c54626433@14323 | 66.3 | 61.7 | 45.8 | 24.8 | 41.6 | 72.6 | 64 | 62.3 | 52.6 |
| 35. | T. aestivum_c55526991@11638 | 54.8 | 51.6 | 61.7 | 31.7 | 53.6 | 52.5 | 45.7 | 48.4 | 43.4 |
| 36. | T. aestivum_c56257751@11019 | 65.3 | 60.7 | 45.8 | 24.4 | 41.6 | 73.4 | 63 | 63 | 52 |
| 37. | T. aestivum_TA06MC00270_56599813 | 64.8 | 60.8 | 46.2 | 25 | 42 | 72.7 | 62.5 | 61.9 | 50.6 |
| 38. | V. vinifera_GSVIVT00016201001 | 43 | 40.8 | 76.5 | 37.3 | 64.4 | 42.2 | 40.1 | 39.4 | 36.8 |
| 39. | Z. mays_ZM07MC15190_65293483 | 47 | 43.4 | 61 | 34.7 | 56.5 | 44.2 | 42.6 | 41.4 | 36.7 |
| 40. | Z. mays_ZM07MC16747_65163049 | 47.4 | 44.1 | 64.6 | 34.9 | 59 | 46.2 | 41.3 | 42.9 | 38.5 |
| 41. | Zea_mays_DQ245645 | 81.9 | 78.5 | 41.2 | 21.6 | 36 | 74.7 | 64.3 | 60.6 | 64.6 |
| 42. | Zea_mays_DQ245844 | 65.1 | 62.3 | 46.9 | 25 | 42.3 | 73.7 | 62.9 | 62.9 | 50.9 |
| 43. | Zea_mays_EU968589 | 69.9 | 69.9 | 41.5 | 22.2 | 36.3 | 79.1 | 67.5 | 68.4 | 58.2 |

| | | 27 | 28 | 29 | 30 | 31 | 32 | 33 | 34 |
|---|---|---|---|---|---|---|---|---|---|
| 1. | O. sativa_LOC_Os12g31800.1 | 40.4 | 47.2 | 35 | 46.5 | 32.8 | 69.3 | 46.3 | 35.8 |
| 2. | A. thaliana_AT4G13850.2 | 54.1 | 41.6 | 65.6 | 36.1 | 59 | 38 | 35.8 | 52.5 |
| 3. | A. thaliana_AT4G13850.3 | 54.1 | 38.5 | 66.2 | 33.6 | 59.2 | 34.9 | 33.5 | 49.2 |
| 4. | A. thaliana_AT4G13850.4 | 47.8 | 33.5 | 61 | 29.5 | 55.8 | 31 | 30.4 | 45.2 |
| 5. | Arabidopsis_thaliana_AJ002892 | 57 | 40.3 | 64.9 | 35.5 | 60.4 | 36.5 | 34.2 | 52.5 |
| 6. | Arabidopsis_thaliana_AY097374 | 49.4 | 36.2 | 63 | 30.7 | 53.3 | 31.8 | 31.1 | 49.1 |
| 7. | Arabidopsis_thaliana_BT002197 | 54.9 | 42.6 | 63.5 | 39 | 58.1 | 40 | 38.1 | 54.1 |
| 8. | Arabidopsis_thaliana_BT006315 | 32.4 | 38.3 | 30.4 | 46.3 | 27.2 | 48 | 45.5 | 30.7 |
| 9. | B. napus_BN06MC14993_44009177 | 53.5 | 39.4 | 64.2 | 33.6 | 57.4 | 33.2 | 32.3 | 50.8 |
| 10. | B. napus_BN06MC33239_51481786 | 34.6 | 27 | 41.8 | 28.6 | 35.8 | 25 | 26 | 31.4 |
| 11. | Bambusa_oldhamii_EU076902 | 50.6 | 39.3 | 56.4 | 36.1 | 66.7 | 37.2 | 35 | 65.7 |
| 12. | E. lagascae_s_el01277_t@402 | 43.9 | 30.3 | 58.9 | 27 | 53.7 | 27.1 | 26.7 | 42.3 |
| 13. | G. max_GM06MC37402_su20g11 | 37.5 | 48.8 | 37.1 | 52.8 | 34.4 | 48.7 | 49.8 | 39.1 |
| 14. | H. vulgare_c63432693hv270303 | 54.7 | 42.3 | 56 | 40.1 | 59.5 | 39 | 37.9 | 79.5 |
| 15. | H. vulgare_HV04MC07973_63432693 | 36 | 27.3 | 41.6 | 23.9 | 45.2 | 22 | 22.3 | 54.3 |
| 16. | H._vulgare_subsp_vulgare_AK249796 | 32.5 | 37 | 30.3 | 44.7 | 28.4 | 47 | 42 | 31.7 |
| 17. | H._vulgare_subsp_vulgare_AK252775 | 55.3 | 42.7 | 56 | 40.3 | 60.1 | 39 | 37.9 | 80.8 |
| 18. | L. usitatissimum_c62280695@9780 | 50.6 | 36.8 | 62.4 | 34 | 52.3 | 33.2 | 33.3 | 52 |
| 19. | Nicotiana_sylvestris_D28862 | 54.1 | 36.2 | 60.7 | 33.6 | 57.4 | 33.3 | 31.9 | 47.5 |
| 20. | Nicotiana_tabacum_AY048972 | 35.4 | 42.9 | 33.2 | 48.7 | 31.4 | 47.4 | 49.6 | 35.3 |
| 21. | O. sativa_LOC_Os07g41120.1 | 18.5 | 22.8 | 17.2 | 24 | 16 | 25.6 | 23.5 | 17.7 |
| 22. | O. sativa_LOC_Os10g17454.2 | 29.1 | 38 | 29.5 | 43.6 | 28.7 | 45.5 | 38.4 | 31.5 |
| 23. | Oryza_sativa_Indica_Group_CT830471 | 54.4 | 38.5 | 56.2 | 37.2 | 68.7 | 35.4 | 36.4 | 63.6 |
| 24. | P. patens_167311 | 44.8 | 32.7 | 51.9 | 32.5 | 49.4 | 29.1 | 28.6 | 42.3 |
| 25. | P. patens_208328 | 47.3 | 29.5 | 47.7 | 31.1 | 45.5 | 28.6 | 30.5 | 43.6 |
| 26. | P. trichocarpa_707174 | 42.4 | 32.3 | 54.8 | 30.2 | 47 | 26.6 | 26.7 | 40.7 |
| 27. | Picea_sitchensis_EF083658 | | 45.2 | 53.8 | 38.4 | 55.4 | 41.7 | 35.4 | 51.4 |
| 28. | Picea_sitchensis_EF086676 | 58.4 | | 39.8 | 47.2 | 38.9 | 48.8 | 42.9 | 41 |
| 29. | Pisum_sativum_U81287 | 72 | 53.4 | | 38.6 | 56.8 | 35.5 | 32.9 | 52 |
| 30. | Populus_trichocarpa_EF148189 | 53.5 | 62.2 | 46.5 | | 36.4 | 51 | 51 | 41.3 |
| 31. | S. bicolor_Sb03g043760.1 | 70.1 | 52.5 | 75.5 | 48.5 | | 36.2 | 33.1 | 55.4 |
| 32. | S. bicolor_Sb08g015580.1 | 49.6 | 61.2 | 46.4 | 68.8 | 45.2 | | 50.9 | 38.3 |
| 33. | Solanum_lycopersicum_BT012756 | 49 | 57.2 | 43.6 | 68.1 | 44.4 | 68.9 | | 37.1 |
| 34. | T. aestivum_c54626433@14323 | 68 | 54.3 | 65.7 | 54.8 | 68 | 49.6 | 48.2 | |
| 35. | T. aestivum_c55526991@11638 | 57.5 | 66.5 | 53.4 | 66 | 50.2 | 71.2 | 62.3 | 57.1 |
| 36. | T. aestivum_c56257751@11019 | 69.4 | 55.2 | 65.9 | 54.4 | 69.4 | 50 | 49.4 | 94.3 |
| 37. | T. aestivum_TA06MC00270_56599813 | 68.2 | 55.7 | 64.8 | 54.8 | 68.8 | 50.4 | 50.6 | 95.5 |
| 38. | V. vinifera_GSVIVT00016201001 | 47.7 | 58.8 | 40.8 | 70 | 40.8 | 70.4 | 69.3 | 48 |
| 39. | Z. mays_ZM07MC15190_65293483 | 47.4 | 60.2 | 45 | 64.9 | 43.8 | 90 | 68.5 | 48.6 |
| 40. | Z. mays_ZM07MC16747_65163049 | 50.2 | 61.5 | 47.4 | 67.6 | 44.9 | 89.2 | 71.2 | 51 |
| 41. | Zea_mays_DQ245645 | 69.4 | 50.2 | 84.9 | 44.4 | 72.8 | 44.8 | 41.6 | 64 |
| 42. | Zea_mays_DQ245844 | 68 | 55.2 | 66.3 | 54.4 | 69.1 | 50.8 | 50.2 | 97.1 |
| 43. | Zea_mays_EU968589 | 68.8 | 52.9 | 71.9 | 48.1 | 85.6 | 46 | 44.7 | 69.1 |

| | | 35 | 36 | 37 | 38 | 39 | 40 | 41 | 42 | 43 |
|---|---|---|---|---|---|---|---|---|---|---|
| 1. | O. sativa_LOC_Os12g31800.1 | 59.8 | 38.7 | 39.1 | 55 | 68.6 | 68.6 | 32.2 | 38.1 | 33.2 |
| 2. | A. thaliana_AT4G13850.2 | 39.7 | 53.9 | 53 | 33.8 | 37.2 | 36.9 | 78.6 | 53 | 59.6 |
| 3. | A. thaliana_AT4G13850.3 | 36.4 | 49.7 | 48.9 | 30.6 | 33.6 | 33.7 | 82.8 | 49.2 | 58.8 |
| 4. | A. thaliana_AT4G13850.4 | 32.6 | 45.1 | 43.8 | 28.2 | 30.4 | 30.6 | 73.6 | 44.1 | 54.2 |
| 5. | Arabidopsis_thaliana_AJ002892 | 39.3 | 52 | 51.7 | 32.4 | 36 | 36.4 | 80.8 | 52.2 | 60.8 |
| 6. | Arabidopsis_thaliana_AY097374 | 32.1 | 49.1 | 48.3 | 29 | 30.8 | 29.4 | 71 | 48.6 | 54.9 |
| 7. | Arabidopsis_thaliana_BT002197 | 40.1 | 56.2 | 55.2 | 35.6 | 38.7 | 39.3 | 76.1 | 55.2 | 60.9 |
| 8. | Arabidopsis_thaliana_BT006315 | 43 | 32.7 | 33 | 50.3 | 47.3 | 47.8 | 29.9 | 32.3 | 28.2 |
| 9. | B. napus_BN06MC14993_44009177@14945 | 34.7 | 50.9 | 50 | 30.3 | 33.9 | 33.2 | 86.1 | 50.8 | 58.2 |
| 10. | B. napus_BN06MC33239_51481786@33086 | 27.2 | 32.9 | 32.4 | 28.2 | 24.1 | 23.7 | 34.9 | 32.6 | 34.4 |
| 11. | Bambusa_oldhamii_EU076902 | 38.1 | 64.9 | 63.8 | 32 | 34.1 | 34.3 | 53.7 | 65.3 | 64.8 |
| 12. | E. lagascae_s_el01277_t@402 | 31.5 | 42.8 | 42 | 24.1 | 27.4 | 24.3 | 59.7 | 42.9 | 48.4 |
| 13. | G. max_GM06MC37402_su20g11@36531 | 45.4 | 38.7 | 39.1 | 53.2 | 47.7 | 47.1 | 33.5 | 39.9 | 38.1 |
| 14. | H. vulgare_c63432693hv270303@6212 | 39.2 | 80.5 | 79.7 | 35.3 | 37.2 | 36.4 | 54.4 | 80.1 | 62.4 |
| 15. | H. vulgare_HV04MC07973_63432693@7969 | 24.1 | 54.3 | 53.4 | 21.9 | 20.7 | 21.1 | 40 | 54.3 | 41.9 |
| 16. | Hordeum_vulgare_subsp_vulgare_AK249796 | 39.7 | 33 | 33.7 | 50.3 | 47.4 | 47.7 | 28.1 | 34 | 28.8 |
| 17. | Hordeum_vulgare_subsp_vulgare_AK252775 | 39.5 | 81.6 | 80.8 | 35.7 | 37.2 | 36.4 | 53.5 | 81.4 | 62.9 |
| 18. | L. usitatissimum_c62280695@9780 | 35.3 | 52 | 50.6 | 32.1 | 34 | 33.2 | 63.3 | 50.3 | 51.9 |
| 19. | Nicotiana_sylvestris_D28862 | 36.6 | 46.9 | 46.6 | 30.9 | 33.2 | 32.2 | 64.9 | 46.9 | 53.2 |
| 20. | Nicotiana_tabacum_AY048972 | 44.4 | 36 | 36 | 54.7 | 47.5 | 44.9 | 29.5 | 36.7 | 31.8 |
| 21. | O. sativa_LOC_Os07g41120.1 | 23.1 | 17.7 | 18.3 | 26.8 | 27 | 26.1 | 15 | 18.5 | 15.4 |
| 22. | O. sativa_LOC_Os10g17454.2 | 38.7 | 33.3 | 33.3 | 51.2 | 41.7 | 44.8 | 27.6 | 33 | 29 |
| 23. | Oryza_sativa_Indica_Group_CT830471 | 35.4 | 63 | 62.5 | 31 | 33.7 | 35 | 56.1 | 63.4 | 68 |
| 24. | P. patens_167311 | 30.5 | 43.1 | 42.6 | 28.4 | 28.9 | 26.7 | 49.7 | 42.9 | 47.6 |
| 25. | P. patens_208328 | 30 | 45.3 | 44.5 | 26.2 | 27 | 26.3 | 46.5 | 45.3 | 44.4 |
| 26. | P. trichocarpa_707174 | 30.4 | 42.3 | 41 | 28.2 | 26.1 | 26.9 | 47.6 | 41.2 | 48.7 |
| 27. | Picea_sitchensis_EF083658 | 39.9 | 54.5 | 53.6 | 36.5 | 38.9 | 40.6 | 52.9 | 53.1 | 52.8 |
| 28. | Picea_sitchensis_EF086676 | 45.7 | 42.7 | 43.6 | 46.6 | 47.5 | 45.9 | 38.9 | 43.2 | 40.8 |
| 29. | Pisum_sativum_U81287 | 38.8 | 53.8 | 52.8 | 33.9 | 34.4 | 34.8 | 65.1 | 54.3 | 56.5 |
| 30. | Populus_trichocarpa_EF148189 | 48.6 | 41.7 | 42.1 | 56.4 | 47.3 | 48.5 | 33.2 | 42.5 | 38.2 |
| 31. | S. bicolor_Sb03g043760.1 | 35.4 | 57.2 | 55.4 | 31.4 | 32.9 | 34.6 | 59.9 | 56.3 | 78.6 |
| 32. | S. bicolor_Sb08g015580.1 | 60.1 | 39.4 | 39.6 | 54.9 | 82.7 | 81.3 | 34 | 40.2 | 37.4 |
| 33. | Solanum_lycopersicum_BT012756 | 43.5 | 38.6 | 39.4 | 56.5 | 48.9 | 51.7 | 33.1 | 39 | 35 |
| 34. | T. aestivum_c54626433@14323 | 40.1 | 91.5 | 92.6 | 36.7 | 34.9 | 36.4 | 51.4 | 94.9 | 59.4 |
| 35. | T. aestivum_c55526991@11638 | | 39.7 | 40.1 | 48.7 | 60.2 | 59.4 | 36 | 40.5 | 36.7 |
| 36. | T. aestivum_c56257751@11019 | 56.6 | | 98.3 | 38.2 | 37.1 | 37.5 | 52 | 96 | 60.1 |
| 37. | T. aestivum_TA06MC00270_56599813@270 | 57.1 | 98.3 | | 38.5 | 37.9 | 38.3 | 51.1 | 97.7 | 59.7 |
| 38. | V. vinifera_GSVIVT00016201001 | 64.6 | 49.1 | 49.5 | | 53.4 | 54.3 | 30.6 | 38.5 | 33.9 |
| 39. | Z. mays_ZM07MC15190_65293483@15154 | 72.5 | 49.4 | 50.6 | 67.1 | | 78 | 33.5 | 38.6 | 34.9 |
| 40. | Z. mays_ZM07MC16747_65163049@16705 | 70.9 | 51 | 52.2 | 67.5 | 86.1 | | 31.6 | 38.7 | 35.8 |
| 41. | Zea_mays_DQ245645 | 50.7 | 64.2 | 63.1 | 39 | 44.6 | 44.1 | | 51.4 | 58.8 |
| 42. | Zea_mays_DQ245844 | 57.1 | 97.1 | 98.3 | 49.1 | 51 | 51 | 64 | | 60.6 |
| 43. | Zea_mays_EU968589 | 51.6 | 70.5 | 70.5 | 41.2 | 44.6 | 47 | 69.9 | 70.9 | |

### Example 4

### Identification of Domains Comprised in Polypeptide Sequences Useful in Performing the Methods of the Invention

### 4.1. C3H-Like Polypeptides

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

### 4.2. SPATULA-Like (SPT) Polypeptides

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The results of the InterPro scan of the polypeptide sequence of SEQ ID NO: 97 are presented as Table C1 below.

### 4.3. IDI2 (Iron Deficiency Induced 2) Polypeptides

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The results of the InterPro scan of the polypeptide sequence as represented by SEQ ID NO: 2 are presented in Table C2.

TABLE C2
InterPro scan results (major accession numbers) of the polypeptide sequence as
represented by SEQ ID NO: 140.

| | | | Amino acid |
|---|---|---|---|
| | | | coordinates on |
| Database | Accession number | Accession name | SEQ ID NO 140 |
| InterPro | IPR000649 | Initiation factor 2B related | |
| HMMPanther | PTHR10233 | TRANSLATION INITIATION FACTOR EIF- | T[28-351] 9.1e−184 |
| | | 2B | |
| HMMPfam | PF01008 | IF-2B | T[48-350] 1.4e−107 |
| InterPro | IPR005251 | Putative translation initiation factor, aIF- | |
| | | 2BI/5-methylthioribose-1-phosphate | |
| | | isomerise | |
| HMMTigr | TIGR00512 | salvage_mtnA: methylthioribose-1-phospha | T[8-350] 5.9e−238 |
| InterPro | IPR011559 | Initiation factor 2B alpha/beta/delta | |
| HMMTigr | TIGR00524 | eIF-2B_rel: eIF-2B alpha/beta/delta-rela | T[34-350] 1.3e−96 |
| InterPro | NULL | NULL | |
| Gene3D | G3DSA:3.40.50.10470 | no description | T[143-350] 2.1e−52 |
| HMMPanther | PTHR10233:SF6 | TRANSLATION INITIATION FACTOR EIF- | T[28-351] 9.1e−184 |
| | | 2B SUBUNIT-RELATED | |
| Superfamily | SSF100950 | NagB/RpiA/CoA transferase-like | T[6-352] 3.2e−108 |

4.4. eIF4F-Like Protein Complex

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom. Table C3 means anyone or more of Table C3a and C3b.

The results of the InterPro scan of the eIF4 isoG and eIF4A polypeptide sequences are presented in Table C3.

TABLE C3a
InterPro scan results (major accession numbers) of the polypeptide sequence as
represented by SEQ ID NO: 241 - InterPro motif search of eIF4isoG (Os04g42140)

| Method | Accession | Domain | start | stop | E-value |
|---|---|---|---|---|---|
| HMMPanther | PTHR23253:SF2 | Eukaryotic initiation | 76 | 792 | 0 |
| | | factor 4F-related | | | |
| superfamily | SSF48371 | ARM repeat | 197 | 438 | 8.20e−71 |
| Gene3D | G3DSA1.25.40.180 | no description | 197 | 438 | 8.50e−73 |
| HMMSmart | SM00544 | no description | 628 | 740 | 1.50e−26 |
| HMMPfam | PF02847 | MA3 | 628 | 740 | 4.20e−30 |
| HMMSmart | SM00543 | no description | 208 | 435 | 4.90e−55 |
| HMMPfam | PF02854 | MIF4G | 208 | 435 | 2.40e−67 |

TABLE C3b
InterPro scan results (major accession numbers) of the polypeptide sequence as
represented by SEQ ID NO: 301 - InterPro motif search of eIF4A (Os06g48750)

| Method | Accession | Domain | start | stop | E-value |
|---|---|---|---|---|---|
| superfamily | SSF52540 | SSF52540 | 78 | 414 | 740e−49 |
| superfamily | SSF52540 | SSF52540 | 41 | 424 | 4.40e−58 |
| HMMPanther | PTHR10967:SF2 | PTHR10967:SF2 | 25 | 414 | 0 |
| HMMPanther | PTHR10967 | PTHR10967 | 25 | 414 | 0 |
| Gene3D | G3DSA:3.40.50.300 | G3DSA:3.40.50.300 | 282 | 400 | 6.50e−32 |
| Gene3D | G3DSA:3.40.50.300 | G3DSA:3.40.50.300 | 28 | 252 | 4.00e−69 |
| ProfileScan | PS51192 | Helicase_ATP_bind_1 | 72 | 242 | 0 |
| ProfileScan | PS51195 | Q_motif | 41 | 69 | 0 |
| HMMSmart | SM00487 | DEXDc | 60 | 257 | 2.70e−56 |
| HMMPfam | PF00270 | DEAD | 65 | 231 | 5.30e−59 |
| Profilescan | PS51194 | Helicase_Cter | 253 | 414 | 0 |
| HMMSmart | SM00490 | HELICc | 294 | 375 | 3.60e−31 |
| HMMPfam | PF00271 | Helicase_C | 299 | 375 | 8.60e−30 |
| Profilescan | PS00039 | Dead_ATP_Helicase | 188 | 196 | 8.00e−05 |

### 4.5. GR-RBP (Glycine Rich-RNA Binding Protein) Polypeptides

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The results of the InterPro scan of the polypeptide sequence as represented by SEQ ID NO: 827 are presented in Table C4.

TABLE C4
InterPro scan results (major accession numbers) of the polypeptide
sequence as represented by SEQ ID NO: 827.

| | | | Amino acid coordinates |
|---|---|---|---|
| Database | Accession number | Accession name | on SEQ ID NO 2 |
| InterPro | IPR000504 | RNA recognition | |
| | | motif, RNP-1 | |
| HMMPfam | PF00076 | RRM_1 | T[33-104] 5.39E−29 |
| HMMSmart | SM00360 | RRM | T[32-105] 8.50E−30 |
| ProfileScan | PS50102 | RRM | T[31-109] 0.0 |
| InterPro | IPR002952 | Eggshell protein | |
| FPrintScan | PR01228 | EGGSHELL | T[36-47] 1.8E−10 T[116-131] |
| | | | 1.8E−10 T[144-154] 1.8E−10 |
| | | | T[170-188] 1.8E−10 |
| InterPro | IPR012677 | Nucleotide- | |
| | | binding, alpha- | |
| | | beta plait | |
| Gene3D | G3DSA:3.30.70.330a_b_plait_nuc_bd | | T[29-147] 1.10E−32 |
| InterPro | IPR015465 | RNA recognition | |
| | | motif, glycine rich | |
| | | protein | |
| HMMPanther | PTHR10432:SF31 RRM_Gly_rich | | T[31-225] 7.90003079443043E−47 |
| InterPro | NULL | NULL | |
| HMMPanther | PTHR10432 | PTHR10432 | T[31-225] 7.90E−47 T[31-225] |
| | | | 7.90E−47 |
| Superfamily | SSF54928 | SSF54928 | T[9-145] 1.6E−32 |

### Example 5

### Topology Prediction of the Polypeptide Sequences Useful in Performing the Methods of the Invention

### 5.1. C3H-Like Polypeptides

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters are selected, such as organism group (non-plant or plant), cutoff sets (none, predefined set of cutoffs, or user-specified set of cutoffs), and the calculation of prediction of cleavage sites (yes or no).

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).

### 5.2. SPATULA-Like (SPT) Polypeptides

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters are selected, such as organism group (non-plant or plant), cutoff sets (none, predefined set of cutoffs, or user-specified set of cutoffs), and the calculation of prediction of cleavage sites (yes or no).

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).

### 5.3. IDI2 (Iron Deficiency Induced 2) Polypeptides

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The results of TargetP 1.1 analysis of the polypeptide sequence as represented by SEQ ID NO: 140 are presented Table D1. The “plant” organism group has been selected, no cutoffs defined, and the predicted length of the transit peptide requested. The subcellular localization of the polypeptide sequence as represented by SEQ ID NO: 140 may be the cytoplasm or nucleus, no transit peptide is predicted.

TABLE D1
TargetP 1.1 analysis of the polypeptide sequence as
represented by SEQ ID NO: 140.

| Name | Len | cTP | mTP | SP | other | Loc | RC | TPlen |
|---|---|---|---|---|---|---|---|---|
| SEQ ID NO: 140 | 367 | 0.047 | 0.338 | 0.070 | 0.421 | — | 5 | — |
| cutoff | | 0.000 | 0.000 | 0.000 | 0.000 | | | |

Abbreviations:
Len, Length;
cTP, Chloroplastic transit peptide;
mTP, Mitochondrial transit peptide,
SP, Secretory pathway signal peptide,
other, Other subcellular targeting,
Loc, Predicted Location;
RC, Reliability class;
TPlen, Predicted transit peptide length.

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).
   5.4. eIF4F-Like Protein Complex

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).

### 5.5. GR-RBP (Glycine Rich-RNA Binding Protein) Polypeptides

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The results of TargetP 1.1 analysis of the polypeptide sequence as represented by SEQ ID NO: 2 are presented Table D2. The “plant” organism group has been selected, no cutoffs defined, and the predicted length of the transit peptide requested. The subcellular localization of the polypeptide sequence as represented by SEQ ID NO: 827 is predicted to be the mitochondrion, a transit peptide does not appear to be present.

TABLE D2
TargetP 1.1 analysis of the polypeptide sequence as
represented by SEQ ID NO: 827.

| Name | Len | cTP | mTP | SP | other | Loc | RC | TPlen |
|---|---|---|---|---|---|---|---|---|
| SEQ ID NO: 827 | 258 | 0.282 | 0.582 | 0.035 | 0.058 | M | 4 | 28 |
| cutoff | | 0.000 | 0.000 | 0.000 | 0.000 | | | |

Abbreviations:
Len, Length;
cTP, Chloroplastic transit peptide;
mTP, Mitochondrial transit peptide,
SP, Secretory pathway signal peptide,
other, Other subcellular targeting,
Loc, Predicted Location;
RC, Reliability class;
TPlen, Predicted transit peptide length.

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).

### Example 6

### Assay Related to the Polypeptide Sequences Useful in Performing the Methods of the Invention

### 6.1. IDI2 (Iron Deficiency Induced 2) Polypeptides

The functionality of eukaryotic initiation factor 2B may be assayed as described by Fabian et al. (1997, 1998). In brief, the baculovirus expression vector system (BEVS) is used to express FLAG epitope tagged alleles for the alpha, beta, gamma, delta, and epsilon subunits of rat eIF2B in Sf21 cells. The eIF2B holoprotein is reconstituted in vivo by coexpression of all five subunits in Sf21 cells and is subsequently purified using a two-step procedure involving an anti-FLAG immunoaffinity column followed by a gel filtration chromatography.

The purified five-subunit eIF2B complex has high Guanine nucleotide Exchange Factor (GEF) activity as assayed by measuring the exchange of [³H]GDP bound to eIF2 for unlabeled GDP using [³H]GDP-bound to eIF2 as a substrate. The labeled binary complex eIF2-[³H]GDP is prepared by incubating tubes containing rat liver eIF2 [about 95% pure] and [³H]GDP (2.5 mM, 10.9 Ci/mmol) in 80 ml assay buffer (62.5 mM MOPS, pH 7.4, 125 mM KCl, 1.25 mM DTT, 0.2 mg/ml BSA) at 30° C. for 10 min. The Mg²⁺ concentration is adjusted to 2 mM and the binary complex is stored on ice before use. To measure GEF activity, assay buffer containing a 100-fold excess of GDP, purified protein or cell lysate (1.25-40 ml), and 2 mM Mg²⁺ is added to a tube followed by labeled binary complex (1-2 μmol) and the mixture is incubated at 37° C. for 0-12 min. The exchange reaction is measured as a decrease in the eIF2 mediated binding of [³H]GDP to nitrocellulose filters with time.

### 6.2. GR-RBP (Glycine Rich-RNA Binding Protein) Polypeptides

RNA-binding activity of GR-RBP proteins can be determined as described by Kwak et al. (2005) for GR-RBP4.

The proteins used for the in vitro nucleic acid binding assay are synthesized by in vitro transcription and translation. The cDNA encoding GR-RBP4 is subcloned into the pET-22b(+) vector (Novagen). The in vitro transcription/translation reaction is performed using the T_{N}T® Quick Coupled Transcription/Translation System with T7 RNA polymerase (Promega). One microgram of DNA is mixed with the reaction mixture containing 40 μl T_{N}T® Quick Master Mix, 2 μl [³⁵S]methionine, and 6 μl nuclease-free water. The reaction mixture is incubated at 30° C. for 90 min. Five microlitres of the in vitro-synthesized protein is mixed with 5 μl of ribohomopolymer-agarose beads or DNA-cellulose beads at a concentration of 1 mg ml⁻¹ in 20 μl of binding buffer (10 mM TRIS-HCl, pH 7.4, 2.5 mM MgCl₂, 0.5% Triton X-100, and 125-1000 mM NaCl) with 1 mg ml⁻¹ heparin. The mixture is incubated on ice for 30 min, and the beads are washed three to four times to remove the unbound-proteins with the binding buffer containing 125-1000 mM NaCl (no heparin). After the last wash, the samples are dried, and resuspended by boiling in 30 μl of SDS loading buffer. The released proteins are separated by SDS-12% PAGE, and the relative intensities of the protein bands are quantified by a Phosphorlmager (Fuji, Japan).

Binding between the [³⁵S]methionine-labelled GR-RBP4 protein and single-stranded DNA (ssDNA), double-stranded DNA (dsDNA), or homoribopolymers (poly(A), poly(C), poly(G), and poly(U)) are tested at different NaCl concentrations. The GR-RBP4 binds strongly to all DNAs and RNAs tested in the presence of 250 mM NaCl. Binding is also observed at high salt concentrations of 1.0 M NaCl. GR-RBP4 has high affinity to ssDNA and dsDNA as well as RNAs. To verify the specificity of this binding assay further, GR-RBP2 and GR-RBP7 as other members of the GR-RBP family, and luciferase as a negative control, are tested. GR-RBP2 binds most strongly to poly(U) as observed by Vermel et al. (Proc. Natl. Acad. Sci. USA 99, 5866-5871, 2002), and GR-RBP7 shows higher affinity to poly(G), poly(U), and ssDNA as observed in many other GR-RBPs (Ludevid et al., The Plant Journal 2, 999-1003, 1992; Hirose et al., Mol. Gen. Gen. 244, 360-366, 1994). No binding is detected for luciferase that contains neither RRM nor a glycine-rich motif. These observations support the reliability of the binding assay, and indicate that GR-RBP4 binds sequence non-specifically to RNAs and DNAs.

### Example 7

### Cloning of the Nucleic Acid Sequence Used in the Methods of the Invention

### 7.1. C3H-Like Polypeptides

The nucleic acid sequence used in the methods of the invention was amplified by PCR using as template a Medicago truncatula cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm10911 (SEQ ID NO: 93; sense, start codon in bold): 5′-ggggacaagtttgtacaaaaaagcaggcttaaacaatgaattctgaatc ctcaccc-3′ and prm10912 (SEQ ID NO: 94; reverse, complementary): 5′-ggggaccactttgtacaagaaagctgggtac aatagaatcaatcttccaattc-3′, which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pC3H-like. Plasmid pDONR201 was purchased from Invitrogen, as part of the Gateway® technology.

The entry clone comprising SEQ ID NO: 1 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 95) for constitutive specific expression was located upstream of this Gateway cassette.

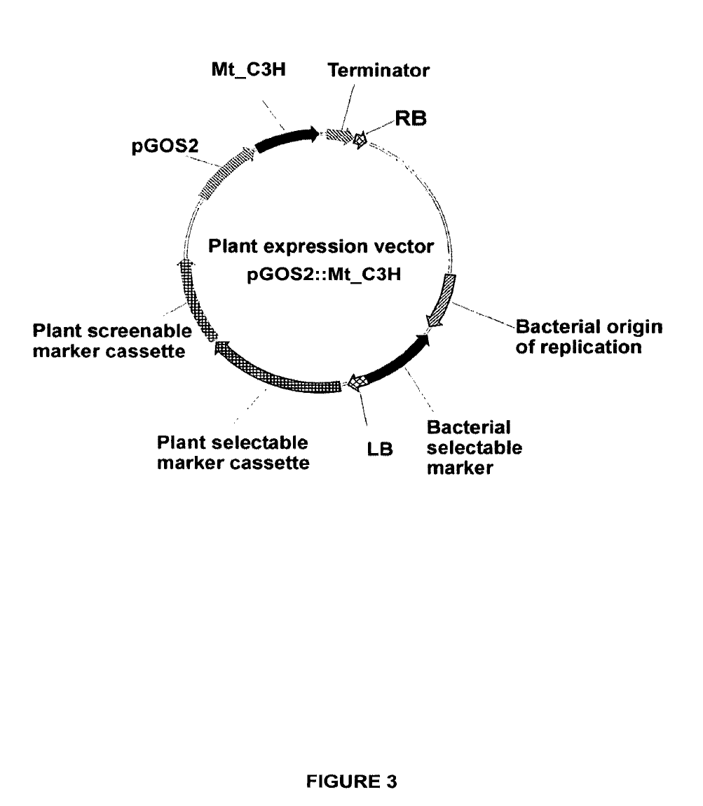

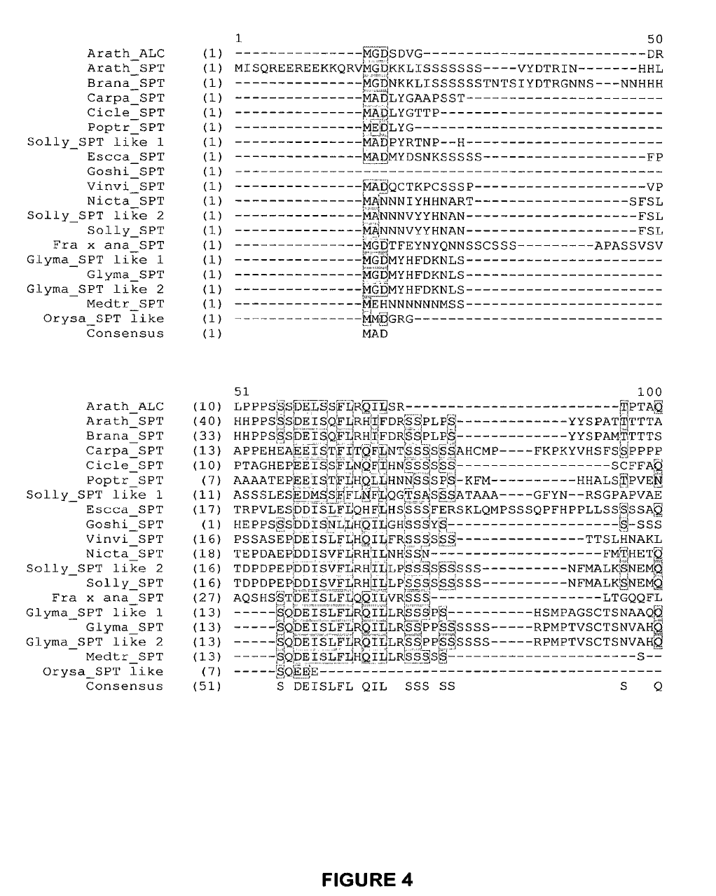

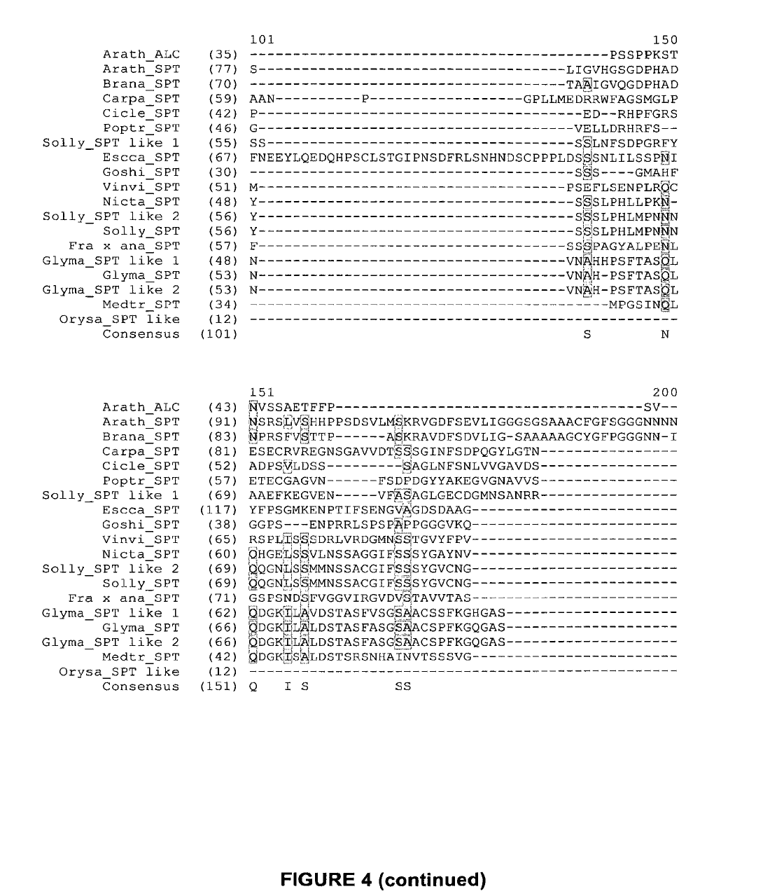

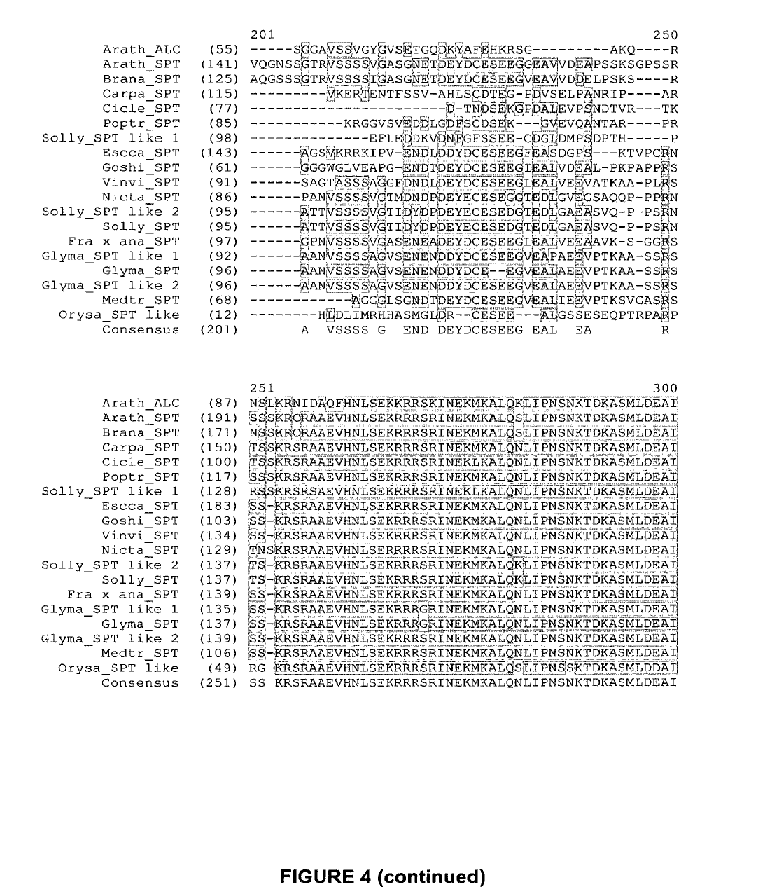

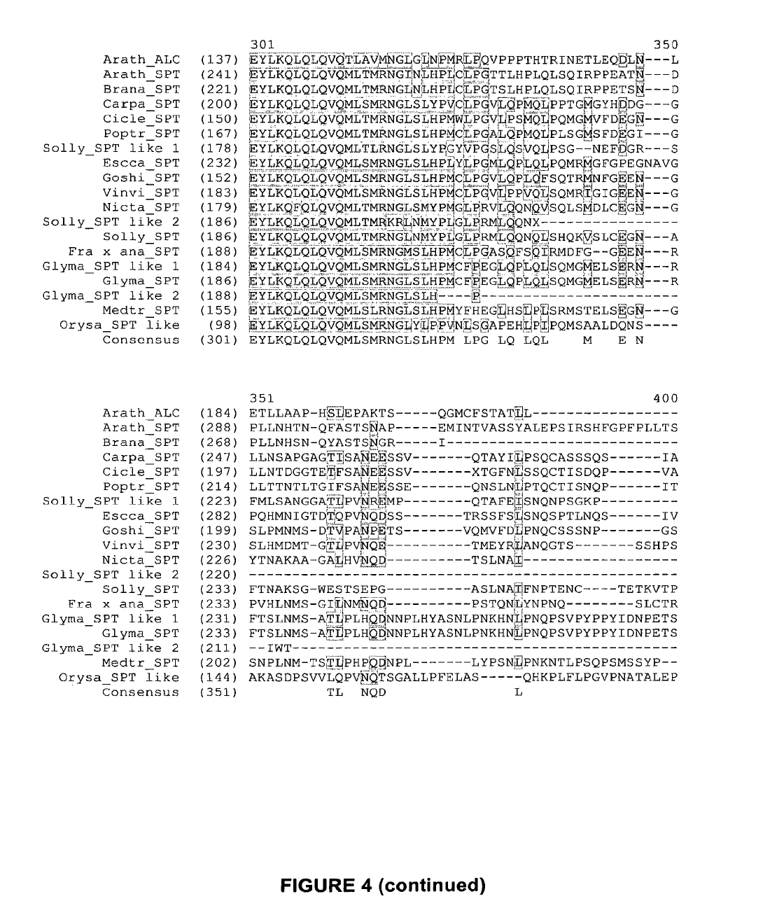

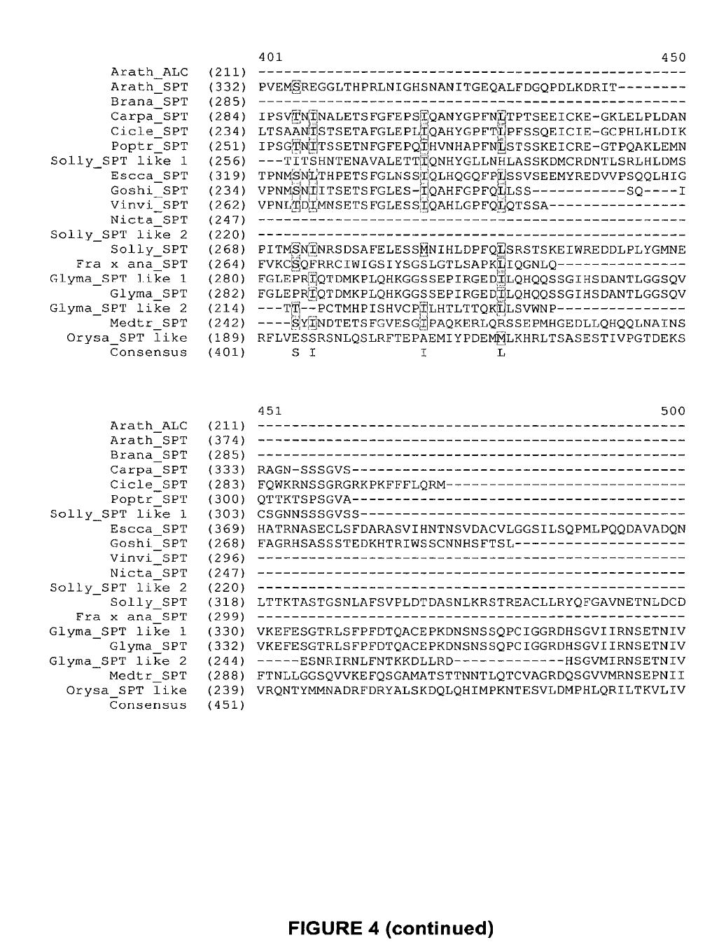

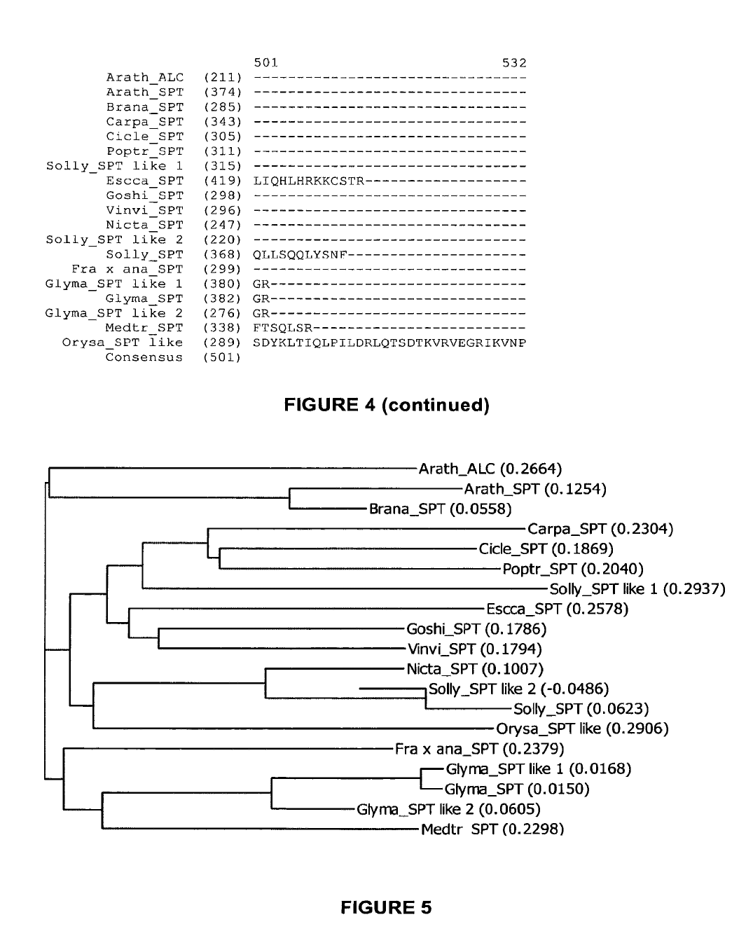

After the LR recombination step, the resulting expression vector pGOS2::C3H-like (FIG. 3) was transformed into Agrobacterium strain LBA4044 according to methods well known in the art.

### 7.2. SPATULA-like (SPT) Polypeptides

The nucleic acid sequence used in the methods of the invention was amplified by PCR using as template a Populus trichocarpa cDNA library. PCR was performed using Hifi Taq DNA polymerase in standard conditions, using 200 ng of template in a 50 μl PCR mix. The primers used were prm11534 (SEQ ID NO: 133; sense, start codon in bold): 5′-ggggacaagtttgta caaaaaagcaggcttaaacaatggaggatctgtacggagc-3′ and prm11535 (SEQ ID NO: 134; reverse, complementary): 5′-ggggaccactttgtacaagaaagctgggttcataactaggccacaccaga-3′, which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pSPT-like. Plasmid pDONR201 was purchased from Invitrogen, as part of the Gateway® technology.

The entry clone comprising SEQ ID NO: 96 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 135) for constitutive specific expression was located upstream of this Gateway cassette.

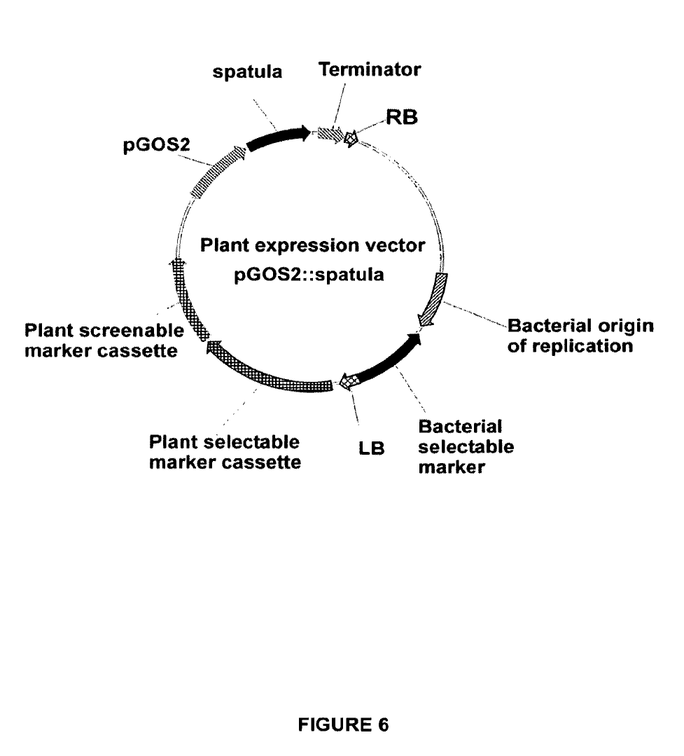

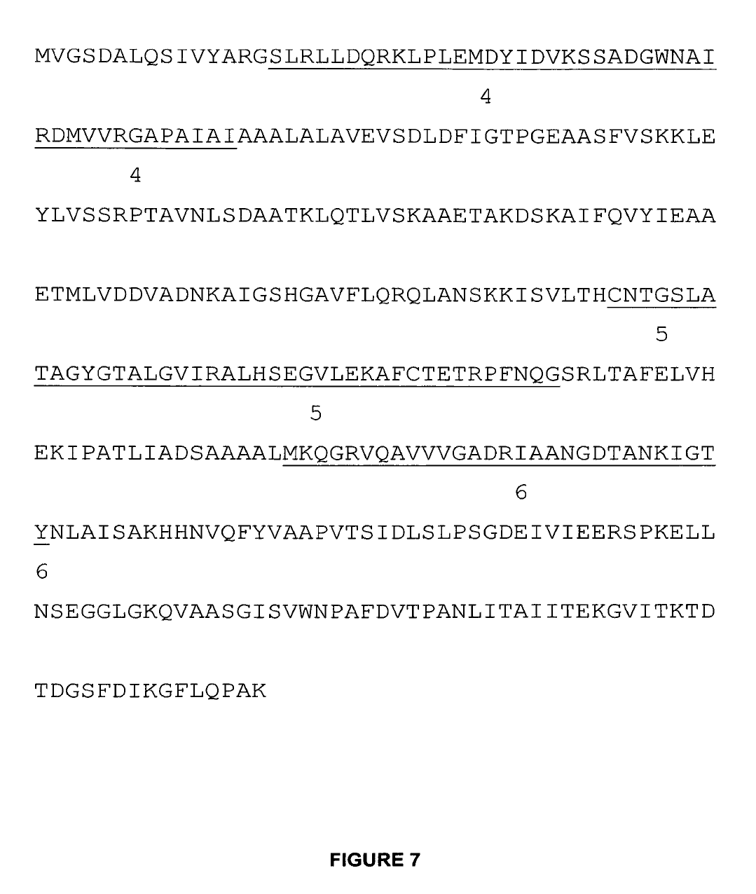

After the LR recombination step, the resulting expression vector pGOS2::SPT-like (FIG. 6) was transformed into Agrobacterium strain LBA4044 according to methods well known in the art.

### 7.3. IDI2 (Iron Deficiency Induced 2) Polypeptides

The nucleic acid sequence used in the methods of the invention was amplified by PCR using as template a custom-made Saccharum officinarum seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm08213 (SEQ ID NO: 147; sense, start codon in bold): 5′-ggggacaagtttgtacaaaaaagcaggctta aacaatggtgggatccgacg-3′ and prm08214 (SEQ ID NO: 148; reverse, complementary): 5′-ggggaccactttgtacaagaaagctgggtgccacgcttgagagtattat t-3′, which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pIDI2. Plasmid pDONR201 was purchased from Invitrogen, as part of the Gateway® technology.

The entry clone comprising SEQ ID NO: 139 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 149) for constitutive specific expression was located upstream of this Gateway cassette.

After the LR recombination step, the resulting expression vector pGOS2::IDI2 (FIG. 9) was transformed into Agrobacterium strain LBA4044 according to methods well known in the art.

7.4. eIF4F-Like Protein Complex

The nucleic acid sequence used in the methods of the invention was amplified by PCR using as template an Oryza sativa cDNA library (in pCMV Sport 6.0; Invitrogen, Paisley, UK). PCR was performed using Hifi Taq DNA polymerase in standard conditions, using 200 ng of template in a 50 μl PCR mix. The primers used were:

For SEQ. ID. NO 240
primer 1 (SEQ ID NO: 810);
(fwd) 5′-ggggacaagtttgtacaaaaaagcaggcttaaacaatggagaaggatcaccag-3′
and
primer 2 (SEQ ID NO: 811):
(rev) 5′-ggggaccactttgtacaagaaagctgggtttatttcagaagtttgttgca-3′,
For SEQ. ID. NO 300
primer 3 (SEQ ID NO: 812);
(fwd) 5′-ggggacaagtttgtacaaaaaagcaggcttaaacaatggcgggaatggca-3′
and
primer 4 (SEQ ID NO: 813):
(rev) 5′-ggggaccactttgtacaagaaagctgggttcaggccccttaacataactc-3′,

which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, peIF4 isoG and peIF4A. Plasmid pDONR201 was purchased from Invitrogen, as part of the Gateway® technology.

The entry clone comprising SEQ ID NO: 240 and SEQ ID NO: 300 were then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 818) for constitutive specific expression was located upstream of this Gateway cassette.

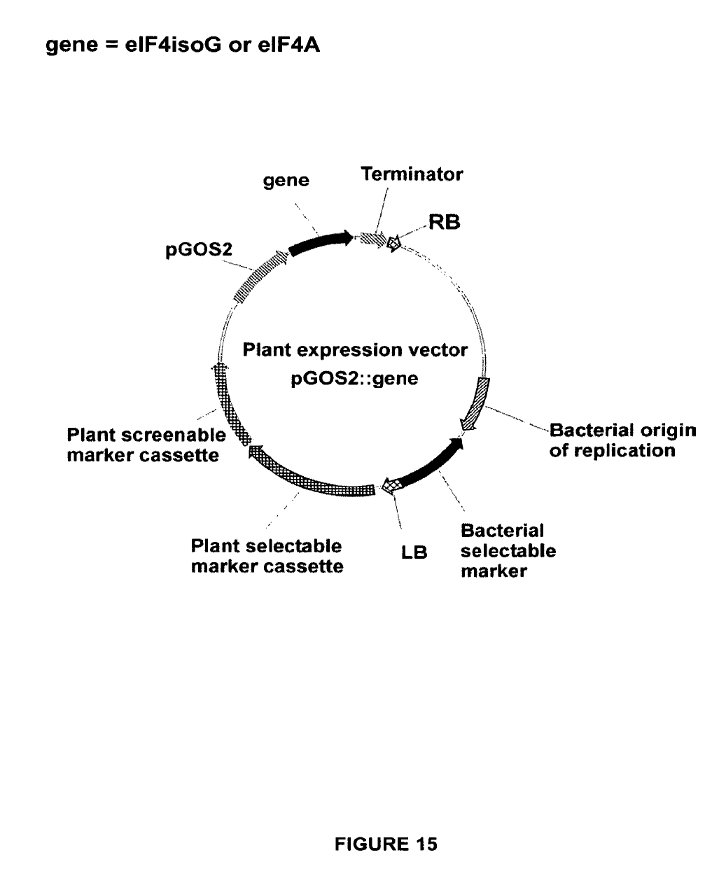

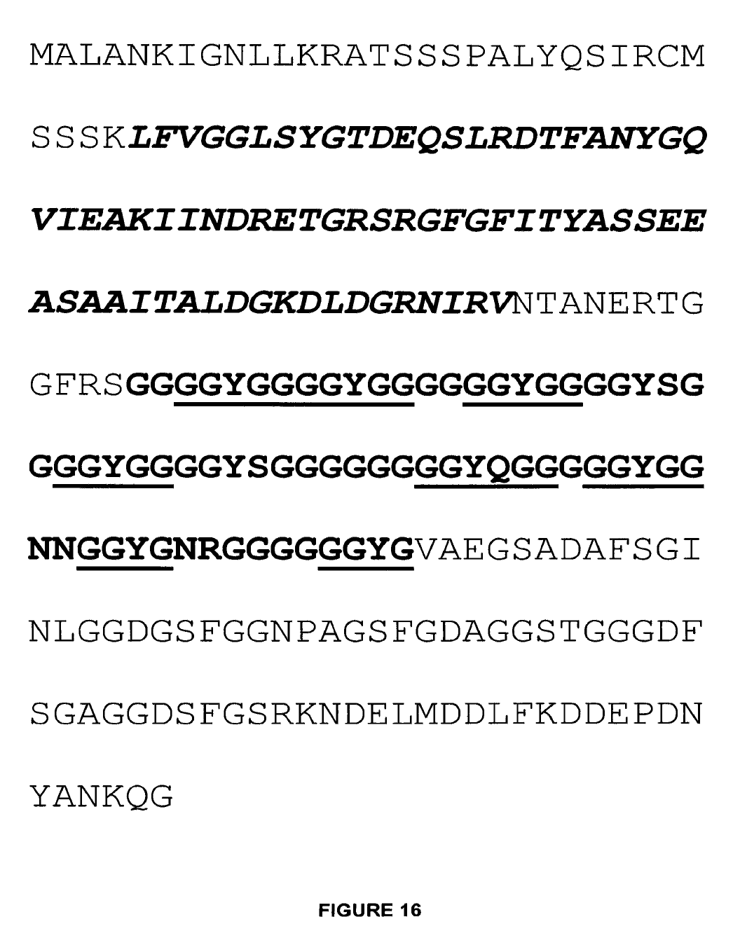

After the LR recombination step, the resulting expression vectors pGOS2::eIF4F4 isoG and pGOS2::eIF4F4A (FIG. 15) were transformed into Agrobacterium strain LBA4044 according to methods well known in the art.

### 7.5. GR-RBP (Glycine Rich-RNA Binding Protein) Polypeptides

The nucleic acid sequence used in the methods of the invention was amplified by PCR using as template a custom-made Oryza sativa seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm10480 (SEQ ID NO: 838; sense, start codon in bold): 5′-ggggacaagtttgtacaaaaaagcag gcttaaacaatggcgttggctaataagatt-3′ and prm10481 (SEQ ID NO: 838; reverse, complementary): 5′-ggggaccactttgtacaagaaagctgggtaggctcgaaggacgtagatta-3′, which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pGR-RBP. Plasmid pDONR201 was purchased from Invitrogen, as part of the Gateway® technology.

The entry clone comprising SEQ ID NO: 826 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 840) for constitutive expression was located upstream of this Gateway cassette.

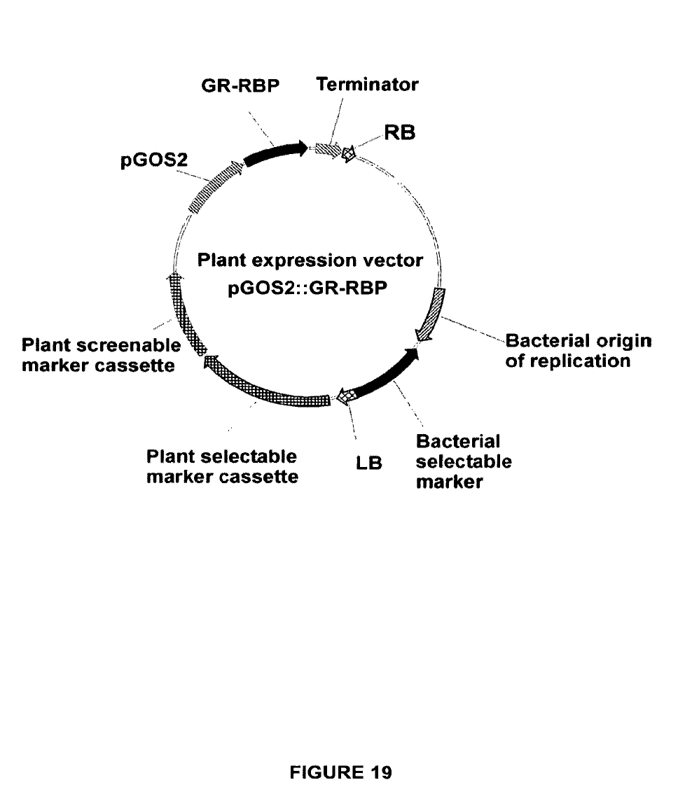

After the LR recombination step, the resulting expression vector pGOS2::GR-RBP (FIG. 19) was transformed into Agrobacterium strain LBA4044 according to methods well known in the art.

### Example 8

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent TO rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges1996, Chan et al. 1993, Hiei et al. 1994).

### Example 9

### Transformation of Other Crops

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M patent U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MSO) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton is transformed using Agrobacterium tumefaciens according to the method described in U.S. Pat. No. 5,159,135. Cotton seeds are surface sterilised in 3% sodium hypochlorite solution during 20 minutes and washed in distilled water with 500 μg/ml cefotaxime. The seeds are then transferred to SH-medium with 50 μg/ml benomyl for germination. Hypocotyls of 4 to 6 days old seedlings are removed, cut into 0.5 cm pieces and are placed on 0.8% agar. An Agrobacterium suspension (approx. 108 cells per ml, diluted from an overnight culture transformed with the gene of interest and suitable selection markers) is used for inoculation of the hypocotyl explants. After 3 days at room temperature and lighting, the tissues are transferred to a solid medium (1.6 g/l Gelrite) with Murashige and Skoog salts with B5 vitamins (Gamborg et al., Exp. Cell Res. 50:151-158 (1968)), 0.1 mg/l 2,4-D, 0.1 mg/l 6-furfurylaminopurine and 750 μg/ml MgCL2, and with 50 to 100 μg/ml cefotaxime and 400-500 μg/ml carbenicillin to kill residual bacteria. Individual cell lines are isolated after two to three months (with subcultures every four to six weeks) and are further cultivated on selective medium for tissue amplification (30° C., 16 hr photoperiod). Transformed tissues are subsequently further cultivated on non-selective medium during 2 to 3 months to give rise to somatic embryos. Healthy looking embryos of at least 4 mm length are transferred to tubes with SH medium in fine vermiculite, supplemented with 0.1 mg/l indole acetic acid, 6 furfurylaminopurine and gibberellic acid. The embryos are cultivated at 30° C. with a photoperiod of 16 hrs, and plantlets at the 2 to 3 leaf stage are transferred to pots with vermiculite and nutrients. The plants are hardened and subsequently moved to the greenhouse for further cultivation.

### Example 10

### Phenotypic Evaluation Procedure

### 10.1 Evaluation Setup

Approximately 35 independent TO rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. Plants grown under non-stress conditions were watered at regular intervals to ensure that water and nutrients were not limiting and to satisfy plant needs to complete growth and development.

Three to four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### Drought Screen (C3H-Like Polypeptides)

Plants from T2 seeds were grown in potting soil under normal conditions until they approached the heading stage. They were then transferred to a “dry” section where irrigation was withheld. Humidity probes were inserted in randomly chosen pots to monitor the soil water content (SWC). When SWC fell below certain thresholds, the plants were automatically re-watered continuously until a normal level was reached again. The plants were then re-transferred to normal conditions. The rest of the cultivation (plant maturation, seed harvest) was the same as for plants not grown under abiotic stress conditions. Growth and yield parameters were recorded as detailed for growth under normal conditions.

### Nitrogen Use Efficiency Screen (IDI2 Polypeptides)

Rice plants from T2 seeds were grown in potting soil under normal conditions except for the nutrient solution. The pots were watered from transplantation to maturation with a specific nutrient solution containing reduced N nitrogen (N) content, usually between 7 to 8 times less. The rest of the cultivation (plant maturation, seed harvest) was the same as for plants not grown under abiotic stress. Growth and yield parameters were recorded as detailed for growth under normal conditions.

### Salt Stress Screen

Plants are grown on a substrate made of coco fibers and argex (3 to 1 ratio). A normal nutrient solution is used during the first two weeks after transplanting the plantlets in the greenhouse. After the first two weeks, 25 mM of salt (NaCl) is added to the nutrient solution, until the plants are harvested. Seed-related parameters are then measured.

### 10.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment—event—segregants). P values were obtained by comparing likelihood ratio test to chi square distributions.

### 10.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

Early vigour was determined by counting the total number of pixels from aboveground plant parts discriminated from the background. This value was averaged for the pictures taken on the same time point from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 11

### Phenotypic Evaluation Procedure

### 11.1. C3H-Like Polypeptides (Drought Stress)

The following parameters were significantly increased in either the T1, T2 or both generations with a p-value from the F-test of <0.05. The % difference between the transgenic plants compared to corresponding nullizygotes is also given.
Above ground biomass: 7%
Root/shoot index: −9.6 (meaning that there are fewer roots than shoots)
Number of thick roots: 5%
Total weight of seeds: at least 17% (more in the T2 generation)
Number of filled seeds: at least 20.3% (more in the T2 generation)
Fill rate: at least 16.2% (more in the T2 generation)
Harvest index: 42.7%
Number of first panicles: 8.9%

A positive tendency was also observed for the following parameters in some individual lines: emergence vigour, root biomass, increased number of thin roots, number of total seeds, increased plant height, each relative to corresponding nullizygotes.

### 11.2. SPATULA-Like (SPT) Polypeptides

The results of the evaluation of transgenic rice plants in the T1 and T2 generations showed a significant increase in Thousand Kernel Weight (TKW) compared to corresponding nullizygotes. There was also a positive tendency towards biomass increase, increased plant height and an increase in the total weight of seeds.

### 11.3. IDI2 (Iron Deficiency Induced 2) Polypeptides (Nitrogen-Limiting Conditions)

Plants were evaluated in both T1 and T2 generation. When grown under nitrogen-limiting conditions, the transgenic plants had an increase in the number of filled seeds, harvest index and in the total weight of seeds; details are given in Table E1 below:

TABLE E1
Data summary for transgenic rice plants; for each parameter, the
overall percent increase is shown for the T1 generation and the
confirmation (T2 generation), for each parameter the p-value is <0.05.

| Parameter | Overall increase in T1 | Overall increase in T2 |
|---|---|---|
| total weight seeds | 37.5% | 19.0% |
| number filled seeds | 36.8% | 16.6% |
| harvest index | 6.0% | 13.5% |

In addition, plants expressing an IDI2 nucleic acid also showed increased biomass (above ground and root biomass), increased early vigour, and an increased total number of seeds, compared to the control plants.

11.4. eIF4F-Like Protein Complex

The results of the evaluation of transgenic rice plants in the T2 generation and expressing a nucleic acid comprising the longest Open Reading Frame in SEQ ID NO: 240 under non-stress conditions are presented below in Table E2. See previous Examples for details on the generations of the transgenic plants. An increase of (at least—more than) 5% was observed for number of flowers per panicle and maximum root thickness.

TABLE E2
results for T2 transgenic rice plants expressing SEQ ID NO: 240

| Parameter | Overall |
|---|---|
| flowers per panicles | 9.7 |
| Root Thick Max | 7.6 |

The results of the evaluation of transgenic rice plants in the T1 generation and expressing a nucleic acid comprising the longest Open Reading Frame in SEQ ID NO: 300 under non-stress conditions are presented below. See previous Examples for details on the generations of the transgenic plants. An increase of (at least—more than) 5% was observed for fill rate, harvest index and maximum root thickness.

TABLE E3
results for T1 transgenic rice plants expressing SEQ ID NO: 300

| Parameter | Overall |
|---|---|
| fill rate | 5.7 |
| harvest index | 7.5 |
| Root Thick Max | 6.9 |

### 11.5. GR-RBP (Glycine Rich-RNA Binding Protein) Polypeptides (Drought Stress)

Plants were evaluated in both T1 and T2 generation. When grown under drought-stress conditions, the transgenic plants had an increase in early vigour and showed an increase in biomass (above ground and roots) and seed yield; details are given in Table E4 below:

TABLE E4
Data summary for transgenic rice plants of the T1 generation; for
each parameter, the overall percent increase is shown, and for
each parameter the p-value is <0.05.

| Parameter | Overall |
|---|---|
| Area Max | 7.0 |
| EmerVigor | 14.3 |
| total weight seeds | 51.5 |
| fill rate | 63.5 |
| harvest index | 45.2 |
| number filled seeds | 51.1 |
| Root Thick Max | 12.2 |

Yield increase and early vigour were again observed in the T2 generation.

Furthermore, when grown under non-stress conditions, an increase was observed in T1 plants for above-ground biomass, fillrate (each more than 5%) and Thousand Kernel Weight (2.2%).

1-124. (canceled)

125. A method for enhancing a yield-related trait in a plant relative to a corresponding control plant, comprising:
(a) modulating expression in a plant of a nucleic acid encoding a C3H-like polypeptide, wherein said C3H-like polypeptide comprises Domain 4 and any one or more of Domains 1, 2, 3 and 5 defined as follows:

| | |
|---|---|
| (i) Domain 1: | C-X₂-C-X₁₂₋₂₃-C-X₂-C-X₂-G-F; |

wherein X is any amino acid and the underlined residues are conserved,

| | |
|---|---|
| (ii) Domain 2: | Y-X₇₋₁₂-L-X₃-P-X₁₀-G; |

wherein X is any amino acid and the underlined residues are conserved,

| | |
|---|---|
| (iii) Domain 3: | S-K-X₆-P; |

wherein X is any amino acid and the underlined residues are conserved,

| | |
|---|---|
| (iv) Domain 4: | RING-C3H2C3 type; |
| and | |
| (v) Domain 5: | DUF1117; |

(b) modulating expression in a plant of a nucleic acid encoding an SPT-like polypeptide comprising each of the following motifs from N-terminus to C-terminus:
(i) Motif I: an amphipathic helix comprising EEISTFLHQLLH, or a motif having at least 60% sequence identity to Motif I;
(ii) Motif II: an acidic domain comprising DLGDFSCDSEK or a motif having at least 60% sequence identity to Motif II; and
(iii) Motif III: a bHLH domain comprising; AAEVHNLSEKRRRSRINEKMKALQNLIPNSNKT DKASMLDEAIEYLKQL or a motif having at least 60% sequence identity to Motif III;
(c) modulating expression in a plant of a nucleic acid encoding an IDI2 polypeptide, wherein said IDI2 polypeptide comprises an IF-2B domain;
(d) modulating the activity in a plant of an eIF4F-like protein complex by modulating and expressing its subunit polypeptides and/or isoforms thereof, and/or by modulating the level of the eIF4F-like protein complex in the plant, wherein said eIF4F-like protein complex comprises the subunits eIF4G, eIF4A, and eIF4E, or isoforms thereof, comprising respectively the following CC domains with the PFam accession numbers:
(i) for eIF4G polypeptides: MA3 (PFam accession number: PF02847) and MIF4G (PFam accession number: PF02854);
(ii) for eIF4A polypeptides: DEAD (PFam accession number: PF00270) and Helicase_C(PFam accession number: PF00271); and
(iii) for eIF4E polypeptydes: IF4E (PFam accession number: PF01652);
or
(e) modulating expression in a plant of a nucleic acid encoding a Glycine-Rich RNA Binding Protein (GR-RBP polypeptide), wherein said GR-RBP polypeptide comprises an RNA Recognition Motif 1 (PFam accession PF00076, RRM 1).

126. The method of claim 125, wherein
(a) the Domain 1 of the C3H-like polypeptide is: CYSCTRFINLSDHTL----------IVCPHCDNGF, or a domain comprising the underlined conserved residues and having at least 60% sequence identity to the non-underlined residues in the Domain 1, wherein “-” is a gap or any residue;
(b) the Domain 2 of the C3H-like polypeptide is: YDDGDG-----SGLRPLPPTVSEFLLGSG, or a domain comprising the underlined conserved residues and having at least 60% sequence identity to the non-underlined residues in the Domain2, wherein “-” is a gap or any residue;
(c) the Domain 3 of the C3H-like polypeptide is: SKAAIESMP, or a domain comprising the underlined conserved residues and having at least 60% sequence identity to the non-underlined residues in the Domain3;
(d) the Domain 4 of the C3H-like polypeptide is: CAVCKEEFELHAEARELPCKHLYHSDCILPWLTVRNSCPVCR, or a domain comprising the underlined conserved residues and having at least 60% sequence identity to the non-underlined residues in the Domain 4;
(e) the Domain 5 of the C3H-like polypeptide is: GLTIWRLPGGGFAVGRFSGGRSA-GESHFPVVYTEMDGGLN, or a domain having at least 60% sequence identity to the Domain 5, wherein “-” is a gap or any residue;
(f) the SPT-like polypeptide further comprises one or more serine-rich regions;
(g) the bHLH domain of the SPT-like polypeptide further comprises one or more nuclear localisation signals (NLS);
(h) the SPT-like polypeptide comprises a beta strand adjacent to the bHLH domain nearest the C-terminal region, wherein the beta strand comprises QLQVQMLTM;
(i) the IDI2 polypeptide comprises one or more of the motifs of SEQ ID NO: 141 to SEQ ID NO: 146;
(j) the eIF4G subunit polypeptide of the eIF4F-like protein complex comprises a CC domain comprising the amino acid sequence of SEQ ID NO: 240, and/or a sequence having at least 50% sequence identity to the eIF4G polypeptide of SEQ ID NO: 241;
(k) the eIF4G subunit polypeptide of the eIF4F-like protein complex comprises the following motifs:
Motif 7: KAV[LF]EPTFCPMYA[QL]LCSDLNEKLP[PS]FPS[ED]EPGGKEITF KRVLLN[NI]CQEAF or a motif having at least 50% sequence identity to Motif 7,
Motif 8: CP[AE]EENVEAIC[QH]FFNTIGKQLDE[SN]PKSRRIND[MVT]YF [SIN][RQ]LKEL[TS][TS]NPQLAPR or a motif having at least 50% sequence identity to Motif 8,
Motif 9: T[AG]P[DE]QE[ML]ERRDKERLVKLRTLGNIRLIGELLKQKMV PEKIVHHIVQEL LG or a motif having at least 50% sequence identity to Motif 9,
or
Motif 10: TPQNF[ED][KR]LFEQVKAVNIDN[AV]VTL[TN]GVISQIF[DE]K ALMEPTFCEMYANFCFH or a motif having at least 50% sequence identity to Motif 10,
Motif 11: IGELYKK[RK]MLTERIMHECIKKLLGQYQ[DN]PDEE[DN][IV]E [AS]LCKLMSTIGEMIDH or a motif having at least 50% sequence identity to Motif 11,
Motif 12: LSNN[MQ][KN]LS SRVRFMLKD[ASV]IDLRKNKWQQRRKVEG PKKIEEVHRDAAQERQ or a motif having at least 50% sequence identity to Motif 12;
(o) the eIF4G subunit polypeptide of the eIF4F-like protein complex is an eIF4 isoG polypeptide and comprises the following motifs:
Motif 7: KAV[LF]EPTFCPMYA[QL]LCSDLNEKLP[PS]FPS[ED]EPGGKE ITFKRVLLN[NI]CQEAF or a motif having at least 50% sequence identity to Motif 7,
Motif 8: CP[AE]EENVEAIC[QH]FFNTIGKQLDE[SN]PKSRRIND[MVT]YF [SIN][RQ]LKEL[TS][TS]NPQLAPR or a motif having at least 50% sequence identity to Motif 8,
Motif 9: T[AG]P[DE]QE[ML]ERRDKERLVKLRTLGNIRLIGELLKQKMV PEKIVHHIVQELLG or a motif having at least 50% sequence identity to Motif 9;
(k) the eIF4A subunit polypeptide of the eIF4F-like protein complex comprises a CC domain comprising the amino acid sequence of SEQ ID NO: 300, and/or an amino acid sequence having at least 50% sequence identity to the eIF4G polypeptide of SEQ ID NO: 301;
(o) the eIF4A subunit polypeptide of the eIF4F-like protein complex comprises the following motifs:
Motif 13: RDELTLEGIKQF[YF]V[NA]V[ED][KR]EEWK[LF][DE]TLCDL Y[ED]TL[AT]ITQ[SA]VIF or a motif having at least 50% sequence identity to Motif 13,
Motif 14: SLVINYDLP[TN][QN][PR]E[NL]Y[LI]HRIGRSGRFGRKGVAINF or a motif having at least 50% sequence identity to Motif 14,
Motif 15: MG[LI][QK]E[ND]LLRGIYAYGFEKPSAIQQR[GA][IV]VP[FI][CI]KG[LR]DVI[QA]QAQSGTGKT[AS][TM][FI] or a motif having at least 50% sequence identity to Motif 15;
(l) the eIF4E subunit polypeptide of the eIF4F-like protein complex comprises a CC domain comprising the amino acid sequence of SEQ ID NO: 560, and/or a sequence having at least 50% sequence identity to the eIF4G polypeptide of SEQ ID NO: 561;
(p) the eIF4E subunit polypeptide of the eIF4F-like protein complex comprises the following motifs:
Motif 16: YTFSTVE[ED]FW[SG]LYNNIH[HR]PSKLAVGADF[HY]CFK[NH]KIEPKWEDP[VI]CANGGKW or a motif having at least 50% sequence identity to Motif 16,
Motif 17: T[SC]WLYTLLA[ML]IGEQFD[HY]GD[ED]ICGAVV[NS]VR or a motif having at least 50% sequence identity to Motif 17,
Motif 18: E[KR]I[AS][LI]WTKNA[AS]NE[AS T]AQ[VL]SIGKQWKEFLD YN[DE][TS]IGFIFH[ED]DA or a motif having at least 50% sequence identity to Motif 18,
or
Motif 19: WCLYDQ[IV]F[KR]PSKLP[GA]NADFHLFKAG[VI]EPKWEDPE CANGGKW or a motif having at least 50% sequence identity to Motif 19,
Motif 20: L[ED]TMWLETLMALIGEQFD[ED][AS][DE][ED]ICGVVASVR or a motif having at least 50% sequence identity to Motif 20,
Motif 21: QDKL[SA]LWT[KR][TN]A[AS]NEA[AV]QM[SG]IG[RK]KWK E[IV]ID or a motif having at least 50% sequence identity to Motif 21; or
(m) the GR-RBP polypeptide comprises one or more of the signature sequences or motifs of SEQ ID NO: 828 to SEQ ID NO: 837.

127. The method of claim 125, wherein the modulated expression or modulated activity is effected by introducing and expressing in the plant
(a) a nucleic acid encoding a C3H-like polypeptide;
(b) a nucleic acid encoding an SPT-like polypeptide;
(c) a nucleic acid encoding an IDI2 polypeptide;
(d) at least one nucleic acid encoding one of the eIF4F subunit polypeptides, a portion thereof, or a nucleic acid capable of hybridizing with said nucleic acid; or
(e) a nucleic acid encoding a GR-RBP polypeptide.

128. The method of claim 125, wherein
(a) the nucleic acid encoding a C3H-like polypeptide comprises:
(i) a nucleotide sequence that encodes any one of the proteins listed in Table A1;
(ii) a portion of the nucleotide sequence of (i); or
(iii) a nucleotide sequence capable of hybridizing with the nucleotide sequence of
(i) or (ii);
(b) the nucleic acid encoding an SPT-like polypeptide comprises:
(i) a nucleotide sequence that encodes any one of the proteins listed in Table A2;
(ii) a portion of the nucleotide sequence of (i); or
(iii) a nucleotide sequence capable of hybridizing with the nucleotide sequence of (i) or (ii);
(c) the nucleic acid encoding an IDI2 polypeptide comprises:
(i) a nucleotide sequence that encodes any one of the proteins listed in Table A3;
(ii) a portion of the nucleotide sequence of (i); or
(iii) a nucleotide sequence capable of hybridizing with the nucleotide sequence of (i) or (ii); or
(d) the nucleic acid encoding a GR-RBP polypeptide comprises:
(i) a nucleotide sequence that encodes any one of the proteins listed in Table A5;
(ii) a portion of the nucleotide sequence of (i); or
(iii) a nucleotide sequence capable of hybridizing with the nucleotide sequence of (i) or (ii).

129. The method of claim 127, wherein the at least one nucleic acid encoding one of the eIF4F subunit polypeptides comprises:
(i) a nucleotide sequence that encodes an eIF4G subunit polypeptide and/or its isoforms;
(ii) a nucleotide sequence that encodes an eIF4A subunit polypeptide and/or its isoforms;
(iii) a nucleotide sequence that encodes an eIF4E subunit polypeptide and/or its isoforms;
(iv) a portion of the nucleotide sequence of (i), (ii), or (iii);
(v) a nucleotide sequence capable of hybridizing with the nucleotide sequence of (i), (ii), or (iii);
(vi) a nucleotide sequence that encodes the eIF4 isoG subunit; or
(vii) a nucleotide sequence that encodes the eIF4 isoE subunit.

130. The method of claim 127, wherein the at least one nucleic acid encoding one of the eIF4F subunit polypeptides, a portion thereof, or a nucleic acid capable of hybridizing with such a nucleic acid, is overexpressed.

131. The method of claim 125, wherein
(a) the nucleic acid encoding a C3H-like polypeptide encodes an orthologue or paralogue of any of the proteins given in Table A1;
(b) the nucleic acid encoding an SPT-like polypeptide encodes an orthologue or paralogue of any of the proteins given in Table A2;
(c) the nucleic acid encoding an IDI2 polypeptide encodes an orthologue or paralogue of any of the proteins given in Table A3; or
(d) the nucleic acid encoding a GR-RBP polypeptide encodes an orthologue or paralogue of any of the proteins given in Table A5.

132. The method of claim 127, wherein the at least one nucleic acid encoding one of the eIF4F subunit polypeptides encodes an orthologue or paralogue of any of the polypeptides given in Table A4.

133. The method of claim 125, wherein the enhanced yield-related trait comprises increased early vigor, increased yield, increased biomass, and/or increased seed yield relative to a corresponding control plant.

134. The method of claim 125, wherein the enhanced yield-related trait is obtained under non-stress conditions.

135. The method of claim 125, wherein the enhanced yield-related trait is obtained under conditions of drought stress, salt stress, or nitrogen deficiency.

136. The method of claim 127, wherein the nucleic acid is operably linked to a constitutive promoter, a GOS2 promoter, or a GOS2 promoter from rice.

137. The method of claim 125, wherein
(a) the nucleic acid encoding a C3H-like polypeptide is of plant origin, from the genus Medicago, or from Medicago truncatuta;
(b) the nucleic acid encoding an SPT-like polypeptide is of plant origin, from the family Salicaceae, from the genus Populus, or from Populus trichocaipa;
(c) the nucleic acid encoding an IDI2 polypeptide is of plant origin, from a monocotyledonous plant, from the family Poaceae, from the genus Saccharum, or from Saccharum officinarum;
(d) the nucleic acid encoding a GR-RBP polypeptide is of plant origin, from a monocotyledonous plant, from the family Poaceae, from the genus Oryza, or from Oryza sativa.

138. The method of claim 127, wherein the at least one nucleic acid encoding one of the eIF4F subunit polypeptides, a portion thereof, or a nucleic acid capable of hybridizing with such a nucleic acid, is of plant origin, from a dicotyledonous plant, from the family Brassicaceae, from the genus Arabidopsis, or from Arabidopsis thaliana.

139. A plant or part thereof, including seeds, obtained by the method of claim 125, wherein the plant or part thereof comprises a recombinant nucleic acid encoding
(a) a C3H-like polypeptide;
(b) an SPT-like polypeptide comprising each of the following motifs from N-terminus to C-terminus:
(i) Motif I: an amphipathic helix comprising EEISTFLHQLLH, or a motif having at least 60% sequence identity to Motif I;
(ii) Motif II: an acidic domain comprising DLGDFSCDSEK or a motif having at least 60% sequence identity to Motif II; and
(iii) Motif III: a bHLH domain comprising: AAEVHNLSEKRRRSRINEKMKALQNLIPNSNKT DKASMLDEAIEYLKQL or a motif having at least 60% sequence identity to Motif III;
(c) an IDI2 polypeptide;
(d) an eIF4F polypeptide subunit; or
(e) a GR-RBP polypeptide.

140. A construct comprising:
(a) a nucleic acid;
(b) one or more control sequences capable of driving expression of the nucleic acid sequence of (a); and optionally
(c) a transcription termination sequence,
wherein said nucleic acid is selected from the group consisting of:
(i) a nucleic acid encoding a C3H-like polypeptide as defined in claim 125;.
(ii) a nucleic acid encoding an SPT-like polypeptide comprising each of the following motifs from N-terminus to C-terminus:
(i) Motif I: an amphipathic helix comprising EEISTFLHQLLH, or a motif having at least 60% sequence identity to Motif I;
(ii) Motif II: an acidic domain comprising DLGDFSCDSEK or a motif having at least 60% sequence identity to Motif II; and
(iii) Motif III: a bHLH domain comprising: AAEVHNLSEKRRRSRINEKMKALQNLIPNSNKT DKASMLDEAIEYLKQL or a motif having at least 60% sequence identity to Motif III;
(iii) a nucleic acid encoding an IDI2 polypeptide, wherein said IDI2 polypeptide comprises an IF-2B domain;
(iv) a nucleic acid encoding a subunit polypeptide and/or isoforms thereof of an eIF4F-like protein complex, wherein said eIF4F-like protein complex comprises the subunits eIF4G, eIF4A, and eIF4E, or isoforms thereof, comprising respectively the following CC domains with the PFam accession numbers:
(i) for eIF4G polypeptides: MA3 (PFam accession number: PF02847) and MIF4G (PFam accession number: PF02854);
(ii) for eIF4A polypeptides: DEAD (PFam accession number: PF00270) and Helicase_C(PFam accession number: PF00271); and
(iii) for eIF4E polypeptydes: IF4E (PFam accession number: PF01652); and
(v) a nucleic acid encoding a Glycine-Rich RNA Binding Protein (GR-RBP polypeptide), wherein said GR-RBP polypeptide comprises a RNA Recognition Motif 1 (PFam accession PF00076, RRM_{—}1).

141. The construct of claim 140, wherein one of the control sequences is a constitutive promoter, a GOS2 promoter, or a GOS2 promoter from rice.

142. A method for making a plant having increased yield, increased biomass, and/or increased seed yield, comprising introducing into a plant or plant cell the construct of claim 140.

143. A plant, plant part, or plant cell transformed with the construct of claim 140.

144. A method for the production of a transgenic plant having increased yield, increased biomass, and/or increased seed yield relative to a corresponding control plant, comprising:
(a) introducing and expressing in a plant a nucleic acid; and
(b) cultivating the plant cell under conditions promoting plant growth and development, wherein said nucleic acid is selected from the group consisting of:
(i) a nucleic acid encoding a C3H-like polypeptide as defined in claim 125;
(ii) a nucleic acid encoding an SPT-like polypeptide comprising each of the following motifs from N-terminus to C-terminus:
(i) Motif I: an amphipathic helix comprising EEISTFLHQLLH, or a motif having at least 60% sequence identity to Motif I;
(ii) Motif II: an acidic domain comprising DLGDFSCDSEK or a motif having at least 60% sequence identity to Motif II; and
(iii) Motif III: a bHLH domain comprising: AAEVHNLSEKRRRSRINEKMKALQNLIPNSNKT DKASMLDEAIEYLKQL or a motif having at least 60% sequence identity to Motif III;
(iii) a nucleic acid encoding an IDI2 polypeptide, wherein said IDI2 polypeptide comprises an IF-2B domain;
(iv) a nucleic acid encoding a subunit polypeptide and/or isoforms thereof of an eIF4F-like protein complex, wherein said eIF4F-like protein complex comprises the subunits eIF4G, eIF4A, and eIF4E, or isoforms thereof, comprising respectively the following CC domains with the PFam accession numbers:
(i) for eIF4G polypeptides: MA3 (PFam accession number: PF02847) and MIF4G (PFam accession number: PF02854);
(ii) for eIF4A polypeptides: DEAD (PFam accession number: PF00270) and Helicase C(PFam accession number: PF00271); and
(iii) for eIF4E polypeptydes: IF4E (PFam accession number: PF01652); and
(v) a nucleic acid encoding a Glycine-Rich RNA Binding Protein (GR-RBP polypeptide), wherein said GR-RBP polypeptide comprises a RNA Recognition Motif 1 (PFam accession PF00076, RRM_{—}1).

145. A transgenic plant having increased yield, increased biomass, and/or increased seed yield relative to a corresponding control plant, resulting from modulated expression of a nucleic acid encoding a polypeptide, or a transgenic plant cell derived from said transgenic plant, wherein said polypeptide comprises:
(a) a C3H-like polypeptide as defined in claim 125;
(b) an SPT-like polypeptide comprising each of the following motifs from N-terminus to C-terminus:
(i) Motif I: an amphipathic helix comprising EEISTFLHQLLH, or a motif having at least 60% sequence identity to Motif I;
(ii) Motif II: an acidic domain comprising DLGDFSCDSEK or a motif having at least 60% sequence identity to Motif II; and
(iii) Motif III: a bHLH domain comprising: AAEVHNLSEKRRRSRINEKMKALQNLIPNSNKT DKASMLDEAIEYLKQL or a motif having at least 60% sequence identity to Motif III;
(c) an IDI2 polypeptide, wherein said IDI2 polypeptide comprises an IF-2B domain;
(d) a subunit polypeptide and/or isoforms thereof of an eIF4F-like protein complex, wherein said eIF4F-like protein complex comprises the subunits eIF4G, eIF4A, and eIF4E, or isoforms thereof, comprising respectively the following CC domains with the PFam accession numbers:
(i) for eIF4G polypeptides: MA3 (PFam accession number: PF02847) and MIF4G (PFam accession number: PF02854);
(ii) for eIF4A polypeptides: DEAD (PFam accession number: PF00270) and Helicase C(PFam accession number: PF00271); and
(iii) for eIF4E polypeptydes: IF4E (PFam accession number: PF01652); and
(e) a Glycine-Rich RNA Binding Protein (GR-RBP polypeptide), wherein said GR-RBP polypeptide comprises a RNA Recognition Motif 1 (PFam accession PF00076, RRM 1).

146. The transgenic plant of claim 145, or a transgenic plant cell derived therefrom, wherein said plant is a crop plant, a monocot, a cereal, rice, maize, wheat, barley, millet, rye, triticale, sorghum, emmer, spelt, secale, einkorn, teff, milo, or oats.

147. Harvestable parts, shoot biomass, and/or seeds of the plant of claim 146.

148. Products derived from the plant of claim 146 and/or from harvestable parts, shoot biomass, and/or seeds of said plant.

149. An isolated nucleic acid molecule comprising:
(i) the nucleic acid sequence of SEQ ID NO: 139, 157, 164, 169, 171, or 186;
(ii) the complement of the nucleic acid sequence of SEQ ID NO: 139, 157, 164, 169, 171, or 186;
(iii) a nucleic acid encoding the amino acid sequence of SEQ ID NO: 140, 202, 209, 214, 216, or 231;
(iv) a nucleic acid encoding an IDI2 polypeptide having at least 50% sequence identity to the amino acid sequence of SEQ ID NO: 140, 202, 209, 214, 216, or 231, and comprising one or more of the motifs 1 to 6;
(v) the nucleic acid sequence of SEQ ID NO: 306;
(vi) the complement of the nucleic acid sequence of SEQ ID NO: 306;
(vii) a nucleic acid sequence that encodes the amino acid sequence of SEQ ID NO: 307 and confers an enhanced yield-related trait to a plant relative to a corresponding control plant;
(viii) a nucleic acid sequence having at least 30% sequence identity with any of the nucleic acid sequences of Table A4 and conferring an enhanced yield-related trait to a plant relative to a corresponding control plant;
(ix) a nucleic acid molecule which hybridizes with any of the nucleic acid sequences of (v) to (viii) under stringent hybridization conditions and confers an enhanced yield-related trait to a plant relative to a corresponding control plant;
(x) a nucleic acid encoding at least one eIF4F subunit polypeptide having at least 50% sequence identity to the amino acid sequence of SEQ ID NO: 307 and any of the other amino acid sequences in Tables A4 and conferring an enhanced yield-related trait to a plant relative to a corresponding control plant;
(xi) the nucleic acid sequence of SEQ ID NO: 848, 849, 851, 852, 853, 854, 857, 862, 873, 874, 875, 876, 878, 879, 893, 897, 898, 900, 901, 905, 928, 931, 932, 933, 934, or 937;
(xii) the complement of the nucleic acid sequence of SEQ ID NO: 848, 849, 851, 852, 853, 854, 857, 862, 873, 874, 875, 876, 878, 879, 893, 897, 898, 900, 901, 905, 928, 931, 932, 933, 934, or 937;
(xiii) a nucleic acid encoding the amino acid sequence of SEQ ID NO: 945, 946, 948, 949, 950, 951, 854, 959, 970, 971, 972, 973, 975, 976, 990, 994, 995, 997, 998, 1002, 1025, 1028, 1029, 1030, 1031, or 1034; or
(xiv) a nucleic acid encoding a GR-RBP polypeptide having at least 50% sequence identity to the amino acid sequence of SEQ ID NO: 945, 946, 948, 949, 950, 951, 854, 959, 970, 971, 972, 973, 975, 976, 990, 994, 995, 997, 998, 1002, 1025, 1028, 1029, 1030, 1031, or 1034, and comprising signature sequence 3 (SEQ ID NO: 830) and signature sequence 4 (SEQ ID NO: 831).

150. An isolated polypeptide comprising:
(i) the amino acid sequence of SEQ ID NO: 140, 202, 209, 214, 216, or 231;
(ii) an amino acid sequence having at least 50% sequence identity to the amino acid sequence of SEQ ID NO: 140, 202, 209, 214, 216, or 231, and comprising one or more of the motifs 1 to 6;
(iii) the amino acid sequence of SEQ ID NO: 307;
(iv) an amino acid sequence having at least 50% sequence identity to the amino acid sequence of SEQ ID NO: 307 and any of the other amino acid sequences in Tables A4 and conferring an enhanced yield-related trait to a plant relative to a corresponding control plant;
(v) the amino acid sequence of SEQ ID NO: 945, 946, 948, 949, 950, 951, 854, 959, 970, 971, 972, 973, 975, 976, 990, 994, 995, 997, 998, 1002, 1025, 1028, 1029, 1030, 1031, or 1034;
(vi) an amino acid sequence having at least 50% sequence identity to the amino acid sequence of SEQ ID NO: 945, 946, 948, 949, 950, 951, 854, 959, 970, 971, 972, 973, 975, 976, 990, 994, 995, 997, 998, 1002, 1025, 1028, 1029, 1030, 1031, or 1034, and comprising signature sequence 3 (SEQ ID NO: 830) and signature sequence 4 (SEQ ID NO: 831); or
(vii) derivatives of any of the amino acid sequences of (i) to (vi) above.
